(19) United States
(12) Patent Application Publication
(10) Pub. No.: US 2026/0132158 A1
(43) Pub. Date: May 14, 2026

(54) PHENYL OXY AMIDE KINASE INHIBITORS

(71) Applicants: THE TRANSLATIONAL GENOMICS RESEARCH INSTITUTE, Phoenix, AZ (US); UNIVERSITY OF UTAH RESEARCH FOUNDATION, Salt Lake City, UT (US)

(72) Inventors: Srinivas Rao KASIBHATLA, San Diego, CA (US); Mohan KAADIGE, Scottsdale, AZ (US); Trason THODE, Phoenix, AZ (US); Sunil SHARMA, Phoenix, AZ (US); Alexis WESTON, Phoenix, AZ (US); Alana WELM, Park City, UT (US); Jaime FORNETTI, Heber City, UT (US)

(21) Appl. No.: 19/118,679

(22) PCT Filed: Oct. 4, 2023
(86) PCT No.: PCT/US2023/075937

§ 371 Date: Apr. 4, 2025

Related U.S. Application Data
(60) Provisional application No. 63/413,543, filed on Oct. 5, 2022.

(51) Int. Cl. C07F 9/6561; A61K 31/4365; A61K 31/4439; A61K 31/444; A61K 31/506; A61K 31/519; A61K 31/5377; A61K 31/675; C07D 401/14; C07D 403/12; C07D 403/14; C07D 413/14; C07D 417/14; C07D 487/04; C07D 495/04; C07F 9/6558

(52) U.S. Cl. CPC C07F 9/6561; A61K 31/4365; A61K 31/4439; A61K 31/444; A61K 31/506; A61K 31/519; A61K 31/5377; A61K 31/675; C07D 401/14; C07D 403/12; C07D 403/14; C07D 413/14; C07D 417/14; C07D 487/04; C07D 495/04; C07F 9/65583

(57) ABSTRACT

Compounds having activity as kinase inhibitors are provided. The compounds have Structure (I), or a stereoisomer, tautomer, or salt thereof, wherein, R¹, R³, and m are as defined herein. Methods associated with preparation and use of such compounds, pharmaceutical compositions comprising such compounds and methods of use of the same for treatment of diseases and disorders are also provided.

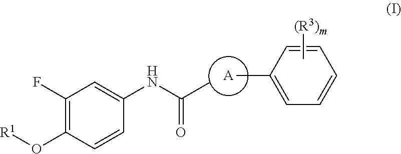

### STATEMENT OF GOVERNMENT INTEREST

This invention was made with government support under grant number W81XWH-18-1-0617 awarded by the United States Department of Defense, Congressionally Directed Medical Research Programs, Breast Cancer Research Program. The government has certain rights in the invention.

### BACKGROUND

### Technical Field

Embodiments of the present disclosure are generally directed to compounds and methods for their preparation and use as therapeutic or prophylactic agents, for example for treatment of cancer or osteoporosis.

### Description of the Related Art

Recepteur d'origine nantais (RON) receptor tyrosine kinase (RTK) and its ligand, serum macrophage-stimulating protein (MSP), are well-established oncogenic drivers for tumorigenesis and metastasis. RON is often found to be alternatively spliced resulting in various isoforms that are constitutively active. RON is therefore an attractive target for cancer therapeutics, including small molecular inhibitors and monoclonal antibodies. Although anti-RON monoclonal antibody therapies have been developed and tested in clinical trials, they were met with limited success.

Accordingly, there is a need to develop small molecule inhibitors that target the RON pathway and thereby effectively treat several pathological diseases, such as cancer and osteoporosis. Embodiments of the present disclosure fulfill this need and provide further related advantages.

### BRIEF SUMMARY

In brief, embodiments of the present disclosure provide compounds, as a stereoisomer, enantiomer, or tautomer thereof or a mixture thereof, or a pharmaceutically acceptable salt, solvate, or prodrug thereof, which can modulate the RON pathway.

One embodiment provides compounds of Structure (I):

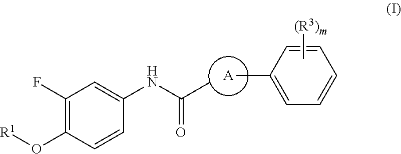

or a stereoisomer, tautomer, or salt thereof, wherein

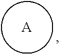

R¹, R³, and m are as defined herein. In another embodiment, pharmaceutical compositions comprising the disclosed compounds, and methods of use of the same for treatment of diseases and disorders are also provided.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to.” In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5%, or ±1% of the indicated range, value, or structure, unless otherwise indicated. The terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the —NO₂ radical.

“Oxo” refers to the ═O substituent.

“Thiol” refers to the —SH substituent.

“Thioxo” refers to the ═S substituent.

“Alkyl” refers to a saturated, straight or branched hydrocarbon chain radical consisting solely of carbon and hydrogen atoms, having, for example, from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂—Cs alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching but does not include carbon atoms belonging to any substituents. Unless stated otherwise specifically in the specification, an alkenyl group is optionally substituted.

The term “alkynyl” refers to unsaturated straight or branched hydrocarbon radical, having 2 to 12 carbon atoms (C₂-C₁₂ alkynyl), two to nine carbon atoms (C₂-C₉ alkynyl), or two to six carbon atoms (C₂-C₆ alkynyl), or any value within these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching but does not include carbon atoms belonging to any substituents. Unless stated otherwise specifically in the specification, an alkynyl group is optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Haloalkoxy” refers to a radical of the formula —ORₐ where Rₐ is a haloalkyl radical as defined herein containing one to twelve carbon atoms (C₁-C₁₂ haloalkoxy), one to eight carbon atoms (C₁-C₈ haloalkoxy) or one to six carbon atoms (C₁-C₆ haloalkoxy), or any value within these ranges. Unless stated otherwise specifically in the specification, a haloalkoxy group is optionally substituted.

“Aminyl” refers to a radical of the formula —NRₐR_{b}, where Rₐ and R_{b} are each independently H or C₁-C₆ alkyl as defined above. When both of Rₐ and R_{b} are H, an “aminyl” group is the same as an “amino” group as defined above. The C₁-C₆ alkyl portion of an aminyl group is optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, or pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Arylalkyl” refers to a radical of the formula —R_{b}R_{c} where R_{b} is an alkylene chain as defined above and R_{c} is one or more aryl radicals as defined above, for example, benzyl, diphenylmethyl, and the like. The alkylene chain part of the aralkyl radical may be optionally substituted as described above for an alkylene chain. The aryl part of the arylalkyl radical may be optionally substituted as described above for an aryl group.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Hydroxylalkyl” or “hydroxyalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxyalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen, phosphorus, and sulfur. Unless stated otherwise specifically in the specification, the heterocyclyl radical is partially or fully saturated and is a monocyclic, bicyclic, tricyclic, or tetracyclic ring system, which may include fused, spirocyclic and/or bridged ring systems. Nitrogen, carbon, phosphorus, and sulfur atoms in a heterocyclyl radical are optionally oxidized, and nitrogen atoms may be optionally quaternized. Examples of such heterocyclyl radicals include, but are not limited to, phosphinane-1-oxide, 1,4,-oxaphosphinane-4-oxide, 1,4-azaphosphinane-4-oxide, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkylene chain. The heterocyclyl part of the heterocyclylalkyl radical may be optionally substituted as defined above for a heterocyclyl group.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen, and sulfur, and at least one aromatic ring. Heteroaryl radicals may be a monocyclic, bicyclic, tricyclic, or tetracyclic ring system, which may include fused or bridged ring systems; and the nitrogen, carbon, or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

“N-heteroaryl” refers to a heteroaryl radical as defined above containing at least one nitrogen. An N-heteroaryl radical may be optionally substituted as described above for heteroaryl radicals.

The terms “pyrazolyl, triazolyl, isoxazolyl, isothiazolyl, pyridinyl, pyrimidinyl, and/or pyridinonyl” refer to rings having the following respective structures:

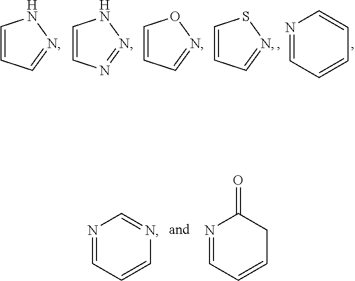

Each of the rings may connect to the remainder of the molecule or parts of the molecule through a single bond, wherein the connection is made by replacing a hydrogen with a single bond. For example, in some embodiments of Structure (I), when

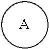

is a pyrazolyl and R² is methyl, then the pyrazolyl has one of the following structures:

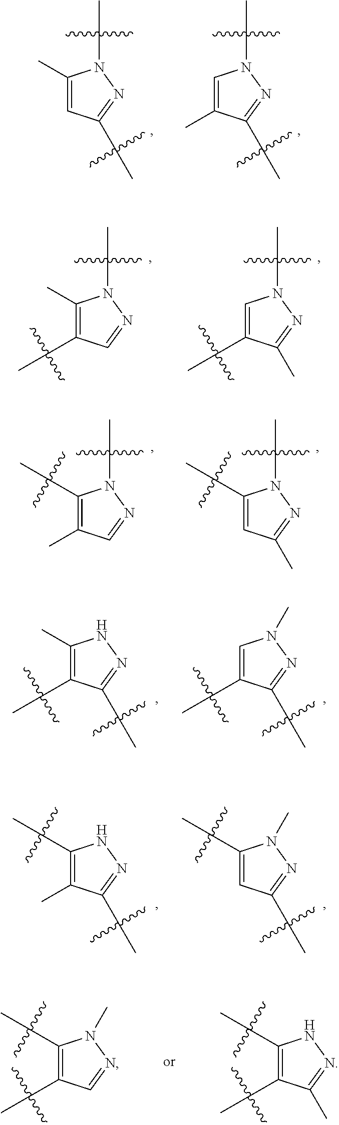

In some embodiments, a pyrazolyl, triazolyl, isoxazolyl, isothiazolyl, pyridinyl, pyrimidinyl, and/or pyridinonyl is optionally substituted with one or more (e.g., 1, 2, or 3) additional substituents. In certain embodiments, a pyrazolyl, triazolyl, isoxazolyl, isothiazolyl, pyridinyl, pyrimidinyl, and/or pyridinonyl does not include any additional substituents.

The term “—NH-aryl-alkyl-heterocyclyl-alkenyl” refers to a radical of the formula —NH—Rₐ—R_{b}—R_{c}—R_{d} where Rₐ is an aryl group as defined above, R_{b} is an alkylene chain as defined above, R_{c} is a heterocyclyl group as defined above, and Rd is an alkenyl radical as defined above. Each portion of the radical (i.e., aryl, alkyl or alkylene, heterocyclyl, and alkenyl) may be optionally substituted as defined above for each respective group. An example of a —NH-aryl-alkyl-heterocyclyl-alkenyl is a radical having one of the following structures:

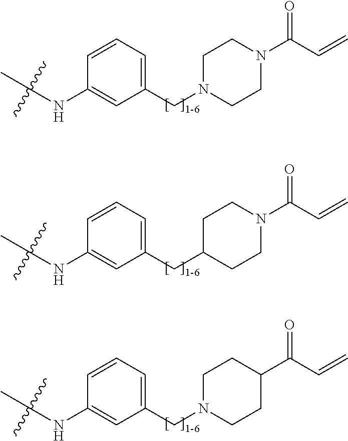

wherein each of the structures above may be optionally substituted with one or more additional substituents (e.g., with oxo on the alkyl or heterocyclyl portion of the radical).

The term “substituted” as used herein means any of the above groups (e.g., alkyl, alkenyl, alkylene, alkylcarbonyl, alkoxy, alkoxyalkyl, aminylalkyl, aryl, cyanoalkyl, cycloalkyl, haloalkyl, heterocyclyl, heterocyclene, heterocyclylalkyl, heteroaryl, heteroarylalkyl and/or hydroxylalkyl) wherein at least one hydrogen atom (e.g., 1, 2, 3 or all hydrogen atoms) is replaced by a bond to a non-hydrogen substituent. Examples of non-hydrogen substituents include, but are not limited to amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl, and/or hydroxylalkyl substituents, each of which may also be optionally substituted with one or more of the above substituents.

In some specific embodiments, optional substituents are independently selected from the group consisting of halo, hydroxyl, cyano, aminyl, C₁-C₆ alkyl, C₂-C₆ alkenyl, C₂-C₆ alkynyl, C₁-C₆ haloalkyl, C₃-C₈ cycloalkyl, C₃-C₈ halocycloalkyl, C₆-C₁₀ aryl, 5- or 6-membered heteroaryl, C₁-C₆ alkoxy, and 3-8 membered heterocyclyl. In some embodiments, optional substituents are independently selected from the group consisting of halo, cyano, optionally substituted C₁-C₆ alkyl, optionally substituted C₁-C₆ haloalkyl, optionally substituted C₁-C₆ alkoxy, optionally substituted C₁-C₆ haloalkoxy, and optionally substituted C₃-C₈ cycloalkyl. In some embodiments, optional substituents are independently selected from halo, cyano, optionally substituted C₁-C₆ alkyl, optionally substituted C₁-C₆ haloalkyl, optionally substituted C₁-C₆ alkoxy, optionally substituted C₁-C₆ haloalkoxy, or optionally substituted C₃-C₈ cycloalkyl, optionally substituted 5-10-membered heterocyclylalkyl, optionally substituted 5-10-membered heterocyclyloxy, and optionally substituted C₆-C₁₀ aryl.

The term “effective amount” or “therapeutically effective amount” refers to that amount of a compound described herein that is sufficient to affect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying, or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I)). Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino, or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body. Embodiments disclosed herein are also meant to encompass all pharmaceutically acceptable compounds of Structure (I), including stereoisomers, enantiomers, or tautomers thereof or a mixture thereof; or a pharmaceutically acceptable salt, solvate, or prodrug thereof.

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some embodiments, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution (“unsubstituted”). When a functional group is described as “optionally substituted,” and in turn, substituents on the functional group are also “optionally substituted” and so on, for the purposes of this disclosure, such iterations are limited to five, four, or three. In some embodiments, such iterations are limited to two. In some embodiments, such iterations are limited to one. In some embodiments, when a functional group is described as “optionally substituted” substituents on the functional group are unsubstituted.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents, or excipients therefor.

“Pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) are a mixture of enantiomers or diastereomers. In other embodiments, the compounds of Structure (I) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent; cyanoalkyl comprises an alkyl backbone with a cyano substituent. Except as described below, all bonds are identified in the chemical structure diagrams herein, except for all bonds on some carbon atoms, which are assumed to be bonded to sufficient hydrogen atoms to complete the valency.

### Compounds

The disclosure provides a compound having the following Structure (I):

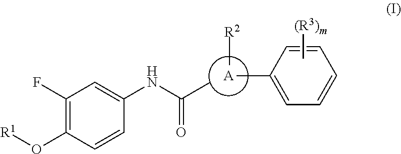

wherein:
   R¹ has one of the following structures:

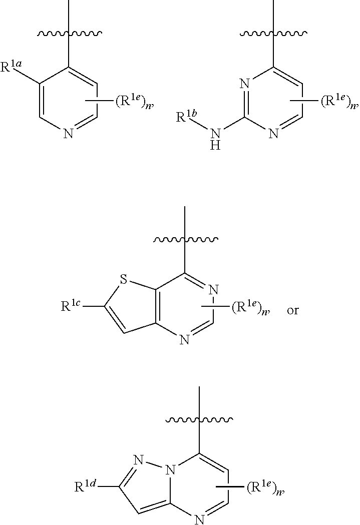

wherein:
   R^{1a} is optionally substituted cycloalkyl, optionally substituted heterocyclyl, optionally substituted aryl, optionally substituted 5-membered heteroaryl, or —P(═O)R^{a}R^{b} wherein Rₐ and R^{b} are each independently alkyl;
   R^{1b} is optionally substituted cycloalkyl, optionally substituted heterocyclyl, optionally substituted aryl, or optionally substituted heteroaryl;
   R^{1c} is optionally substituted cycloalkyl, optionally substituted heterocyclyl, optionally substituted aryl, optionally substituted heteroaryl, or —P(═O)R^{c}R^{d} wherein R^{c} and R^{d} are each independently alkyl;
   R^{1d} is halo, optionally substituted cycloalkyl, optionally substituted heterocyclyl, optionally substituted aryl, optionally substituted heteroaryl, or —P(═O)R^{e}R^{f} wherein R^{e} and R^{f} are each independently alkyl;
   each occurrence of R^{1e} is independently alkyl, halo, haloalkyl, cycloalkyl, hydroxyl, amino, or cyano;
   R² is alkyl, haloalkyl, or —P(═O)R^{g}R^{h} wherein R^{g} and R^{h} are each independently alkyl;
   each occurrence of R³ is independently alkyl, halo, haloalkyl, alkoxy, haloalkoxy, cycloalkyl, hydroxyl, amino, or cyano;

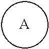

is pyrazolyl, triazolyl, isoxazolyl, isothiazolyl, pyridinyl, pyrimidinyl, or pyridinonyl;
n is 0, 1, 2, or 3; and
m is 0, 1, 2, 3, 4, or 5,
as a stereoisomer, enantiomer, or tautomer thereof or a mixture thereof; or a pharmaceutically acceptable salt, solvate, or prodrug thereof.

One embodiment provides a compound having the following Structure (I):

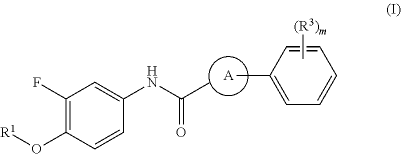

wherein:
   R¹ has one of the following structures:

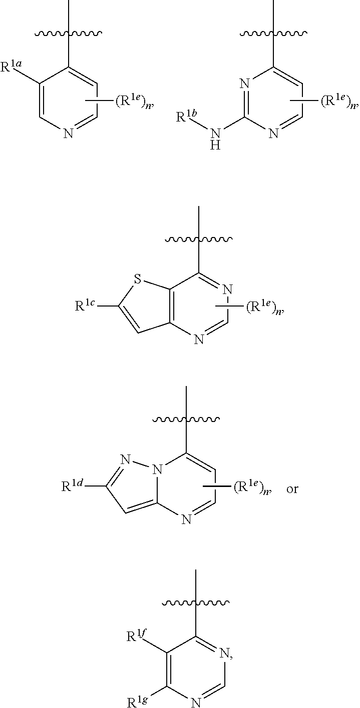

wherein:
   R^{1a} is optionally substituted cycloalkyl, optionally substituted heterocyclyl, optionally substituted aryl, optionally substituted 5-membered heteroaryl, or —P(═O)R^{a}R^{b} wherein R^{a} and R^{b} are each independently alkyl or R^{a} and R^{b} join, together with the P atom to which they are attached to form optionally substituted heterocyclyl;
   R^{1b} is optionally substituted cycloalkyl, optionally substituted heterocyclyl, optionally substituted aryl, optionally substituted heteroaryl, unsubstituted arylalkyl, or optionally substituted heterocyclylalkyl;
   R^{1c} is optionally substituted cycloalkyl, optionally substituted heterocyclyl, optionally substituted aryl, optionally substituted heteroaryl, or —P(═O)R^{c}R^{d} wherein R^{c} and R^{d} are each independently alkyl;
   R^{1d} is halo, optionally substituted cycloalkyl, optionally substituted heterocyclyl, optionally substituted aryl, optionally substituted heteroaryl, or —P(═O)R^{e}R^{f} wherein R^{c} and R^{f} are each independently alkyl;
   each occurrence of R^{1e} is independently alkyl, halo, haloalkyl, cycloalkyl, hydroxyl, amino, or cyano;
   R^{1f} is —P(═O)R^{e}R^{f} wherein R^{e} and R^{f} are each independently alkyl;
   R^{1g} is hydrogen or amino;
   each occurrence of R³ is independently alkyl, halo, haloalkyl, alkoxy, haloalkoxy, cycloalkyl hydroxyl, amino, or cyano;

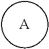

has one of the following structures:

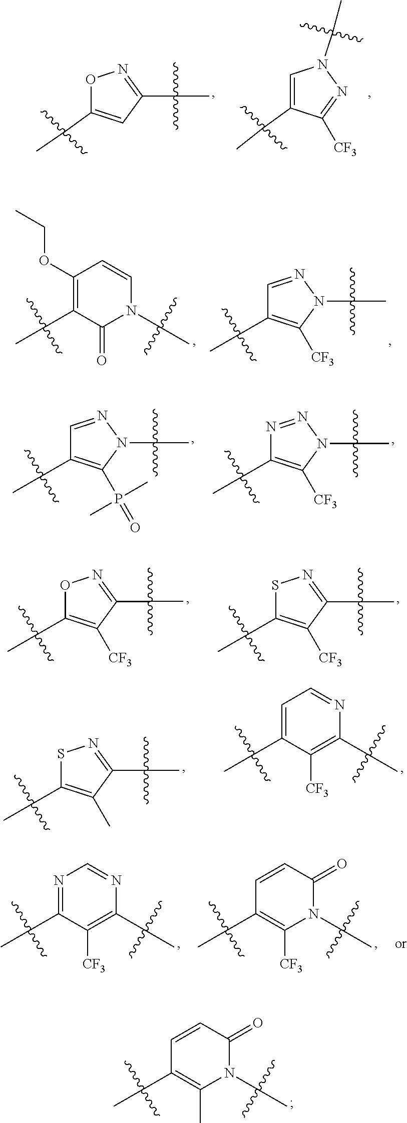

n is 0, 1, 2, or 3; and
m is 0, 1, 2, 3, 4, or 5,
as a stereoisomer, enantiomer, or tautomer thereof or a mixture thereof, or a pharmaceutically acceptable salt, solvate, or prodrug thereof.

In some embodiments, R¹ has the following structure:

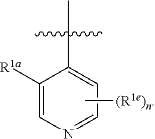

In some embodiments, R^{1a} is optionally substituted 5-membered heteroaryl. In certain embodiments, R^{1a} is optionally substituted 5-membered N-heteroaryl. In some embodiments, R^{1a} is optionally substituted imidazolyl or optionally substituted pyrazolyl. In some embodiments, R^{1a} has one of the following structures:

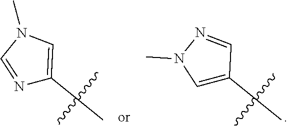

In some embodiments, R^{1a} is —P(═O)R^{a}R^{b}. In some embodiments, R^{a} is methyl, ethyl, n-propyl, or isopropyl. In some embodiments, R^{b} is methyl, ethyl, n-propyl, or isopropyl. In certain embodiments, Ria has the following structure:

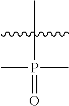

In some embodiments, Ria has one of the following structures:

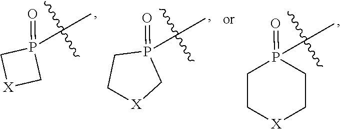

wherein:
   X is —CH₂—, —N(R^{a′})—, or —O—; and
   R^{a} is hydrogen or C₁-C₆ alkyl.

In certain embodiments, R^{1a} is optionally substituted cycloalkyl, optionally substituted heterocyclyl, or optionally substituted aryl. In some embodiments, R^{1a} is optionally substituted with alkyl, halo, haloalkyl, cycloalkyl, hydroxyl, amino, or cyano. In some embodiments, R^{1a} is unsubstituted.

In some embodiments, R¹ has the following structure:

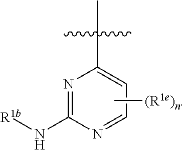

In certain embodiments, R^{1b} is optionally substituted cycloalkyl, optionally substituted heterocyclyl, optionally substituted aryl, unsubstituted arylalkyl, or optionally substituted heterocyclylalkyl. In some embodiments, R^{1b} is optionally substituted cyclohexyl, optionally substituted piperidinyl, optionally substituted phenyl, unsubstituted benzyl, or optionally substituted —(CH₂)₂-morpholino. In certain embodiments, R^{1b} is cyclohexyl, piperidinyl, or phenyl substituted with one or more substituents selected from the group consisting of alkyl, alkoxy, haloalkoxy, halo, —P(═O)—(CH₃)₂, —NH—C(═O)-alkenyl, —NH—C(═O)-alkenyl-N(CH₃)₂, —NH—C(═O)-alkyl-N(CH₃)—C(═O) alkenyl-N(CH₃)₂, optionally substituted heterocyclyl, and optionally substituted heterocyclylalkyl.

In some embodiments, R^{1b} is optionally substituted aryl. In certain embodiments, R^{1b} is optionally substituted phenyl. In some embodiments, R^{1b} is a substituted phenyl. In some embodiments, R^{1b} is phenyl substituted with a heterocyclyl.

In certain embodiments, R^{1b} has one of the following structures:

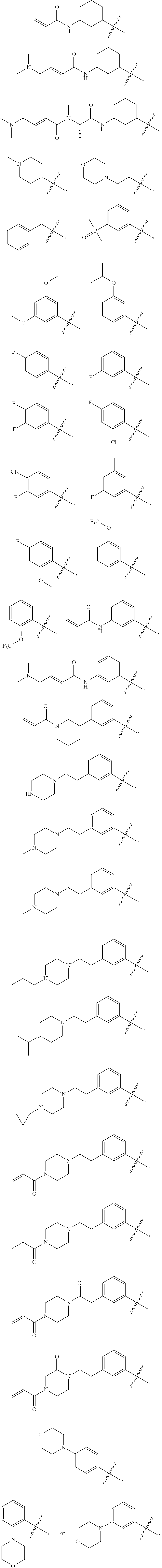

In some embodiments, R^{1b} has one of the followings structures:

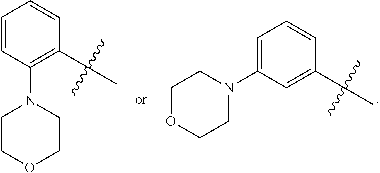 [SMILES: *c1ccccc1N1CCOCC1 ; *c1cccc(N2CCOCC2)c1]

In certain embodiments, R^{1b} is optionally substituted cycloalkyl, optionally substituted heterocyclyl, or optionally substituted heteroaryl. In some embodiments, R^{1b} is optionally substituted with alkyl, halo, haloalkyl, cycloalkyl, hydroxyl, amino, or cyano.

In some embodiments, R¹ has the following structure:

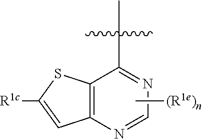

In certain embodiments, R^{1c} is optionally substituted 5-membered heteroaryl. In some embodiments, R^{1c} is optionally substituted 5-membered N-heteroaryl. In certain embodiments, R^{1c} is optionally substituted imidazolyl or optionally substituted pyrazolyl. In some embodiments, R^{1c} has one of the following structures:

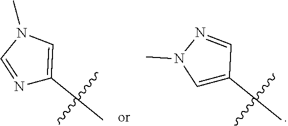

In some embodiments, R^{1c} is —P(═O)R^{c}R^{d}. In some embodiments, R is methyl, ethyl, n-propyl, or isopropyl. In some embodiments, R^{d} is methyl, ethyl, n-propyl, or isopropyl. In some embodiments, R^{1c} has the following structure:

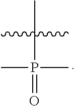

In some embodiments, R^{1c} has one of the following structures:

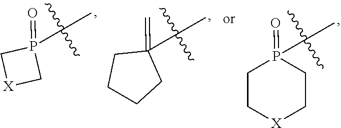

wherein:
   X is —CH₂—, —N(R^{c′})—, or —O—; and
   R^{c′} is hydrogen or C₁-C₆ alkyl.

In some embodiments, R¹ has the following structure:

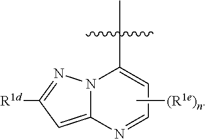

In certain embodiments, R^{1d} is optionally substituted 5-membered heteroaryl. In some embodiments, R^{1d} is optionally substituted 5-membered N-heteroaryl. In certain embodiments, R^{1d} is optionally substituted imidazolyl or optionally substituted pyrazolyl. In some embodiments, R^{1d} has the following structure:

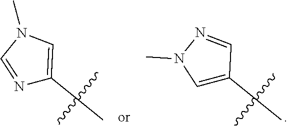

In some embodiments, R^{1c} is —P(═O)R^{e}R^{f}. In some embodiments, R^{e} is methyl, ethyl, n-propyl, or isopropyl. In some embodiments, R is methyl, ethyl, n-propyl, or isopropyl. In certain embodiments, R^{1d} has the following structure:

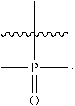

In some embodiments, R^{1d} has one of the following structures:

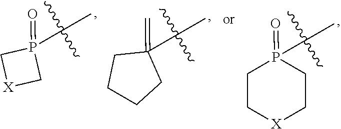

wherein:
   X is —CH₂—, —N(R^{d′})—, or —O—; and
   R^{d′} is hydrogen or C₁-C₆ alkyl.

In certain embodiments, R^{1d} is fluoro, chloro, bromo, or iodo. In some embodiments, R^{1d} is chloro or fluoro. In some embodiments, R^{1d} is bromo.

In some embodiments, n is 0. In certain embodiments, n is 1, 2, 3 and each occurrence of R^{1e} is independently halo, hydroxyl, or amino. In some embodiments, n is 1 and R^{1e} is amino. In some embodiments, n is 0. In certain embodiments, n is 1, 2, 3 and each occurrence of R^{1e} is independently halo, hydroxyl, amino or —NH-aryl-alkyl-heterocyclyl-alkenyl. In some embodiments, n is 1 and R^{1e} is amino or has the following structure:

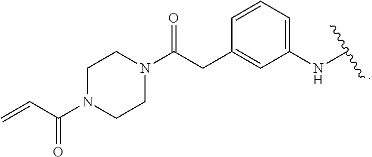 [SMILES: *Nc1cccc(CC(=O)N2CCN(C(=O)C=C)CC2)c1]

In certain embodiments, R¹ has the following structure:

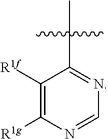

In some embodiments, R^{1f} has the following structure:

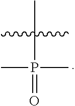

In some embodiments, Rif has one of the following structures:

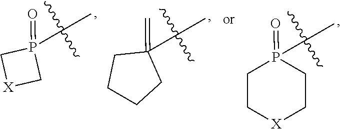

wherein:
   X is —CH₂—, —N(R^{f′})—, or —O—; and
   R^{f} is hydrogen or C₁-C₆ alkyl.

In some embodiments, R^{1g} is amino. In some embodiments, R^{1g} is hydrogen.

In certain embodiments, R¹ has one of the following structures:

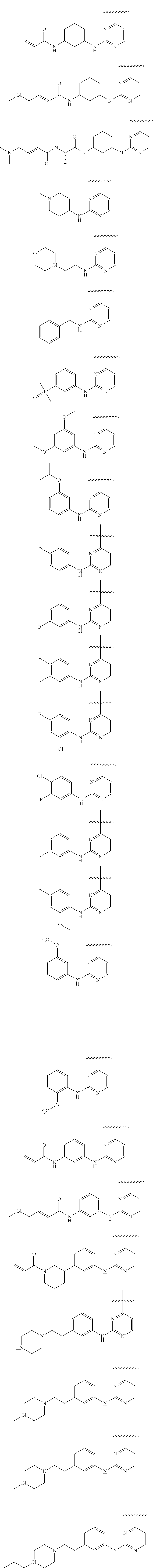

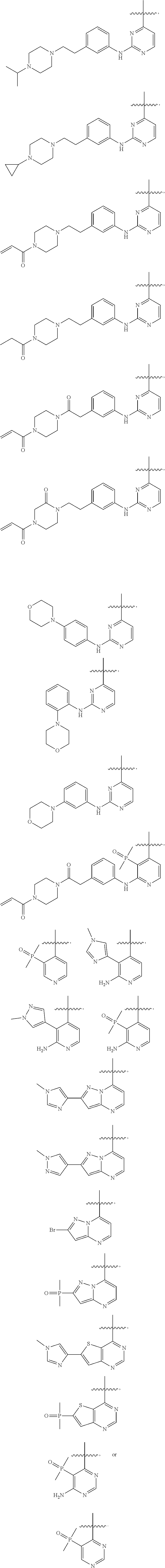

In some embodiments, R¹ has one of the following structures:

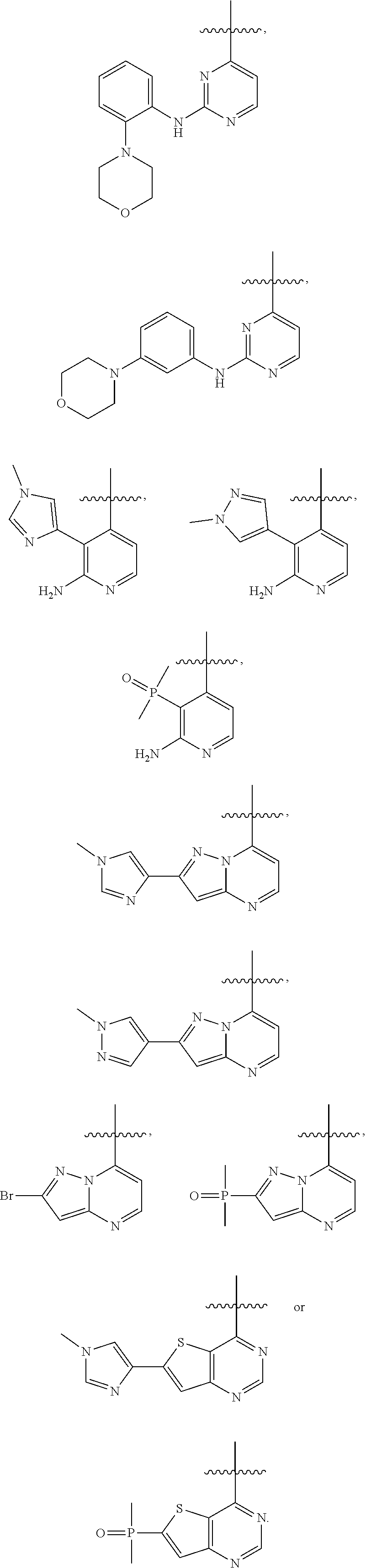

In certain embodiments, R¹ has one the following structures:

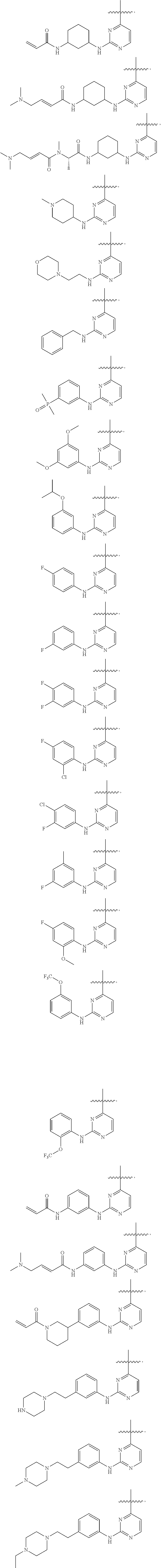

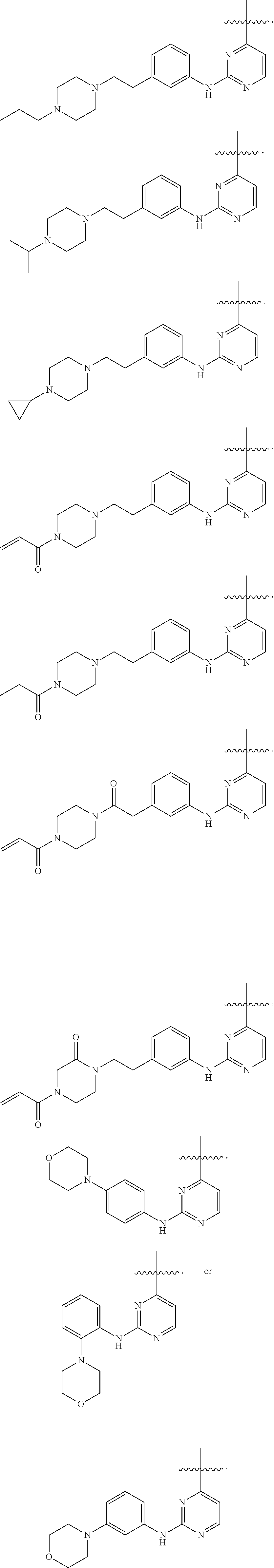

In some embodiments, R¹ has one the following structures:

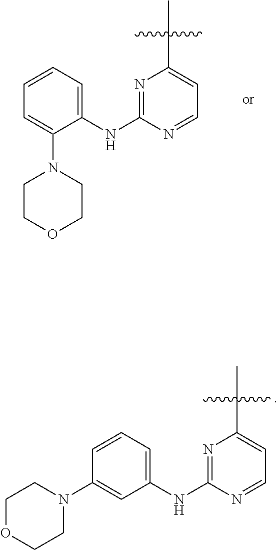 [SMILES: *c1ccnc(Nc2ccccc2N2CCOCC2)n1 ; *c1ccnc(Nc2cccc(N3CCOCC3)c2)n1]

In certain embodiments, R¹ has one of the following structures:

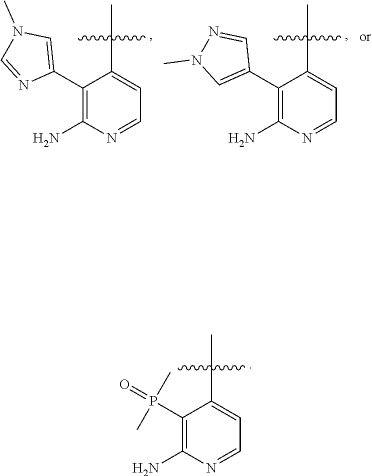

In certain embodiments, R¹ has one of the following structures:

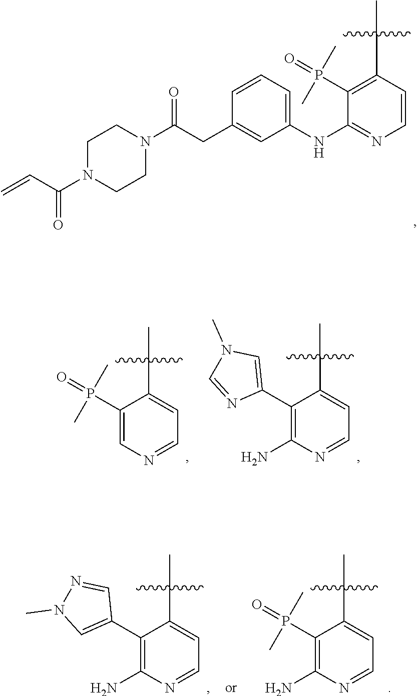

In some embodiments, R¹ has one of the following structures:

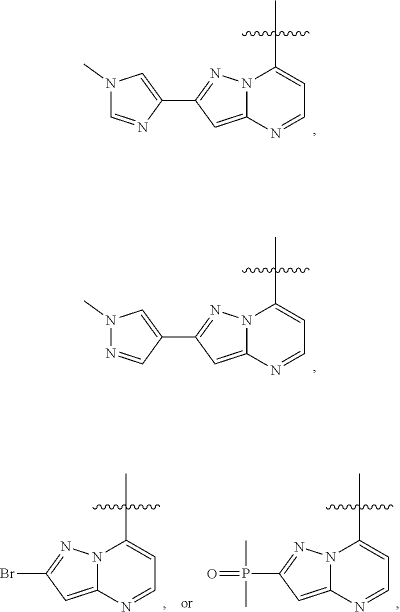 [SMILES: *c1ccnc2cc(-c3cn(C)cn3)nn12 ; *c1ccnc2cc(-c3cnn(C)c3)nn12 ; *c1ccnc2cc(Br)nn12 ; *c1ccnc2cc(P(C)(C)=O)nn12]

In certain embodiments, R¹ has one of the following structures:

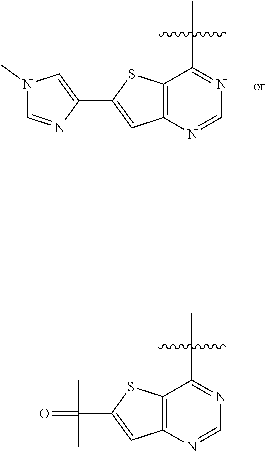

In some embodiments, R¹ has one of the following structures:

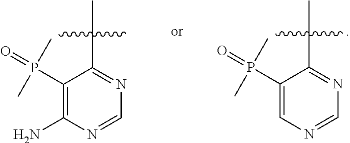 [SMILES: *c1ncnc(N)c1P(C)(C)=O ; *c1ncncc1P(C)(C)=O]

In some embodiments, R² is haloalkyl. In certain embodiments, R² is trifluoromethyl. In some embodiments, R² is alkyl. In certain embodiments, R² is methyl. In some embodiments, R² has the following structure:

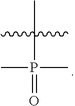

In some embodiments, R² has one of the following structures:

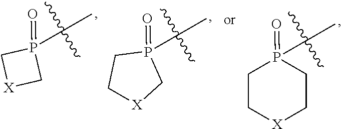

wherein:
   X is —CH₂—, —N(R^{2′})—, or —O—; and
   R^{2a′} is hydrogen or C₁-C₆ alkyl.

In certain embodiments,

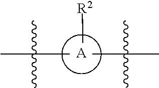

has one of the following structures:

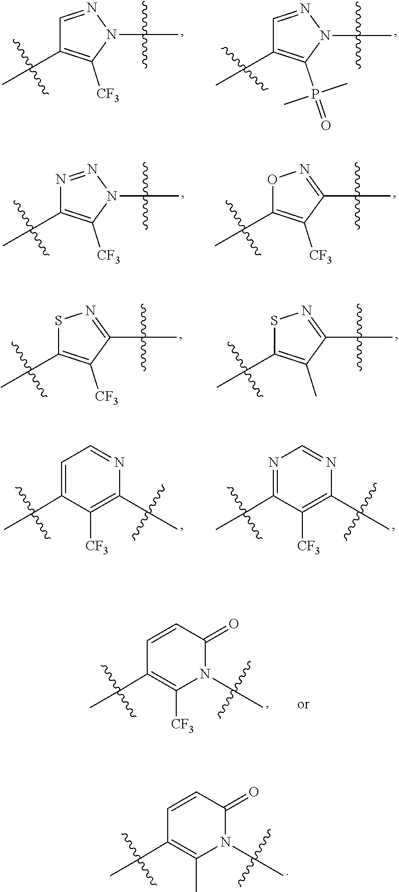

In some embodiments,

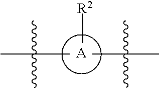

has one of the following structures:

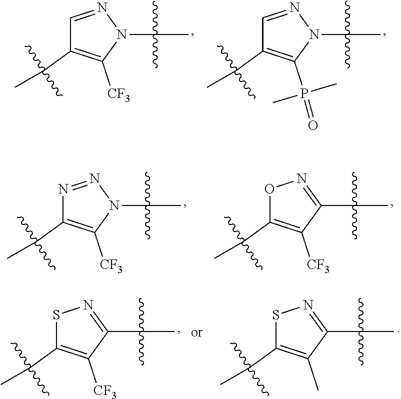

In certain embodiments,

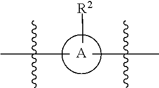

has one of the following structures:

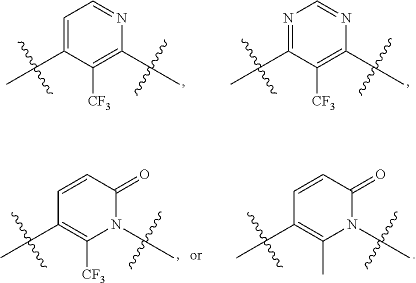

In some embodiments, m is 0. In certain embodiments, m is 1, 2, 3, 4, or 5. In some embodiments, each occurrence of R³ is independently halo or haloalkyl. In some embodiments, each occurrence of R³ is halo. In certain embodiments, m is 1 and R³ is fluoro.

In some embodiments, the

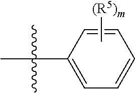

portion of Structure (I) has the following structure:

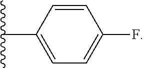 [SMILES: *c1ccc(F)cc1]

One embodiment provides a compound having one of the structures set forth in Table 1 as a stereoisomer, enantiomer, or tautomer thereof or a mixture thereof or a pharmaceutically acceptable salt, solvate, or prodrug thereof. Compounds in Table 1 were prepared as described in the Examples or methods known in the art and analyzed by mass spectrometry (MS) and/or nuclear magnetic resonance spectroscopy (NMR).

TABLE 1
Representative compounds of Structure (I)

| No. | Structure | Name |
|---|---|---|
| 1 | 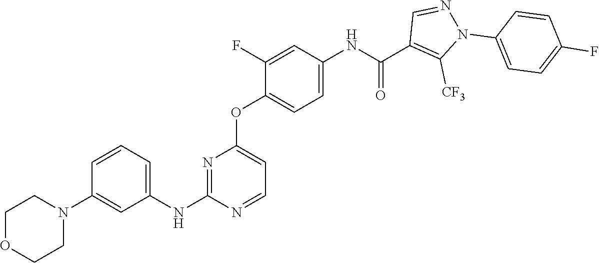 [SMILES: O=C(Nc1ccc(Oc2ccnc(Nc3cccc(N4CCOCC4)c3)n2)c(F)c1)c1cnn(-c2ccc(F)cc2)c1C(F)(F)F] | N-(3-fluoro-4-((2-((3- morpholinophenyl)amino)pyrimidin- 4-yl)oxy)phenyl)-1-(4- fluorophenyl)-5- (trifluoromethyl)-1H-pyrazole-4- carboxamide |
| 2 | 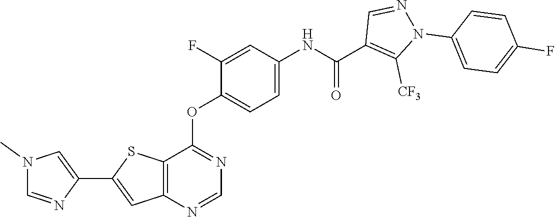 [SMILES: Cn1cnc(-c2cc3ncnc(Oc4ccc(NC(=O)c5cnn(-c6ccc(F)cc6)c5C(F)(F)F)cc4F)c3s2)c1] | N-(3-fluoro-4-((6-(1-methyl-1H- imidazol-4-yl)thieno[3,2- d]pyrimidin-4-yl)oxy)phenyl)-1- (4-fluorophenyl)-5- (trifluoromethyl)-1H-pyrazole-4- carboxamide |
| 3 | 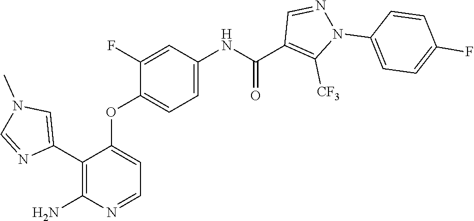 [SMILES: Cn1cnc(-c2c(Oc3ccc(NC(=O)c4cnn(-c5ccc(F)cc5)c4C(F)(F)F)cc3F)ccnc2N)c1] | N-(4-((2-amino-3-(1-methyl-1H- imidazol-4-yl)pyridin-4-yl)oxy)- 3-fluorophenyl)-1-(4- fluorophenyl)-5- (trifluoromethyl)-1H-pyrazole-4- carboxamide |
| 4 | 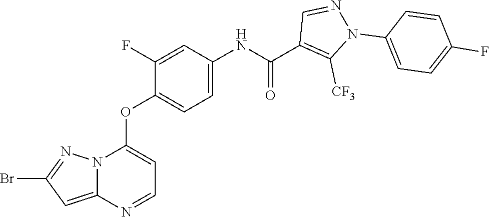 [SMILES: O=C(Nc1ccc(Oc2ccnc3cc(Br)nn23)c(F)c1)c1cnn(-c2ccc(F)cc2)c1C(F)(F)F] | N-(4-((2-bromopyrazolo[1,5- a]pyrimidin-7-yl)oxy)-3- fluorophenyl)-1-(4- fluorophenyl)-5- (trifluoromethyl)-1H-pyrazole-4- carboxamide |
| 5 | 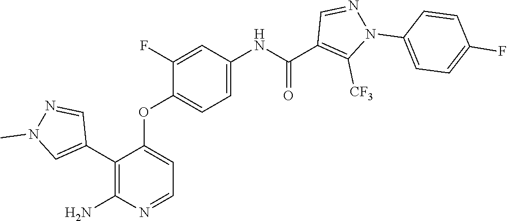 [SMILES: Cn1cc(-c2c(Oc3ccc(NC(=O)c4cnn(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-1-(4- fluorophenyl)-5- (trifluoromethyl)-1H-pyrazole-4- carboxamide |
| 6 | 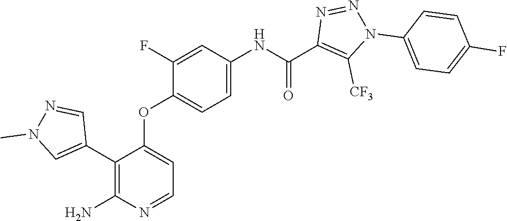 [SMILES: Cn1cc(-c2c(Oc3ccc(NC(=O)c4nnn(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-1-(4- fluorophenyl)-5- (trifluoromethyl)-1H-1,2,3- triazole-4-carboxamide |
| 7 | 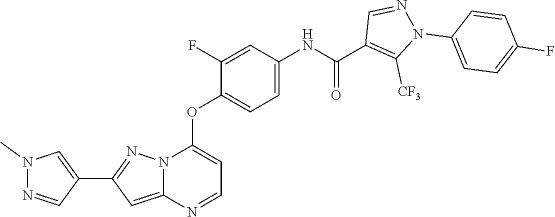 [SMILES: Cn1cc(-c2cc3nccc(Oc4ccc(NC(=O)c5cnn(-c6ccc(F)cc6)c5C(F)(F)F)cc4F)n3n2)cn1] | N-(3-fluoro-4-((2-(1-methyl-1H- pyrazol-4-yl)pyrazolo[1,5- a]pyrimidin-7-yl)oxy)phenyl)-1- (4-fluorophenyl)-5- (trifluoromethyl)-1H-pyrazole-4- carboxamide |
| 8 | 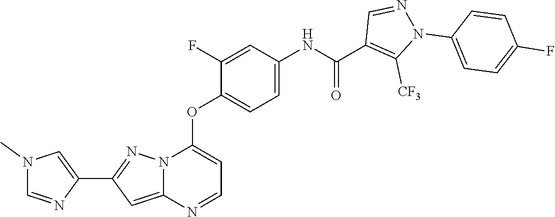 [SMILES: Cn1cnc(-c2cc3nccc(Oc4ccc(NC(=O)c5cnn(-c6ccc(F)cc6)c5C(F)(F)F)cc4F)n3n2)c1] | N-(3-fluoro-4-((2-(1-methyl-1H- imidazol-4-yl)pyrazolo[1,5- a]pyrimidin-7-yl)oxy)phenyl)-1- (4-fluorophenyl)-5- (trifluoromethyl)-1H-pyrazole-4- carboxamide |
| 9 | 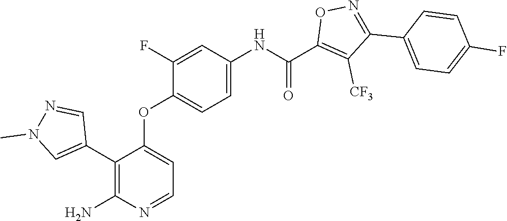 [SMILES: Cn1cc(-c2c(Oc3ccc(NC(=O)c4onc(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-3-(4- fluorophenyl)-4- (trifluoromethyl)isoxazole-5- carboxamide |
| 10 | 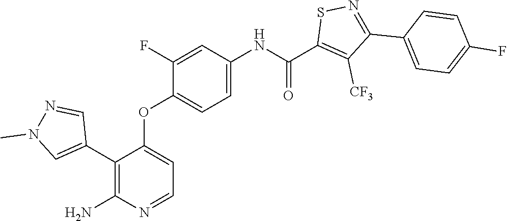 [SMILES: Cn1cc(-c2c(Oc3ccc(NC(=O)c4snc(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-3-(4- fluorophenyl)-4- (trifluoromethyl)isothiazole-5- carboxamide |
| 11 | 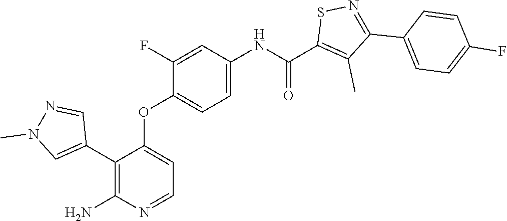 [SMILES: Cc1c(-c2ccc(F)cc2)nsc1C(=O)Nc1ccc(Oc2ccnc(N)c2-c2cnn(C)c2)c(F)c1] | N-(4-((2-amino-3-(1-methyl-1H- pyrazol-4-yl)pyridin-4-yl)oxy)- 3-fluorophenyl)-3-(4- fluorophenyl)-4- methylisothiazole-5- carboxamide |
| 12 | 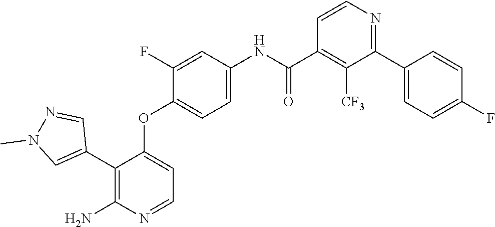 [SMILES: Cn1cc(-c2c(Oc3ccc(NC(=O)c4ccnc(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-2-(4- fluorophenyl)-3- (trifluoromethyl)isonicotinamide |
| 13 | 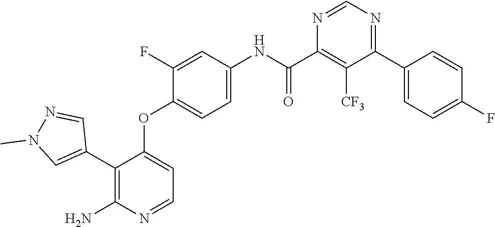 [SMILES: Cn1cc(-c2c(Oc3ccc(NC(=O)c4ncnc(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-6-(4- fluorophenyl)-5- (trifluoromethyl)pyrimidine-4- carboxamide |
| 14 | 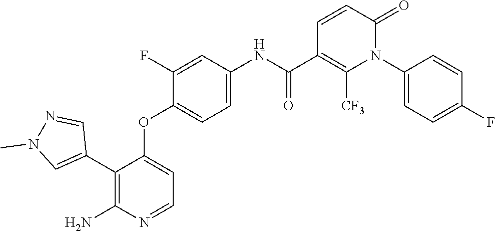 [SMILES: Cn1cc(-c2c(Oc3ccc(NC(=O)c4ccc(=O)n(-c5ccc(F)cc5)c4C(F)(F)F)cc3F)ccnc2N)cn1] | N-(4-((2-amino-3-(1-methyl-1H- pyrazol-4-yl)pyridin-4-yl)oxy)- 3-fluorophenyl)-1-(4- fluorophenyl)-6-oxo-2- (trifluoromethyl)-1,6- dihydropyridine-3-carboxamide |
| 15 | 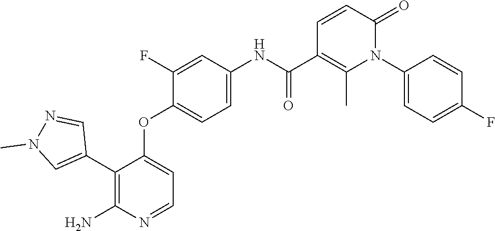 [SMILES: Cc1c(C(=O)Nc2ccc(Oc3ccnc(N)c3-c3cnn(C)c3)c(F)c2)ccc(=O)n1-c1ccc(F)cc1] | N-(4-((2-amino-3-(1-methyl-1H- pyrazol-4-yl)pyridin-4-yl)oxy)- 3-fluorophenyl)-1-(4- fluorophenyl)-2-methyl-6-oxo- 1,6-dihydropyridine-3- carboxamide |
| 16 | 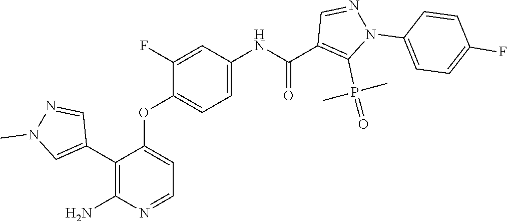 [SMILES: Cn1cc(-c2c(Oc3ccc(NC(=O)c4cnn(-c5ccc(F)cc5)c4P(C)(C)=O)cc3F)ccnc2N)cn1] | N-(4-((2-amino-3-(1-methyl-1H- pyrazol-4-yl)pyridin-4-yl)oxy)- 3-fluorophenyl)-5- (dimethylphosphoryl)-1-(4- fluorophenyl)-1H-pyrazole-4- carboxamide |
| 17 | 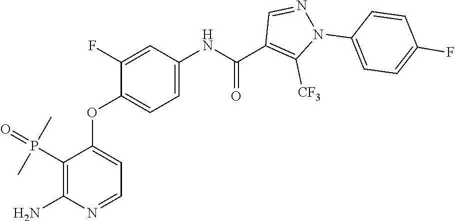 [SMILES: CP(C)(=O)c1c(Oc2ccc(NC(=O)c3cnn(-c4ccc(F)cc4)c3C(F)(F)F)cc2F)ccnc1N] | N-(4-((2-amino-3- (dimethylphosphoryl)pyridin-4- yl)oxy)-3-fluorophenyl)-1-(4- fluorophenyl)-5- (trifluoromethyl)-1H-pyrazole-4- carboxamide |
| 18 | 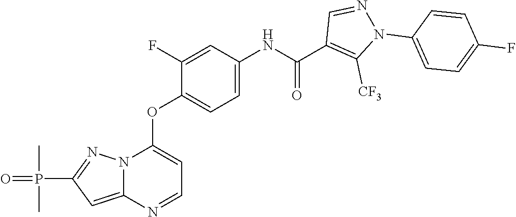 [SMILES: CP(C)(=O)c1cc2nccc(Oc3ccc(NC(=O)c4cnn(-c5ccc(F)cc5)c4C(F)(F)F)cc3F)n2n1] | N-(4-((2- (dimethylphosphoryl)pyrazolo [1,5-a]pyrimidin-7-yl)oxy)-3- fluorophenyl)-1-(4- fluorophenyl)-5- (trifluoromethyl)-1H-pyrazole-4- carboxamide |
| 19 | 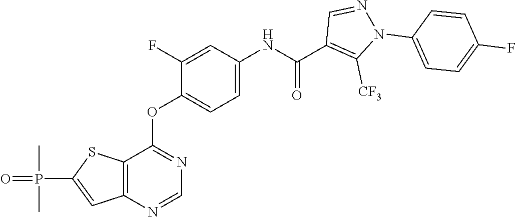 [SMILES: CP(C)(=O)c1cc2ncnc(Oc3ccc(NC(=O)c4cnn(-c5ccc(F)cc5)c4C(F)(F)F)cc3F)c2s1] | N-(4-((6- (dimethylphosphoryl)thieno[3,2- d]pyrimidin-4-yl)oxy)-3- fluorophenyl)-1-(4- fluorophenyl)-5- (trifluoromethyl)-1H-pyrazole-4- carboxamide |
| 20 | 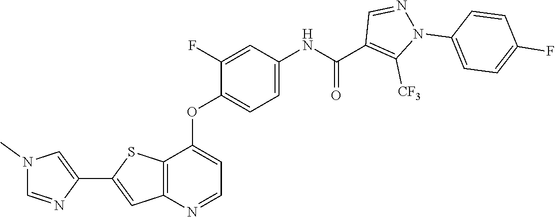 [SMILES: Cn1cnc(-c2cc3nccc(Oc4ccc(NC(=O)c5cnn(-c6ccc(F)cc6)c5C(F)(F)F)cc4F)c3s2)c1] | N-(3-fluoro-4-((2-(1-methyl-1H- imidazol-4-yl)thieno[3,2- b]pyridin-7-yl)oxy)phenyl)-1-(4- fluorophenyl)-5- (trifluoromethyl)-1H-pyrazole-4- carboxamide |
| 21 | 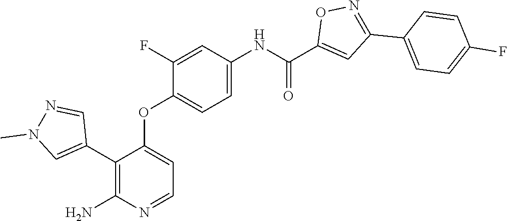 [SMILES: Cn1cc(-c2c(Oc3ccc(NC(=O)c4cc(-c5ccc(F)cc5)no4)cc3F)ccnc2N)cn1] | N-(4-((2-amino-3-(1-methyl-1H- pyrazol-4-yl)pyridin-4-yl)oxy)- 3-fluorophenyl)-3-(4- fluorophenyl)isoxazole-5- carboxamide |
| 22 | 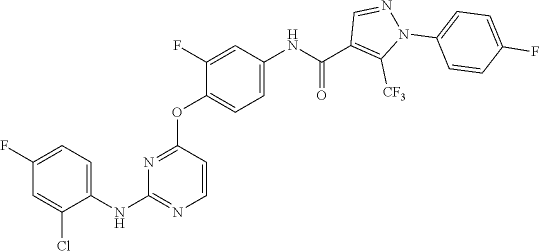 [SMILES: O=C(Nc1ccc(Oc2ccnc(Nc3ccc(F)cc3Cl)n2)c(F)c1)c1cnn(-c2ccc(F)cc2)c1C(F)(F)F] | N-(4-((2-((2-chloro-4- fluorophenyl)amino)pyrimidin- 4-yl)oxy)-3-fluorophenyl)-1-(4- fluorophenyl)-5- (trifluoromethyl)-1H-pyrazole-4- carboxamide |
| 23 | 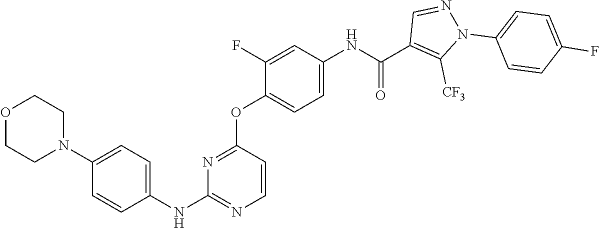 [SMILES: O=C(Nc1ccc(Oc2ccnc(Nc3ccc(N4CCOCC4)cc3)n2)c(F)c1)c1cnn(-c2ccc(F)cc2)c1C(F)(F)F] | N-(3-fluoro-4-((2-((4- morpholinophenyl)amino)pyrimidin- 4-yl)oxy)phenyl)-1-(4- fluorophenyl)-5- (trifluoromethyl)-1H-pyrazole-4- carboxamide |
| 24 | 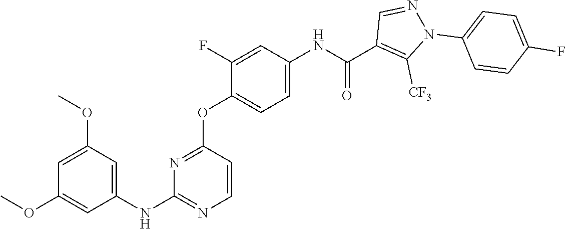 [SMILES: COc1cc(Nc2nccc(Oc3ccc(NC(=O)c4cnn(-c5ccc(F)cc5)c4C(F)(F)F)cc3F)n2)cc(OC)c1] | N-(4-((2-((3,5- dimethoxyphenyl)amino)pyrimidin- 4-yl)oxy)-3-fluorophenyl)-1- (4-fluorophenyl)-5- (trifluoromethyl)-1H-pyrazole-4- carboxamide |
| 25 | 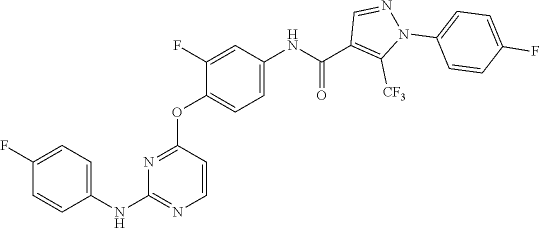 [SMILES: O=C(Nc1ccc(Oc2ccnc(Nc3ccc(F)cc3)n2)c(F)c1)c1cnn(-c2ccc(F)cc2)c1C(F)(F)F] | N-(3-fluoro-4-((2-((4- fluorophenyl)amino)pyrimidin- 4-yl)oxy)phenyl)-1-(4- fluorophenyl)-5- (trifluoromethyl)-1H-pyrazole-4- carboxamide |
| 26 | 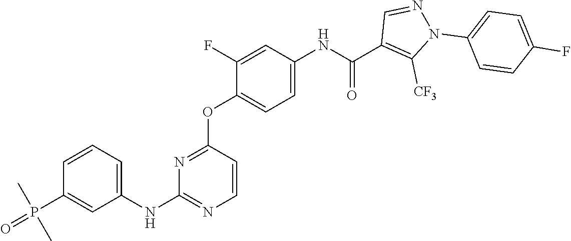 [SMILES: CP(C)(=O)c1cccc(Nc2nccc(Oc3ccc(NC(=O)c4cnn(-c5ccc(F)cc5)c4C(F)(F)F)cc3F)n2)c1] | N-(4-((2-((3- (dimethylphosphoryl)phenyl) amino)pyrimidin-4-yl)oxy)-3- fluorophenyl)-1-(4- fluorophenyl)-5- (trifluoromethyl)-1H-pyrazole-4- carboxamide |
| 27 | 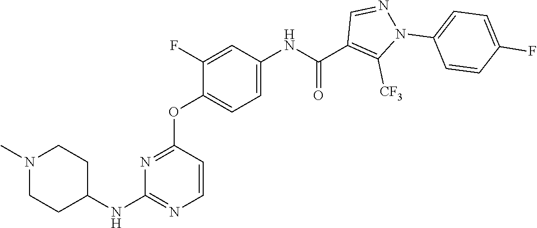 [SMILES: CN1CCC(Nc2nccc(Oc3ccc(NC(=O)c4cnn(-c5ccc(F)cc5)c4C(F)(F)F)cc3F)n2)CC1] | N-(3-fluoro-4-((2-((1- methylpiperidin-4- yl)amino)pyrimidin-4- yl)oxy)phenyl)-1-(4- fluorophenyl)-5- (trifluoromethyl)-1H-pyrazole-4- carboxamide |
| 28 | 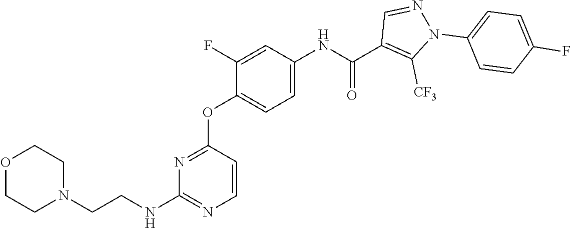 [SMILES: O=C(Nc1ccc(Oc2ccnc(NCCN3CCOCC3)n2)c(F)c1)c1cnn(-c2ccc(F)cc2)c1C(F)(F)F] | N-(3-fluoro-4-((2-((2- morpholinoethyl)amino)pyrimidin- 4-yl)oxy)phenyl)-1-(4- fluorophenyl)-5- (trifluoromethyl)-1H-pyrazole-4- carboxamide |
| 29 | 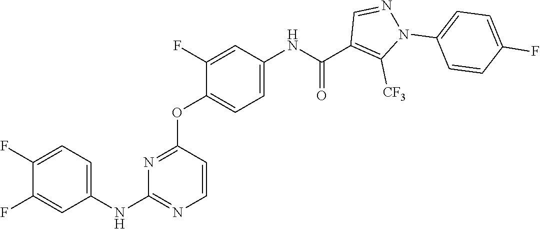 [SMILES: O=C(Nc1ccc(Oc2ccnc(Nc3ccc(F)c(F)c3)n2)c(F)c1)c1cnn(-c2ccc(F)cc2)c1C(F)(F)F] | N-(4-((2-((3,4- difluorophenyl)amino)pyrimidin- 4-yl)oxy)-3-fluorophenyl)-1-(4- fluorophenyl)-5- (trifluoromethyl)-1H-pyrazole-4- carboxamide |
| 30 | 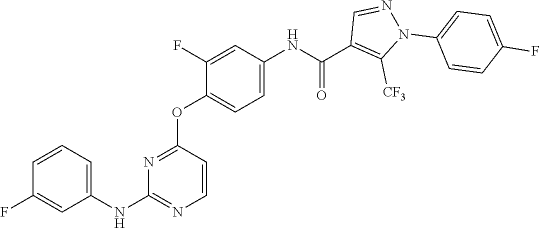 [SMILES: O=C(Nc1ccc(Oc2ccnc(Nc3cccc(F)c3)n2)c(F)c1)c1cnn(-c2ccc(F)cc2)c1C(F)(F)F] | N-(3-fluoro-4-((2-((3- fluorophenyl)amino)pyrimidin- 4-yl)oxy)phenyl)-1-(4- fluorophenyl)-5- (trifluoromethyl)-1H-pyrazole-4- carboxamide |
| 31 | 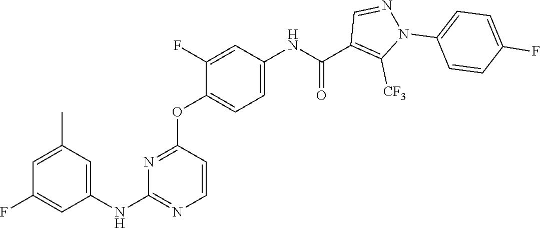 [SMILES: Cc1cc(F)cc(Nc2nccc(Oc3ccc(NC(=O)c4cnn(-c5ccc(F)cc5)c4C(F)(F)F)cc3F)n2)c1] | N-(3-fluoro-4-((2-((3-fluoro-5- methylphenyl)amino)pyrimidin- 4-yl)oxy)phenyl)-1-(4- fluorophenyl)-5- (trifluoromethyl)-1H-pyrazole-4- carboxamide |
| 32 | 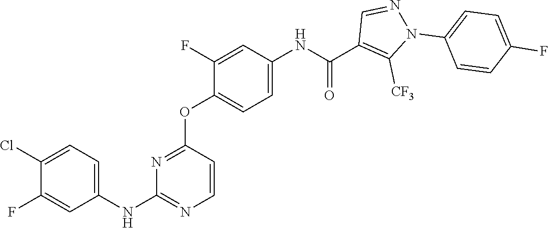 [SMILES: O=C(Nc1ccc(Oc2ccnc(Nc3ccc(Cl)c(F)c3)n2)c(F)c1)c1cnn(-c2ccc(F)cc2)c1C(F)(F)F] | N-(4-((2-((4-chloro-3- fluorophenyl)amino)pyrimidin- 4-yl)oxy)-3-fluorophenyl)-1-(4- fluorophenyl)-5- (trifluoromethyl)-1H-pyrazole-4- carboxamide |
| 33 | 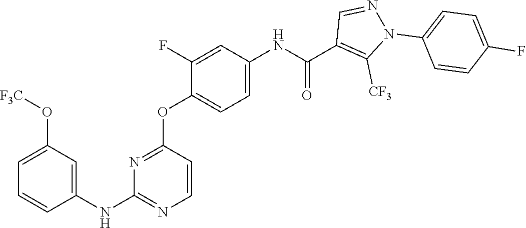 [SMILES: O=C(Nc1ccc(Oc2ccnc(Nc3cccc(OC(F)(F)F)c3)n2)c(F)c1)c1cnn(-c2ccc(F)cc2)c1C(F)(F)F] | N-(3-fluoro-4-((2-((3- (trifluoromethoxy)phenyl)amino) pyrimidin-4-yl)oxy)phenyl)-1- (4-fluorophenyl)-5- (trifluoromethyl)-1H-pyrazole-4- carboxamide |
| 34 | 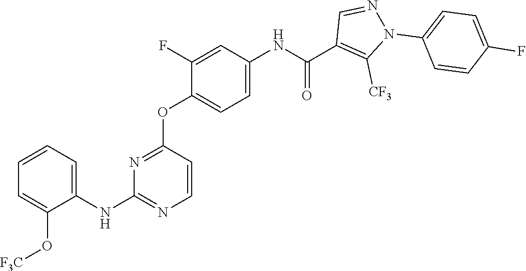 [SMILES: O=C(Nc1ccc(Oc2ccnc(Nc3ccccc3OC(F)(F)F)n2)c(F)c1)c1cnn(-c2ccc(F)cc2)c1C(F)(F)F] | N-(3-fluoro-4-((2-((2- (trifluoromethoxy)phenyl)amino) pyrimidin-4-yl)oxy)phenyl)-1- (4-fluorophenyl)-5- (trifluoromethyl)-1H-pyrazole-4- carboxamide |
| 35 | 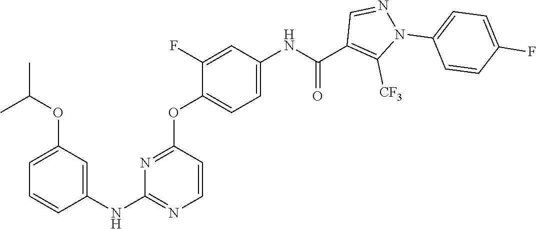 [SMILES: CC(C)Oc1cccc(Nc2nccc(Oc3ccc(NC(=O)c4cnn(-c5ccc(F)cc5)c4C(F)(F)F)cc3F)n2)c1] | N-(3-fluoro-4-((2-((3- isopropoxyphenyl)amino)pyrimidin- 4-yl)oxy)phenyl)-1-(4- fluorophenyl)-5- (trifluoromethyl)-1H-pyrazole-4- carboxamide |
| 36 | 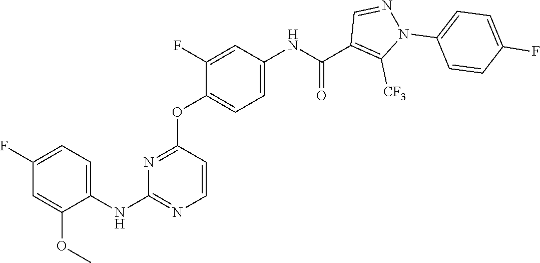 [SMILES: COc1cc(F)ccc1Nc1nccc(Oc2ccc(NC(=O)c3cnn(-c4ccc(F)cc4)c3C(F)(F)F)cc2F)n1] | N-(3-fluoro-4-((2-((4-fluoro-2- methoxyphenyl)amino)pyrimidin- 4-yl)oxy)phenyl)-1-(4- fluorophenyl)-5- (trifluoromethyl)-1H-pyrazole-4- carboxamide |
| 37 | 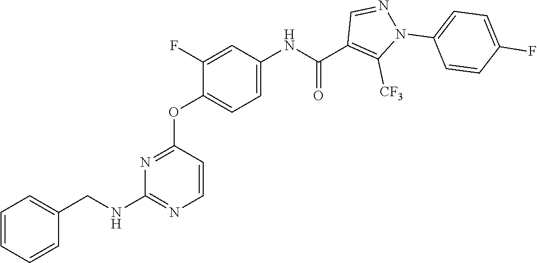 [SMILES: O=C(Nc1ccc(Oc2ccnc(NCc3ccccc3)n2)c(F)c1)c1cnn(-c2ccc(F)cc2)c1C(F)(F)F] | N-(4-((2- (benzylamino)pyrimidin-4- yl)oxy)-3-fluorophenyl)-1-(4- fluorophenyl)-5- (trifluoromethyl)-1H-pyrazole-4- carboxamide |
| 38 | 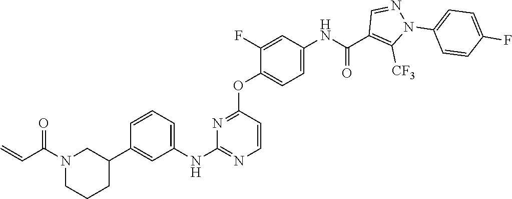 [SMILES: C=CC(=O)N1CCCC(c2cccc(Nc3nccc(Oc4ccc(NC(=O)c5cnn(-c6ccc(F)cc6)c5C(F)(F)F)cc4F)n3)c2)C1] | N-(4-((2-((3-(1- acryloylpiperidin-3- yl)phenyl)amino)pyrimidin-4- yl)oxy)-3-fluorophenyl)-1-(4- fluorophenyl)-5- (trifluoromethyl)-1H-pyrazole-4- carboxamide |
| 39 | 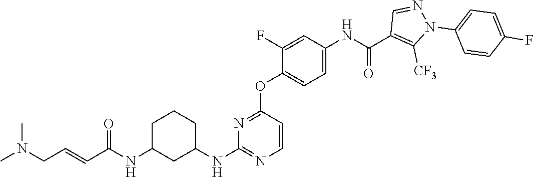 [SMILES: CN(C)C/C=C/C(=O)NC1CCCC(Nc2nccc(Oc3ccc(NC(=O)c4cnn(-c5ccc(F)cc5)c4C(F)(F)F)cc3F)n2)C1] | (E)-N-(4-((2-((3-(4- (dimethylamino)but-2- enamido)cyclohexyl)amino) pyrimidin-4-yl)oxy)-3- fluorophenyl)-1-(4- fluorophenyl)-5- (trifluoromethyl)-1H-pyrazole-4- carboxamide |
| 40 | 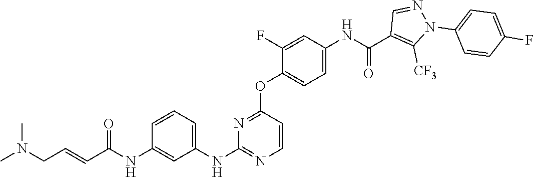 [SMILES: CN(C)C/C=C/C(=O)Nc1cccc(Nc2nccc(Oc3ccc(NC(=O)c4cnn(-c5ccc(F)cc5)c4C(F)(F)F)cc3F)n2)c1] | (E)-N-(4-((2-((3-(4- (dimethylamino)but-2- enamido)phenyl)amino)pyrimidin- 4-yl)oxy)-3-fluorophenyl)-1- (4-fluorophenyl)-5- (trifluoromethyl)-1H-pyrazole-4- carboxamide |
| 41 | 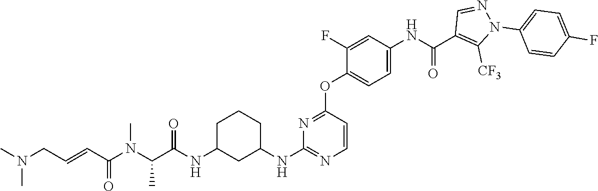 [SMILES: C[C@@H](C(=O)NC1CCCC(Nc2nccc(Oc3ccc(NC(=O)c4cnn(-c5ccc(F)cc5)c4C(F)(F)F)cc3F)n2)C1)N(C)C(=O)/C=C/CN(C)C] | N-(4-((2-((3-((S)-2-((E)-4- (dimethylamino)-N-methylbut-2- enamido)propanamido)cyclohexyl) amino)pyrimidin-4-yl)oxy)-3- fluorophenyl)-1-(4- fluorophenyl)-5- (trifluoromethyl)-1H-pyrazole-4- carboxamide |
| 42 | 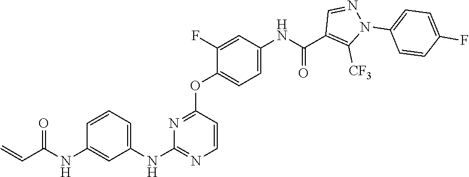 [SMILES: C=CC(=O)Nc1cccc(Nc2nccc(Oc3ccc(NC(=O)c4cnn(-c5ccc(F)cc5)c4C(F)(F)F)cc3F)n2)c1] | N-(4-((2-((3- acrylamidophenyl)amino)pyrimidin- 4-yl)oxy)-3-fluorophenyl)-1- (4-fluorophenyl)-5- (trifluoromethyl)-1H-pyrazole-4- carboxamide |
| 43 | 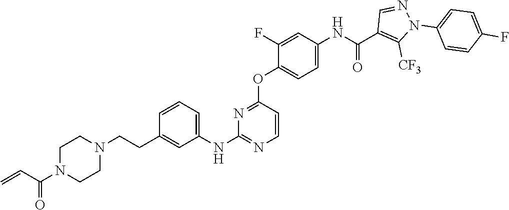 [SMILES: C=CC(=O)N1CCN(CCc2cccc(Nc3nccc(Oc4ccc(NC(=O)c5cnn(-c6ccc(F)cc6)c5C(F)(F)F)cc4F)n3)c2)CC1] | N-(4-((2-((3-(2-(4- acryloylpiperazin-1- yl)ethyl)phenyl)amino)pyrimidin- 4-yl)oxy)-3-fluorophenyl)-1-(4- fluorophenyl)-5- (trifluoromethyl)-1H-pyrazole-4- carboxamide |
| 44 | 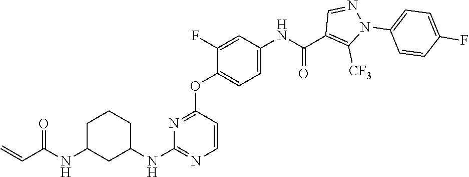 [SMILES: C=CC(=O)NC1CCCC(Nc2nccc(Oc3ccc(NC(=O)c4cnn(-c5ccc(F)cc5)c4C(F)(F)F)cc3F)n2)C1] | N-(4-((2-((3- acrylamidocyclohexyl)amino) pyrimidin-4-yl)oxy)-3- fluorophenyl)-1-(4- fluorophenyl)-5- (trifluoromethyl)-1H-pyrazole-4- carboxamide |
| 45 | 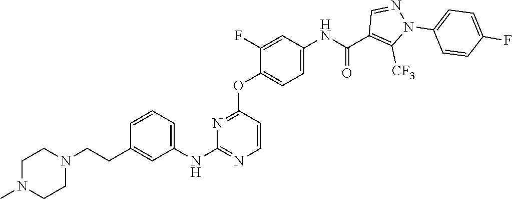 [SMILES: CN1CCN(CCc2cccc(Nc3nccc(Oc4ccc(NC(=O)c5cnn(-c6ccc(F)cc6)c5C(F)(F)F)cc4F)n3)c2)CC1] | N-(3-fluoro-4-((2-((3-(2-(4- methylpiperazin-1- yl)ethyl)phenyl)amino)pyrimidin- 4-yl)oxy)phenyl)-1-(4- fluorophenyl)-5- (trifluoromethyl)-1H-pyrazole-4- carboxamide |
| 46 | 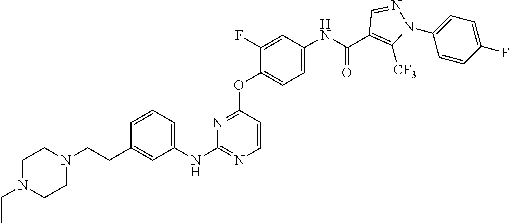 [SMILES: CCN1CCN(CCc2cccc(Nc3nccc(Oc4ccc(NC(=O)c5cnn(-c6ccc(F)cc6)c5C(F)(F)F)cc4F)n3)c2)CC1] | N-(4-((2-((3-(2-(4- ethylpiperazin-1- yl)ethyl)phenyl)amino)pyrimidin- 4-yl)oxy)-3-fluorophenyl)-1-(4- fluorophenyl)-5- (trifluoromethyl)-1H-pyrazole-4- carboxamide |
| 47 | 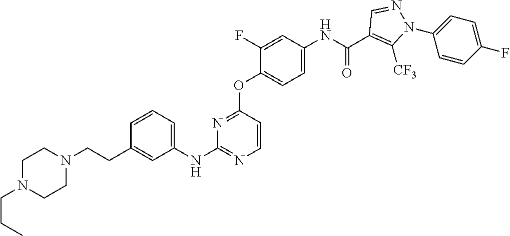 [SMILES: CCCN1CCN(CCc2cccc(Nc3nccc(Oc4ccc(NC(=O)c5cnn(-c6ccc(F)cc6)c5C(F)(F)F)cc4F)n3)c2)CC1] | N-(3-fluoro-4-((2-((3-(2-(4- propylpiperazin-1- yl)ethyl)phenyl)amino)pyrimidin- 4-yl)oxy)phenyl)-1-(4- fluorophenyl)-5- (trifluoromethyl)-1H-pyrazole-4- carboxamide |
| 48 | 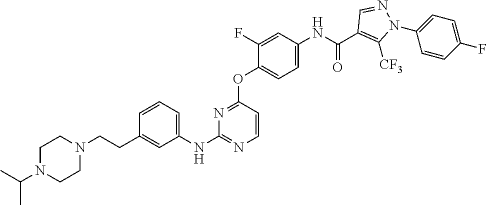 [SMILES: CC(C)N1CCN(CCc2cccc(Nc3nccc(Oc4ccc(NC(=O)c5cnn(-c6ccc(F)cc6)c5C(F)(F)F)cc4F)n3)c2)CC1] | N-(3-fluoro-4-((2-((3-(2-(4- isopropylpiperazin-1- yl)ethyl)phenyl)amino)pyrimidin- 4-yl)oxy)phenyl)-1-(4- fluorophenyl)-5- (trifluoromethyl)-1H-pyrazole-4- carboxamide |
| 49 | 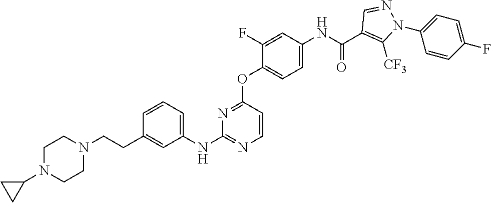 [SMILES: O=C(Nc1ccc(Oc2ccnc(Nc3cccc(CCN4CCN(C5CC5)CC4)c3)n2)c(F)c1)c1cnn(-c2ccc(F)cc2)c1C(F)(F)F] | N-(4-((2-((3-(2-(4- cyclopropylpiperazin-1- yl)ethyl)phenyl)amino)pyrimidin- 4-yl)oxy)-3-fluorophenyl)-1-(4- fluorophenyl)-5- (trifluoromethyl)-1H-pyrazole-4- carboxamide |
| 50 | 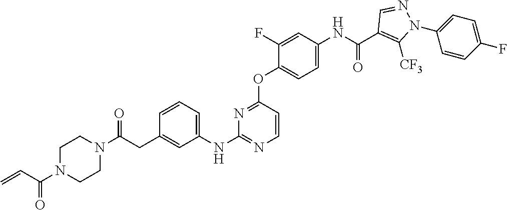 [SMILES: C=CC(=O)N1CCN(C(=O)Cc2cccc(Nc3nccc(Oc4ccc(NC(=O)c5cnn(-c6ccc(F)cc6)c5C(F)(F)F)cc4F)n3)c2)CC1] | N-(4-((2-((3-(2-(4- acryloylpiperazin-1-yl)-2- oxoethyl)phenyl)amino)pyrimidin- 4-yl)oxy)-3-fluorophenyl)-1- (4-fluorophenyl)-5- (trifluoromethyl)-1H-pyrazole-4- carboxamide |
| 51 | 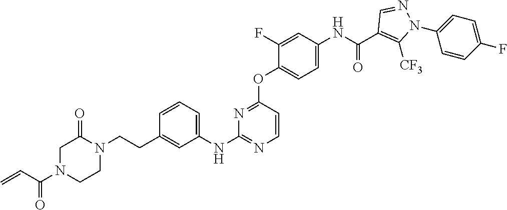 [SMILES: C=CC(=O)N1CCN(CCc2cccc(Nc3nccc(Oc4ccc(NC(=O)c5cnn(-c6ccc(F)cc6)c5C(F)(F)F)cc4F)n3)c2)C(=O)C1] | N-(4-((2-((3-(2-(4-acryloyl-2- oxopiperazin-1- yl)ethyl)phenyl)amino)pyrimidin- 4-yl)oxy)-3-fluorophenyl)-1-(4- fluorophenyl)-5- (trifluoromethyl)-1H-pyrazole-4- carboxamide |
| 52 | 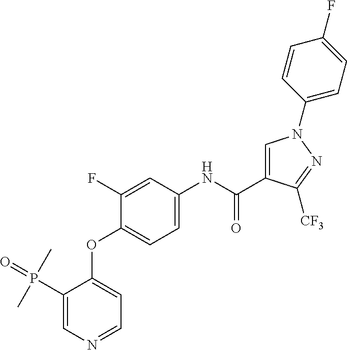 [SMILES: CP(C)(=O)c1cnccc1Oc1ccc(NC(=O)c2cn(-c3ccc(F)cc3)nc2C(F)(F)F)cc1F] | N-(4-((3- (dimethylphosphoryl)pyridin-4- yl)oxy)-3-fluorophenyl)-1-(4- fluorophenyl)-3- (trifluoromethyl)-1H-pyrazole-4- carboxamide |
| 53 | 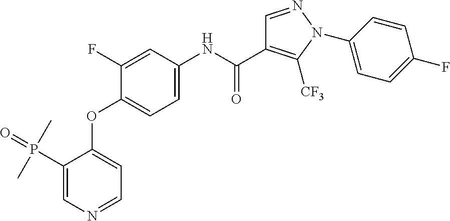 [SMILES: CP(C)(=O)c1cnccc1Oc1ccc(NC(=O)c2cnn(-c3ccc(F)cc3)c2C(F)(F)F)cc1F] | N-(4-((3- (dimethylphosphoryl)pyridin-4- yl)oxy)-3-fluorophenyl)-1-(4- fluorophenyl)-5- (trifluoromethyl)-1H-pyrazole-4- carboxamide |
| 54 | 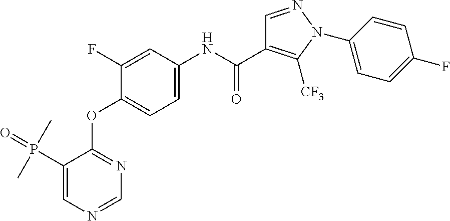 [SMILES: CP(C)(=O)c1cncnc1Oc1ccc(NC(=O)c2cnn(-c3ccc(F)cc3)c2C(F)(F)F)cc1F] | N-(4-((5- (dimethylphosphoryl)pyrimidin- 4-yl)oxy)-3-fluorophenyl)-1-(4- fluorophenyl)-5- (trifluoromethyl)-1H-pyrazole-4- carboxamide |
| 55 | 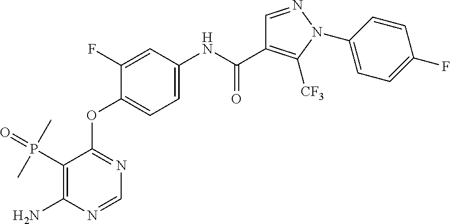 [SMILES: CP(C)(=O)c1c(N)ncnc1Oc1ccc(NC(=O)c2cnn(-c3ccc(F)cc3)c2C(F)(F)F)cc1F] | N-(4-((6-amino-5- (dimethylphosphoryl)pyrimidin- 4-yl)oxy)-3-fluorophenyl)-1-(4- fluorophenyl)-5- (trifluoromethyl)-1H-pyrazole-4- carboxamide |
| 56 | 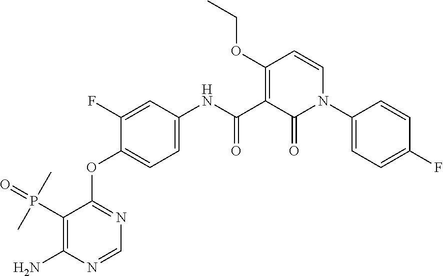 [SMILES: CCOc1ccn(-c2ccc(F)cc2)c(=O)c1C(=O)Nc1ccc(Oc2ncnc(N)c2P(C)(C)=O)c(F)c1] | N-(4-((6-amino-5- (dimethylphosphoryl)pyrimidin- 4-yl)oxy)-3-fluorophenyl)-4- ethoxy-1-(4-fluorophenyl)-2- oxo-1,2-dihydropyridine-3- carboxamide |
| 57 | 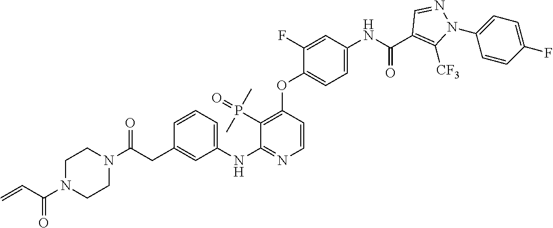 [SMILES: C=CC(=O)N1CCN(C(=O)Cc2cccc(Nc3nccc(Oc4ccc(NC(=O)c5cnn(-c6ccc(F)cc6)c5C(F)(F)F)cc4F)c3P(C)(C)=O)c2)CC1] | N-(4-((2-((3-(2-(4- acryloylpiperazin-1-yl)-2- oxoethyl)phenyl)amino)-3- (dimethylphosphoryl)pyridin-4- yl)oxy)-3-fluorophenyl)-1-(4- fluorophenyl)-5- (trifluoromethyl)-1H-pyrazole-4- carboxamide |
| 58 | 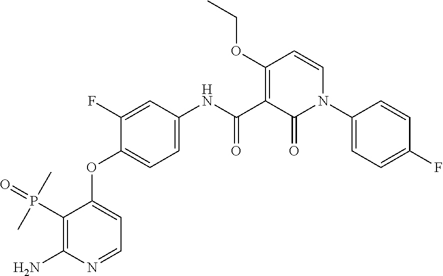 [SMILES: CCOc1ccn(-c2ccc(F)cc2)c(=O)c1C(=O)Nc1ccc(Oc2ccnc(N)c2P(C)(C)=O)c(F)c1] | N-(4-((2-amino-3- (dimethylphosphoryl)pyridin-4- yl)oxy)-3-fluorophenyl)-4- ethoxy-1-(4-fluorophenyl)-2- oxo-1,2-dihydropyridine-3- carboxamide |
| 59 | 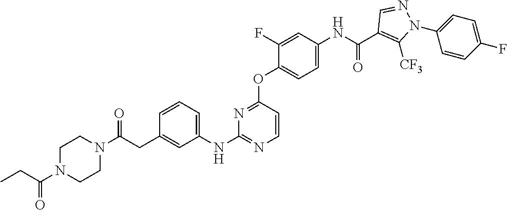 [SMILES: CCC(=O)N1CCN(C(=O)Cc2cccc(Nc3nccc(Oc4ccc(NC(=O)c5cnn(-c6ccc(F)cc6)c5C(F)(F)F)cc4F)n3)c2)CC1] | N-(3-fluoro-4-((2-((3-(2-(4- propionylpiperazin-1- yl)ethyl)phenyl)amino)pyrimidin- 4-yl)oxy)phenyl)-1-(4- fluorophenyl)-5- (trifluoromethyl)-1H-pyrazole-4- carboxamide |
| 60 | 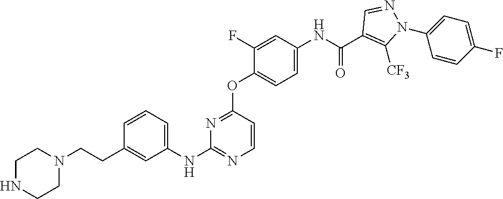 [SMILES: O=C(Nc1ccc(Oc2ccnc(Nc3cccc(CCN4CCNCC4)c3)n2)c(F)c1)c1cnn(-c2ccc(F)cc2)c1C(F)(F)F] | N-(3-fluoro-4-((2-((3-(2- (piperazin-1- yl)ethyl)phenyl)amino)pyrimidin- 4-yl)oxy)phenyl)-1-(4- fluorophenyl)-5- (trifluoromethyl)-1H-pyrazole-4- carboxamide |

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions

Other embodiments are directed to pharmaceutical compositions. The pharmaceutical composition comprises anyone (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of at least one compound of Structure (I) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to inter-subject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I), and a pharmaceutically acceptable carrier.

Provided herein are pharmaceutical compositions comprising one or more compounds selected from compounds of Structure (I) and pharmaceutically acceptable diluent(s), excipient(s), and carrier(s). In certain embodiments, the compounds described are administered as pharmaceutical compositions in which one or more compounds selected from compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

A pharmaceutical composition, as used herein, refers to a mixture of one or more compounds selected from compounds of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) provided herein are administered in a pharmaceutical composition to a mammal having a disease, disorder, or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) are formulated for trans-mucosal administration. In specific embodiments, trans-mucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions, or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent, or excipient, and one or more compounds selected from compounds of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass un-solvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) illustratively takes the form of a liquid where the agents are present in solution, in suspension or both. Typically, when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium, or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-re-closable containers. Alternatively, multiple-dose re-closable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds selected from compounds of Structure (I) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v, or v/v.

In some embodiments, the amount the one or more compounds selected from compounds of Structure (I) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial, and/or tube labels listing contents and/or instructions for use, and package inserts with instructions for use. A set of instructions will also typically be included. A label is optionally on or associated with the container. For example, a label is on a container when letters, numbers or other characters forming the label are attached, molded, or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or the pack or dispenser device is accompanied by instructions for administration. Or the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods

RON belongs to a subfamily of receptor tyrosine kinases. RON is encoded by the MST1R gene. RON is also known as macrophage stimulating protein receptor. RON is activated by a serum derived growth factor macrophage stimulating protein (MSP). RON gene transcription is essential for embryonic development and critical in regulating certain physiological processes. Activation of RON leads to the activation of common receptor tyrosine kinase downstream-signaling pathways, such as activation of MAPK, PI3K, RAS-ERK, and beta-catenin. Furthermore, RON participates in crosstalk with other signaling pathways such as Insulin-like growth factor 1 (IGF1R) and EGF receptor (EGFR) that are common tumorigenic mechanisms. Certain embodiments provide methods or compositions for use in regulating these signaling pathways.

Embodiments of the present disclosure are useful as modulators of the RON pathway in a host species. RON inhibitors may also inhibit the activity of the RON pathway, including MSP, MBD4, SRC, and PI3K. Therefore, the compounds of Structure (I) are also useful in the treatment of conditions mediated by these kinases.

The host or patient can belong to any mammalian species, for example a primate species, particularly humans; rodents, including mice, rats, and hamsters; rabbits; horses, cows, dogs, cats, etc. Animal models are of interest for experimental investigations, providing a model for treatment of human disease.

A method of treating a disease or disorder, the method comprising administering the compound of the disclosure as a stereoisomer, enantiomer, or tautomer thereof or a mixture thereof or a pharmaceutically acceptable salt, solvate, or prodrug thereof or the pharmaceutical composition of the disclosure to a subject in need thereof.

In some embodiments, the disease is cancer. In certain embodiments, the cancer is skin cancer, breast cancer, prostate cancer, lung cancer, pancreatic cancer, colon cancer, bone cancer, bladder cancer, rectal cancer, stomach cancer, esophageal cancer, tracheal cancer, throat cancer, neck cancer, liver cancer, kidney cancer, brain cancer, thyroid cancer, testicle cancer, ovarian cancer, and cervical cancer. In some embodiments, the cancer is carcinoma, sarcoma, lymphoma, leukemia, blastoma, or germ cell tumor. In some embodiments, the cancer is bone cancer. In certain embodiments, the cancer includes a bone tumor.

In certain embodiments, the disease is osteolysis or osteoporosis. Inhibiting or treating osteolysis may include reducing bone turn over, reducing the progression of bone loss, inhibiting osteoclasts, or a combination thereof. In some embodiments, the subject has been diagnosed with a condition selected from inflammation, cyst, cancer, cancer with bone metastasis, and cancer-mediated bone destruction. Provided herein are methods for treating osteoporosis in a subject in need thereof. Treating osteoporosis may include preventing osteoporosis, reducing the progression of osteoporosis, or a combination thereof.

In some embodiments, the inhibitor may act independently of the RANKL and/or TGFβ pathways. In certain embodiments, a compound may not affect the function of RANKL and/or TGFβ. In some embodiments, the compound may be selective or specific for RON. As used herein, a RON-selective inhibitor inhibits the activity of RON greater than it inhibits the activity of another protein, such as, for example, another receptor tyrosine kinase, under physiological conditions. Receptor tyrosine kinases other than RON include, for example, Met and Axl. For example, a RON-selective inhibitor may inhibit RON greater than it inhibits Met under physiological conditions. For example, a RON-selective inhibitor may inhibit RON greater than it inhibits Axl under physiological conditions.

In some embodiments, a compound of the disclosure may inhibit the activity of RON at least 2 times, at least 3 times, at least 4 times, at least 5 times, at least 10 times, at least 15 times, at least 20 times, at least 25 times, at least 50 times, or at least 100 times more than it inhibits the activity of another receptor tyrosine kinase, (e.g., Met or Axl).

In some embodiments, a compound of the disclosure may have an IC₅₀ for another receptor tyrosine kinase that is at least 2 times, at least 3 times, at least 4 times, at least 5 times, at least 10 times, at least 15 times, at least 20 times, at least 25 times, at least 50 times, or at least 100 times greater than the IC₅₀s for RON.

Embodiments of the disclosure also relate to the use of compounds according to Structure (I) and/or physiologically acceptable salts thereof for the prophylactic or therapeutic treatment and/or monitoring of diseases that are caused, mediated and/or modulated by RON pathway activity. Furthermore, embodiments of the disclosure relate to the use of compounds according to Structure (I) and/or physiologically acceptable salts thereof to produce a medicament for the prophylactic or therapeutic treatment and/or monitoring of diseases that are caused, mediated and/or modulated RON pathway activity. In certain embodiments, the disclosure provides the use of a compound according to Structure (I) or physiologically acceptable salts thereof, to produce a medicament for the prophylactic or therapeutic treatment of a RON-mediated disorder.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, p-adrenergic agonists, ipratropium, glucocorticoids, methylxanthines, sodium channel blockers, opioid receptor agonists, calcium channel blockers, membrane stabilizers and leukotriene inhibitors.

Other embodiments of the disclosure pertain to combinations in which at least one anti-inflammatory compound is an anti-monoclonal antibody (such as eculizumab or pexelizumab), a TNF antagonist, such as entanercept, or infliximab, which is an anti-TNF alpha monoclonal antibody.

The disclosed compounds of Structure (I) can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories—
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-3024, VAL-0834;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine; Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase3, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide;
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein, or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, a few hours apart, or a few days apart.

In some embodiments, the compounds of Structure (I) are administered as a monotherapy.

For identification of a signal transduction or a mechanistic pathway and for detection of interactions between various signal transduction pathways, various scientists have developed suitable models or model systems, for example cell culture models and models of transgenic animals. For the determination of certain stages in the signal transduction cascade, interacting compounds can be utilized to modulate the signal. The compounds of embodiments of the disclosure can also be used as reagents for testing kinase dependent signal transduction pathways in animals and/or cell culture models or in the clinical diseases mentioned in this application.

The methods of embodiments of embodiments of the disclosure can be performed either in vitro or in vivo. The susceptibility of a particular cell to treatment with the compounds of Structure (I) can be particularly determined by in vitro tests, whether during research or clinical application. Typically, a culture of the cell is combined with a compound at various concentrations for a period which is sufficient to allow the active agents to inhibit kinase activity, usually between about one hour and one week. In-vitro treatment can be carried out using cultivated cells from a biopsy sample or cell line.

In some embodiments, the IC₅₀ of the compounds of Structure (I) to inhibit kinase activity was determined by the concentration of the compound required to inhibit 50% of the activity of the kinase. The compounds of Structure (I) exhibited potency values of IC₅₀ of less than about 5 mM, preferably less than about 1 mM and even more preferably less than about 0.100 mM as described in further detail in the Examples.

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5^{th} edition (Wiley, December 2000)) or prepared as described herein.

### EXAMPLES

The following examples are provided for exemplary purposes.

### Abbreviations

° C. (degree Celsius); ¹H NMR (proton Nuclear Magnetic Resonance); ACN (acetonitrile); Boc (tert-butyloxycarbonyl); (Boc)₂O (di-tert-butyl dicarbonate); DCM (dichloromethane); DIPEA (N,N-diisopropylethylamine); DMAP (4-dimethylaminopyridine); DMF (NN-dimethylformamide); DMSO-d₆ (deuterated dimethylsulfoxide); eq (equivalent); EtOAc (ethyl acetate); EtOH (ethanol); g (gram); h (hour); HCl (hydrochloric acid); HPLC (High Performance Liquid Chromatography); LCMS (Liquid Chromatography Mass Spectrometry); MeOH (methanol); mg (milligram); min (minute); mL (milliliter); μL (microliter); mmol (millimole); Pd(PPh₃)₄ or “tetrakis” (tetrakis(triphenylphosphine)palladium(0)); SM (starting material); TEA (triethylamine); TFA (trifluoroacetic acid); THE (tetrahydrofuran); TLC (Thin Layer Chromatography); UPLC (Ultra performance liquid chromatography).

### Intermediate Example 1

### Synthesis of N-(4-((2-Chloropyrimidin-4-Yl)Oxy)-3-Fluorophenyl)-1-(4-Fluorophenyl)-5-(Trifluoromethyl)-1H-Pyrazole-4-Carboxamide

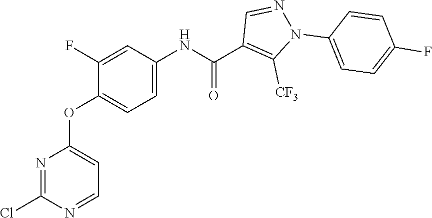 [SMILES: O=C(Nc1ccc(Oc2ccnc(Cl)n2)c(F)c1)c1cnn(-c2ccc(F)cc2)c1C(F)(F)F]

### Step 1: Synthesis of ethyl 1-(4-fluorophenyl)-5-(trifluoromethyl)-1H-pyrazole-4-carboxylate

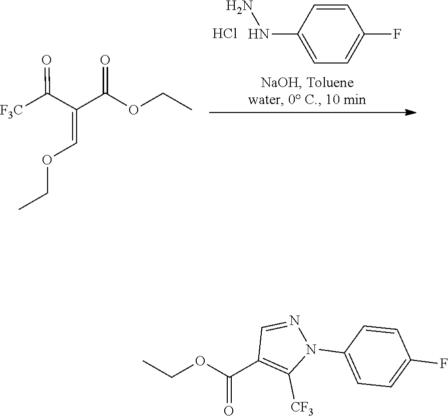 [SMILES: CCO/C=C(/C(=O)OCC)C(=O)C(F)(F)F ; Cl ; NNc1ccc(F)cc1 ; CCOC(=O)c1cnn(-c2ccc(F)cc2)c1C(F)(F)F]

To a stirred solution of ethyl (2E)-2-(ethoxymethylidene)-4,4,4-trifluoro-3-oxobutanoate (7.39 g, 30.8 mmol, 1.0 eq.) and 4-fluorophenylhydrazine hydrochloride (5 g, 30.8 mmol, 1.0 eq.) in Toluene (25 mL) was added 10% aqueous sodium hydroxide solution (25 mL) dropwise at 0° C. After that, the mixture was stirred at the same temperature for 10 minutes. Progress of the reaction was monitored by TLC. After SM consumed on TLC, concentrated hydrochloric acid was added to the reaction solution to make it acidic, and then diluted with water (100 mL). Then the mixture was extracted with ethyl acetate (300 mL×3) and combined organic layers dried over Na₂SO₄ and concentrated under reduced pressure to obtain the title compound ethyl 1-(4-fluorophenyl)-5-(trifluoromethyl)-1H-pyrazole-4-carboxylate (9.2 g, 98.99% yield) as a brown oil.

LCMS: 303.1 (M+H)⁺. ¹H NMR (400 MHz, DMSO-d₆): δ 8.29 (s, 1H), 7.66-7.62 (m, 2H), 7.44 (t, J=8.80 Hz, 2H), 4.31 (q, J=7.20 Hz, 2H), 1.29 (t, J=7.20 Hz, 3H).

### Step 2: Synthesis of 2-methyl-5-[(3-phenyloxetan-3-yl) amino]pyridine-4-carboxylic acid

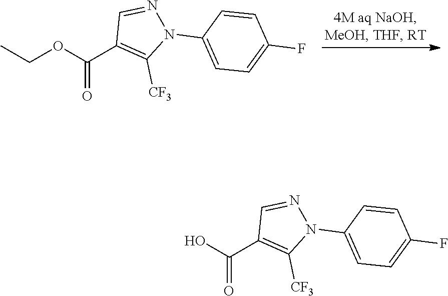 [SMILES: CCOC(=O)c1cnn(-c2ccc(F)cc2)c1C(F)(F)F ; O=C(O)c1cnn(-c2ccc(F)cc2)c1C(F)(F)F]

To a stirred solution of ethyl 2-methyl-5-[(3-phenyloxetan-3-yl)amino]pyridine-4-carboxylate (9 g, 29.8 mmol, 1.0 eq) in THF: MeOH (1:1) (90 mL) was added sodium hydroxide (5.96 g, 149 mmol, 5.0 eq) dissolved in water (50 mL). Then reaction mixture stirred at room temperature for 2 h. Progress of reaction was monitored by TLC. After completion of reaction, the reaction mixture was concentrated under reduced pressure and then diluted with water (200 mL). The reaction mixture was acidified with conc. HCl under cooling and extracted with ethyl acetate (3×300 mL). The combined organic layer was dried over Na₂SO₄ and concentrated under reduced pressure to obtained the title compound 2-methyl-5-[(3-phenyloxetan-3-yl)amino]pyridine-4-carboxylic acid (6 g, 73.49% yield) as off white solid.

LCMS: 273.1 (M−H)⁺. ¹H NMR (400 MHz, DMSO-d₆): δ 13.37 (brs, 1H), 8.24 (s, 1H), 7.65-7.62 (m, 2H), 7.43 (t, J=8.40 Hz, 2H).

### Step 3: Synthesis of N-(3-fluoro-4-hydroxyphenyl)-1-(4-fluorophenyl)-5-(trifluoromethyl)-1H-pyrazole-4-carboxamide

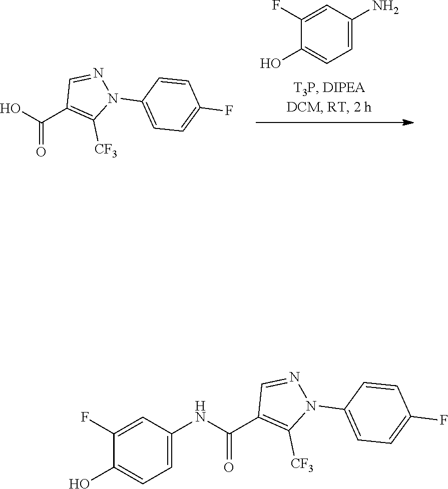 [SMILES: Nc1ccc(O)c(F)c1 ; O=C(O)c1cnn(-c2ccc(F)cc2)c1C(F)(F)F ; O=C(Nc1ccc(O)c(F)c1)c1cnn(-c2ccc(F)cc2)c1C(F)(F)F]

To a stirred solution of 1-(4-fluorophenyl)-5-(trifluoromethyl)-1H-pyrazole-4-carboxylic acid (5.5 g, 20.1 mmol, 1.0 eq) and 4-amino-2-fluorophenol (3.83 g, 30.1 mmol, 1.5 eq) and DIPEA (7.16 mL, 40.1 mmol, 2 eq) in DCM (100 mL) was added T3P (50% in EtOAc) (7.72 mL, 30.1 mmol, 3 eq) at room temperature. Then the reaction mixture stirred for 2 h at room temperature. Progress of the reaction was monitored by TLC. After completion of reaction, water (200 mL) was added to reaction mixture and extracted with ethyl acetate (350 mL×3). Combined organic layer was dried over anhydrous Na₂SO₄ and concentrated under reduced pressure to get brown color gummy liquid crude product. The crude compound was purified by flash column using (100-200 silica mesh) 30% EtOAc in hexane as an eluent to afford title compound N-(3-fluoro-4-hydroxyphenyl)-1-(4-fluorophenyl)-5-(trifluoromethyl)-1H-pyrazole-4-carboxamide (4 g, 52% yield) as off white solid.

¹H NMR (400 MHz, DMSO-d₆): δ 10.45 (s, 1H), 9.72 (s, 1H), 8.28 (s, 1H), 7.63-7.57 (m, 3H), 7.47-7.43 (m, 2H), 7.24 (d, J=8.4 Hz, 1H), 6.93 (t, J=9.2 Hz, 1H). LCMS: 384.25 (M+H)⁺.

### Step 4: Synthesis of N-{4-[(2-chloropyrimidin-4-yl) oxy]-3-fluorophenyl}-1-(4-fluorophenyl)-5-(trifluoromethyl)-1H-pyrazole-4-carboxamide

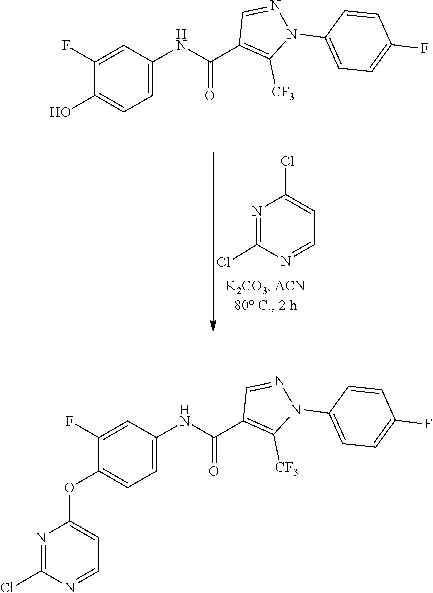

To a solution of N-(3-fluoro-4-hydroxyphenyl)-1-(4-fluorophenyl)-5-(trifluoromethyl)-1H-pyrazole-4-carboxamide (2 g, 5.22 mmol, 1 eq) and 2,4-dichloropyrimidine (777 mg, 5.22 mmol, 1 eq) in DMF (20 mL) was added K₂CO₃ (517 mg, 5.22 mmol, 1 eq). Then the reaction mixture was stirred at 80° C. for 2 h. The reaction progress was monitored by TLC & LCMS. After completion of reaction, water (100 mL) was added to reaction mixture and extracted with ethyl acetate (3×150 mL). Combined organic layer was dried over anhydrous Na₂SO₄ and concentrated under reduced pressure to get brown color gummy liquid crude product. The crude compound was purified by flash column by using (100-200 silica mesh) 50% EtOAc in hexane as a eluent to afford title compound N-{4-[(2-chloropyrimidin-4-yl)oxy]-3-fluorophenyl}-1-(4-fluorophenyl)-5-(trifluoromethyl)-1H-pyrazole-4-carboxamide (1.5 g, 57.98% yield) as off brown solid.

¹H NMR (400 MHz, DMSO-d₆): δ 10.85 (s, 1H), 8.70 (d, J=5.60 Hz, 1H), 8.35 (s, 1H), 7.88 (d, J=12.40 Hz, 1H), 7.66-7.63 (m, 2H), 7.54 (d, J=8.80 Hz, 1H), 7.47 (t, J=8.80 Hz, 3H), 7.36 (d, J=5.60 Hz, 1H). LCMS: 496.53 (M+H)⁺.

### Intermediate Example 2

### Synthesis of N-(4-((2-((3-Aminocyclohexyl)Amino)Pyrimidin-4-Yl)Oxy)-3-Fluorophenyl)-1-(4-Fluorophenyl)-5-(Trifluoromethyl)-1H-Pyrazole-4-Carboxamide

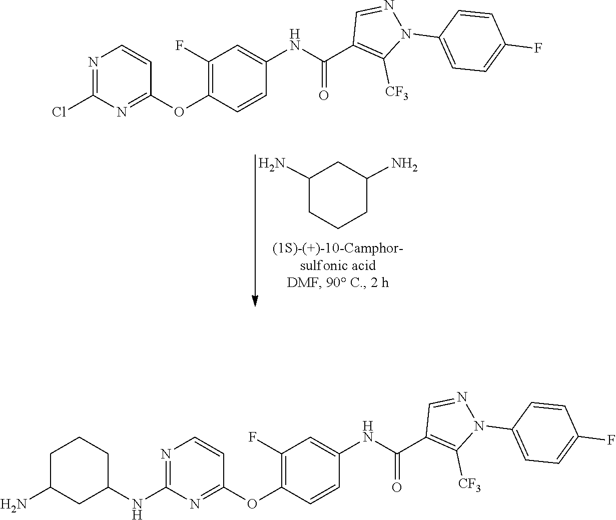

To a stirred solution of N-(4-((2-chloropyrimidin-4-yl)oxy)-3-fluorophenyl)-1-(4-fluorophenyl)-5-(trifluoromethyl)-1H-pyrazole-4-carboxamide (1 g, 2.02 mmol, 1 eq) in DMF (10 mL) was added (1S)-(+)-10-Camphorsulfonic acid (1.41 g, 6.05 mmol, 3 eq) followed by 1,3-cyclohexanediamine (461 mg, 4.03 mmol, 2 eq). The reaction mixture was stirred for 3 h at 90° C. The reaction progress was monitored by LCMS. After completion of reaction, reaction mixture was cool to room temperature and ice-cold water (50 mL) was added and extracted with ethyl acetate (3×30 mL). The combined organic layer wash with brine solution (30 mL), then dried over Na₂SO₄ and concentrated under reduced pressure to get crude compound. The crude compound was purified by prep-HPLC using 0.1% FA in acetonitrile to afford N-(4-((2-((3-aminocyclohexyl)amino)pyrimidin-4-yl)oxy)-3-fluorophenyl)-1-(4-fluorophenyl)-5-(trifluoromethyl)-1H-pyrazole-4-carboxamide (200 mg, 17.29% yield) as off white solid.

¹H NMR (400 MHz, DMSO) δ 10.89 (s, 1H), 8.35 (s, 2H), 8.21-8.18 (m, 1H), 7.83-7.80 (m, 1H), 7.65-7.61 (m, 2H), 7.49-7.44 (m, 3H), 7.34 (t, J=7.2 Hz, 1H), 7.15 (m, 1H), 6.28 (m, 1H), 3.77-3.74 (m, 2H), 1.97-1.70 (m, 3H), 1.59-1.47 (m, 2H), 1.37-1.31 (m, 1H), 1.16-1.07 (m, 1H), LCMS: 574.51 (M+H)⁺.

### Intermediate Example 3

### Synthesis of N-(4-((2-((3-Aminophenyl) Amino) Pyrimidin-4-Yl) Oxy)-3-Fluorophenyl)-1-(4-Fluorophenyl)-5-(Trifluoromethyl)-1H-Pyrazole-4-Carboxamide

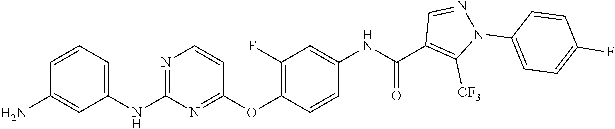 [SMILES: Nc1cccc(Nc2nccc(Oc3ccc(NC(=O)c4cnn(-c5ccc(F)cc5)c4C(F)(F)F)cc3F)n2)c1]

### Step 1: Synthesis of N-(3-fluoro-4-((2-((3-nitrophenyl) amino) pyrimidin-4-yl) oxy) phenyl)-1-(4-fluorophenyl)-5-(trifluoromethyl)-1H-pyrazole-4-carboxamide

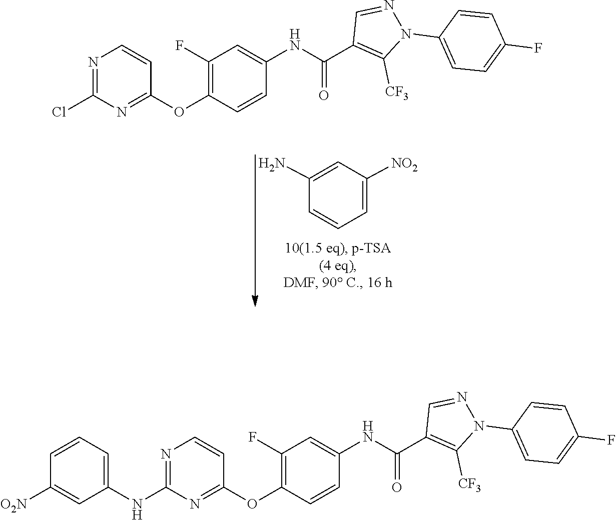

To a stirred solution m-Nitro aniline (418 mg, 3.03 mmol, 1.5 eq) and N-{4-[(2-chloropyrimidin-4-yl) oxy]-3-fluorophenyl}-1-(4-fluorophenyl)-5-(trifluoromethyl)-1H-pyrazole-4-carboxamide (1 g, 40.3 μmol, 1 eq) was added 4-methylbenzene-1-sulfonic acid hydrate (1.53 g, 8.07 μmol, 4 eq) at room temperature. Then the reaction mixture stirred for 10 h at 90° C. Progress of the reaction was monitored by TLC & LCMS. After completion of reaction, RM was cool to room temperature and ice-cold water (50 mL) was added then extracted with ethyl acetate (3×50 mL). Combined organic layer wash with brine solution and dried over Na₂SO₄ and concentrated under reduced pressure to get crude compound. Crude residue obtained was purified by column chromatography to afford title compound N-{3-fluoro-4-[2-(m-nitro phenyl amino)-4-pyrimidinyloxy]phenyl}-1-(p-fluorophenyl)-5-(trifluoromethyl)-4-pyrazolecarboxamide (630 mg, 52.28% yield).

¹H NMR (400 MHz, DMSO) δ 10.83 (s, 1H), 10.12 (s, 1H), 8.50 (d, J=5.6 Hz, 2H), 8.35 (s, 1H), 8.01-7.78 (m, 2H), 7.71 (d, J=8.1 Hz, 1H), 7.72-7.59 (m, 2H), 7.59-7.10 (m, 5H), 6.70 (d, J=5.6 Hz, 1H). LCMS: 598.1 (M+H)⁺.

### Step 2: Synthesis of N-(4-((2-((3-aminophenyl) amino) pyrimidin-4-yl) oxy)-3-fluorophenyl)-1-(4-fluorophenyl)-5-(trifluoromethyl)-1H-pyrazole-4-carboxamide

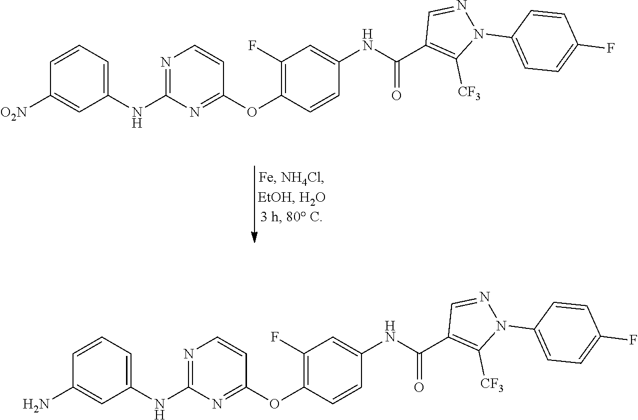

To stirred a solution of N-(3-fluoro-4-((2-((3-nitrophenyl)amino)pyrimidin-4-yl)oxy)phenyl)-1-(4-fluorophenyl)-5-(trifluoromethyl)-1H-pyrazole-4-carboxamide (0.26 g, 435 μmol, 1eq) in ethanol (4 mL) and water (2 mL) and DMF (1 mL) at 0° C., iron (243 mg, 4.35 mmol, 10 eq) and ammonium chloride (466 mg, 8.7 mmol, 20 eq) were added. Reaction mixture then stirred at 80° C. for 1 h. Progress of reaction was monitored by TLC. After completion of reaction, reaction mixture was diluted with EtOH, then filtered on a pad of diatomaceous earth (i.e., Celite®) and wash with water DMF (2:1). Water (10 mL) and sodium bicarbonate solution (3 mL) was added to filtrate and extracted with 10% MeOH in DCM (3×20 mL). Combined organic layers dried over Na₂SO₄ and concentrated under reduced pressure to get crude compound. Crude residue obtained was purified by column chromatography to afford title compound of N-(4-((2-((3-aminophenyl)amino)pyrimidin-4-yl)oxy)-3-fluorophenyl)-1-(4-fluorophenyl)-5-(trifluoromethyl)-1H-pyrazole-4-carboxamide (200 mg, 83.33% yield) as green color gummy solid.

¹H NMR (400 MHz, DMSO) δ 10.83 (s, 1H), 9.34 (s, 1H), 8.35 (d, J=5.6 Hz, 2H), 7.87 (d, J=12.8 Hz, 1H), 7.64 (dd, J=8.8, 4.8 Hz, 2H), 7.59-7.33 (m, 4H), 6.81-6.59 (m, 3H), 6.50 (d, J=5.6 Hz, 1H), 6.11 (d, J=8.4 Hz, 1H), 4.73 (s, 2H). LCMS: 568.2 (M+H)⁺.

### Synthetic Example 1

### Synthesis of N-(3-Fluoro-4-((2-((3-Morpholinophenyl) Amino) Pyrimidin-4-Yl) Oxy) Phenyl)-1-(4-Fluorophenyl)-5-(Trifluoromethyl)-1H-Pyrazole-4-Carboxamide

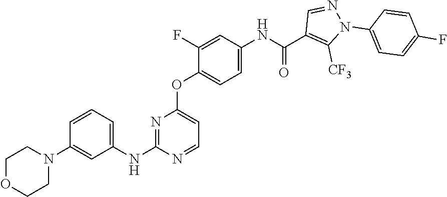 [SMILES: O=C(Nc1ccc(Oc2ccnc(Nc3cccc(N4CCOCC4)c3)n2)c(F)c1)c1cnn(-c2ccc(F)cc2)c1C(F)(F)F]

### Step 1: Synthesis of ethyl 1-(4-fluorophenyl)-5-(trifluoromethyl)-1H-pyrazole-4-carboxylate

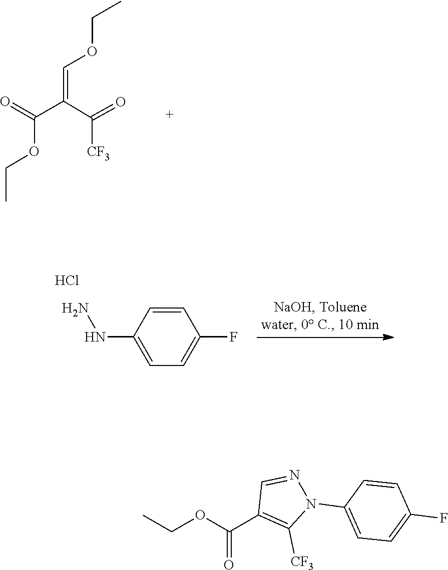 [SMILES: CCO/C=C(/C(=O)OCC)C(=O)C(F)(F)F ; Cl ; NNc1ccc(F)cc1 ; CCOC(=O)c1cnn(-c2ccc(F)cc2)c1C(F)(F)F]

To a stirred solution of ethyl (2E)-2-(ethoxymethylidene)-4,4,4-trifluoro-3-oxobutanoate (7.39 g, 30.8 mmol, 1 eq) and 4-fluorophenylhydrazine hydrochloride (5 g, 30.8 mmol, 1 eq) in toluene (25 mL) was ice-cooled and added 10% aqueous sodium hydroxide solution (2.5 g sodium hydroxide in 25 mL water) dropwise. After that, the mixture was stirred at the same temperature for 10 minutes. Add concentrated hydrochloric acid to the reaction solution to make it acidic, and then water (100 mL) was added, and the mixture was extracted with ethyl acetate (30 mL×3) then combining organic layers. After drying with Na₂SO₄, the solvent was removed under reduced pressure to obtain the ethyl 1-(4-fluorophenyl)-5-(trifluoromethyl)-1H-pyrazole-4-carboxylate as a brown oil (10 g, crude). LCMS: 303.1 (M+H)⁺. ¹H NMR (400 MHz, DMSO-d₆): δ=8.29 (s, 1H), 7.65-7.62 (m, 2H), 7.45-7.41 (m, 2H), 4.31 (q, J=14 Hz, 7.2 Hz, 2H), 1.29 (t, J=7.2 Hz, 2H).

### Step 2: Synthesis of 2-methyl-5-[(3-phenyloxetan-3-yl) amino]pyridine-4-carboxylic acid

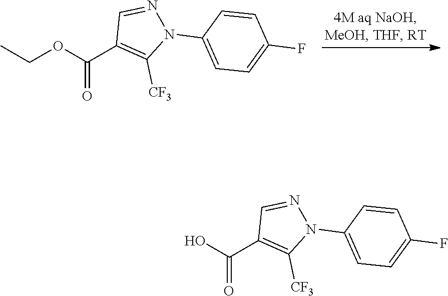 [SMILES: CCOC(=O)c1cnn(-c2ccc(F)cc2)c1C(F)(F)F ; O=C(O)c1cnn(-c2ccc(F)cc2)c1C(F)(F)F]

To a stirred solution of ethyl 2-methyl-5-[(3-phenyloxetan-3-yl) amino]pyridine-4-carboxylate (10 g, 33.1 mmol, 1.0 eq.) in THF: MeOH (50 mL:50 mL), was added and sodium hydroxide (6.62 g, 165 mmol, 5.0 eq) was dissolved in water (50 mL) added to reaction mixture at room temperature and stirred for 2 h. After complete conversion by TLC, the reaction mixture was concentrated under reduced pressure and diluted with water. The reaction mixture was acidified with conc. HCl under cooling and extracted with ethyl acetate. The organic layer was died and concentrate under reduced pressure to obtained the title compound 2-methyl-5-[(3-phenyloxetan-3-yl)amino]pyridine-4-carboxylic acid as off white solid (6 g, 66.14%). ¹H NMR (400 MHz, DMSO-d₆): δ=13.39 (br.s, 1H), 8.24 (s, 1H), 7.65-7.62 (m, 2H), 7.45-7.41 (m, 2H).

### Step 3: Synthesis of N-(3-fluoro-4-hydroxyphenyl)-1-(4-fluorophenyl)-5-(trifluoromethyl)-1H-pyrazole-4-carboxamide

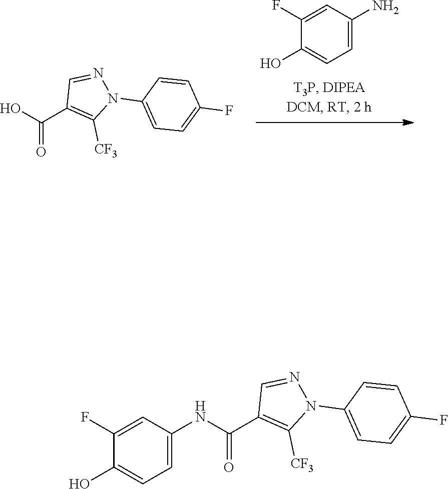 [SMILES: Nc1ccc(O)c(F)c1 ; O=C(O)c1cnn(-c2ccc(F)cc2)c1C(F)(F)F ; O=C(Nc1ccc(O)c(F)c1)c1cnn(-c2ccc(F)cc2)c1C(F)(F)F]

A mixture of 1-(4-fluorophenyl)-5-(trifluoromethyl)-1H-pyrazole-4-carboxylic acid (260 mg, 948 μmol, 1 eq), 4-amino-2-fluorophenol (181 mg, 1.42 mmol, 1.5 eq) and di isopropyl ethyl amine (339 μL, 1.9 mmol, 2 eq) in DCM (2.6 mL) was added tripropyl-1,3,5,2λ⁵,4λ⁵,6λ⁵-trioxatriphosphinane-2,4,6-trione (365 μL, 1.42 mmol, 1.5 eq) at room temperature. Then the reaction mixture was stirred at room temperature for 16 h. Then the reaction mixture was quenched with water (25 mL) and extracted with ethyl acetate (30 mL×3). The combined organic layer was washed with 0.5 M HCl (25 mL), then dried by anhydrous Na₂SO₄ filtered and concentrated to get crude product as brown color gummy liquid. The crude product was purified by flash column by using 100-200 silica mesh with 30% of EtOAc in n-hexane mobile phase to obtain N-(3-fluoro-4-hydroxyphenyl)-1-(4-fluorophenyl)-5-(trifluoromethyl)-1H-pyrazole-4-carboxamide as off white solid (200 mg, 35.77%). LCMS: 384.29 (M+H)⁺. ¹H NMR (400 MHz, DMSO-d₆): δ=10.44 (s, 1H), 9.71 (s, 1H), 8.27 (s, 1H), 7.63-7.57 (m, 3H), 7.47-7.43 (m, 2H), 7.23 (d, J=10 Hz, 1H), 6.93 (t, J=9.6 Hz, 1H).

### Step 4: Synthesis of N-{4-[(2-chloropyrimidin-4-yl) oxy]-3-fluorophenyl}-1-(4-fluorophenyl)-5-(trifluoromethyl)-1H-pyrazole-4-carboxamide

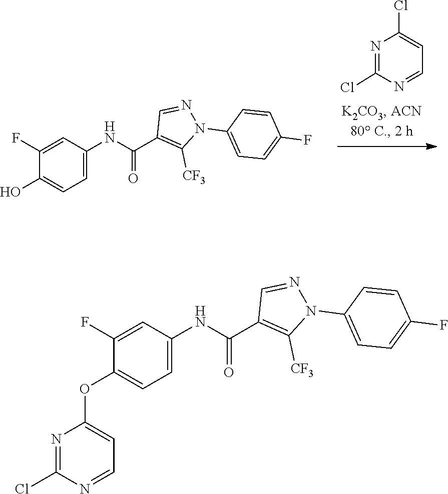

To a solution of N-(3-fluoro-4-hydroxyphenyl)-1-(4-fluorophenyl)-5-(trifluoromethyl)-1H-pyrazole-4-carboxamide (300 mg, 783 μmol, 1 eq) and 2,4-dichloropyrimidine (233 mg, 1.57 mmol, 2 eq) in acetonitrile (20 mL) was added K₂CO₃ (85.3 mg, 861 μmol, 1.1 eq) at room temperature. Then the reaction mixture was stirred at 80° C. for 2 h. After 2 h, the reaction mixture was cooled to room temperature and ice water (30 mL) was added, then extract with EtOAc (3×50 mL). The combined organic layers were dried over Na₂SO₄, filtered and concentrated under reduced pressure to obtained brown color gummy crude compound. The crude was purified by flash column by using 100-200 silica mesh with 30% of EtOAc in hexane mobile phase to obtain N-{4-[(2-chloropyrimidin-4-yl)oxy]-3-fluorophenyl}-1-(4-fluorophenyl)-5-(trifluoromethyl)-1H-pyrazole-4-carboxamide (300 mg, 77.31%). LCMS: 496.1 (M+H)⁺. ¹H NMR (400 MHz, DMSO-d₆): δ=10.85 (s, 1H), 8.70 (d, J=6 Hz, 1H), 8.35 (s, 1H), 7.87 (m, 1H), 7.66-7.63 (m, 2H), 7.54 (m, 1H), 7.49-7.44 (m, 3H), 7.36 (d, J=5.6 Hz, 1H).

### Step 5: Synthesis of N-(3-fluoro-4-((2-((3-morpholinophenyl) amino) pyrimidin-4-yl) oxy) phenyl)-1-(4-fluorophenyl)-5-(trifluoromethyl)-1H-pyrazole-4-carboxamide

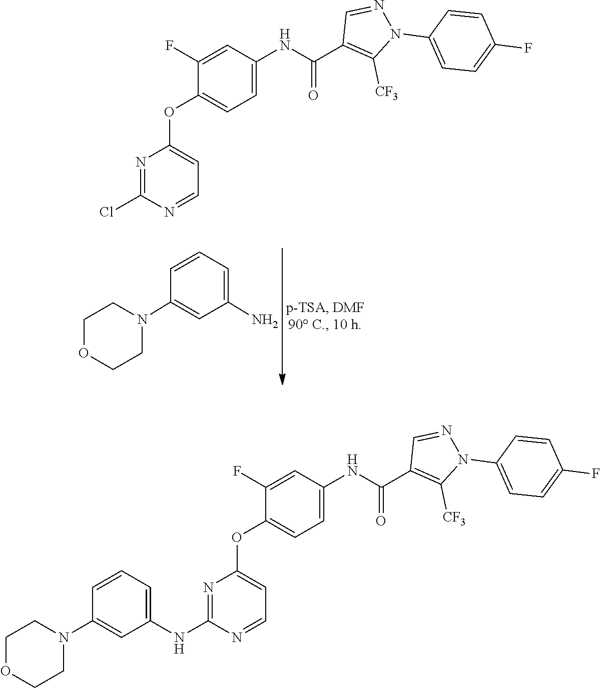

A mixture of N-{4-[(2-chloropyrimidin-4-yl)oxy]-3-fluorophenyl}-1-(4-fluorophenyl)-5-(trifluoromethyl)-1H-pyrazole-4-carboxamide (150 mg, 303 μmol, 1 eq), and 3-(morpholin-4-yl)aniline (53.9 mg, 303 μmol, 1 eq) in DMF (5 mL) was added 4-methylbenzene-1-sulfonic acid hydrate (230 mg, 1.21 mmol, 4 eq) at room temperature. Then the reaction mixture was stirred at 90° C. for 16 h. The reaction was cooled to room temperature, quenched with water (25 mL) and extract with ethyl acetate (30 mL×3), the combined organic layers were dried over Na2SO4, filtered, and concentrated under reduced pressure to obtained light brown color gummy crude compound. The crude was purified by flash column by using 100-200 silica mesh with 50% of EtOAc in n-hexane mobile phase to obtain N-{3-fluoro-4-[(2-{[3-(morpholin-4-yl)phenyl]amino}pyrimidin-4-yl)oxy]phenyl}-1-(4-fluorophenyl)-5-(trifluoromethyl)-1H-pyrazole-4-carboxamide (40 mg, 20.74%). LCMS: 638.71 (M+H)⁺. ¹H NMR (400 MHz, DMSO-d₆): δ=10.84 (s, 1H), 9.44 (s, 1H), 8.38 (d, J=5.6 Hz, 1H), 8.34 (s, 1H), 7.86 (dd, J=12.4 Hz, 2 Hz, 1H), 7.66-7.63 (m, 2H), 7.53-7.40 (m, 4H), 7.08 (br.s, 1H), 7.02-7.00 (m, 1H), 6.93 (t, J 15=8 Hz, 1H), 6.54 (d, J=5.6 Hz, 1H), 6.48 (d, J=7.6 Hz, 1H), 3.69 (t, J=4.4 Hz, 4H), 2.93 (t, J=4.8 Hz, 4H). HPLC: 95.73%.

### Synthetic Example 2

### Synthesis of N-(3-Fluoro-4-{[6-(1-Methyl-1H-Imidazol-4-Yl)Thieno[3,2-D]Pyrimidin-4-Yl]Oxy}Phenyl)-1-(4-Fluorophenyl)-5-(Trifluoromethyl)-1H-Pyrazole-4-Carboxamide

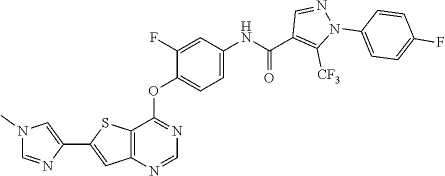 [SMILES: Cn1cnc(-c2cc3ncnc(Oc4ccc(NC(=O)c5cnn(-c6ccc(F)cc6)c5C(F)(F)F)cc4F)c3s2)c1]

### Step 1: Synthesis of N-[4-({6-bromothieno [3,2-d]pyrimidin-4-yl}oxy)-3-fluorophenyl]-1-(4-fluorophenyl)-5-(trifluoromethyl)-1H-pyrazole-4-carboxamide

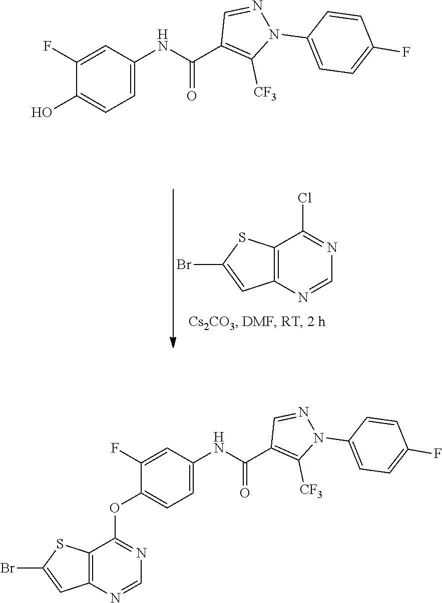 [SMILES: O=C(Nc1ccc(O)c(F)c1)c1cnn(-c2ccc(F)cc2)c1C(F)(F)F ; Clc1ncnc2cc(Br)sc12 ; O=C(Nc1ccc(Oc2ncnc3cc(Br)sc23)c(F)c1)c1cnn(-c2ccc(F)cc2)c1C(F)(F)F]

To stirred a solution of N-(3-fluoro-4-hydroxyphenyl)-1-(4-fluorophenyl)-5-(trifluoromethyl)-1H-pyrazole-4-carboxamide (307 mg, 802 μmol, 1 eq) in DMF (5 mL) was added dipotassium carbonate (522 mg, 1.6 mmol, 2 eq) stirred at room temperature for 0.5 h then 6-bromo-4-chlorothieno[3,2-d]pyrimidine (0.2 g, 802 μmol, 1 eq) was added. The reaction mixture was stirred at room temperature for 2 h. After 2 h the reaction mixture was quenched with ice water (30 mL), solid precipitate was formed and filter the solid dried under reduced pressure to obtained N-[4-({6-bromothieno[3,2-d]pyrimidin-4-yl}oxy)-3-fluorophenyl]-1-(4-fluorophenyl)-5-(trifluoromethyl)-1H-pyrazole-4-carboxamide as an off white solid (400 mg, 83.68%). LCMS: 596.59 (M)⁺, 598.59 (M+2)⁺. ¹H NMR (400 MHz, DMSO-d₆): δ=8.72 (s, 1H), 8.28 (s, 1H), 7.99 (s, 1H), 7.93-7.87 (m, 1H), 7.64-7.60 (m, 2H), 7.47-7.41 (m, 5H).

### Step 2: Synthetic scheme for 1-methyl-4-(tributylstannyl)-1H-imidazole

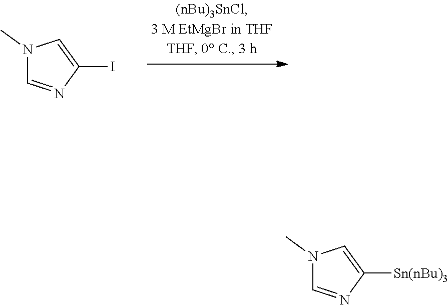

To a stirred solution of 4-iodo-1-methylimidazole (2 g, 9.62 mmol, 1 eq) in DCM (5 mL) was added 3M ethyl magnesium bromide (2.66 mL, 7.69 mmol, 0.8 eq) dropwise successively, at room temperature under nitrogen protection. After 30 minutes tributyl(chloro)stannane (3.29 mL, 11.5 mmol, 1.2 eq) was added and stirred at room temperature for 16 h. Then the reaction mixture was quenched with saturated aqueous ammonium chloride solution (50 mL), diluted with water (30 mL), then extract with ethyl acetate (3×100 mL). The combined organic layers were, dried over anhydrous sodium sulfate, filtered, and evaporated to dryness under reduced pressure to obtained 1-methyl-4-(tributylstannyl)-1H-imidazole and used directly for the next step without next step (3.3 g, 92.47%). LCMS: 373.2 (M+H)⁺.

### Step 3: Synthesis of N-(3-fluoro-4-{[6-(1-methyl-1H-imidazol-4-yl) thieno [3, 2-d]pyrimidin-4-yl]oxy}phenyl)-1-(4-fluorophenyl)-5-(trifluoromethyl)-1H-pyrazole-4-carboxamide

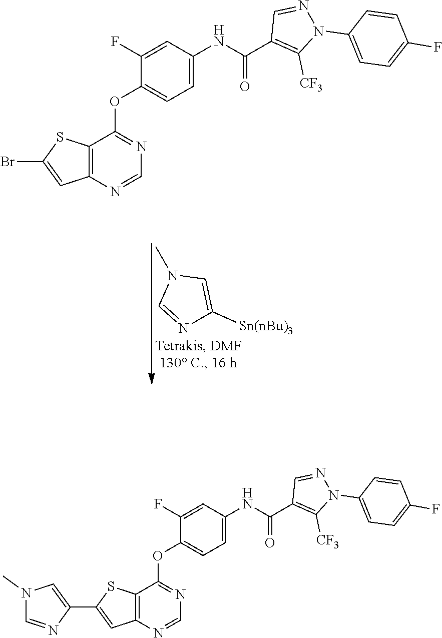

To a stirred solution of N-[4-({6-bromothieno[3,2-d]pyrimidin-4-yl}oxy)-3-fluorophenyl]-1-(4-fluorophenyl)-5-(trifluoromethyl)-1H-pyrazole-4-carboxamide (0.2 g, 335 μmol, 1 eq) and 1-methyl-4-(tributylstannyl)-1H-imidazole (187 mg, 503 μmol, 1.5 eq) in DMF (3 mL) was degasses with nitrogen for 10 minutes. Then tetrakis(triphenylphosphane) palladium (38 mg, 33.5 μmol, 0.1 eq) was added and again degassed with nitrogen for 3 minutes. Then the reaction was stirred at 130° C. for 16 h. Then the reaction mixture cooled to room temperature, quenched with water (20 mL) and extract with ethyl acetate (30 mL×3). The combined organic layers were dried over Na₂SO₄ filtered and concentrate under reduced pressure to get crude compound. The crude was purified by flash column by using 100-200 silica mesh with 10% of MeOH in DCM mobile phase to obtain N-(3-fluoro-4-{[6-(1-methyl-1H-imidazol-4-yl)thieno[3,2-d]pyrimidin-4-yl]oxy}phenyl)-1-(4-fluorophenyl)-5-(trifluoromethyl)-1H-pyrazole-4-carboxamide as an off-white solid (79 mg, 39.42%). LCMS: 598.5 (M+H)⁺. ¹H NMR (400 MHz, DMSO-d₆): δ=10.85 (s, 1H), 8.64 (s, 1H), 8.37 (s, 1H), 8.02 (s, 1H), 7.90-7.87 (m, 1H), 7.81-7.80 (m, 2H), 7.67-7.63 (m, 2H), 7.55-7.51 (m, 2H), 7.49-7.45 (m, 2H), 3.76 (s, 3H). HPLC: 98.81%.

### Synthetic Example 3

### Synthesis of N-(4-{[2-Amino-3-(1-Methyl-1H-Imidazol-4-Yl) Pyridin-4-Yl]Oxy}-3-Fluorophenyl)-1-(4-Fluorophenyl)-5-(Trifluoromethyl)-1H-Pyrazole-4-Carboxamide

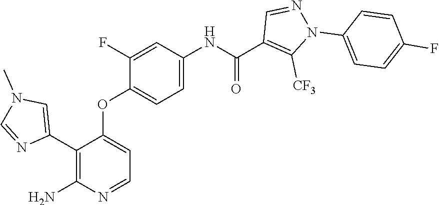 [SMILES: Cn1cnc(-c2c(Oc3ccc(NC(=O)c4cnn(-c5ccc(F)cc5)c4C(F)(F)F)cc3F)ccnc2N)c1]

### Step 1: Synthesis of 4-(2-fluoro-4-nitrophenoxy)-3-iodopyridin-2-amine

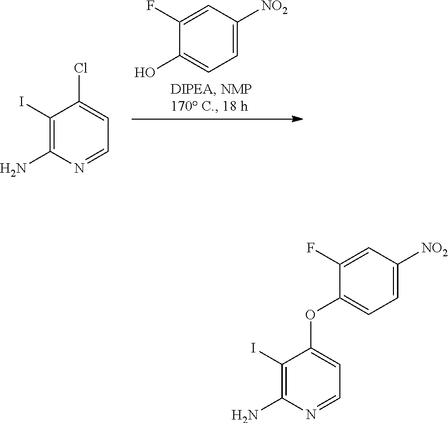 [SMILES: Nc1nccc(Cl)c1I ; O=[N+]([O-])c1ccc(O)c(F)c1 ; Nc1nccc(Oc2ccc([N+](=O)[O-])cc2F)c1I]

A mixture of 4-chloro-3-iodopyridin-2-amine (1.5 g, 5.89 mmol, 1 eq) and 2-fluoro-4-nitrophenol (1.85 g, 11.8 mmol, 2 eq) and DIPA (1.54 mL, 8.84 mmol, 1.5 eq) in NMP (5 mL) was placed in a glass pressure vessel and heated rapidly to 170° C. The heating was continued for 18 h. The volatile components were distilled off under reduced pressure and the viscous residue poured into ice-water (300 mL). The mixture was adjusted pH to 7.5 with saturated aq. NaHCO₃ solution then extracted with ethyl acetate (3×100 mL), the combined ethyl acetate layers were, washed with brine, then dried over Na₂SO₄, filtered, and concentrate to obtained crude. The crude was purified by combi-flash using (40 g) with gradient elution of 10-15% EtOAc in n-hexane to afford the 4-(2-fluoro-4-nitrophenoxy)-3-iodopyridin-2-amine as yellow solid (1.1 g, 49.7%). LCMS: 376.23 (M+H)⁺. ¹H NMR (400 MHz, DMSO-d₆): δ=8.40 (dd, J=10.8 Hz, 2.4 Hz, 1H), 8.14-8.12 (m, 1H), 7.88 (d, J=5.6 Hz, 1H), 7.33 (t, J=8.4 Hz, 1H), 6.41 (br.s, 2H), 6.20 (d, J=5.6 Hz, 1H).

### Step 2: Synthesis of 4-(2-fluoro-4-nitrophenoxy)-3-(1-methyl-1H-imidazol-4-yl) pyridin-2-amine

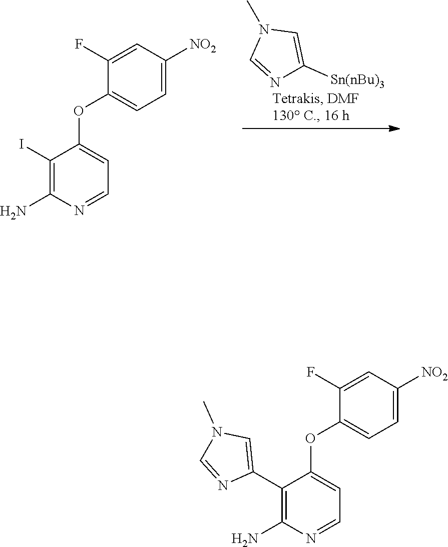

To a stirred solution of 4-(2-fluoro-4-nitrophenoxy)-3-iodopyridin-2-amine (600 mg, 1.6 mmol, 1 eq) and 1-methyl-4-(tributylstannyl)-1H-imidazole (891 mg, 2.4 mmol, 1.5 eq) in DMF (5 mL) was degassed with nitrogen for 15 minutes. After 15 minutes tetrakis(triphenylphosphine)palladium(0) (185 mg, 160 μmol, 0.1 eq) was added to the reaction mixture and heated to 130° C. for 16 h. Then the reaction mixture was cooled to room temperature, quenched with water (20 mL) and extract with ethyl acetate (30 mL×3). The combined organic layers were dried over Na₂SO₄ filtered and concentrate under reduced pressure to get crude compound. The crude was purified by flash column by using 100-200 silica mesh with 50% of EtOAc in hexane mobile phase to obtain 4-(2-fluoro-4-nitrophenoxy)-3-(1-methyl-1H-imidazol-4-yl)pyridin-2-amine as a pale brown color solid (160 mg, 30.38%). LCMS: 330.1 (M+H)⁺. ¹H NMR (400 MHz, DMSO-d₆): δ=13.38 (s, 1H), 11.23 (s, 1H), 9.12 (t, J=8.8 Hz, 1H), 8.14-8.09 (m, 2H), 7.96 (t, J=5.6 Hz, 2H), 7.85 (s, 1H), 6.68 (d, J=5.6 Hz, 1H), 3.78 (s, 3H).

### Step 3: Synthesis of 4-(4-amino-2-fluorophenoxy)-3-(1-methyl-1H-imidazol-4-yl) pyridin-2-amine

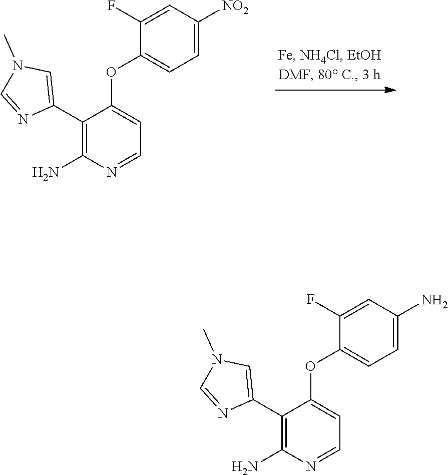 [SMILES: Cn1cnc(-c2c(Oc3ccc([N+](=O)[O-])cc3F)ccnc2N)c1 ; Cn1cnc(-c2c(Oc3ccc(N)cc3F)ccnc2N)c1]

To a stirred solution of 4-(2-fluoro-4-nitrophenoxy)-3-(1-methyl-1H-imidazol-4-yl)pyridin-2-amine (0.1 g, 304 μmol, 1 eq) in mixture of ethanol (1 mL) and DMF (1 mL) was added Iron powder (170 mg, 3.04 mmol, 10 eq) and NH₄Cl (325 mg, 6.07 mmol, 20 eq) at 0° C. Then the reaction was stirred at 80° C. for 3 h. Then the reaction mixture was cooled to room temperature diluted with ethanol and filtered through a pad of diatomaceous earth (i.e., Celite®). The filtrated was concentrated diluted with water (20 mL) and extract with ethyl acetate (30 mL×3). The combined organic layers were dried over Na₂SO₄ filtered and concentrate under reduced pressure to get crude compound. The crude was purified by flash column by using 100-200 silica mesh with 50% of EtOAc in hexane mobile phase to obtain 4-(4-amino-2-fluorophenoxy)-3-(1-methyl-1H-imidazol-4-yl)pyridin-2-amine as a pale brown color solid (30 mg, 22%). LCMS: 300.39 (M+H)⁺.

### Step 4: Synthesis of N-(4-{[2-amino-3-(1-methyl-1H-imidazol-4-yl)pyridin-4-yl]oxy}-3-fluorophenyl)-1-(4-fluorophenyl)-5-(trifluoromethyl)-1H-pyrazole-4-carboxamide

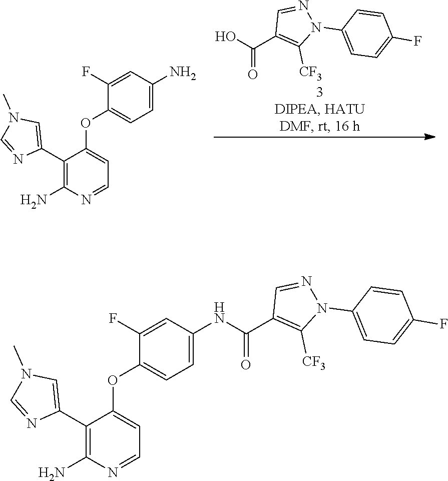 [SMILES: Cn1cnc(-c2c(Oc3ccc(N)cc3F)ccnc2N)c1 ; O=C(O)c1cnn(-c2ccc(F)cc2)c1C(F)(F)F ; Cn1cnc(-c2c(Oc3ccc(NC(=O)c4cnn(-c5ccc(F)cc5)c4C(F)(F)F)cc3F)ccnc2N)c1]

A mixture of 1-(4-fluorophenyl)-5-(trifluoromethyl)-1H-pyrazole-4-carboxylic acid (27.5 mg, 0.1 mmol, 1 eq), 4-(4-amino-2-fluorophenoxy)-3-(1-methyl-1H-imidazol-4-yl)pyridin-2-amine (30 mg, 0.1 mmol, 1 eq) in DMF (3 mL) was added DIPEA (0.035 mL, 0.2 mmol, 2 eq) and HATU (47.2 mg, 0.2 mmol, 2 eq.) at room temperature. Then the reaction mixture stirred at room temperature for 16 h. Then the reaction mixture was quenched with ice water (20 mL) and extract with ethyl acetate (30 mL×3). The combined organic layers were dried over Na₂SO₄ filtered and concentrate under reduced pressure to get crude compound. The crude was purified by flash column by using 100-200 silica mesh with 10% MeOH in DCM mobile phase to obtain N-(4-{[2-amino-3-(1-methyl-1H-imidazol-4-yl)pyridin-4-yl]oxy}-3-fluorophenyl)-1-(4-fluorophenyl)-5-(trifluoromethyl)-1H-pyrazole-4-carboxamide (20 mg, 35.92%). LCMS: 556.65 (M+H)⁺. ¹H NMR (400 MHz, DMSO-d₆): δ=12.19 (s, 1H), 10.88 (s, 1H), 10.55 (s, 1H), 8.62 (t, J=9.2 Hz, 1H), 8.31 (s, 1H), 7.88 (s, 1H), 7.80 (d, J=5.6 Hz, 1H), 7.78 (s, 1H), 7.73 (dd, J=13.6 Hz, 2 Hz, 1H), 7.64-7.61 (m, 2H), 7.45 (t, J=8.8 Hz, 2H), 7.31 (d, J=10.4 Hz, 1H), 6.48 (d, J=5.6 Hz, 1H), 3.77 (s, 3H). HPLC: 99.23%.

### Synthetic Example 4

### Synthesis of N-(4-((2-Bromopyrazolo [1, 5-A]Pyrimidin-7-Yl) Oxy)-3-Fluorophenyl)-1-(4-Fluorophenyl)-5-(Trifluoromethyl)-1H-Pyrazole-4-Carboxamide

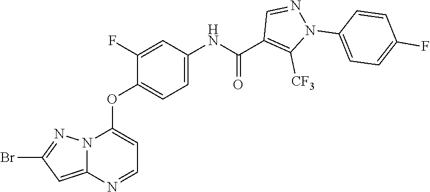 [SMILES: O=C(Nc1ccc(Oc2ccnc3cc(Br)nn23)c(F)c1)c1cnn(-c2ccc(F)cc2)c1C(F)(F)F]

### Step 1: Synthesis of N-(4-((2-bromopyrazolo [1,5-a]pyrimidin-7-yl) oxy)-3-fluorophenyl)-1-(4-fluorophenyl)-5-(trifluoromethyl)-1H-pyrazole-4-carboxamide

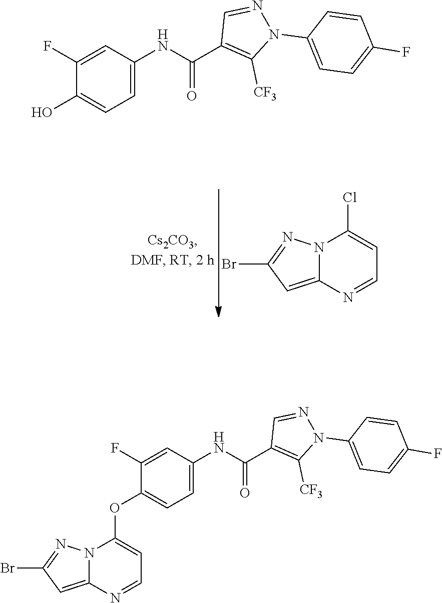 [SMILES: O=C(Nc1ccc(O)c(F)c1)c1cnn(-c2ccc(F)cc2)c1C(F)(F)F ; Clc1ccnc2cc(Br)nn12 ; O=C(Nc1ccc(Oc2ccnc3cc(Br)nn23)c(F)c1)c1cnn(-c2ccc(F)cc2)c1C(F)(F)F]

To a stirred solution of N-(3-fluoro-4-hydroxyphenyl)-1-(4-fluorophenyl)-5-(trifluoromethyl)-1H-pyrazole-4-carboxamide (330 mg, 860 μmol, 1 eq) in DMF (5 mL) was added cesium carbonate (561 mg, 1.72 mmol, 2 eq) at RT. The reaction mixture was stirred for 0.5 h at room temperature. Then 2-bromo-7-chloropyrazolo [1,5-a]pyrimidine (200 mg, 860 μmol, 1 eq) was added to the reaction mixture and stirred at room temperature for 2 2 hh. After 2 h the reaction mixture was quenched with ice water (3 mL), extract with ethyl acetate (3×5 mL), the combined organic layers were dried over Na₂SO₄ filtered and concentrate under reduced pressure to get crude compound. The crude was purified by flash column by using 100-200 silica mesh with 50% of EtOAc in hexane mobile phase to obtain N-(4-((2-bromopyrazolo[1,5-a]pyrimidin-7-yl)oxy)-3-fluorophenyl)-1-(4-fluorophenyl)-5-(trifluoromethyl)-1H-pyrazole-4-carboxamide as an off-white solid (200 mg, 40.13%).%). LCMS: 579.62 (M)⁺. ¹H NMR (400 MHz, DMSO-d₆): δ=10.95 (s, 1H), 8.44 (d, J=4.8 Hz, 1H), 8.36 (s, 1H), 8.02 (dd, J=12.8 Hz, 2 Hz, 1H), 7.70-7.62 (m, 4H), 7.47 (t, J=8.8 Hz, 2H), 7.01 (s, 1H), 6.35 (d, J=4.8 Hz, 1H). HPLC: 98.31%.

### Synthetic Example 5

### Synthesis of N-(4-{[2-Amino-3-(1-Methyl-1H-Pyrazol-4-Yl) Pyridin-4-Yl]Oxy}-3-Fluorophenyl)-1-(4-Fluorophenyl)-5-(Trifluoromethyl)-1H-Pyrazole-4-Carboxamide

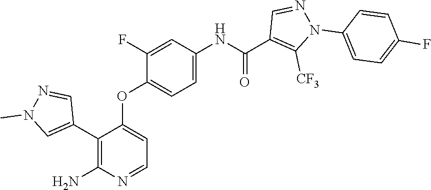 [SMILES: Cn1cc(-c2c(Oc3ccc(NC(=O)c4cnn(-c5ccc(F)cc5)c4C(F)(F)F)cc3F)ccnc2N)cn1]

### Step 1: Synthesis of 4-(4-amino-2-fluorophenoxy)-3-(1-methyl-1H-pyrazol-4-yl) pyridin-2-amine

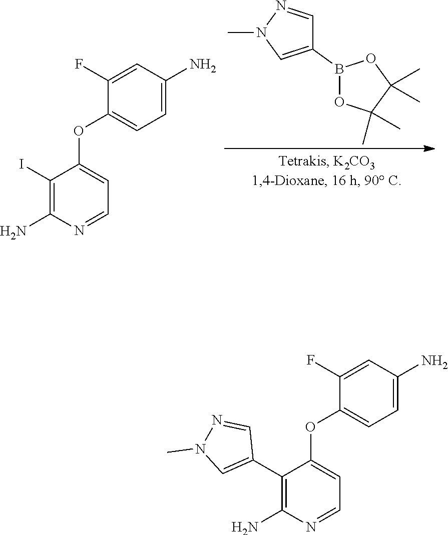

To a stirred solution of 4-(4-amino-2-fluorophenoxy)-3-iodopyridin-2-amine (170 mg, 493 μmol, 1 eq) and 1-methyl-4-(4,4,5,5-tetramethyl-1,3,2-dioxaborolan-2-yl)-1H-pyrazole (154 mg, 739 μmol, 1.5 eq) in 1,4-dioxane (5 mL) and water (2 mL) was added potassium carbonate (204 mg, 1.48 mmol, 3 eq) and degassed for 5 minutes with nitrogen gas. Then tetrakis(triphenylphosphane) palladium (57 mg, 49.3 μmol, 0.1 eq) was added at room temperature, and again degassed with nitrogen gas. Then the reaction mixture was stirred at 90° C. for 16 h. The progress of the reaction was monitored by TLC/LCMS. After completion of the reaction, the reaction mixture was concentrated, diluted with ethyl acetate (20 mL), and washed with sat NaHCO₃ solution (3 mL) and water (10 mL). The organic layer was dried over Na₂SO₄, filtered, and concentrated under reduced pressure to get crude compound. The crude was purified by flash column by using 100-200 silica mesh with 5% of MeOH in DCM mobile phase to obtain 4-(4-amino-2-fluorophenoxy)-3-(1-methyl-1H-pyrazol-4-yl)pyridin-2-amine (65 mg, 44.09%). LCMS: 300.28 (M+H)⁺. ¹H NMR (400 MHz, DMSO-d₆): δ=7.87 (s, 1H), 7.70 (d, J=5.6 Hz, 1H), 7.60 (s, 1H), 6.89 (t, J=9.2 Hz, 1H), 6.47 (dd, J=13.2 Hz, 2.4 Hz, 1H), 6.37 (dd, J=8.8 Hz, 2 Hz, 1H), 5.84 (d, J=5.6 Hz, 1H), 5.75 (s, 2H), 5.37 (s, 2H), 3.88 (s, 3H).

### Step 2: Synthesis of N-(4-{[2-amino-3-(1-methyl-1H-pyrazol-4-yl) pyridin-4-yl]oxy}-3-fluorophenyl)-1-(4-fluorophenyl)-5-(trifluoromethyl)-1H-pyrazole-4-carboxamide

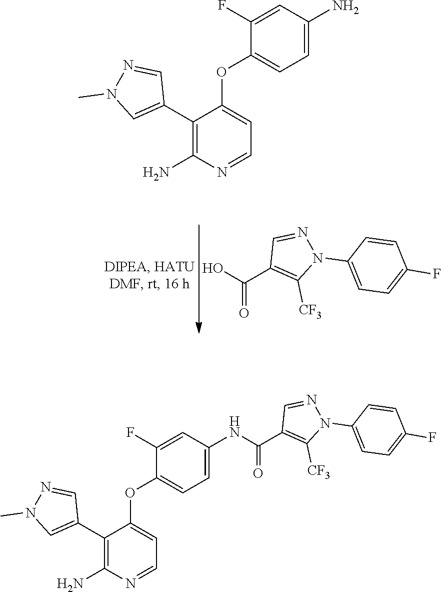 [SMILES: Cn1cc(-c2c(Oc3ccc(N)cc3F)ccnc2N)cn1 ; O=C(O)c1cnn(-c2ccc(F)cc2)c1C(F)(F)F ; Cn1cc(-c2c(Oc3ccc(NC(=O)c4cnn(-c5ccc(F)cc5)c4C(F)(F)F)cc3F)ccnc2N)cn1]

To stirred a solution of 4-(4-amino-2-fluorophenoxy)-3-(1-methyl-1H-pyrazol-4-yl)pyridin-2-amine (65 mg, 217 μmol, 1 eq) and 1-(4-fluorophenyl)-5-(trifluoromethyl)-1H-pyrazole-4-carboxylic acid (59.5 mg, 217 μmol, 1 eq) in DMF (2 mL) was added DIPEA (77.5 μL, 434 μmol, 2 eq) and HATU (102 mg, 434 μmol, 2eq) at room temperature. Then the reaction mixture stirred at room temperature for 16 h. Then the reaction mixture was quenched with ice water (20 mL) and extract with ethyl acetate (30 mL×3). The combined organic layers were dried over Na₂SO₄ filtered and concentrate under reduced pressure to get crude compound. The crude was purified by flash column by using 100-200 silica mesh with 10% MeOH in DCM mobile phase to obtain N-(4-{[2-amino-3-(1-methyl-1H-pyrazol-4-yl)pyridin-4-yl]oxy}-3-fluorophenyl)-1-(4-fluorophenyl)-5-(trifluoromethyl)-1H-pyrazole-4-carboxamide (25 mg, 20.72%). LCMS: 556.56 (M+H)⁺. ¹H NMR (400 MHz, DMSO-d₆): δ=10.79 (s, 1H), 8.33 (s, 1H), 7.90 (s, 1H), 7.85 (dd, J=12.8 Hz, 2 Hz, 1H), 7.75 (d, J=5.6 Hz, 1H), 7.65-7.61 (m, 3H), 7.49-7.44 (m, 3H), 7.27 (t, J=8.8 Hz, 1H), 5.93 (d, J=5.6 Hz, 1H), 5.69 (s, 2H), 3.89 (s, 3H). HPLC: 99.65%.

### Synthetic Example 6

### Synthesis of N-(4-((2-Amino-3-(1-Methyl-1H-Pyrazol-4-Yl) Pyridin-4-Yl) Oxy)-3-Fluorophenyl)-1-(4-Fluorophenyl)-5-(Trifluoromethyl)-1H-1, 2, 3-Triazole-4-Carboxamide

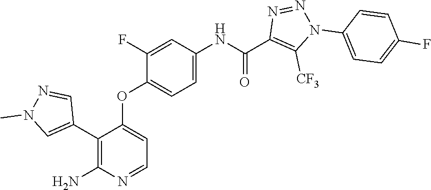 [SMILES: Cn1cc(-c2c(Oc3ccc(NC(=O)c4nnn(-c5ccc(F)cc5)c4C(F)(F)F)cc3F)ccnc2N)cn1]

### Step 1: Synthesis of 1-azido-4-fluorobenzene

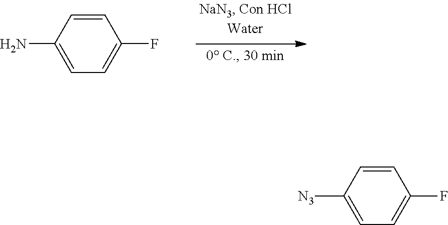 [SMILES: Nc1ccc(F)cc1 ; [N-]=[N+]=Nc1ccc(F)cc1]

To a stirred solution of 4-fluoroaniline (1 g, 9 mmol, 1 eq) in 10 mL of 15% HCl (5 mL water:5 mL concentrated HCl) was added NaNO₂ (745 mg, 10.8 mmol, 1.2 eq) in water (30 mL) solution drop wise at 0° C. After the completion of addition, the reaction mixture was stirred at this temperature for 30 min. Then sodium azide (1.17 g, 18 mmol, 2 eq) in water (5 mL) solution was added to the above reaction mixture in a dropwise manner at 0° C. After addition the reaction was stirred at 0° C. for 1 h. The progress of the reaction was monitored by TLC. After completion of the reaction, extract the product with ethyl acetate (50 mL), followed by washed with water up to neutral pH. Then the organic layer was dried over anhydrous sodium sulfate and then concentrate by distillation at room temperature to obtain 1-azido-4-fluorobenzene as a crude and this was used to next step without any further purification (1 g, crude). ¹H NMR (400 MHz, CDCl₃): δ=7.10-7.04 (m, 2H), 7.03-6.99 (m, 2H).

### Step 2: Synthesis of ethyl 1-(4-fluorophenyl)-5-(trifluoromethyl)-1H-1, 2, 3-triazole-4-carboxylate

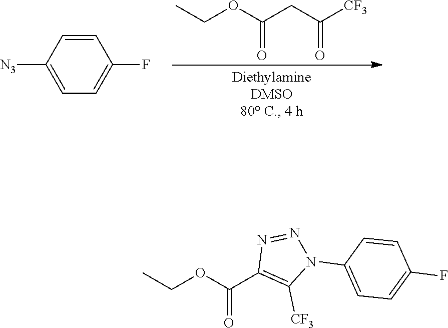 [SMILES: [N-]=[N+]=Nc1ccc(F)cc1 ; CCOC(=O)CC(=O)C(F)(F)F ; CCOC(=O)c1nnn(-c2ccc(F)cc2)c1C(F)(F)F]

To a stirred solution of appropriate 1-azido-4-fluorobenzene (1 g, 7.29 mmol, 1 eq), ethyl 4, 4, 4-trifluoro-3-oxobutanoate (1.09 mL, 7.29 mmol, 1 eq) and diethyl amine (377 μL, 3.65 mmol, 0.5 eq) in DMSO (10 mL) was heated at 80° C. for 4 h. After 4 h the reaction mixture was poured into ice water, extracted with dichloromethane (50 mL) and separated the organic phase, washed with brine, dry over Na₂SO₄, filter and concentrate to obtained crude product. The crude was purified by flash column by using 100-200 silica mesh with 20% of EtOAc in hexane mobile phase to obtain ethyl 1-(4-fluorophenyl)-5-(trifluoromethyl)-1H-1,2,3-triazole-4-carboxylate as break red color oil (1.4 g, 63.31%). LCMS: 304.36 (M+H)⁺. ¹H NMR (400 MHz, CDCl₃): δ=7.51-7.48 (m, 2H), 7.32-7.28 (m, 2H), 4.53 (q, J=14.4 Hz, 7.2 Hz, 2H), 1.47 (t, J=7.2 Hz, 3H).

### Step 3: Synthesis of 1-(4-fluorophenyl)-5-(trifluoromethyl)-1H-1,2,3-triazole-4-carboxylic acid

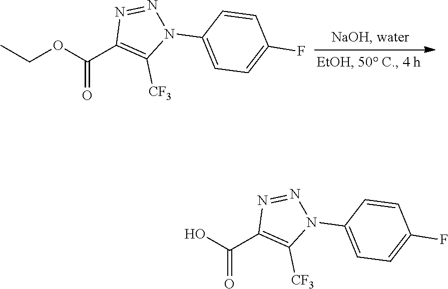 [SMILES: CCOC(=O)c1nnn(-c2ccc(F)cc2)c1C(F)(F)F ; O=C(O)c1nnn(-c2ccc(F)cc2)c1C(F)(F)F]

To a stirred solution of ethyl 1-(4-fluorophenyl)-5-(trifluoromethyl)-1H-1,2,3-triazole-4-carboxylate (1.4 g, 4.62 mmol, 1.0 eq) in EtOH:H₂O (10 mL:10 mL) was added sodium hydroxide (369 mg, 9.23 mmol, 2.0 eq) at room temperature and stirred at 50° C. for 4 h. After complete conversion by TLC, the reaction mixture was concentrated under reduced pressure to get crude residue, the crude residue was diluted with water and washed with DCM, water layer was acidified with 10% HCl solution under cooling to obtained precipitate, the precipitate was collected by filtration and dried under reduced pressure get the 1-(4-fluorophenyl)-5-(trifluoromethyl)-1H-1,2,3-triazole-4-carboxylic acid as off white solid (1.15 g, 90.52%). LCMS: 274.0 (M−H)⁺. ¹H NMR (400 MHz, DMSO-d₆): δ=14.14 (br, 1H), 7.82-7.79 (m, 2H), 7.54-7.49 (m, 2H).

### Step 4: Synthesis of N-(4-((2-amino-3-(1-methyl-1H-pyrazol-4-yl) pyridin-4-yl) oxy)-3-fluorophenyl)-1-(4-fluorophenyl)-5-(trifluoromethyl)-1H-1, 2, 3-triazole-4-carboxamide

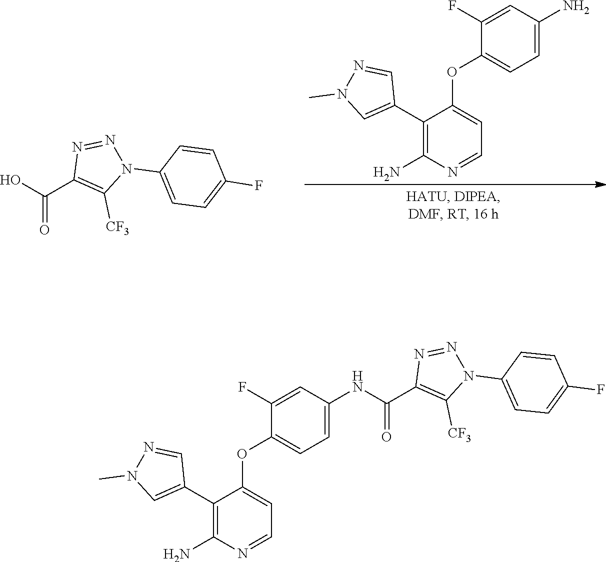 [SMILES: O=C(O)c1nnn(-c2ccc(F)cc2)c1C(F)(F)F ; Cn1cc(-c2c(Oc3ccc(N)cc3F)ccnc2N)cn1 ; Cn1cc(-c2c(Oc3ccc(NC(=O)c4nnn(-c5ccc(F)cc5)c4C(F)(F)F)cc3F)ccnc2N)cn1]

A mixture of 1-(4-fluorophenyl)-5-(trifluoromethyl)-1H-1,2,3-triazole-4-carboxylic acid (91.9 mg, 334 μmol, 1 eq) and 4-(4-amino-2-fluorophenoxy)-3-(1-methyl-1H-pyrazol-4-yl)pyridin-2-amine (100 mg, 334 μmol, 1.2 eq) in DMF (5 mL) was added DIPEA (119 μL, 668 μmol, 2 eq) and HATU (157 mg, 668 μmol, 2eq) at room temperature. Then the reaction mixture stirred at room temperature for 16 h. Then the reaction mixture was quenched with ice water (20 mL) and extract with ethyl acetate (30 mL×3). The combined organic layers were dried over Na₂SO₄ filtered and concentrate under reduced pressure to get crude compound. The crude was purified by flash column by using 100-200 silica mesh with 10% MeOH in DCM mobile phase to obtain N-(4-((2-amino-3-(1-methyl-1H-pyrazol-4-yl)pyridin-4-yl)oxy)-3-fluorophenyl)-1-(4-fluorophenyl)-5-(trifluoromethyl)-1H-1,2,3-triazole-4-carboxamide as an off-white solid (85 mg, 45%). LCMS: 557.46 (M+H)⁺. ¹H NMR (400 MHz, DMSO-d₆): δ=11.25 (s, 1H), 7.95 (dd, J=12.8 Hz, 2.4 Hz, 1H), 7.91 (s, 1H), 7.85-7.82 (m, 2H), 7.76 (d, J=5.6 Hz, 1H), 7.70 (d, J=8.8 Hz, 1H), 7.62 (s, 1H), 7.55 (t, J=8.8 Hz, 2H), 7.29 (t, J=8.8 Hz, 1H), 5.95 (d, J=6 Hz, 1H), 5.70 (s, 2H), 3.89 (s, 3H). HPLC: 99.39%.

### Synthetic Example 7

### Synthesis of N-(3-Fluoro-4-{[2-(1-Methyl-1H-Pyrazol-4-Yl)Pyrazolo[1,5-A]Pyrimidin-7-Yl]Oxy}Phenyl)-1-(4-Fluorophenyl)-5-(Trifluoromethyl)-1H-Pyrazole-4-Carboxamide

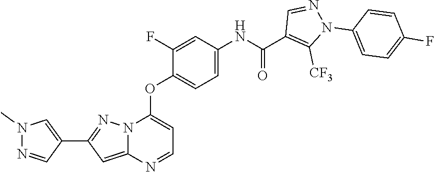 [SMILES: Cn1cc(-c2cc3nccc(Oc4ccc(NC(=O)c5cnn(-c6ccc(F)cc6)c5C(F)(F)F)cc4F)n3n2)cn1]

### Step 1: Synthesis of 3-(1-methyl-1H-imidazol-4-yl)-1H-pyrazol-5-amine

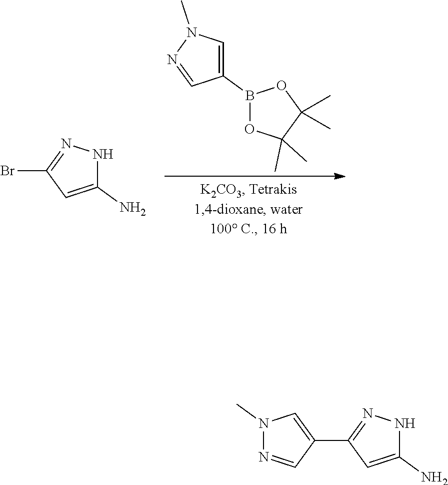

To a stirred solution of 3-bromo-1H-pyrazol-5-amine (1 g, 6.17 mmol, 1 eq) and 1-methyl-4-(4,4,5,5-tetramethyl-1,3,2-dioxaborolan-2-yl)-1H-pyrazole (1.28 g, 6.17 mmol, 1 eq) in 1,4-dioxane (20 mL) and water (5 mL) was added dipotassium carbonate (1.71 g, 12.3 mmol, 2 eq). Then the reaction mixture was degassed with nitrogen for 5 min, tetrakis(triphenylphosphane) palladium (357 mg, 309 μmol, 0.05 eq) was added and again degassed for 3 min. After degassed the reaction mixture was stirred at 100° C. for 16 h. The progress of the reaction was m monitored by TLC. After completion of the reaction filter the reaction mixture through a pad of diatomaceous earth (i.e., Celite®) and filtrate was concentrated under reduced pressure to obtained crude product. The crude was purified by flash column by using 100-200 silica mesh with 10 to 20% MeOH in DCM mobile phase to obtain 3-(1-methyl-1H-imidazol-4-yl)-1H-pyrazol-5-amine as off white solid (290 mg, 28.79%). LCMS: 164.1 (M+H)⁺. ¹H NMR (400 MHz, DMSO-d₆): δ=11.48 (br, 1H), 7.87 (s, 1H), 7.63 (s, 1H), 5.49 (s, 1H), 4.59 (br.s, 2H), 3.83 (s, 3H).

### Step 2: Synthesis of ethyl 2-(1-methyl-1H-imidazol-4-yl)-7-oxo-4H, 7H-pyrazolo [1,5-a]pyrimidine-6-carboxylate

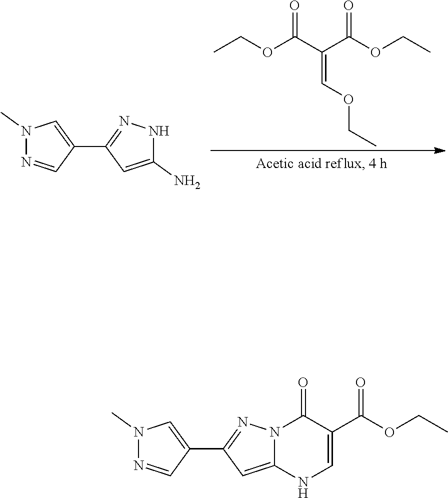 [SMILES: Cn1cc(-c2cc(N)[nH]n2)cn1 ; CCOC=C(C(=O)OCC)C(=O)OCC ; CCOC(=O)c1c[nH]c2cc(-c3cnn(C)c3)nn2c1=O]

To a suspension of 3-(1-methyl-1H-imidazol-4-yl)-1H-pyrazol-5-amine (140 mg, 858 μmol, 1 eq) in acetic acid (3 mL) was added 1-ethyl 3-propyl (2Z)-2-(ethoxymethylidene)propanedioate (269 μL, 1.29 mmol, 1.5 eq) at room temperature, the resulting suspension was heated at 120° C. for 4 h. The suspension was cooled to room temperature and concentrated under vacuum to obtained crude. The crude was triturated with cooled ethanol, solid precipitate was filtered and washed with cooled ethanol dried under vacuum to obtained ethyl 2-(1-methyl-1H-imidazol-4-yl)-7-oxo-4H,7H-pyrazolo[1,5-a]pyrimidine-6-carboxylate an off-white solid (100 mg, 40.57%). ¹H NMR (400 MHz, DMSO-d₆): δ=13.09 (s, 1H), 8.55 (s, 1H), 8.26 (s, 1H), 7.92 (s, 1H), 6.50 (s, 1H), 4.23 (q, J=14.4 Hz, 6.8 Hz, 2H), 3.89 (s, 3H), 1.29 (t, J=6.8 Hz, 3H).

### Step 3: Synthesis of 2-(1-methyl-1H-imidazol-4-yl)-7-oxo-4H,7H-pyrazolo[1,5-a]pyrimidine-6-carboxylic acid

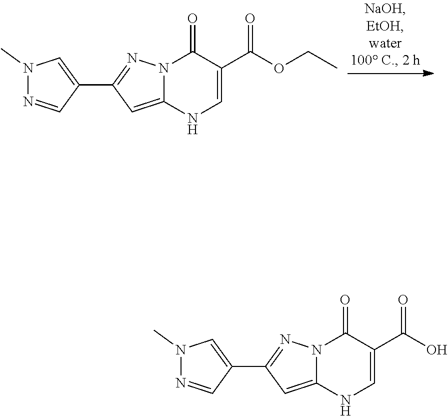

To a suspension of ethyl 2-(1-methyl-1H-imidazol-4-yl)-7-oxo-4H,7H-pyrazolo[1,5-a]pyrimidine-6-carboxylate (210 mg, 731 μmol, 1 eq) in EtOH (3 mL) at room temperature was added NaOH (73.1 mg, 1.83 mmol, 2.5 eq) dissolved in 2 mL of water. The resulting suspension was heated to 100° C. for 3.5 h. The suspension was cooled to room temperature and EtOH was concentrated under vacuum, diluted with water (10 mL) acidified by using 5% citric acid solution (5 mL) to form white suspension. The suspension was stirred an additional 30 min, filtered, washed with water and dried the solid compound to yield 2-(1-methyl-1H-imidazol-4-yl)-7-oxo-4H,7H-pyrazolo[1,5-a]pyrimidine-6-carboxylic acid (190 mg, 100%) as a white solid. ¹H NMR (400 MHz, DMSO-d₆): δ=12.85 (br, 1H), 8.66 (s, 1H), 8.29 (s, 1H), 7.95 (s, 1H), 6.60 (s, 1H), 3.89 (s, 3H).

### Step 4: Synthesis of 2-(1-methyl-1H-imidazol-4-yl)-4H,7H-pyrazolo[1,5-a]pyrimidin-7-one

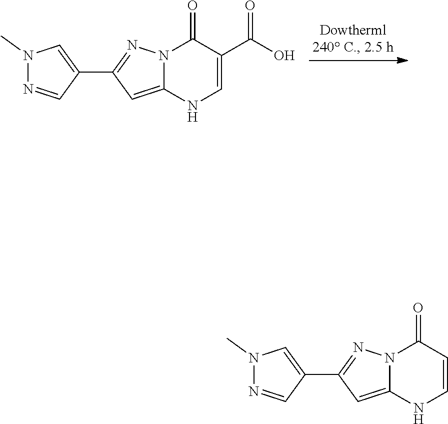 [SMILES: Cn1cc(-c2cc3[nH]cc(C(=O)O)c(=O)n3n2)cn1 ; Cn1cc(-c2cc3[nH]ccc(=O)n3n2)cn1]

A mixture of 2-(1-methyl-1H-imidazol-4-yl)-7-oxo-4H,7H-pyrazolo[1,5-a]pyrimidine-6-carboxylic acid (3.1 g, 12 mmol) in Dowtherm (2 mL) was heated to 240° C. for 2.5 h. At that point, the mixture was cooled to room temperature and diluted with hexanes (100 mL). The tan precipitate was filtered, re-suspended and stirred in hexanes (100 mL), filtered, and washed with hexanes (100 mL) to obtained 2-(1-methyl-1H-imidazol-4-yl)-4H,7H-pyrazolo[1,5-a]pyrimidin-7-one an off-white solid (110 mg, 69.73%). LCMS: 216.1 (M+H)⁺. ¹H NMR (400 MHz, DMSO-d₆): δ=12.30 (s, 1H), 8.20 (s, 1H), 7.87 (s, 1H), 7.81 (t, J=5.6 Hz, 1H), 6.35 (s, 1H), 5.65 (d, J=7.2 Hz, 1H), 3.88 (s, 3H).

### Step 5: Synthesis of 2-bromo-7-chloropyrazolo [1,5-a]pyrimidine

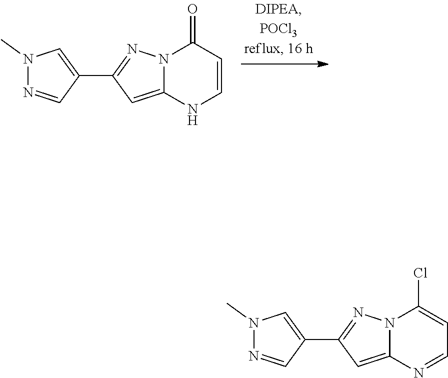 [SMILES: Cn1cc(-c2cc3[nH]ccc(=O)n3n2)cn1 ; Cn1cc(-c2cc3nccc(Cl)n3n2)cn1]

To a 2-(1-methyl-1H-imidazol-4-yl)-4H, 7H-pyrazolo [1,5-a]pyrimidin-7-one (110 mg, 511 μmol, 1 eq) were added POCl₃ (5 mL) and N,N-di isopropyl ethylamine (196 μL, 1.12 mmol, 2.2 eq) slowly at room temperature. The reaction mixture was heated to reflux for 16 h. The mixture was cooled to room temperature, and most of the solvent was evaporated. The resultant residue was diluted with ethyl acetate (100 mL) and poured slowly into water (100 mL) and pH was adjusted to 7.5 with saturated NaHCO₃ solution then stirred for 15 min. The organic layer was washed with brine (100 mL), dried (Na₂SO₄), filtered, and concentrated to obtained crude compound. The crude was purified by flash column by using 100-200 silica mesh with 5% MeOH in DCM mobile phase to afford the 2-bromo-7-chloropyrazolo [1,5-a]pyrimidine (50 mg, 41.87%) as off white solid. LCMS: 234.1 (M+H)⁺. ¹H NMR (400 MHz, DMSO-d₆): δ=8.45 (d, J=4.8 Hz, 1H), 8.32 (s, 1H), 7.98 (s, 1H), 7.33 (d, J=4.8 Hz, 1H), 7.09 (s, 1H), 3.92 (s, 3H).

### Step 6: Synthesis of N-(3-fluoro-4-{[2-(1-methyl-1H-pyrazol-4-yl)pyrazolo[1,5-a]pyrimidin-7-yl]oxy}phenyl)-1-(4-fluorophenyl)-5-(trifluoromethyl)-1H-pyrazole-4-carboxamide

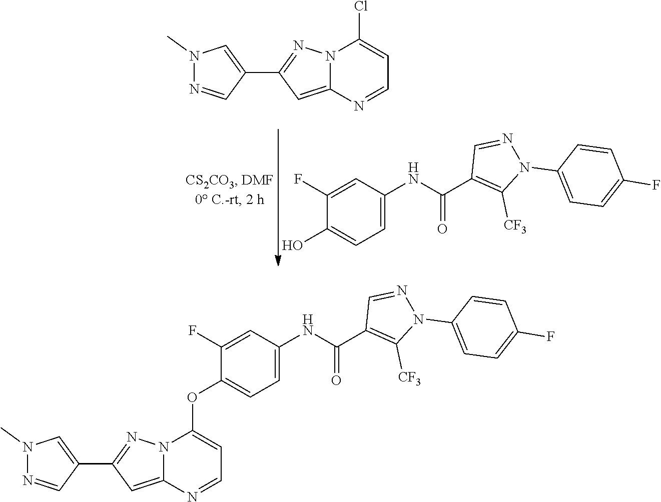 [SMILES: Cn1cc(-c2cc3nccc(Cl)n3n2)cn1 ; O=C(Nc1ccc(O)c(F)c1)c1cnn(-c2ccc(F)cc2)c1C(F)(F)F ; Cn1cc(-c2cc3nccc(Oc4ccc(NC(=O)c5cnn(-c6ccc(F)cc6)c5C(F)(F)F)cc4F)n3n2)cn1]

To a stirred solution of N-(3-fluoro-4-hydroxyphenyl)-1-(4-fluorophenyl)-5-(trifluoromethyl)-1H-pyrazole-4-carboxamide (65.6 mg, 171 μmol, 1 eq) in DMF (5 mL) was added cesium carbonate (112 mg, 342 μmol, 2 eq) at room temperature then stirred for 0.5 h. Then 4-{7-chloropyrazolo[1,5-a]pyrimidin-2-yl}-1-methyl-1H-pyrazole (40 mg, 171 μmol, 1 eq) was added to the reaction mixture at room temperature. Again, the reaction mixture was stirred at room temperature for 2 h. Then ice water (10 mL) was added to the reaction mixture and extract with ethyl acetate (3×10 mL), the combined organic layers were dried over Na₂SO₄, filtered, and concentrate under reduced pressure to get crude compound. The crude was purified by combi-flash using (12 g) with gradient elution of 60% ethyl acetate in Hexane to afford the N-(3-fluoro-4-{[2-(1-methyl-1H-pyrazol-4-yl)pyrazolo[1,5-a]pyrimidin-7-yl]oxy}phenyl)-1-(4-fluorophenyl)-5-(trifluoromethyl)-1H-pyrazole-4-carboxamide as white solid (30 mg, 30.19%). LCMS: 581.53 (M+H)⁺. ¹H NMR (400 MHz, DMSO-d₆): δ=10.94 (s, 1H), 8.36-8.32 (m, 3H), 8.02-7.99 (m, 2H), 7.71-7.61 (m, 4H), 7.47 (t, J=8.8 Hz, 2H), 6.96 (s, 1H), 6.18 (d, J=5.2 Hz, 1H), 3.91 (s, 3H). HPLC: 99.90%.

### Synthetic Examples 8-10

In a similar manner as described herein, utilizing the appropriately substituted starting materials and intermediates, the following compounds were prepared:

| Example | | | |
|---|---|---|---|
| No. | Structure | LCMS | ¹H-NMR |
| 8 | 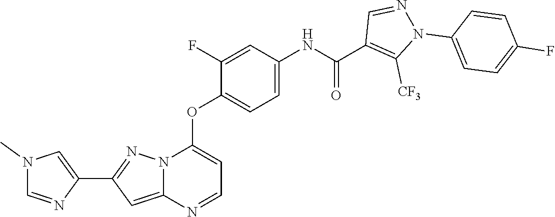 [SMILES: Cn1cnc(-c2cc3nccc(Oc4ccc(NC(=O)c5cnn(-c6ccc(F)cc6)c5C(F)(F)F)cc4F)n3n2)c1] | 581.49 (M + H)⁺ | ¹H NMR (400 MHz, DMSO-d₆): δ 10.96 (s, 1H), 8.36-8.32 (m, 2H), 8.02 (d, J = 12.80 Hz, 2H), 7.77 (s, 1H), 7.72 (s, 1H), 7.69-7.64 (m, 4H), 7.47 (t, J = 8.4 Hz, 2H), 6.89 (s, 1H), 6.19 (d, J = 5.2 Hz, 1H), 3.73 (s, 3H). |
| | Compound 8 | | |
| 9 | 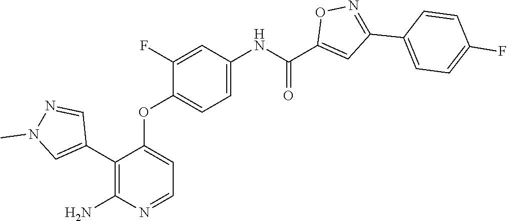 [SMILES: Cn1cc(-c2c(Oc3ccc(NC(=O)c4cc(-c5ccc(F)cc5)no4)cc3F)ccnc2N)cn1] | 489.2 (M + H)⁺ | ¹H NMR (400 MHz, DMSO-d₆): δ 11.09 (s, 1H), 8.05 (dd, J = 5.2 Hz, 8.8 Hz, 2H), 7.92- 7.88 (m, 2H), 7.86 (s, 1H), 7.77 (d, J = 5.6 Hz, 1H), 7.64 (d, J = 10.0 Hz, 1H), 7.61 (s, 1H), 7.42 (t, J = 8.8 Hz, 2H), 7.30 (t, J = 8.8 Hz, 1H), 5.95 (d, J = 5.6 Hz, 1H), 5.70 (s, 2H), 3.89 (s, 3H). |
| | Compound 21 | | |
| 10 | 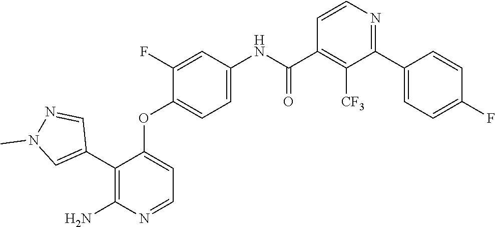 [SMILES: Cn1cc(-c2c(Oc3ccc(NC(=O)c4ccnc(-c5ccc(F)cc5)c4C(F)(F)F)cc3F)ccnc2N)cn1] | 567.2 (M + H)⁺ | ¹H NMR (400 MHz, DMSO-d₆): δ 11.05 (s, 1H), 9.03 (d, J = 5.2 Hz, 1H), 7.90 (s, 1H), 7.83- 7.75 (m, 3H), 7.61 (s, 1H), 7.57-7.53 (m, 2H), 7.43 (d, J = 8.8 Hz, 1H), 7.37 (t, J = 8.8 Hz, 2H), 7.27 (t, J = 8.8 Hz, 1H), 5.94 (d, J = 5.6 Hz, 1H), 5.69 (s, 2H), 3.89 (s, 3H). |
| | Compound 12 | | |

### Synthetic Example 11

### Synthesis of N-(3-Fluoro-4-((2-((2-Morpholinoethyl) Amino) Pyrimidin-4-Yl) Oxy) Phenyl)-1-(4-Fluorophenyl)-5-(Trifluoromethyl)-1H-Pyrazole-4-Carboxamide (Compound 28)

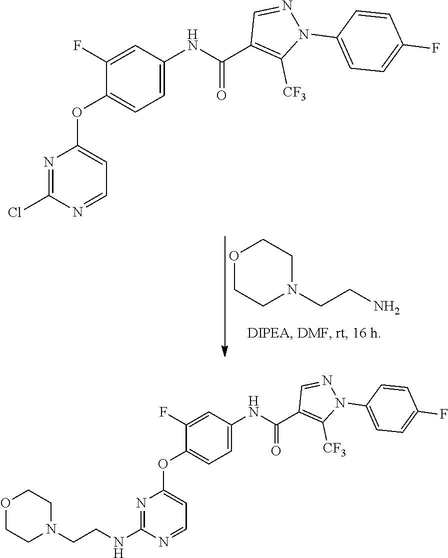 [SMILES: O=C(Nc1ccc(Oc2ccnc(Cl)n2)c(F)c1)c1cnn(-c2ccc(F)cc2)c1C(F)(F)F ; NCCN1CCOCC1 ; O=C(Nc1ccc(Oc2ccnc(NCCN3CCOCC3)n2)c(F)c1)c1cnn(-c2ccc(F)cc2)c1C(F)(F)F]

A mixture of N-{4-[(2-chloropyrimidin-4-yl) oxy]-3-fluorophenyl}-1-(4-fluorophenyl)-5-(trifluoromethyl)-1H-pyrazole-4-carboxamide (0.2 g, 403 μmol, 1 eq) and 2-(morpholin-4-yl) ethan-1-amine (52.5 mg, 403 μmol, 1 eq) in DMF (5 mL) was added ethylbis(propan-2-yl)amine (215 μL, 1.21 mmol, 3 eq) at room temperature. Then the reaction mixture was stirred for 16 h at room temperature. Progress of the reaction was monitored by TLC & LCMS. After completion of reaction, ice-water (20 mL) was added to reaction mixture and extracted with ethyl acetate (3×25 mL). Combined organic layer was dried over Na₂SO₄ and concentrated under reduced pressure to get crude compound. The crude residue was purified by Prep-HPLC to afford title compound N-{3-fluoro-4-[(2-{[2-(morpholin-4-yl)ethyl]amino}pyrimidin-4-yl)oxy]phenyl}-1-(4-fluorophenyl)-5-(trifluoromethyl)-1H-pyrazole-4-carboxamide (35 mg, 14.72% yield) as a white solid.

¹H NMR (400 MHz, DMSO-d₆): δ 10.78 (s, 1H), 8.32 (s, 1H), 8.17 (s, 1H), 7.84-7.81 (m, 1H), 7.65-7.62 (m, 2H), 7.46 (t, J=8.80 Hz, 3H), 7.34 (t, J=8.80 Hz, 1H), 6.27 (d, J=5.60 Hz, 1H), 3.47 (m, 5H), 3.04 (m, 2H), 2.15 (m, 4H). LCMS: 590.2 (M+H)⁺.

### Synthetic Example 12

### Synthesis of N-(3-Fluoro-4-((2-((1-Methylpiperidin-4-Yl)Amino)Pyrimidin-4-Yl)Oxy)Phenyl)-1-(4-Fluorophenyl)-5-(Trifluoromethyl)-1H-Pyrazole-4-Carboxamide (Compound 27)

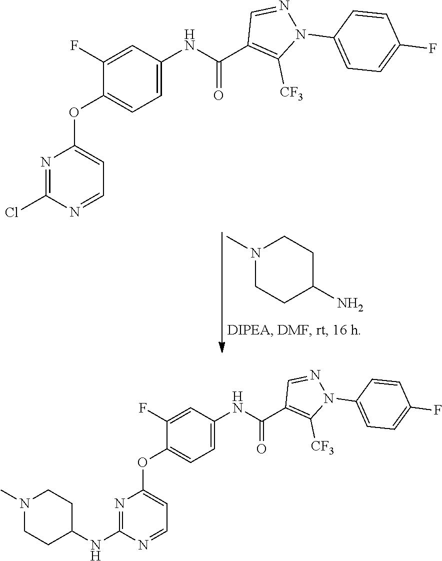

A mixture of N-{4-[(2-chloropyrimidin-4-yl)oxy]-3-fluorophenyl}-1-(4-fluorophenyl)-5-(trifluoromethyl)-1H-pyrazole-4-carboxamide (0.2 g, 403 μmol, 1 eq) and 1-methylpiperidin-4-amine (46.1 mg, 403 μmol, 1 eq) in DMF (5 mL) was added ethylbis(propan-2-yl)amine (215 μL, 1.21 mmol, 3 eq) at room temperature. The reaction mixture stirred at room temperature for 16 h. Progress of the reaction was monitored by TLC & LCMS. After completion of reaction, ice-water (20 mL) was added to reaction mixture and extracted with ethyl acetate (3×25 mL). Combined organic layer was dried over Na₂SO₄ and concentrated under reduced pressure to get crude compound. The crude compound was purified by Prep-HPLC to afford title compound N-[3-fluoro-4-({2-[(1-methylpiperidin-4-yl)amino]pyrimidin-4-yl}oxy)phenyl]-1-(4-fluorophenyl)-5-(trifluoromethyl)-1H-pyrazole-4-carboxamide (45 mg, 19.45% yield) as a white solid.

¹H NMR (400 MHz, DMSO-d₆): δ 10.79 (s, 1H), 8.33 (s, 1H), 8.18 (s, 1H), 7.81 (d, J=10.80 Hz, 1H), 7.65-7.62 (m, 2H), 7.49-7.44 (m, 3H), 7.34 (t, J=8.80 Hz, 1H), 7.24-7.01 (m, 1H), 6.25 (brs, 1H), 3.68-3.66 (m, 1H), 2.76-2.67 (m, 2H), 2.21-2.09 (m, 3H), 2.01-1.97 (m, 1H), 1.79-1.62 (m, 3H), 1.47-1.45 (m, 1H), 1.39-1.34 (m, 1H). LCMS: 574.2 (M+H)⁺.

### Synthetic Example 13

In a similar manner as described above, utilizing the appropriately substituted starting materials and intermediates, the following compound was prepared

| Example | | | |
|---|---|---|---|
| No. | Structure | LCMS | ¹H-NMR |
| 13 | 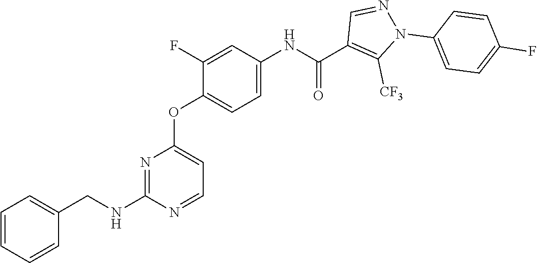 [SMILES: O=C(Nc1ccc(Oc2ccnc(NCc3ccccc3)n2)c(F)c1)c1cnn(-c2ccc(F)cc2)c1C(F)(F)F] | 567.2 (M + H⁺) | ¹H NMR (400 MHz, DMSO-d₆): δ 10.81 (s, 1H), 8.34 (s, 1H), 8.17 (d, J = 5.20 Hz, 1H), 7.82 (d, J = 12.40 Hz, 1H), 7.65-7.61 (m, 2H), 7.48-7.44 (m, 3H), 7.32-7.18 (m, 5H), 6.94 (s, 1H), 6.27 (s, 1H), 4.20 (s, 1H), 4.08 (s, 1H). |
| | Compound 37 | | |

### Synthetic Example 14

### Synthesis of N-(4-((2-((3-(Dimethylphosphoryl)Phenyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 26)

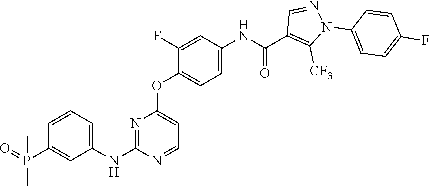 [SMILES: CP(C)(=O)c1cccc(Nc2nccc(Oc3ccc(NC(=O)c4cnn(-c5ccc(F)cc5)c4C(F)(F)F)cc3F)n2)c1]

### Step 1: Synthesis of N-(4-((2-((3-(dimethylphosphoryl)phenyl)amino)pyrimidin-4-yl)oxy)-3-fluorophenyl)-1-(4-fluorophenyl)-5-(trifluoromethyl)-1H-pyrazole-4-carboxamide

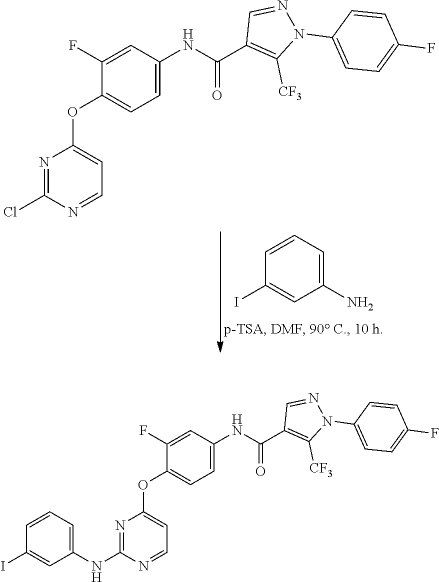 [SMILES: O=C(Nc1ccc(Oc2ccnc(Cl)n2)c(F)c1)c1cnn(-c2ccc(F)cc2)c1C(F)(F)F ; Nc1cccc(I)c1 ; O=C(Nc1ccc(Oc2ccnc(Nc3cccc(I)c3)n2)c(F)c1)c1cnn(-c2ccc(F)cc2)c1C(F)(F)F]

To a stirred solution of N-{4-[(2-chloropyrimidin-4-yl)oxy]-3-fluorophenyl}-1-(4-fluorophenyl)-5-(trifluoromethyl)-1H-pyrazole-4-carboxamide (200 mg, 403 μmol, 1 eq) and 3-iodoaniline (88.4 mg, 403 μmol, 1 eq) in DMF (20 mL), 4-methylbenzene-1-sulfonic acid hydrate (307 mg, 1.61 mmol, 4 eq) was added at room temperature. The reaction mixture stirred for 10 h at 90° C. Progress of the reaction was monitored by TLC & LCMS. After completion of reaction, reaction mixture was cool to room temperature and ice-cold water (50 mL) was added to reaction mixture and extracted with ethyl acetate (3×50 mL). Combined organic layer wash with brine solution and dried over Na₂SO₄ and concentrated under reduced pressure to get crude compound. The crude compound was purified by combi flash using 20-25% EtOAc in n-Hexane as eluent to afford title compound N-[3-fluoro-4-({2-[(3-iodophenyl)amino]pyrimidin-4-yl}oxy)phenyl]-1-(4-fluorophenyl)-5-(trifluoromethyl)-1H-pyrazole-4-carboxamide (150 mg, 54.81% yield) as white solid.

¹H NMR (400 MHz, DMSO-d₆): δ 10.82 (s, 1H), 9.77 (s, 1H), 8.43 (d, J=5.60 Hz, 1H), 8.34 (s, 1H), 7.94 9s, 1H), 7.89 (dd, J=2.00 Hz, 12.80 Hz, 1H), 7.66-7.63 (m, 2H), 7.55-7.53 (m, 1H), 7.49-7.41 (m, 4H), 7.20 (d, J=8.00 Hz, 1H), 6.89 (t, J=8.0 Hz, 1H), 6.63 (d, J=5.6 Hz, 1H).

### Step 2: Synthesis of N-(4-((2-((3-(dimethylphosphoryl)phenyl)amino)pyrimidin-4-yl)oxy)-3-fluorophenyl)-1-(4-fluorophenyl)-5-(trifluoromethyl)-1H-pyrazole-4-carboxamide

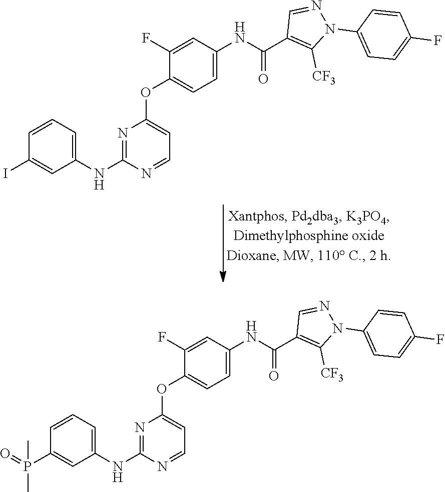 [SMILES: O=C(Nc1ccc(Oc2ccnc(Nc3cccc(I)c3)n2)c(F)c1)c1cnn(-c2ccc(F)cc2)c1C(F)(F)F ; CP(C)(=O)c1cccc(Nc2nccc(Oc3ccc(NC(=O)c4cnn(-c5ccc(F)cc5)c4C(F)(F)F)cc3F)n2)c1]

To a stirred solution of N-[3-fluoro-4-({2-[(3-iodophenyl)amino]pyrimidin-4-yl}oxy)phenyl]-1-(4-fluorophenyl)-5-(trifluoromethyl)-1H-pyrazole-4-carboxamide (100 mg, 14.7 μmol, 1 eq) in 1,4-dioxane (3 mL) was added tripotassium phosphate (34.4 mg, 162 μmol, 1.1 eq) and purged with N₂ gas for 10 min. After that [5-(diphenylphosphanyl)-9,9-dimethyl-9H-xanthen-4-yl]diphenylphosphane (8.53 mg, 14.7 μmol, 0.1 eq) and dimethyl(oxo)-λ⁵-phosphanylium (28.4 mg, 369 μmol, 2.5 eq) were added. Reaction mixture was purged with N₂ gas for 3 min and then Pd₂(dba)₃ (6.75 mg, 7.37 μmol, 0.05 eq) was added. Reaction mixture was irradiated in microwave at 110° C. for 2 hr. Reaction progress was monitored by TLC & LCMS. After completion of reaction, the reaction mixture was cool to room temperature and filter through a pad of diatomaceous earth (i.e., Celite®). Filtrate was concentrated under reduced pressure to obtained crude. Crude compound was purified by prep-HPLC to afford title compound N-{4-[(2-{[3-(dimethylphosphoryl)phenyl]amino}pyrimidin-4-yl)oxy]-3-fluorophenyl}-1-(4-fluorophenyl)-5-(trifluoromethyl)-1H-pyrazole-4-carboxamide (45 mg, 48.57% yield) as off white solid.

¹H NMR (400 MHz, DMSO-d₆): δ 10.85 (s, 1H), 9.76 (s, 1H), 8.42 (d, J=5.60 Hz, 1H), 8.36 (s, 1H), 7.89-7.81 (m, 2H), 7.72-7.63 (m, 3H), 7.54-7.42 (m, 4H), 7.26-7.21 (m, 2H), 6.61 (d, J=5.60 Hz, 1H), 1.56 (d, J=13.20 Hz, 6H), LCMS: 629.3 (M+H)⁺.

### Synthetic Example 15

### Synthesis of N-[3-Fluoro-4-({2-[(4-Fluorophenyl)Amino]Pyrimidin-4-Yl}Oxy)Phenyl]-1-(4-Fluorophenyl)-5-(Trifluoromethyl)-1H-Pyrazole-4-Carboxamide (Compound 25)

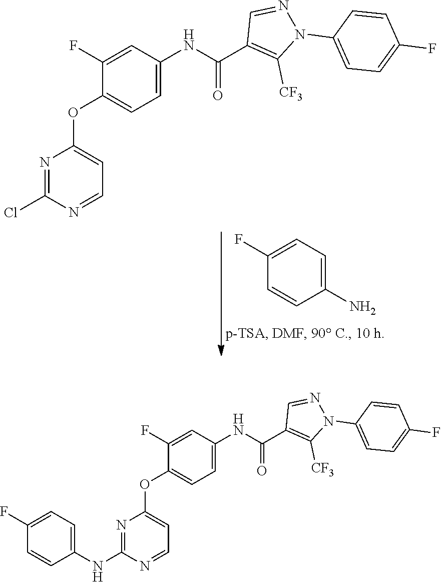 [SMILES: O=C(Nc1ccc(Oc2ccnc(Cl)n2)c(F)c1)c1cnn(-c2ccc(F)cc2)c1C(F)(F)F ; Nc1ccc(F)cc1 ; O=C(Nc1ccc(Oc2ccnc(Nc3ccc(F)cc3)n2)c(F)c1)c1cnn(-c2ccc(F)cc2)c1C(F)(F)F]

To a stirred solution of N-{4-[(2-chloropyrimidin-4-yl)oxy]-3-fluorophenyl}-1-(4-fluorophenyl)-5-(trifluoromethyl)-1H-pyrazole-4-carboxamide (200 mg, 403 μmol, 1 eq) and 4-fluoroaniline (44.8 mg, 403 μmol, 1 eq) in DMF (8 mL) was added 4-methylbenzene-1-sulfonic acid hydrate (307 mg, 1.61 mmol, 4 eq) at room temperature. The reaction mixture stirred at 90° C. for 10 h. Progress of the reaction was monitored by TLC & LCMS. After completion of reaction, water (50 mL) was added to reaction mixture and extracted with ethyl acetate (3×75 mL). Combined organic layer was dried over Na₂SO₄ and concentrated under reduced pressure to get crude compound. The crude compound was purified by prep-HPLC to afford title compound N-[3-fluoro-4-({2-[(4-fluorophenyl)amino]pyrimidin-4-yl}oxy)phenyl]-1-(4-fluorophenyl)-5-(trifluoromethyl)-1H-pyrazole-4-carboxamide (20 mg, 8.69% yield) as a white color solid.

¹H NMR (400 MHz, DMSO-d₆): δ 10.86 (s, 1H), 9.69 (s, 1H), 8.39 (d, J=5.20 Hz, 1H), 8.36 (s, 1H), 7.86 (dd, J=2.40 Hz, 12.60 Hz, 1H), 7.66-7.63 (m, 2H), 7.53-7.39 (m, 6H), 6.90 (t, J=8.80 Hz, 1H), 6.58 (d, J=5.60 Hz, 1H), LCMS: 571.2 (M+H)⁺.

### Synthetic Examples 16-26

In a similar manner as described herein, utilizing the appropriately substituted starting materials and intermediates, the following compounds were prepared:

| Example | | | |
|---|---|---|---|
| No. | Structure | LCMS | ¹H-NMR |
| 16 | 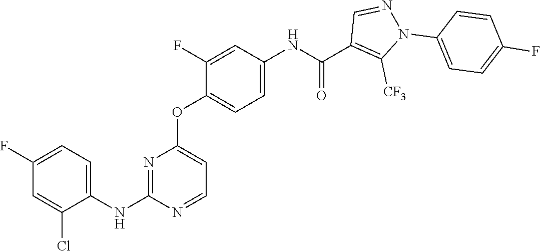 [SMILES: O=C(Nc1ccc(Oc2ccnc(Nc3ccc(F)cc3Cl)n2)c(F)c1)c1cnn(-c2ccc(F)cc2)c1C(F)(F)F] | 605.6 (M + H⁺) | ¹H NMR (400 MHz, DMSO-d₆): δ 10.80 (s, 1H), 8.93 (s, 1H), 8.33 (s, 1H), 8.30 (d, J = 5.60 Hz, 1H), 7.82 (d, J = 12.40 Hz, 1H), 7.66-7.63 (m, 2H), 7.49-7.45 (m, 3H), 7.40-7.36 (m, 2H), 7.05-7.01 (m, 1H), 6.52 (d, J = 5.60 Hz, 1H). |
| | Compound 22 | | |
| 17 | 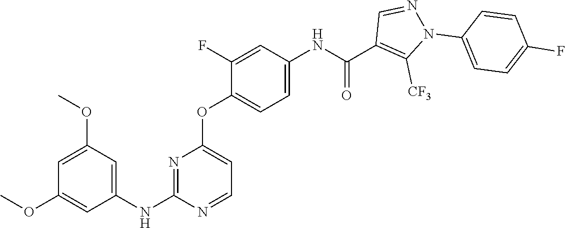 [SMILES: COc1cc(Nc2nccc(Oc3ccc(NC(=O)c4cnn(-c5ccc(F)cc5)c4C(F)(F)F)cc3F)n2)cc(OC)c1] | 613.2 (M + H⁺) | ¹H NMR (400 MHz, DMSO-d₆): δ 10.83 (s, 1H), 9.54 (s, 1H), 8.40 (d, J = 5.20 Hz, 1H), 8.35 (s, 1H), 7.86 (dd, J = 2.00 Hz, 12.00 Hz, 1H), 7.66- 7.63 (m, 2H), 7.53- 7.39 (m, 4H), 6.79 (s, 2H), 6.58 (d, J = 5.60 Hz, 1H), 6.05 (s, 1H), 3.58 (s, 6H). |
| | Compound 24 | | |
| 18 | 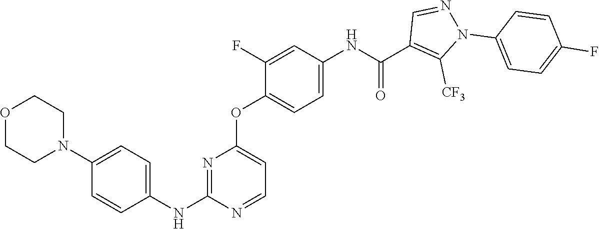 [SMILES: O=C(Nc1ccc(Oc2ccnc(Nc3ccc(N4CCOCC4)cc3)n2)c(F)c1)c1cnn(-c2ccc(F)cc2)c1C(F)(F)F] | 638.2 (M + H⁺) | ¹H NMR (400 MHz, DMSO-d₆): δ 10.83 (s, 1H), 9.43 (s, 1H), 8.36 - 8.32 (m, 2H), 7.88 (d, J = 12.80 Hz, 1H), 7.65-7.62 (m, 2H), 7.53-7.46 (m, 3H), 7.40 (t, J = 8.80 Hz, 1H), 7.27 (brs, 2H), 6.68-6.66 (m, 2H), 6.49 (d, J = 5.20 Hz, 1H), 3.63 (m, 4H), 2.93 (m, 4H). |
| | Compound 23 | | |
| 19 | 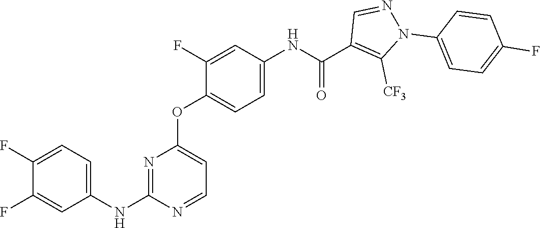 [SMILES: O=C(Nc1ccc(Oc2ccnc(Nc3ccc(F)c(F)c3)n2)c(F)c1)c1cnn(-c2ccc(F)cc2)c1C(F)(F)F] | 589.2 (M + H⁺) | ¹H NMR (400 MHz, DMSO-d₆): δ 10.86 (s, 1H), 9.90 (s, 1H), 8.45 (d, J = 5.60 Hz, 1H), 8.35 (s, 1H), 7.90 (dd, J = 2.00 Hz, 12.80 Hz, 1H), 7.67- 7.64 (m, 2H), 7.55 (d, J = 8.40 Hz, 2H), 7.51-7.42 (m, 3H), 7.18 (m, 2H), 6.66 (d, J = 9.60 Hz, 1H). |
| | Compound 29 | | |
| 20 | 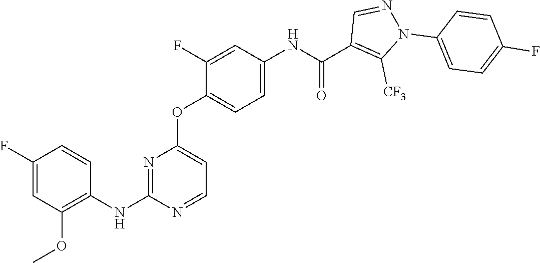 [SMILES: COc1cc(F)ccc1Nc1nccc(Oc2ccc(NC(=O)c3cnn(-c4ccc(F)cc4)c3C(F)(F)F)cc2F)n1] | 601.2 (M + H⁺) | ¹H NMR (400 MHz, DMSO-d₆): δ 10.84 (s, 1H), 8.36-8.33 (m, 2H), 8.14 (s, 1H), 7.86 (dd, J = 2.00 Hz, 12.80 Hz, 1H), 7.67- 7.64 (m, 2H), 7.51- 7.46 (m, 4H), 7.39 (t, J = 8.80 Hz, 1H), 6.90 (dd. J = 2.80 Hz, 10.80 Hz, 1H), 6.55 (d, J = 5.60 Hz, 1H), 6.49 (t, J = 7.60 Hz, 1H), 3.79 (s, 3H). |
| | Compound 36 | | |
| 21 | 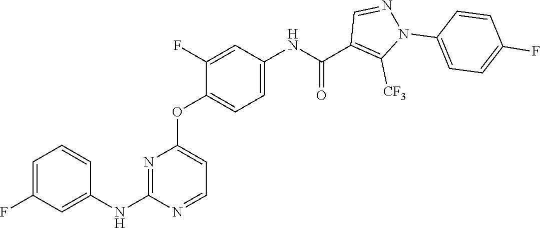 [SMILES: O=C(Nc1ccc(Oc2ccnc(Nc3cccc(F)c3)n2)c(F)c1)c1cnn(-c2ccc(F)cc2)c1C(F)(F)F] | 571.2 (M + H⁺) | ¹H NMR (400 MHz, DMSO-d₆): δ 10.87 (s, 1H), 9.88 (s, 1H), 8.44 (d, J = 5.60 Hz, 1H), 8.35 (s, 1H), 7.90 (dd, J = 2.00 Hz, 12.80 Hz, 1H), 7.66- 7.63 (m, 2H), 7.57- 7.54 (m, 1H), 7.49- 7.38 (m, 4H), 7.22 (d, J = 8.00 Hz, 1H), 7.16-7.09 (m, 1H), 6.66-6.62 (m, 2H). |
| | Compound 30 | | |
| 22 | 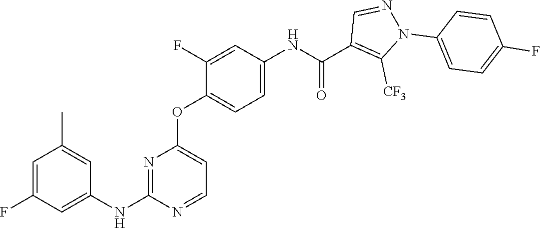 [SMILES: Cc1cc(F)cc(Nc2nccc(Oc3ccc(NC(=O)c4cnn(-c5ccc(F)cc5)c4C(F)(F)F)cc3F)n2)c1] | 585.52 (M + H⁺) | ¹H NMR (400 MHz, DMSO-d₆): δ 10.83 (s, 1H), 9.79 (s, 1H), 8.43 (d, J = 5.60 Hz, 1H), 7.91 (dd, J = 2.40 Hz, 12.80 Hz, 1H), 7.66-7.63 (m, 2H), 7.56 (dd, J = 1.60 Hz, 8.80 Hz, 1H), 7.49-7.41 (m, 3H), 7.09 (s, 2H), 6.64 (d, J = 5.60 Hz, 1H), 6.49 (d, J = 9.20 Hz, 1H), 2.09 (s, 3H). |
| | Compound 31 | | |
| 23 | 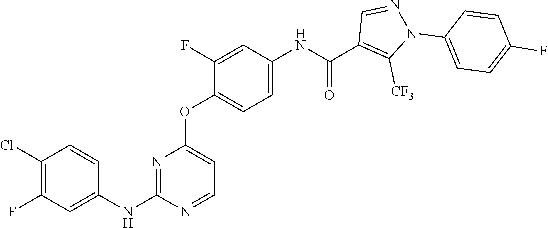 [SMILES: O=C(Nc1ccc(Oc2ccnc(Nc3ccc(Cl)c(F)c3)n2)c(F)c1)c1cnn(-c2ccc(F)cc2)c1C(F)(F)F] | 605.1 (M + H⁺) | ¹H NMR (400 MHz, DMSO-d₆): δ 10.89 (s, 1H), 10.04 (s, 1H), 8.47 (d, J = 5.60 Hz, 1H), 8.34 (s, 1H), 7.91 (dd, J = 2.40 Hz, 12.80 Hz, 1H), 7.67- 7.56 (m, 4H), 7.50- 7.43 (m, 3H), 7.32- 7.23 (m, 2H), 6.70 (d, J = 5.60 Hz, 1H). |
| | Compound 32 | | |
| 24 | 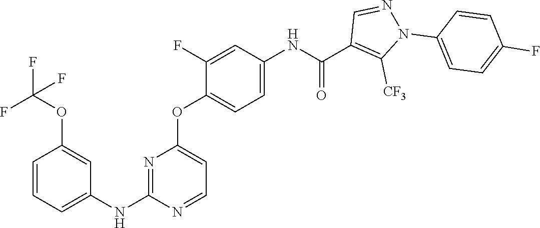 [SMILES: O=C(Nc1ccc(Oc2ccnc(Nc3cccc(OC(F)(F)F)c3)n2)c(F)c1)c1cnn(-c2ccc(F)cc2)c1C(F)(F)F] | 637.2 (M + H⁺) | ¹H NMR (400 MHz, DMSO-d₆): δ 10.86 (s, 1H), 9.96 (s, 1H), 8.46 (d, J = 5.60 Hz, 1H), 8.32 (s, 1H), 7.89 (dd, J = 2.00 Hz, 12.80 Hz, 1H), 7.66- 7.63 (m, 2H), 7.60- 7.53 (m, 2H), 7.50- 7.41 (m, 4H), 7.23 (t, J = 8.00 Hz, 1H), 6.82 (d, J = 8.00 Hz, 1H), 6.67 (d, J = 5.60 Hz, 1H). |
| | Compound 33 | | |
| 25 | 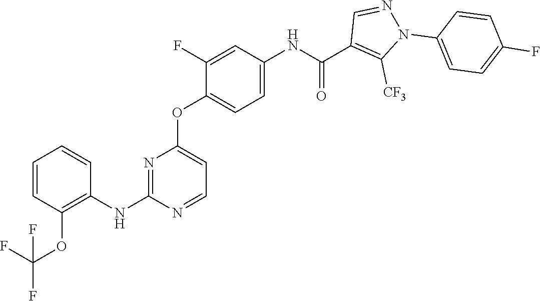 [SMILES: O=C(Nc1ccc(Oc2ccnc(Nc3ccccc3OC(F)(F)F)n2)c(F)c1)c1cnn(-c2ccc(F)cc2)c1C(F)(F)F] | 637.2 (M + H⁺) | ¹H NMR (400 MHz, DMSO-d₆): δ 10.82 (s, 1H), 9.11 (s, 1H), 8.36 (s, 1H), 8.35 (s, 1H), 7.85 (dd, J = 2.40 Hz, 12.60 Hz, 1H), 7.67-7.63 (m, 2H), 7.58 (dd, J = 1.60, 7.80 Hz, 1H), 7.50-7.45 (m, 3H), 7.43 (t, J = 8.00 Hz, 1H), 7.30-7.28 (m, 1H), 7.17-7.07 (m, 2H), 6.57 (d, J = 5.60 Hz, 1H). |
| | Compound 34 | | |
| 26 | 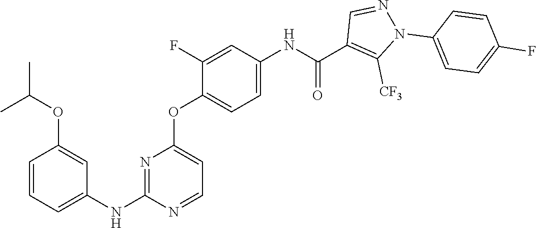 [SMILES: CC(C)Oc1cccc(Nc2nccc(Oc3ccc(NC(=O)c4cnn(-c5ccc(F)cc5)c4C(F)(F)F)cc3F)n2)c1] | 611.2 (M + H⁺) | ¹H NMR (400 MHz, DMSO-d₆): δ 10.85 (s, 1H), 9.57 (s, 1H), 8.41 (d, J = 5.20 Hz, 1H), 8.35 (s, 1H), 7.88 (dd, J = 2.40 Hz, 12.60 Hz, 1H), 7.67- 7.64 (m, 2H), 7.55-7.52 (m, 1H), 7.51-7.41 (m, 3H), 7.15 (s, 1H), 7.06 (d, J = 8.40 Hz, 1H), 6.97 (t, J = 8.40 Hz, 1H), 6.58 (d, J = 5.60 Hz, 1H), 6.43 (dd, J = |
| | | | 2.00 Hz, 8.20 Hz, 1H), |
| | Compound 35 | | 4.42-4.36 (m, 1H), 1.21 |
| | | | (d, J = 6.0 Hz, 6H). |

### Synthetic Example 27

### Synthetic Scheme for N-(4-((2-Amino-3-(Dimethyl Phosphoryl) Pyridin-4-Yl)Oxy)-3-Fluorophenyl)-1-(4-Fluorophenyl)-5-(Trifluoromethyl)-1H-Pyrazole-4-Carboxamide (Compound 17)

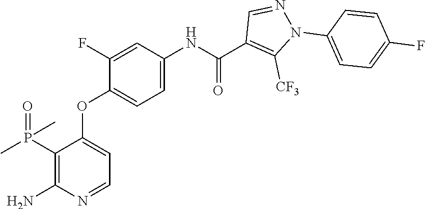 [SMILES: CP(C)(=O)c1c(Oc2ccc(NC(=O)c3cnn(-c4ccc(F)cc4)c3C(F)(F)F)cc2F)ccnc1N]

### Step 1: Synthesis of 4-(2-fluoro-4-nitrophenoxy)-3-iodopyridin-2-amine

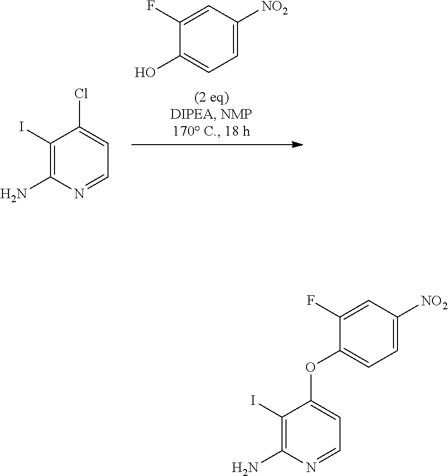 [SMILES: O=[N+]([O-])c1ccc(O)c(F)c1 ; Nc1nccc(Cl)c1I ; Nc1nccc(Oc2ccc([N+](=O)[O-])cc2F)c1I]

A mixture of 4-chloro-3-iodopyridin-2-amine (80 g, 314 mmol) and 2-fluoro-4-nitrophenol (98.8 g, 2 eq., 629 mmol) in N-Methyl-2-pyrrolidone (240 mL) was added ethylbis(propan-2-yl)amine (82.1 mL, 1.5 eq., 472 mmol) was placed in a glass pressure vessel (Sealed tube) and heated rapidly at 170° C. for 18 h. Progress of the reaction was monitored by TLC. After completion of reaction, the volatile components were distilled off under reduced pressure and the viscous residue poured into ice-water (500 mL) and then pH of the mixture was adjusted to 7.5 with saturated aq. NaHCO₃ solution. Aqueous layer was extracted with ethyl acetate (3×600 mL). The EtOAc phase was separated, washed with brine solution (100 mL), and then dried over Na₂SO₄. The organic layer was concentrated under vacuum to get crude residue. The crude compound was purified by column chromatography using silica 60-120 (with elution of 10-15% EtOAc in n-Hexane) to afford the title compound 4-(2-fluoro-4-nitrophenoxy)-3-iodopyridin-2-amine (40 g, 33.92% yield) as an off yellow solid.

LCMS: 376.00 (M+H)⁺. ¹H NMR (400 MHz, DMSO-d₆): δ=8.39 (dd, J=10.8 Hz, 2.4 Hz, 1H), 8.12 (d, J=8.8 Hz, 1H), 7.87 (d, J=5.2 Hz, 1H), 7.33 (t, J=7.6 Hz, 1H), 6.41 (s, 2H), 6.19 (d, J=5.6 Hz, 1H).

### Step 2: Synthesis of 4-(4-amino-2-fluorophenoxy)-3-iodopyridin-2-amine

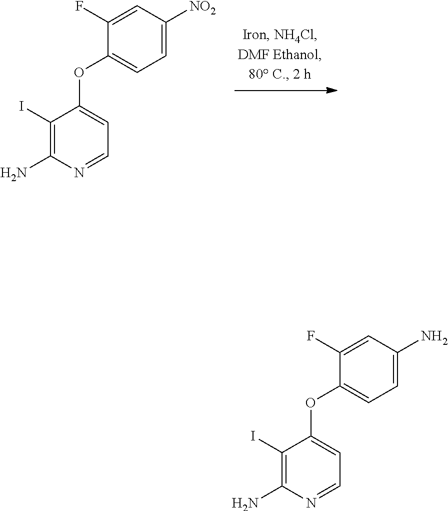 [SMILES: Nc1nccc(Oc2ccc([N+](=O)[O-])cc2F)c1I ; Nc1ccc(Oc2ccnc(N)c2I)c(F)c1]

To a stirred solution of 4-(2-fluoro-4-nitrophenoxy)-3-iodopyridin-2-amine (40 g, 10.7 mmol, 1 eq) in ethanol (400 mL), water (40 mL) and DMF (40 mL) was added was added iron powder (59.6 g, 107 mmol, 10 eq) and ammonium chloride (2.28 g, 214 mmol, 20 eq) at 0° C. Then the reaction mixture was stirred at 80° C. for 1h. Filter the hot reaction mixture through a pad of diatomaceous earth (i.e., Celite®), washed the filter cake with DMF and MeOH. The filtrate was concentrated then diluted with water (300 mL) and sodium bicarbonate solution (100 mL) and extracted with 10% MeOH in DCM (3×100 mL). The combined organic layers were dried over Na₂SO₄, filtered, and concentrated under reduced pressure to get crude compound. The crude compound was purified by flash column by using 100-200 silica mesh with 30% of EtOAc in hexane as eluent to obtain 4-(4-amino-2-fluorophenoxy)-3-iodopyridin-2-amine (22 g, 59.76% yield).

LCMS: 346.00 (M+H)⁺. ¹H NMR (400 MHz, DMSO-d₆): δ=7.70 (d, J=5.6 Hz, 1H), 6.94 (t, J=9.2 Hz, 1H), 6.49 (dd, J=13.2 Hz, 2.4 Hz, 1H), 6.40 (dd, J=8.8 Hz, 2.0 Hz, 1H), 6.13 (s, 2H), 5.71 (d, J=5.6 Hz, 1H), 5.45 (s, 2H).

### Step 3: Synthesis of (2-amino-4-(4-amino-2-fluorophenoxy) pyridin-3-yl) dimethyl phosphine oxide

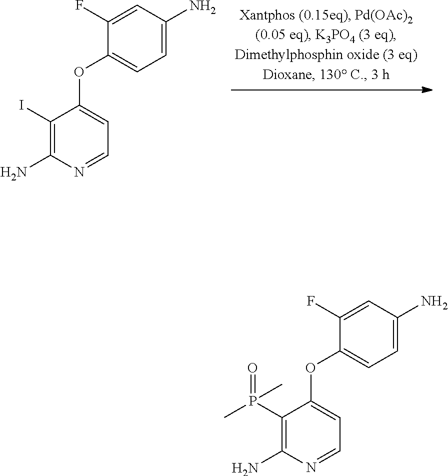 [SMILES: Nc1ccc(Oc2ccnc(N)c2I)c(F)c1 ; CP(C)(=O)c1c(Oc2ccc(N)cc2F)ccnc1N]

To a stirred solution of 4-(4-amino-2-fluorophenoxy)-3-iodopyridin-2-amine (22 g, 63.7 mmol) in 1,4-dioxane (220 mL) was added tripotassium phosphate (21.6 g, 1.6 eq., 102 mmol). After that [5-(diphenylphosphanyl)-9,9-dimethyl-9H-xanthen-4-yl]diphenylphosphane (5.53 mg, 0.15 eq., 9.56 mmol) was added to above reaction mixture and purged with N₂ gas for 30 min. After 30 min dimethyl(oxo)-λ⁵-phosphanylium (14.7 g, 3 eq., 191 mmol) and palladium(2+) diacetate (2.15 g, 0.15 eq., 9.56 mmol) was added to reaction mixture at room temperature and again purged with N₂ gas for 30 min. The reaction mixture stirred at 130° C. for 3 h. Progress of the reaction was monitored by TLC & LCMS. After completion of reaction, reaction mixture was concentrated to get crude residue. The crude compound was purified by combi flash using 3% MeOH in DCM as eluent to afford title compound 4-(4-amino-2-fluorophenoxy)-3-(dimethyl phosphoryl) pyridin-2-amine (9 g, 47.82% yield) as a pale green color liquid.

LCMS: 296.23 (M+H)⁺. ¹H NMR (400 MHz, DMSO-d₆): δ=7.87 (d, J=5.6 Hz, 1H), 6.97 (t, J=8.8 Hz, 1H), 6.49 (d, J=13.2 Hz, 1H), 6.40 (d, J=8.0 Hz, 1H), 5.77-5.71 (s, 1H), 5.48 (s, 2H), 1.77 (d, J=18.8 Hz, 6H).

### Step 4: Synthesis of N-(4-((2-amino-3-(dimethyl phosphoryl) pyridin-4-yl) oxy)-3-fluorophenyl)-1-(4-fluorophenyl)-5-(trifluoromethyl)-1H-pyrazole-4-carboxamide

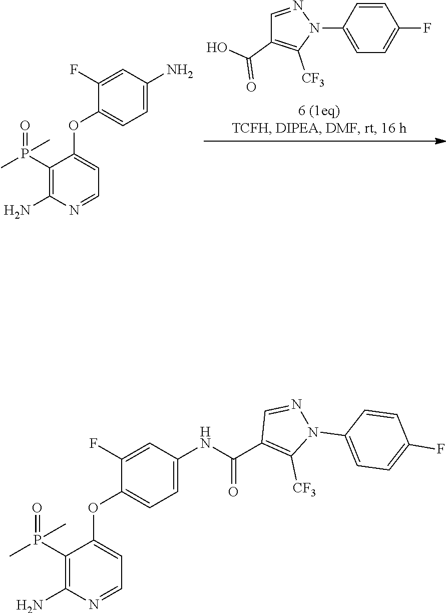 [SMILES: CP(C)(=O)c1c(Oc2ccc(N)cc2F)ccnc1N ; O=C(O)c1cnn(-c2ccc(F)cc2)c1C(F)(F)F ; CP(C)(=O)c1c(Oc2ccc(NC(=O)c3cnn(-c4ccc(F)cc4)c3C(F)(F)F)cc2F)ccnc1N]

To a stirred solution of [2-amino-4-(4-amino-2-fluorophenoxy)-3-pyridyl]dimethyl phosphine oxide (9 g, 30.5 mmol) and 1-(p-fluorophenyl)-5-(trifluoromethyl)-4-pyrazolecarboxylic acid (10 g, 1.2 eq., 36.6 mmol) in tetrahydrofuran (100 mL, 10 vol.) was added chloro(dimethyl amino)methylene bis(methyl)azanium hexafluoridophosphate(1-) (25.7 g, 3 eq., 91.4 mmol) and N-ethylbis(isopropyl)amine (27.2 mL, 5 eq., 152 mmol) at 0° C. Then the reaction mixture was stirred at room temperature for 16 h. Progress of the reaction was monitored by TLC & LCMS. After completion of reaction, water (100 mL) was added to reaction mixture and extracted with ethyl acetate (3×150 mL). The EtOAc layer was separated, washed with brine (100 mL), and then dried over Na₂SO₄, filtered, and concentrated under vacuum to get crude residue. The crude compound was purified by column chromatography using silica 60-120 (with elution of 2-3% MeOH in DCM) to afford the desired compound (60% purity by LCMS). Again, compound was purified by reverse phase chromatography (using 0.1% TFA in water and acetonitrile as eluent) to afford the N-{4-[2-amino-3-(dimethylphosphoryl)-4-pyridyloxy]-3-fluorophenyl}-1-(p-fluorophenyl)-5-(trifluoromethyl)-4-pyrazolecarboxamide methane (1/1) (1.2 g, 2.11 mmol) as a white colour solid.

LCMS: 552.58 (M+H)⁺. ¹H NMR (400 MHz, DMSO-d₆): δ=10.92 (s, 1H), 8.35 (s, 1H), 8.04 (d, J=6.8 Hz, 1H), 7.94 (dd, J=12.4 Hz, 2.4 Hz, 1H), 7.65-7.57 (m, 3H), 7.53-7.44 (m, 3H), 6.25-6.23 (m, 1H), 1.91 (d, J=14.0 Hz, 6H).

### Synthetic Example 28

### Synthesis of N-(4-((2-((3-(1-Acryloylpiperidin-3-Yl)Phenyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 38)

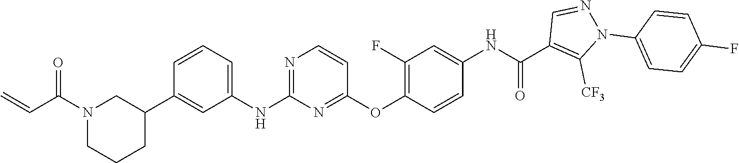 [SMILES: C=CC(=O)N1CCCC(c2cccc(Nc3nccc(Oc4ccc(NC(=O)c5cnn(-c6ccc(F)cc6)c5C(F)(F)F)cc4F)n3)c2)C1]

### Step 1: Synthesis of tert-butyl 5-(m-nitrophenyl)-1,2,3,6-tetrahydro-1-pyridinecarboxylate

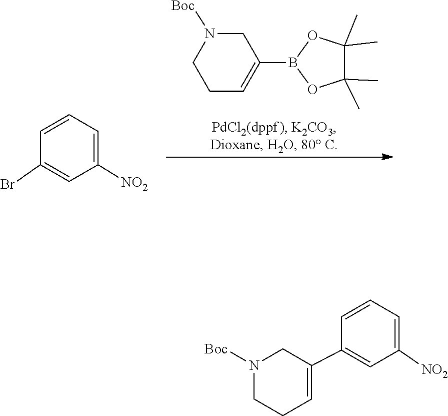 [SMILES: CC(C)(C)OC(=O)N1CCC=C(B2OC(C)(C)C(C)(C)O2)C1 ; O=[N+]([O-])c1cccc(Br)c1 ; CC(C)(C)OC(=O)N1CCC=C(c2cccc([N+](=O)[O-])c2)C1]

To a stirred solution of m-bromonitrobenzene (1 g, 4.95 mmol, 1 eq) and tert-butyl 5-(4,4,5,5-tetramethyl-1,3,2-dioxaborolan-2-yl)-1,2,3,6-tetrahydro-1-pyridinecarboxylate (1.84 g, 1.2 eq., 5.94 mmol) and dipotassium carbonate (205 mg, 3 eq., 1.49 mmol) in 1,4-dioxane (6 mL, 70.3 mmol) and water (4 mL, 222 mmol) and reaction mixture was purged with argon gas for 5 min. After that [1,1′-Bis(diphenylphosphino)ferrocene]dichloropalladium(II) (362 mg, 0.1 eq., 495 μmol) was added. Again, reaction mixture was degassed with argon for 3 min and allowed to stir at 80° C. for 4 h. Progress of reaction was monitored by TLC & LCMS. After completion of reaction, reaction mixture was quenched with water (100 mL) and extracted with ethyl acetate (3×50 mL). Combined organic layer was dried over Na₂SO₄ and concentrated under reduced pressure to get crude compound. The crude compound was purified by combi-flash on silica column using 0-50% EtOAc in hexanes to afford the title compound tert-butyl 5-(m-nitrophenyl)-1,2,3,6-tetrahydro-1-pyridinecarboxylate (1.1 g, 3.61 mmol), 73.01%) as solid.

¹H NMR (400 MHz, DMSO) δ 8.23 (s, 1H), 8.50 (d, J=8.0 Hz, 2H), 7.67 (d, J=7.6 Hz, 1H), 7.51 (d, J=8.0 Hz, 1H), 6.37-6.35 (m, 1H), 4.30 (s, 2H), 3.57 (t, J=5.6 Hz, 2H), 2.36 (s, 1H), 1.50 (s, 9H).

### Step 2: Synthesis of tert-butyl 3-(m-aminophenyl)-1-piperidinecarboxylate

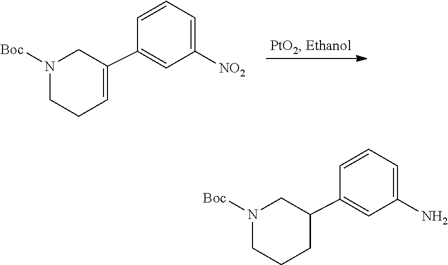 [SMILES: CC(C)(C)OC(=O)N1CCC=C(c2cccc([N+](=O)[O-])c2)C1 ; CC(C)(C)OC(=O)N1CCCC(c2cccc(N)c2)C1]

To a stirred solution of tert-butyl 5-(m-nitrophenyl)-1,2,3,6-tetrahydro-1-pyridinecarboxylate (1.2 g, 3.94 mmol) in ethanol (10 mL, 171 mmol) platinum dioxide (0.4 g, 1.76 mmol) was added at 0° C. under inert condition. The reaction temperature was increased to rt and stirred for overnight under H₂ gas. Progress of the reaction was monitored by TLC & crude LCMS. The reaction mass was concentrated in vacuo. Then, it was dissolved in ethyl acetate (100 mL) and washed with brine solution (100 mL). The collected organic layer was dried over Na₂SO₄ and concentrated in vacuum to obtain the crude. The compound was purified by flash chromatography using 5-50% EA in hexane to get desired compound tert-butyl 3-(m-aminophenyl)-1-piperidinecarboxylate (0.8 g, 73.41%).

¹H NMR (400 MHz, DMSO) δ 6.93 (t, J=7.6 Hz, 1H), 6.42-6.37 (m, 3H), 4.98 (s, 2H), 3.97-3.95 (m, 2H), 2.71-2.66 (m, 2H), 2.42-2.32 (s, 1H), 1.85-1.82 (m, 1H) 1.70-1.60 (m, 1H), 1.59-1.45 (m, 2H), 1.40 (s, 9H).

### Step 3: Synthesis of N-(4-{2-[m-(1-acryloyl-3-piperidyl)phenylamino]-4-pyrimidinyloxy}-3-fluorophenyl)-1-(p-fluorophenyl)-5-(trifluoromethyl)-4-pyrazolecarboxamide

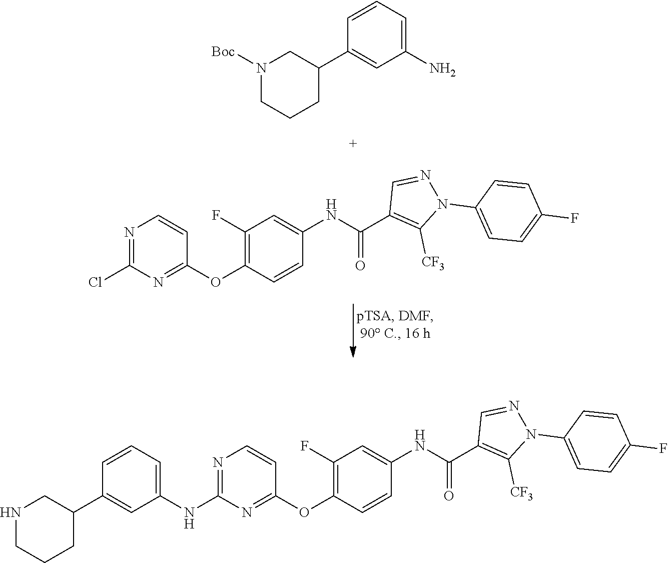 [SMILES: CC(C)(C)OC(=O)N1CCCC(c2cccc(N)c2)C1 ; O=C(Nc1ccc(Oc2ccnc(Cl)n2)c(F)c1)c1cnn(-c2ccc(F)cc2)c1C(F)(F)F ; O=C(Nc1ccc(Oc2ccnc(Nc3cccc(C4CCCNC4)c3)n2)c(F)c1)c1cnn(-c2ccc(F)cc2)c1C(F)(F)F]

To a stirred solution of N-[4-(2-chloro-4-pyrimidinyloxy)-3-fluorophenyl]-1-(p-fluorophenyl)-5-(trifluoromethyl)-4-pyrazolecarboxamide (0.5 g, 1.01 mmol) and tert-butyl 3-(m-aminophenyl)-1-piperidinecarboxylate (362 mg, 1.3 eq, 1.31 mmol) in dimethylformamide (10 mL, 129 mmol), 4-methylbenzene-1-sulfonic acid hydrate (767 mg, 4 eq, 4.03 mmol) was added. Then, the reaction mixture stirred for 10 h at 90° C. Then the reaction was monitored by TLC & LCMS. Then to the reaction mixture iced water (40 mL) was added and extract with ethyl acetate (3×75 mL). The combined organic layers were dried over anhydrous Na₂SO₄ and concentrate under reduced pressure to get crude. The crude was purified by flash chromatography using 30% o ethyl acetate in hexane to afford title compound N-(3-fluoro-4-{2-[m-(3-piperidyl)phenylamino]-4-pyrimidinyloxy}phenyl)-1-(p-fluorophenyl)-5-(trifluoromethyl)-4-pyrazolecarboxamide (180 mg, 28.08% yield).

¹H NMR (400 MHz, DMSO) δ 10.89 (s, 1H), 9.62 (s, 1H), 8.41-8.38 (m, 2H), 7.88 (d, J=13.2 Hz, 1H), 7.66-7.62 (m, 2H), 7.54 (d, J=7.2 Hz, 1H), 7.50-7.40 (m, 6H), 7.35 (s, 1H), 7.11 (d, J=8.0 Ha, 2H), 7.07-7.05 (m, 1H), 6.80 (d, J=7.6 Hz, 1H), 6.58 (d, J=5.2 Hz, 1H), 3.26 (s, 2H), 2.92-2.77 (m, 2H), 2.28 (s, 2H), 2.42-2.32 (s, 1H), 1.87-1.69 (m, 2H) 1.64-1.56 (m, 1H), LCMS: 636.56 (M+H⁺).

### Step 4: Synthesis of N-(4-{2-[m-(1-acryloyl-3-piperidyl)phenylamino]-4-pyrimidinyloxy}-3-fluorophenyl)-1-(p-fluorophenyl)-5-(trifluoromethyl)-4-pyrazolecarboxamide

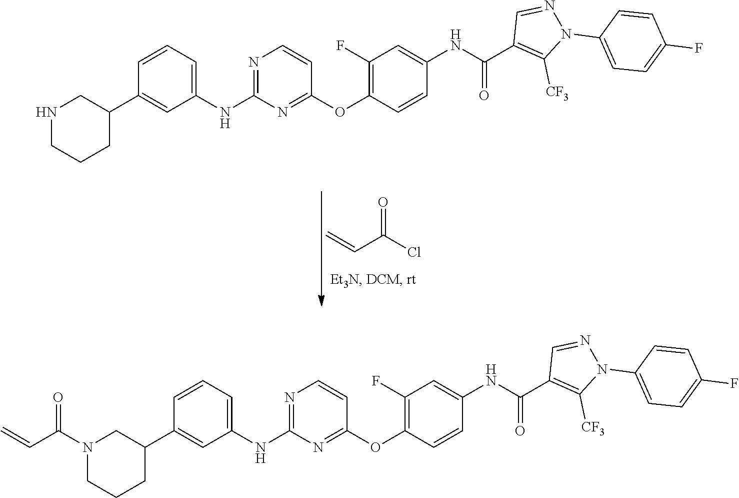

To a stirred solution of N-(3-fluoro-4-{2-[m-(3-piperidyl)phenylamino]-4-pyrimidinyloxy}phenyl)-1-(p-fluorophenyl)-5-(trifluoromethyl)-4-pyrazolecarboxamide (150 mg, 236 μmol) in dichloromethane (15 mL, 234 mmol), triethylamine (98.7 μL, 3 eq, 708 μmol) was added dropwise at 0° C. After 5 min, acryloyl chloride (17.2 μL, 0.9 eq., 212 μmol) was added under inert condition dropwise. The reaction temperature was stirred at for 6 h. Progress of the reaction was monitored by LCMS. After completion of reaction, reaction mixture was concentrated under reduced pressure to afford crude residue. The crude compound was purified by Prep-HPLC to afford title compound N-(4-{2-[m-(1-acryloyl-3-piperidyl)phenylamino]-4-pyrimidinyloxy}-3-fluorophenyl)-1-(p-fluorophenyl)-5-(trifluoromethyl)-4-pyrazolecarboxamide (12 mg, 7.37% yield).

¹H NMR (400 MHz, DMSO) δ 10.16 (s, 1H), 8.30-8.27 (m, 2H), 7.62 (d, J=9.2 Hz, 1H), 7.54-7.49 (m, 2H), 7.46-7.42 (m, 2H), 7.22-7.16 (m, 4H), 7.09 (s, 1H), 6.85-6.83 (m, 2H), 6.74-6.66 (m, 1H), 6.48 (d, J=5.6 Hz, 1H), 5.76 (dd, J=1.6 Hz, 10.6 Hz, 1H), 4.75-4.65 (m, 1H), 4.12-3.90 (m, 1H), 3.16-2.99 (m, 1H), 2.79 (t, J=8.0 Hz, 1H), 2.64-2.58 (m, 1H), 2.02-1.96 (m, 1H) 1.90-1.86 (m, 1H), 1.55 (m, 2H), LCMS: 690.72 (M+H⁺).

### Synthetic Example 29

### Synthesis of (E)-N-(4-((2-((3-(4-(Dimethylamino)but-2-Enamido)Phenyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 40)

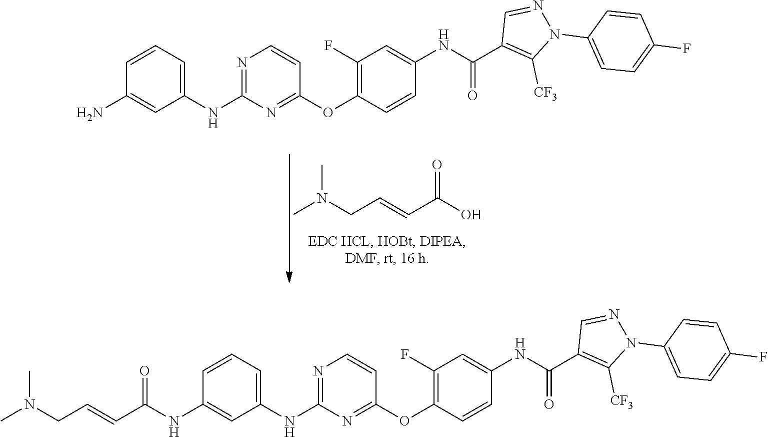

To a stirred solution of N-{4-[2-(m-aminophenylamino)-4-pyrimidinyloxy]-3-fluorophenyl}-2-(p-fluorophenyl)-3-(trifluoromethyl)-1,2λ⁵-diazole-4-carboxamide (100 mg, 176 μmol, 1 eq) and (E)-4-(dimethyl amino)-2-butenoic acid (27.3 mg, 211 μmol, 1.2 eq) in DMF (5 mL) were added 2-methyl-2,6,8-triaza-6,7-decadiene-hydrogen chloride (1/1) (50.7 mg, 264 μmol, 1.5 eq) and 1H-1,2,3-benzotriazol-1-ol to water (1:1) (40.5 mg, 264 μmol, 1.5 eq) and then N-ethylbis (isopropyl)amine (92.1 μL, 529 μmol, 3 eq) was added at room temperature. The reaction mass was stirred at room temperature for 16 h. Progress of the reaction was monitored by TLC & LCMS. After completion of reaction, water (10 mL) was added to reaction mixture and extracted with ethyl acetate (3×20 mL). Combined organic layer was dried over Na₂SO₄ and concentrated under reduced pressure to get crude compound. The crude was purified by prep-HPLC to afford title compound N-[4-(2-{m-[(E)-3-(dimethyl amino)-1-propenylcarbonylamino]phenylamino}-4-pyrimidinyloxy)-3-fluorophenyl]-1-(p-fluorophenyl)-5-(trifluoromethyl)-4-pyrazolecarboxamide (20 mg, 16.72% yield) as white color solid.

¹H NMR (400 MHz, DMSO) δ 10.85 (s, 1H), 10.13 (s, 1H), 9.66 (s, 1H), 8.46-8.35 (m, 2H), 7.86 (d, J=12.7 Hz, 1H), 7.74 (s, 1H), 7.65 (dd, J=8.7, 4.9 Hz, 2H), 7.58-7.33 (m, 4H), 7.25 (d, J=7.8 Hz, 2H), 6.99 (t, J=8.1 Hz, 1H), 6.72 (dd, J=15.3, 6.8 Hz, 1H), 6.56 (d, J=5.6 Hz, 1H), 6.39 (d, J=15.7 Hz, 1H), 3.57 (s, 2H), LCMS: 679.68. (M+H)⁺.

### Synthetic Example 30

### Synthesis of N-(4-((2-((3-Acrylamidophenyl) Amino) Pyrimidin-4-Yl) Oxy)-3-Fluorophenyl)-1-(4-Fluorophenyl)-5-(Trifluoromethyl)-1H-Pyrazole-4-Carboxamide (Compound 42)

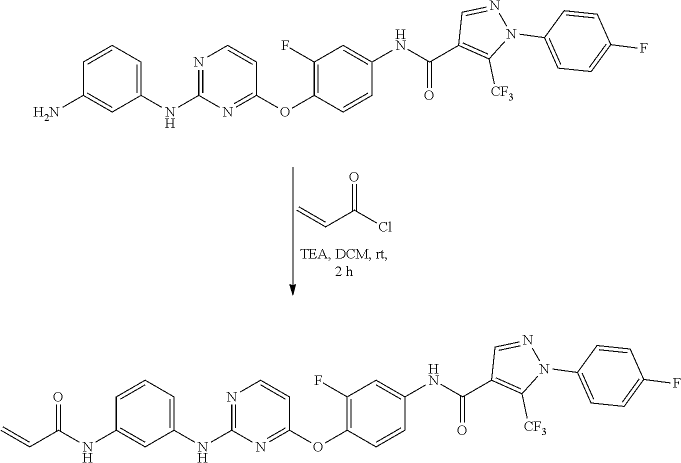

To a stirred solution of N-{4-[2-(m-aminophenylamino)-4-pyrimidinyloxy]-3-fluorophenyl}-2-(p-fluorophenyl)-3-(trifluoromethyl)-1,2λ⁵-diazole-4-carboxamide (150 mg, 264 μmol, 1 eq), DCM (7.5 mL) and was added trimethylamine (110 μL, 792 μmol, 3 eq). After stirring of 5 min acryloyl chloride (1.71 μL, 21.1 μmol, 1.2 eq) was added at room temperature. The reaction mixture was stirred for 10 min at RT. Progress of reaction was monitored by TLC & LCMS. After completion of reaction, reaction was quenched with water (15 mL) and extracted with ethyl acetate (3×30 mL). Combined organic layer was dried over Na₂SO₄ and concentrated under reduced pressure to get crude compound. The crude was purified by prep-HPLC to get title compound N-(3-fluoro-4-{2-[m-(vinylcarbonylamino) phenylamino]-4-pyrimidinyloxy}phenyl)-1-(p-fluorophenyl)-5-(trifluoromethyl)-4-pyrazolecarboxamide (40 mg, 24.35% yield) as a white color solid.

¹H NMR (400 MHz, DMSO) δ 10.83 (s, 1H), 10.03 (s, 1H), 9.66 (s, 1H), 8.40-8.35 (m, 2H), 7.86 (dd, J=12.6, 2.4 Hz, 1H), 7.73 (s, 1H), 7.66-7.63 (m, 2H), 7.52-7.39 (m, 4H), 7.26-7.23 (m, 2H), 6.99 (t, J=8.0 Hz, 1H), 6.56 (d, J=5.6 Hz, 1H), 6.47-6.40 (m, 1H), 6.22 (dd, J=17.0, 2.0 Hz, 1H), 5.71 (dd, J=10.0, 2.0 Hz, 1H), LCMS: 622.59 (M+H)⁺.

### Synthetic Example 31

### Synthesis of N-(4-((2-((3-((S)-2-((E)-4-(Dimethylamino)-N-Methylbut-2-Enamido) Propanamido) Cyclohexyl) Amino) Pyrimidin-4-Yl) Oxy)-3-Fluorophenyl)-1-(4-Fluorophenyl)-5-(Trifluoromethyl)-1H-Pyrazole-4-Carboxamide (Compound 41)

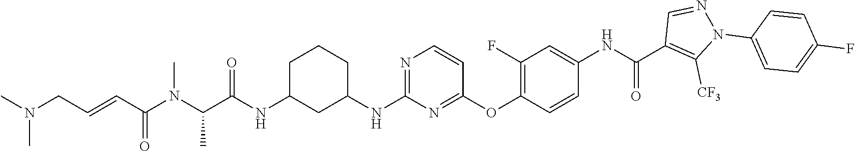 [SMILES: C[C@@H](C(=O)NC1CCCC(Nc2nccc(Oc3ccc(NC(=O)c4cnn(-c5ccc(F)cc5)c4C(F)(F)F)cc3F)n2)C1)N(C)C(=O)/C=C/CN(C)C]

### Step 1: Synthesis of tert-butyl (E)-N-(4-(dimethylamino) but-2-enoyl)-N-methyl-L-alaninate

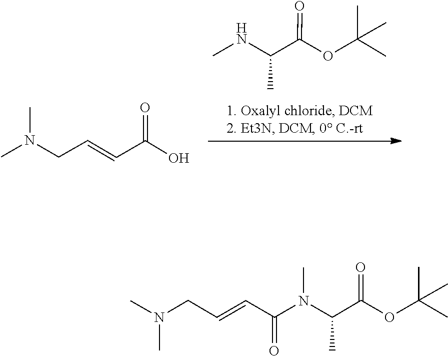

To a stirred solution of (E)-4-(dimethylamino)but-2-enoic acid (100 mg, 0.774 mmol, 1 eq) in DCM (5 mL) was added Oxalyl chloride (0.1 mL, 1.16 mmol, 1.5 eq) followed by DMF (cat.) at 0° C. The reaction mixture was stirred at room temperature for 1 h. Progress of reaction was monitored by TLC. After consumption of starting material on TLC, this solution was added to a stirred solution of tert-butyl methyl-L-alaninate (123 mg, 0.774 mmol, 1 eq) and trimethylamine (0.32 mL, 2.32 mmol, 3 eq) in DCM (5 mL) at 0° C. The reaction mixture was stirred for 2 h until the complete consumption of starting material. Reaction progress was monitored by TLC. After completion of reaction, reaction mixture was diluted with water (20 mL) and extracted with DCM (2×20 mL). The combined organic layer was dried over Na₂SO₄ and concentrated under reduced pressure. The crude compound was purified by flash column chromatography using 2-3% MeOH in DCM to afford title compound tert-butyl (E)-N-(4-(dimethylamino)but-2-enoyl)-N-methyl-L-alaninate (70 mg, 33% yield) as pale yellow syrup.

¹H NMR (400 MHz, DMSO) δ 6.66-6.53 (m, 2H), 4.85-4.74 (m, 1H), 3.08-3.03 (m, 2H), 2.95 (s, 2H), 2.72 (s, 1H), 2.17 (s, 6H), 1.38 (s, 9H), 1.33-1.26 (m, 3H), LCMS: 271.2 (M+H)⁺.

### Step 2: Synthesis of (E)-N-(4-(dimethylamino) but-2-enoyl)-N-methyl-L-alanine

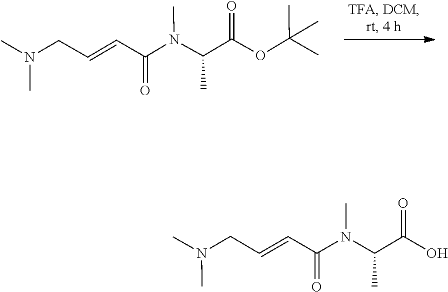

To a stirred solution of tert-butyl (E)-N-(4-(dimethylamino)but-2-enoyl)-N-methyl-L-alaninate (50 mg, 0.185 mmol, 1 eq) in 1,4-dioxane (2 mL) at 0° C. was added 4M HCl in dioxane (0.5 mL). The reaction mixture was stirred at room temperature for 4h. Volatiles were evaporated under reduced pressure to get crude residue as brown solid of (E)-N-(4-(dimethylamino)but-2-enoyl)-N-methyl-L-alanine-HCl salt (50 mg).

¹H NMR (400 MHz, DMSO) δ 9.90 (s, 1H), 6.86 (d, J=14.8 Hz, 1H), 6.66-6.50 (m, 1H), 4.96-4.78 (m, 1H), 3.91-3.83 (m, 2H), 2.99 (s, 2H), 2.78 (s, 6H), 1.39-1.30 (m, 3H), LCMS: 215.2 (M+H)⁺.

### Step 3: Synthesis of N-(4-((2-((3-((S)-2-((E)-4-(dimethylamino)-N-methylbut-2-enamido)propanamido) cyclohexyl)amino)pyrimidin-4-yl)oxy)-3-fluorophenyl)-1-(4-fluorophenyl)-5-(trifluoromethyl)-1H-pyrazole-4-carboxamide

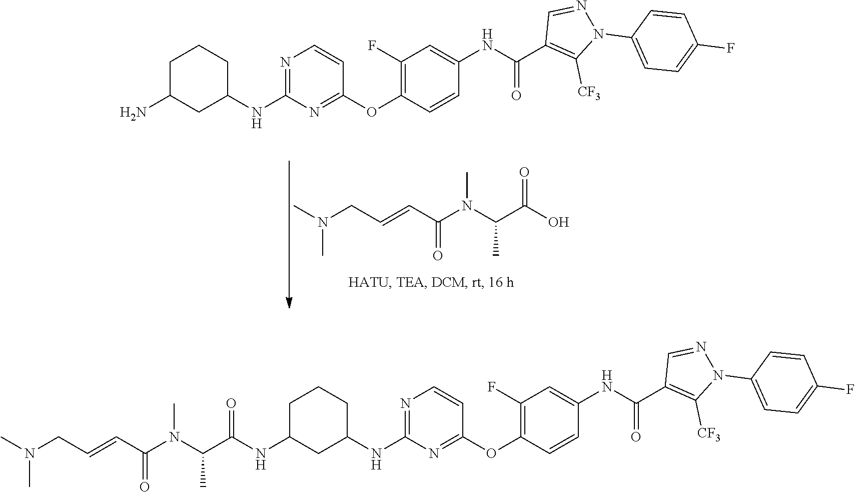 [SMILES: NC1CCCC(Nc2nccc(Oc3ccc(NC(=O)c4cnn(-c5ccc(F)cc5)c4C(F)(F)F)cc3F)n2)C1 ; C[C@@H](C(=O)O)N(C)C(=O)/C=C/CN(C)C ; C[C@@H](C(=O)NC1CCCC(Nc2nccc(Oc3ccc(NC(=O)c4cnn(-c5ccc(F)cc5)c4C(F)(F)F)cc3F)n2)C1)N(C)C(=O)/C=C/CN(C)C]

To a stirred solution of N-(4-((2-((3-aminocyclohexyl)amino)pyrimidin-4-yl)oxy)-3-fluorophenyl)-1-(4-fluorophenyl)-5-(trifluoromethyl)-1H-pyrazole-4-carboxamide (90 mg, 0.157 mmol, 1 eq) in DCM (3 mL) was added triethylamine (47.6 mg, 0.471 mmol, 3 eq) and HATU (119 mg, 0.314 mmol, 2 eq) and the reaction mixture was stirred for 5 min then (E)-N-(4-(dimethylamino)but-2-enoyl)-N-methyl-L-alanine (33.6 mg, 0.157 mmol, 1 eq) was added to reaction mixture. The reaction mixture was stirred for 16 h at room temperature. Progress of reaction was monitored by TLC. After completion of reaction, reaction mixture was quenched with water (15 mL) and extracted with DCM (3×20 mL). The combined organic layer washed with brine solution (20 mL), then dried over Na₂SO₄ concentrated under reduced pressure to get crude residue. The crude compound was purified by prep-HPLC using 0.1% FA in acetonitrileACN to afford white solid of N-(4-((2-((3-((S)-2-((E)-4-(dimethylamino)-N-methylbut-2-enamido)propanamido)cyclohexyl)amino)pyrimidin-4-yl)oxy)-3-fluorophenyl)-1-(4-fluorophenyl)-5-(trifluoromethyl)-1H-pyrazole-4-carboxamide (35 mg, 28.97% yield).

¹H NMR (400 MHz, DMSO) δ 10.78 (s, 1H), 8.33-8.30 (m, 1H), 8.18 (d, J=5.2 Hz, 1H), 7.92-7.72 (m, 2H), 7.65-7.61 (m, 2H), 7.46 (t, J=8.8 Hz, 3H), 7.33 (t, J=8.8 Hz, 3H), 7.06 (m, 1H), 6.59-6.49 (m, 2H), 6.24 (m, 1H), 4.99-4.52 (m, 1H), 4.04-3.93 (m, 1H), 3.75-3.58 (m, 1H), 3.01-2.90 (m, 5H), 2.14 (s, 6H), 1.90-1.46 (m, 5H), 1.25-1.09 (m, 6H), LCMS: 768.83 (M−H)⁺.

### Synthetic Example 32

### Synthesis of (E)-N-(4-((2-((3-(4-(Dimethylamino)but-2-Enamido)Cyclohexyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 39)

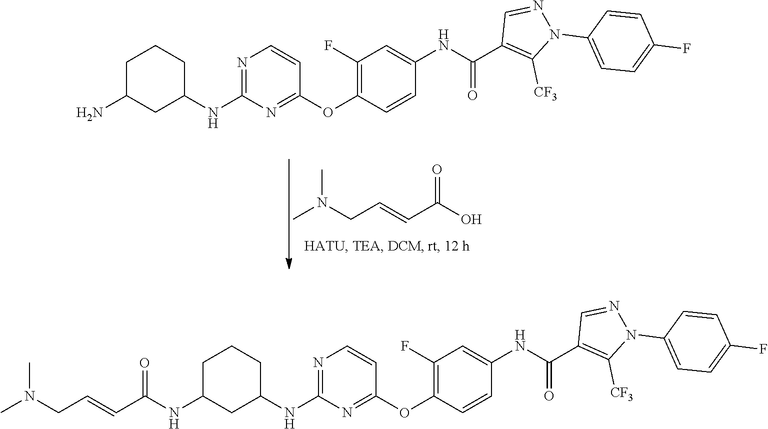

To a stirred solution of N-(4-((2-((3-aminocyclohexyl)amino)pyrimidin-4-yl)oxy)-3-fluorophenyl)-1-(4-fluorophenyl)-5-(trifluoromethyl)-1H-pyrazole-4-carboxamide (90 mg, 0.157 mmol, 1 eq) in DCM (3 mL) was added triethylamine (47.6 mg, 0.471 mmol, 3 eq) followed by HATU (119 mg, 0.314 mmol, 2 eq) and the reaction mixture was stirred for 5 min then added (E)-4-(dimethylamino)but-2-enoic acid (31.1 mg, 0.188 mmol, 1.2 eq). The reaction mixture was stirred at room temperature for 12h. Reaction mixture was quenched with water (15 mL) and extracted with DCM (2×20 mL). The combined organic layer wash with brine solution (20 mL), then dried over Na₂SO₄ concentrated under reduced pressure. The crude compound was purified by prep-HPLC using 0.1% FA in acetonitrile to afford (E)-N-(4-((2-((3-(4-(dimethylamino)but-2-enamido)cyclohexyl)amino)pyrimidin-4-yl)oxy)-3-fluorophenyl)-1-(4-fluorophenyl)-5-(trifluoromethyl)-1H-pyrazole-4-carboxamide (50 mg, 46.54% yield) as white solid.

¹H NMR (400 MHz, DMSO) δ 10.78 (s, 1H), 8.34 (s, 1H), 8.18 (d, J=5.6 Hz, 1H), 7.90 (d, J=8.0 Hz, 1H), 7.81-7.76 (m, 1H), 7.65-7.61 (m, 2H), 7.48-7.44 (m, 3H), 7.37-7.32 (m, 1H), 7.05-6.99 (m, 1H), 6.54-6.47 (m, 2H), 6.24-6.13 (m, 1H), 5.98 (d, J=15.2 Hz, 1H), 4.06-4.05 (m, 1H), 3.78-3.57 (m, 1H), 2.97-2.94 (m, 2H), 2.12 (d, J=4.0 Hz, 6H), 1.95-1.90 (m, 1H), 1.87-1.49 (m, 4H), 1.35-1.28 (m, 1H), 1.23-1.16 (m, 1H), 1.10-0.99 (m, 1H), LCMS: 685.85 (M+H)⁺.

### Synthetic Example 33

### Synthesis of N-(4-((2-((3-Acrylamidocyclohexyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 44)

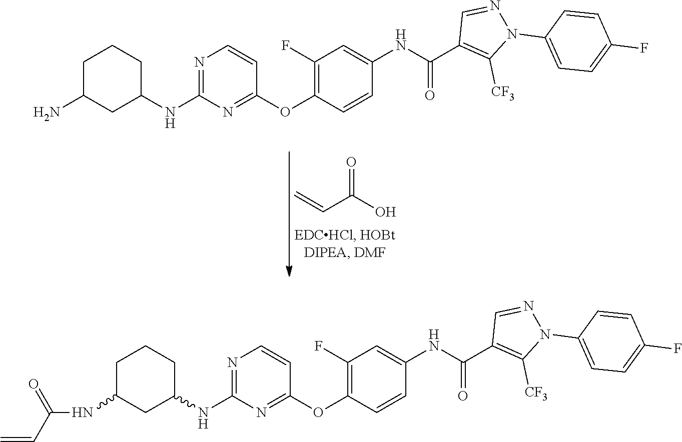

To a stirred solution of N-(4-((2-((3-aminocyclohexyl)amino)pyrimidin-4-yl)oxy)-3-fluorophenyl)-1-(4-fluorophenyl)-5-(trifluoromethyl)-1H-pyrazole-4-carboxamide (200 mg, 0.349 mmol, 1 eq) in DMF (5 mL) was added DIPEA (135 mg, 1.05 mmol, 3 eq), EDCI (100 mg, 0.523 mmol, 1.5 eq) and HOBt (70.7 mg, 0.523 mmol, 1.5 eq) and acrylic acid (27.6 mg, 0.384 mmol, 1.1 eq). The reaction mixture was stirred for 16 h at room temperature. Progress of reaction was monitored by TLC. After completion of reaction, reaction mixture was quenched with water (30 mL) and extracted with ethyl acetate (3×25 mL). The combined organic layer was dried over Na₂SO₄ and concentrated under reduced pressure. The crude compound obtained, was purified by prep-HPLC using 0.1% TFA in acetonitrile and water to afford title compound N-(4-((2-((3-acrylamidocyclohexyl)amino)pyrimidin-4-yl)oxy)-3-fluorophenyl)-1-(4-fluorophenyl)-5-(trifluoromethyl)-1H-pyrazole-4-carboxamide (Peak 1 & Peak 2) as white solids.

Conclusion: In prep-HPLC 2-peaks were isolated, Peak 1 (10 mg) & Peak 2 (15 mg).

Peak 1 Analysis: ¹H NMR (400 MHz, DMSO) δ 10.82 (s, 1H), 8.35 (s, 1H), 8.21 (s, 1H), 8.04 (s, 1H), 7.82 (d, J=11.6 Hz, 2H), 7.65-7.61 (m, 2H), 7.49-7.44 (m, 3H), 7.37 (t, J=8.8 Hz, 1H), 6.34 (m, 2H), 6.07-6.02 (m, 1H), 5.54 (d, J=10.0 Hz, 1H), 4.06 (m, 2H), 1.75 (s, 1H), 1.64-1.47 (m, 7H), LCMS: 628.60 (M+H)⁺.

Peak 2 Analysis: ¹H NMR (400 MHz, DMSO) δ 10.80 (s, 1H), 8.35 (s, 1H), 8.21 (d, J=5.6 Hz, 1H), 8.02 (d, J=7.6 Hz, 1H), 7.80 (d, J=12.4 Hz, 1H), 7.65-7.61 (m, 2H), 7.48-7.44 (m, 3H), 7.37 (t, J=8.8 Hz, 1H), 6.34 (m, 1H), 6.21-6.14 (m, 1H), 6.07-6.02 (m, 1H), 5.54 (dd, J=2.4, 9.6 Hz, 1H), 3.74 (m, 2H), 3.24-3.16 (m, 1H), 1.98-1.90 (m, 2H), 1.75-1.71 (m, 3H), 1.28-1.23 (m, 2H), 1.20-1.13 (m, 2H), 1.11-0.99 (m, 2H), LCMS: 628.64 (M+H)⁺.

### Synthetic Example 34

### Synthetic Scheme for N-(4-((3-(Dimethylphosphoryl)Pyridin-4-Yl)Oxy)-3-Fluorophenyl)-1-(4-Fluorophenyl)-3-(Trifluoromethyl)-1H-Pyrazole-4-Carboxamide (Compound 52) and N-(4-((3-(Dimethylphosphoryl)Pyridin-4-Yl)Oxy)-3-Fluorophenyl)-1-(4-Fluorophenyl)-5-(Trifluoromethyl)-1H-Pyrazole-4-Carboxamide (Compound 53)

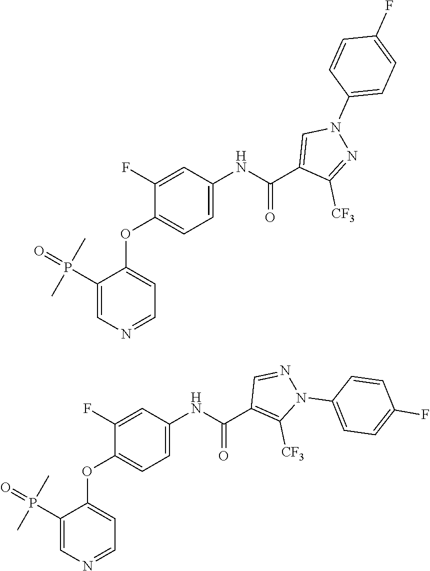 [SMILES: CP(C)(=O)c1cnccc1Oc1ccc(NC(=O)c2cn(-c3ccc(F)cc3)nc2C(F)(F)F)cc1F ; CP(C)(=O)c1cnccc1Oc1ccc(NC(=O)c2cnn(-c3ccc(F)cc3)c2C(F)(F)F)cc1F]

### Step 1: Synthesis of 4-(2-fluoro-4-nitrophenoxy)-3-iodopyridine

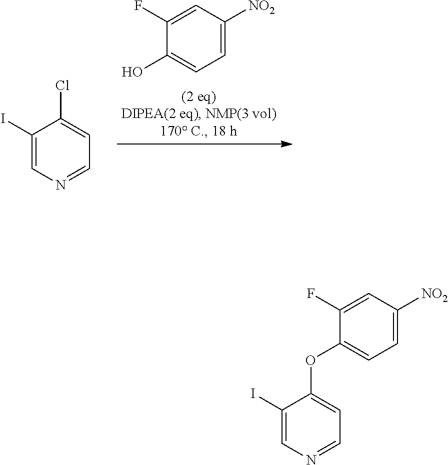 [SMILES: O=[N+]([O-])c1ccc(O)c(F)c1 ; Clc1ccncc1I ; O=[N+]([O-])c1ccc(Oc2ccncc2I)c(F)c1]

A mixture of 4-chloro-3-iodopyridine (1 g, 4.18 mmol) and 2-fluoro-4-nitrophenol (984 mg, 1.5 eq., 6.26 mmol) in N-Methyl-2-pyrrolidone (15 mL) was added N,N-ethylbis(propan-2-yl)amine (2.18 mL, 3 eq, 12.5 mmol) in a glass pressure vessel (Sealed tube). The reaction mixture was heated at 170° C. for 18 h. Progress of the reaction was monitored by TLC. After completion of reaction, the volatile components were distilled off under reduced pressure and the viscous residue poured into ice-water (500 mL) the pH of the mixture was adjusted to ˜7.5 with saturated aq. NaHCO₃ solution and then extracted with ethyl acetate (100 mL). The EtOAc layer was separated, washed with brine (30 mL) and then dried over Na₂SO₄. The organic layer was concentrated under reduced pressure to get crude residue. The crude compound was purified by using by column chromatography using silica 60-120 (10-15% EtOAc in n-Hexane as eluent) to afford the title compound 4-(2-fluoro-4-nitrophenoxy)-3-iodopyridine (0.6 g, 1.67 mmol) as a yellow solid.

¹H NMR (400 MHz, DMSO-d₆): δ=8.97 (s, 1H), 8.48-8.43 (m, 2H), 8.17 (d, J=8.80 Hz, 1H), 7.50 (t, J=8.40 Hz, 1H), 7.07 (d, J=5.60 Hz, 1H), LCMS: 361.0 (M+H)⁺.

### Step 2: Synthesis of 3-fluoro-4-((3-iodopyridin-4-yl)oxy)aniline

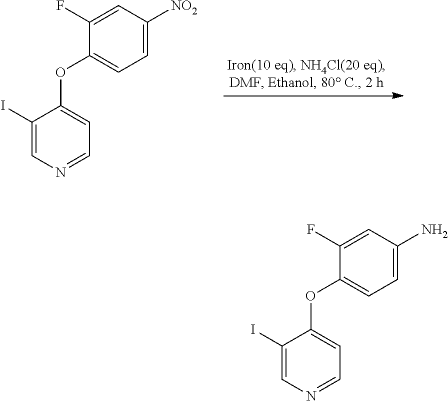 [SMILES: O=[N+]([O-])c1ccc(Oc2ccncc2I)c(F)c1 ; Nc1ccc(Oc2ccncc2I)c(F)c1]

To a stirred solution of 4-(2-fluoro-4-nitrophenoxy)-3-iodopyridine (0.4 g, 1.11 mmol) in ethanol (4 mL, 68.5 mmol), water (0.5 mL, 27.8 mmol) and dimethylformamide (0.5 mL, 6.46 mmol) at 0° C., iron (620 mg, 10 eq., 11.1 mmol) and ammonium chloride (1.19 g, 20 eq., 22.2 mmol) were added. The reaction mixture then stirred at 80° C. for 1 h. Progress of the reaction was monitored by TLC. After completion of reaction, reaction mixture was diluted with EtOH (10 mL) and filtered through a pad of diatomaceous earth (i.e., Celite®) and washed with water to DMF (2:1). Filtrate was concentrated and then aq. Layer was quenched with aq. sodium bicarbonate solution (50 mL) and extracted with 10% MeOH in DCM (3×200 mL). Combined organic layer was dried over Na₂SO₄ and concentrated under reduced pressure to get crude compound. The crude compound was purified by silica gel (100-200 mesh) column chromatography using 10% MeOH in DCM as a eluent to afford title compound 3-fluoro-4-(3-iodo-4-pyridyloxy)aniline (250 mg, 757 μmol) colorless semisolid.

¹H NMR (400 MHz, DMSO-d₆): δ=8.79 (s, 1H), 8.31 (d, J=5.20 Hz, 1H), 7.01 (t, J=8.80 Hz, 1H), 6.56 (d, J=5.60 Hz, 1H), 6.53-6.49 (m, 1H), 6.42 (d, J=8.40 Hz, 1H), 5.52 (s, 2H), LCMS: 331.0 (M+H)⁺.

### Step 3: Synthesis of(4-(4-amino-2-fluorophenoxy)pyridin-3-yl)dimethylphosphine oxide

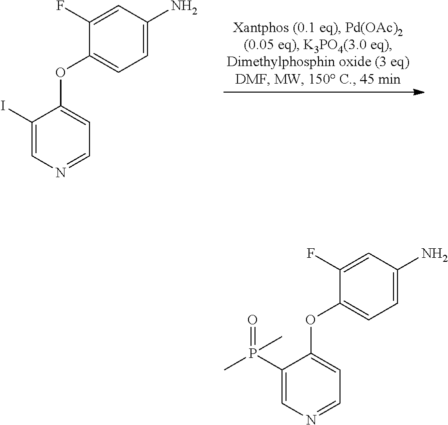 [SMILES: Nc1ccc(Oc2ccncc2I)c(F)c1 ; CP(C)(=O)c1cnccc1Oc1ccc(N)cc1F]

To a stirred solution of 3-fluoro-4-(3-iodo-4-pyridyloxy)aniline (0.4 g, 1.21 mmol) in 1,4-dioxane (8.36 mL, 98.1 mmol) was added tripotassium phosphate (772 mg, 3 eq., 3.64 mmol). The reaction mixture was purged with N₂ gas for 20 min. After that dimethyl(oxo)-as-phosphanylium (280 mg, 3 eq., 3.64 mmol) and palladium(2+) diacetate (136 mg, 0.5 eq., 606 μmol) were added. The reaction mixture again purged with N₂ gas for 10 min. Then the reaction mixture was stirred at 120° C. for 6 h. Reaction progress was monitored by TLC & LCMS. After completion of reaction, excess of dioxane was evaporated under reduced pressure to get the crude residue. The crude compound was purified by column chromatography on silica gel (100-200 mesh) using 10% MeOH in DCM as a eluent to afford title compound [4-(4-amino-2-fluorophenoxy)-3-pyridyl]dimethylphosphine oxide (250 mg, 892 μmol) as semisolid.

¹H NMR (400 MHz, DMSO-d₆): δ=8.75 (d, J=7.60 Hz, 1H), 8.57 (d, J=5.60 Hz, 1H), 7.04 (t, J=8.80 Hz, 1H), 6.65-6.64 (m, 1H), 6.52 (d, J=13.2 Hz, 1H), 6.45-6.39 (m, 1H), 5.54 (s, 2H), 1.77 (d, J=13.6 Hz, 6H), LCMS: 281.1 (M+H)⁺.

### Step 4: Synthesis of N-(4-((3-(dimethylphosphoryl)pyridin-4-yl)oxy)-3-fluorophenyl)-1-(4-fluorophenyl)-3-(trifluoromethyl)-1H-pyrazole-4-carboxamide and N-(4-((3-(dimethylphosphoryl)pyridin-4-yl)oxy)-3-fluorophenyl)-1-(4-fluorophenyl)-5-(trifluoromethyl)-1H-pyrazole-4-carboxamide

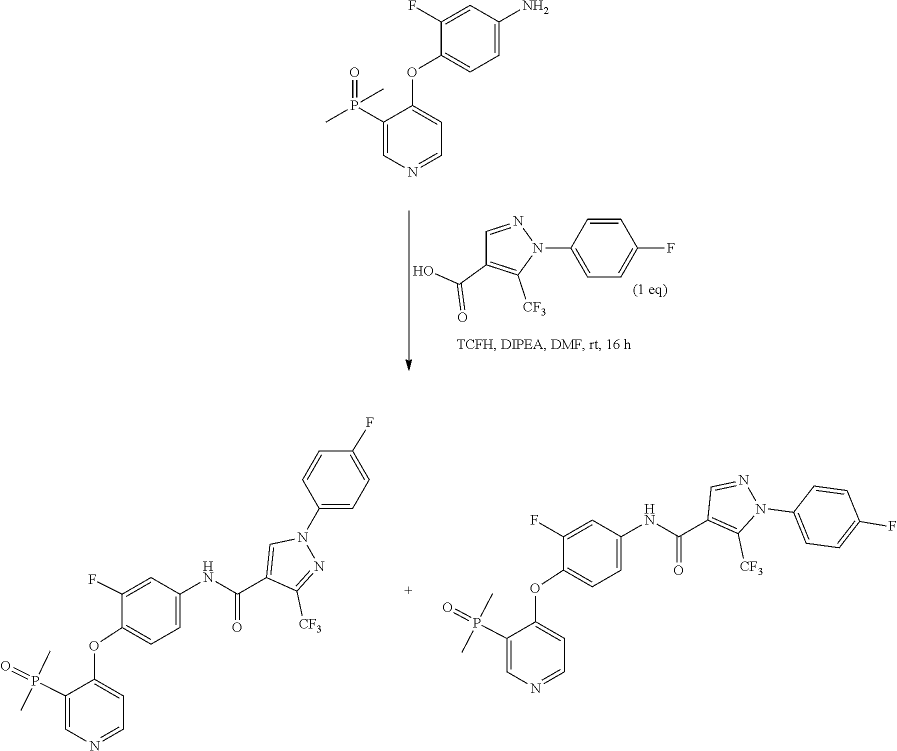 [SMILES: CP(C)(=O)c1cnccc1Oc1ccc(N)cc1F ; O=C(O)c1cnn(-c2ccc(F)cc2)c1C(F)(F)F ; CP(C)(=O)c1cnccc1Oc1ccc(NC(=O)c2cn(-c3ccc(F)cc3)nc2C(F)(F)F)cc1F ; CP(C)(=O)c1cnccc1Oc1ccc(NC(=O)c2cnn(-c3ccc(F)cc3)c2C(F)(F)F)cc1F]

To a stirred solution of [4-(4-amino-2-fluorophenoxy)-3-pyridyl]dimethylphosphine oxide (0.3 g, 1.07 mmol) and 1-(p-fluorophenyl)-5-(trifluoromethyl)-4-pyrazolecarboxylic acid (352 mg, 1.2 eq., 1.28 mmol) in tetrahydrofuran (135 mL, 1.66 mol) was added chloro(dimethylamino)methylenebis(methyl)azanium hexafluoridophosphate(1-) (601 mg, 2 eq., 2.14 mmol) and N-ethylbis(isopropyl)amine (956 μL, 5 eq., 5.35 mmol) at 0° C. Then the reaction mixture was stirred at room temperature for 16 h. Progress of the reaction was monitored by TLC & LCMS. After completion of reaction, reaction mixture was diluted with water (50 mL) and extracted in EtOAc (3×50 mL). Combined organic layer was dried over sodium sulphate and concentrated under reduced pressure to get crude residue. The crude compound was purified on silica gel (100-200 mesh) using 15% MeOH in DCM as an eluent to afford fraction 1 (70 mg, with purity 62% by LCMS) and fraction 2 (170 mg, with 42% purity by LCMS). Fraction 1 was again purified by Prep-HPLC using 0.1% FA in water and acetonitrile to afford N-{4-[3-(dimethylphosphoryl)-4-pyridyloxy]-3-fluorophenyl}-1-(p-fluorophenyl)-5-(trifluoromethyl)-4-pyrazolecarboxamide (7 mg, 13.1 μmol) as white solid and fraction 2 was again purified by Prep-HPLC using 0.1% FA in water and acetonitrile to afford N-(4-((3-(dimethylphosphoryl)pyridin-4-yl)oxy)-3-fluorophenyl)-1-(4-fluorophenyl)-3-(trifluoromethyl)-1H-pyrazole-4-carboxamide (41 mg, 13.1 μmol) as white solid.

Compound 52 Analysis: ¹H NMR (400 MHz, DMSO-d₆): δ=10.89 (s, 1H), 8.81 (d, J=7.20 Hz, 1H), 8.60 (d, J=6.00 Hz, 1H), 8.35 (s, 1H), 7.94 (dd, J=2.00 Hz, 12.80 Hz, 1H), 7.66-7.57 (m, 3H), 7.52-7.44 (m, 3H), 7.76 (t, J=4.80 Hz, 1H), 1.81 (d, J=14.00 Hz, 6H), LCMS: 537.2 (M+H)⁺.

Compound 53 Analysis: ¹H NMR (400 MHz, DMSO-d₆): δ=10.32 (s, 1H), 8.60 (s, 1H), 8.48 (d, J=7.60 Hz, 1H), 8.30 (d, J=6.80 Hz, 1H), 7.73-7.69 (m, 2H), 7.49-7.42 (m, 3H), 7.39-7.36 (m, 1H), 7.14-7.11 (m, 1H), 1.86 (d, J=13.60 Hz, 6H), LCMS: 537.1 (M+H)⁺.

### Synthetic Example 35

### Synthesis of N-{4-[5-(Dimethylphosphoryl)-4-Pyrimidinyloxy]-3-Fluorophenyl}-1-(P-Fluorophenyl)-5-(Trifluoromethyl)-4-Pyrazolecarboxamide (Compound 54)

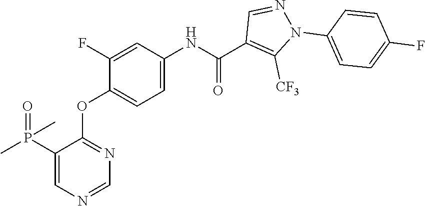 [SMILES: CP(C)(=O)c1cncnc1Oc1ccc(NC(=O)c2cnn(-c3ccc(F)cc3)c2C(F)(F)F)cc1F]

### Step 1: Synthesis of 3-fluoro-4-(5-iodo-4-pyrimidinyloxy)aniline

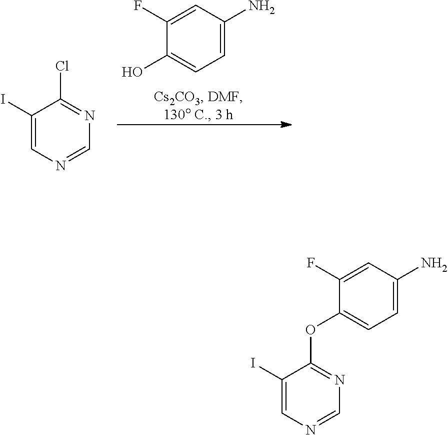 [SMILES: Clc1ncncc1I ; Nc1ccc(O)c(F)c1 ; Nc1ccc(Oc2ncncc2I)c(F)c1]

To a mixture of 4-chloro-5-iodopyrimidine (1 g, 4.16 mmol, 1 eq) and 4-amino-2-fluorophenol (793 mg, 1.5 eq, 6.24 mmol) in dimethylformamide (20 mL) was added dicesium carbonate (2.03 g, 1.5 eq., 6.24 mmol) was placed in a glass pressure vessel (Sealed tube) and heated rapidly to 150° C. The heating was continued for 3 h. Reaction progress was monitored by TLC. After completion of reaction, the reaction mixture was quenched with ice cold water (30 mL) and extracted with Ethyl acetate (3×30 mL). The combined organic layer wash with brine solution (30 mL) dried with Na₂SO₄ and concentrated under reduced pressure to get the crude residue. The crude compound was purified by flash column by using 100-200 silica mesh with 2-3% MeOH in DCM as eluent to afford title compound 3-fluoro-4-(5-iodo-4-pyrimidinyloxy)aniline (0.4 g, 29.05% yield) as off-white solid.

¹H NMR (400 MHz, DMSO-d₆): δ=9.01 (s, 1H), 8.64 (s, 1H), 6.97 (t, J=8.80 Hz, 1H), 6.46 (dd, J=2.40 Hz, 13.20 Hz, 1H), 6.38 (dd, J=1.20 Hz, 8.60 Hz, 1H), 5.42 (s, 2H), LCMS: 332.0 (M+H)⁺.

### Step 2: Synthesis of [4-(4-amino-2-fluorophenoxy)-5-pyrimidinyl]dimethylphosphine oxide

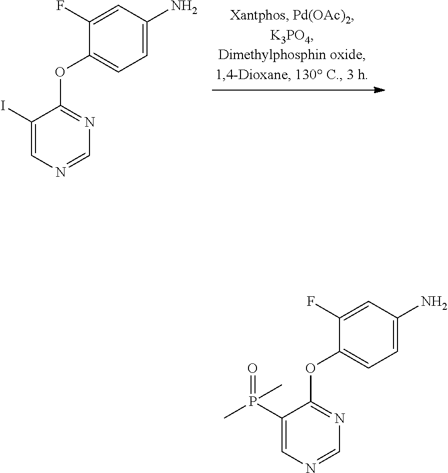 [SMILES: Nc1ccc(Oc2ncncc2I)c(F)c1 ; CP(C)(=O)c1cncnc1Oc1ccc(N)cc1F]

To a stirred solution of 3-fluoro-4-(5-iodo-4-pyrimidinyloxy)aniline (0.4 g, 1.21 mmol, 1 eq) in 1,4-dioxane (7.69 mL) was added tripotassium phosphate (410 mg, 1.6 eq., 1.93 mmol). The reaction mixture was degassed with Argon gas for 20 min. Then [5-(diphenylphosphanyl)-9,9-dimethyl-9H-xanthen-4-yl]diphenylphosphane (105 mg, 0.15 eq., 0.181 mmol) and dimethyl(oxo)-λ⁵-phosphanylium (0.244 mL, 3 eq., 3.62 mmol) were added. Then reaction mixture again degassed with Argon gas for 10 min. After that palladium(2+) diacetate (13.6 mg, 0.05 eq., 0.060 mmol) was added at room temperature under Argon atmosphere. Reaction mixture was stirred at 130° C. for 3 h. Reaction progress was monitored by TLC. After completion of reaction, the reaction mixture was concentrated under reduced pressure to get crude compound. The crude compound was purified by combi-flash using 3% MeOH in DCM as eluent to afford title compound [4-(4-amino-2-fluorophenoxy)-5-pyrimidinyl]dimethylphosphine oxide (150 mg, 44.15% yield) as a pale green color solid.

¹H NMR (400 MHz, DMSO-d₆): δ=8.90-8.87 (m, 2H), 6.02 (t, J=8.80 Hz, 1H), 6.48 (dd, J=2.40 Hz, 13.20 Hz, 1H), 6.40 (dd, J=2.40 Hz, 8.60 Hz, 1H), 5.44 (s, 2H), 1.81 (d, J=14.00 Hz, 6H), LCMS: 282.1 (M+H)⁺.

### Step 3: Synthesis of N-{4-[5-(dimethylphosphoryl)-4-pyrimidinyloxy]-3-fluorophenyl}-1-(p-fluorophenyl)-5-(trifluoromethyl)-4-pyrazolecarboxamide

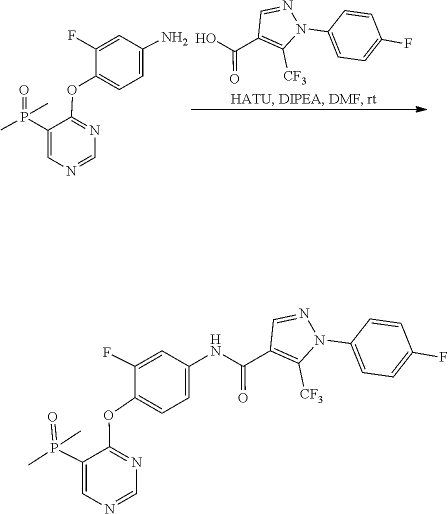 [SMILES: CP(C)(=O)c1cncnc1Oc1ccc(N)cc1F ; O=C(O)c1cnn(-c2ccc(F)cc2)c1C(F)(F)F ; CP(C)(=O)c1cncnc1Oc1ccc(NC(=O)c2cnn(-c3ccc(F)cc3)c2C(F)(F)F)cc1F]

To a stirred solution of 1-(4-fluorophenyl)-5-(trifluoromethyl)-1H-pyrazole-4-carboxylic acid (97.5 mg, 0.356 mmol, 1 eq) in dimethylformamide (5 mL) were added N,N-ethylbis(propan-2-yl)amine (0.191 mL, 3 eq., 1.07 mmol) and [bis(dimethylamino)methylidene]({3H-[1,2,3]triazolo[4,5-b]pyridin-3-yl})oxidanium (167 mg, 2 eq., 0.711 mmol). The reaction mixture was stirred for 5 min then [4-(4-amino-2-fluorophenoxy)-5-pyrimidinyl]dimethylphosphine oxide (0.1 g, 0.356 mmol, 1 eq) was added. The reaction mixture was stirred at room temperature for 16 h. Reaction progress was monitored by TLC. After completion of reaction, reaction mixture was quenched with ice cold water (10 mL) and extracted with EtOAc (3×15 mL). The combined organic layer was washed with brine solution (10 mL) and dried over Na₂SO₄ and concentrated under reduced pressure to get crude compound. The crude compound was purified by Prep-HPLC using 0.1% TFA in acetonitrile and water to afford the title compound N-{4-[5-(dimethylphosphoryl)-4-pyrimidinyloxy]-3-fluorophenyl}-1-(p-fluorophenyl)-5-(trifluoromethyl)-4-pyrazolecarboxamide (8 mg, 4.19% yield) as white solid.

¹H NMR (400 MHz, DMSO-d₆): δ=10.84 (s, 1H), 8.96-8.92 (m, 2H), 8.35 (s, 1H), 7.86 (dd, J=2.40 Hz, 12.60 Hz, 1H), 7.65-7.62 (m, 2H), 7.54 (dd, J=2.00 Hz, 9.00 Hz, 1H), 7.50-7.44 (m, 3H), 1.85 (d, J=14.00 Hz, 6H), LCMS: 538.54 (M+H)⁺.

### Synthetic Example 36

### Synthesis of N-{4-[6-Amino-5-(Dimethylphosphoryl)-4-Pyrimidinyloxy]-3-Fluorophenyl}-1-(P-Fluorophenyl)-5-(Trifluoromethyl)-4-Pyrazole Carboxamide (Compound 55)

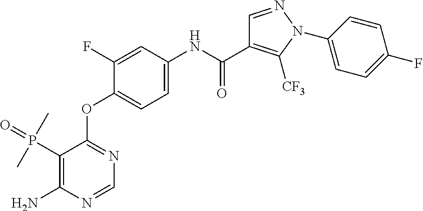 [SMILES: CP(C)(=O)c1c(N)ncnc1Oc1ccc(NC(=O)c2cnn(-c3ccc(F)cc3)c2C(F)(F)F)cc1F]

### Step 1: Synthesis of 6-(4-amino-2-fluorophenoxy)-5-iodo-4-pyrimidinamine

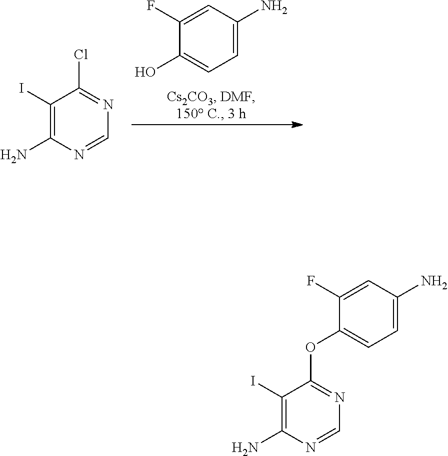 [SMILES: Nc1ncnc(Cl)c1I ; Nc1ccc(O)c(F)c1 ; Nc1ccc(Oc2ncnc(N)c2I)c(F)c1]

To a stirred mixture of 6-chloro-5-iodo-4-pyrimidinylamine (1 g, 3.91 mmol, 1 eq) and 4-amino-2-fluorophenol (746 mg, 1.5 eq, 5.87 mmol) in dimethylformamide (40 mL) was added dicesium carbonate (1.91 g, 1.5 eq, 5.87 mmol) was placed in a glass pressure vessel (Sealed tube) and heated rapidly to 150° C. The heating was continued for 3 h. Reaction progress was monitored by TLC. After completion of reaction, the reaction mixture was quenched with ice cold water (30 mL) and extracted with Ethyl acetate (3×30 mL). The combined organic layer wash with brine solution (30 mL) dried with Na₂SO₄ and concentrated under reduced pressure to get crude compound. The crude compound was purified by flash column by using 100-200 silica mesh with 2-3% MeOH in DCM as eluent to afford title compound 6-(4-amino-2-fluorophenoxy)-5-iodo-4-pyrimidinamine (0.7 g, 51.66% yield) as off-white solid.

¹H NMR (400 MHz, DMSO-d₆): δ=7.87 (s, 1H), 6.87 (t, J=8.80 Hz, 1H), 6.42 (dd, J=2.40 Hz, 12.80 Hz, 1H), 6.34 (dd, J=1.60 Hz, 8.60 Hz, 1H), 5.32 (s, 2H), LCMS: 347.0 (M+H)⁺.

### Step 2: Synthesis of [6-amino-4-(4-amino-2-fluorophenoxy)-5-pyrimidinyl]dimethylphosphine oxide

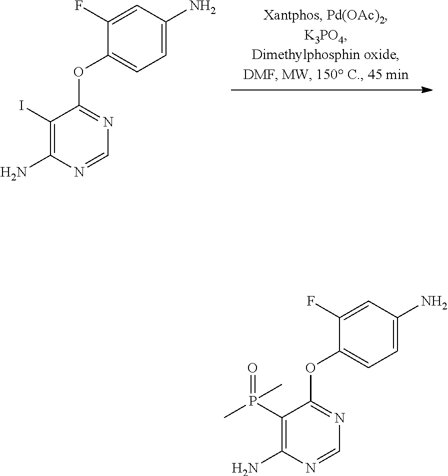 [SMILES: Nc1ccc(Oc2ncnc(N)c2I)c(F)c1 ; CP(C)(=O)c1c(N)ncnc1Oc1ccc(N)cc1F]

To a stirred solution of 6-(4-amino-2-fluorophenoxy)-5-iodo-4-pyrimidinamine (0.7 g, 2.02 mmol, 1 eq) in 1,4-dioxane (6.98 mL) was added tripotassium phosphate (687 mg, 1.6 eq, 3.24 mmol) and degassed with argon gas for 20 min. Then [5-(diphenylphosphanyl)-9,9-dimethyl-9H-xanthen-4-yl]diphenylphosphane (176 mg, 0.15 eq, 0.303 mmol) and dimethyl(oxo)-λ⁵-phosphanylium (0.408 mL, 3 eq., 6.07 mmol) was added and reaction mixture was again degassed with argon gas for 10 min. After that palladium(2+) diacetate (22.7 mg, 0.05 eq, 0.101 mmol) was added to reaction mixture at room temperature under Argon atmosphere. Reaction mixture was stirred at 130° C. for 3 h. Reaction progress was monitored by TLC. After completion of reaction, reaction mixture was concentrated under reduced pressure to get crude residue. The crude compound was then purified by combi flash, the spot was eluted with 3% MeOH in DCM to afford title compound [6-amino-4-(4-amino-2-fluorophenoxy)-5-pyrimidinyl]dimethylphosphine oxide (0.4 g, 66.76%) as a pale green color solid.

¹H NMR (400 MHz, DMSO-d₆): δ=8.61 (s, 1H), 8.06 (d, J=2.00 Hz, 1H), 7.38 (brs, 1H), 6.91 (t, J=8.80 Hz, 1H), 6.42 (dd, J=2.40 Hz, 13.20 Hz, 1H), 6.34 (dd, J=1.60 Hz, 8.80 Hz, 1H), 5.35 (s, 2H), LCMS: 297.1 (M+H)⁺.

### Step 3: Synthesis of N-{4-[6-amino-5-(dimethylphosphoryl)-4-pyrimidinyloxy]-3-fluorophenyl}-1-(p-fluorophenyl)-5-(trifluoromethyl)-4-pyrazolecarboxamide

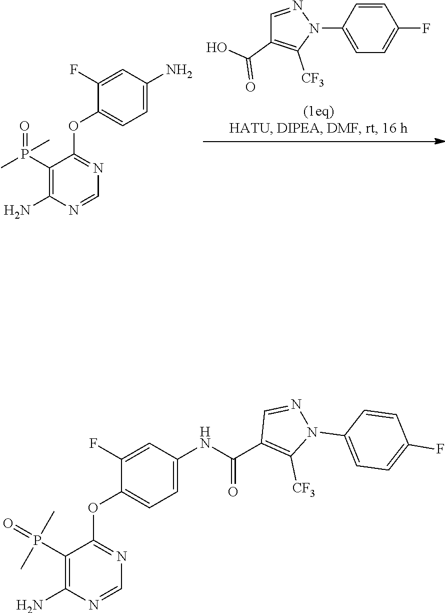 [SMILES: CP(C)(=O)c1c(N)ncnc1Oc1ccc(N)cc1F ; O=C(O)c1cnn(-c2ccc(F)cc2)c1C(F)(F)F ; CP(C)(=O)c1c(N)ncnc1Oc1ccc(NC(=O)c2cnn(-c3ccc(F)cc3)c2C(F)(F)F)cc1F]

To a stirred solution of 1-(4-fluorophenyl)-5-(trifluoromethyl)-1H-pyrazole-4-carboxylic acid (185 mg, 0.675 mmol) in dimethylformamide (6.67 mL) were added N,N-ethylbis(propan-2-yl)amine (0.362 mL, 3 eq., 2.03 mmol) and [bis(dimethylamino)methylidene]({3H-[1,2,3]triazolo[4,5-b]pyridin-3-yl})oxidanium (318 mg, 2 eq., 1.35 mmol). The reaction mixture was stirred for 5 min at rt and then [6-amino-4-(4-amino-2-fluorophenoxy)-5-pyrimidinyl]dimethylphosphine oxide (0.2 g, 0.675 mmol, 1 eq) was added. The reaction mixture was stirred at room temperature for 16 h. Reaction progress was monitored by TLC. After completion of reaction, reaction mixture was quenched with ice cold water (15 mL) and extracted with EtOAc (3×15 mL). The combined organic layer was washed with brine solution (15 mL) and dried over Na₂SO₄ and then concentrated under reduced pressure to get crude residue. The crude compound was purified by Prep-HPLC using 0.1% TFA in acetonitrile and water to get the title compound of N-{4-[6-amino-5-(dimethylphosphoryl)-4-pyrimidinyloxy]-3-fluorophenyl}-1-(p-fluorophenyl)-5-(trifluoromethyl)-4-pyrazolecarboxamide (40 mg, 10.73% yield) as white solid.

¹H NMR (400 MHz, DMSO-d₆): δ=10.77 (s, 1H), 8.67 (d, J=3.2 Hz, 1H), 8.34 (s, 1H), 8.08 (d, J=2.00 Hz, 1H), 7.79 (dd, J=2.40 Hz, 12.60 Hz, 1H), 7.65-7.61 (m, 2H), 7.53-7.44 (m, 4H), 7.37 (t, J=8.80 Hz, 1H), 1.83 (d, J=13.60 Hz, 6H), LCMS: 553.55 (M+H)⁺.

### Synthetic Example 37

### Synthesis of N-{4-[6-Amino-5-(Dimethylphosphoryl)-4-Pyrimidinyloxy]-3-Fluorophenyl}-4-Ethoxy-1-(P-Fluorophenyl)-2-Oxo-1,2-Dihydronicotinamide (Compound 56)

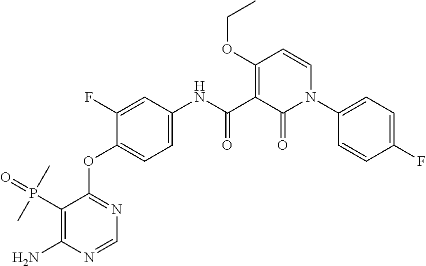 [SMILES: CCOc1ccn(-c2ccc(F)cc2)c(=O)c1C(=O)Nc1ccc(Oc2ncnc(N)c2P(C)(C)=O)c(F)c1]

### Step 1: Synthesis of ethyl (E)-2-cyano-3-ethoxybut-2-enoate

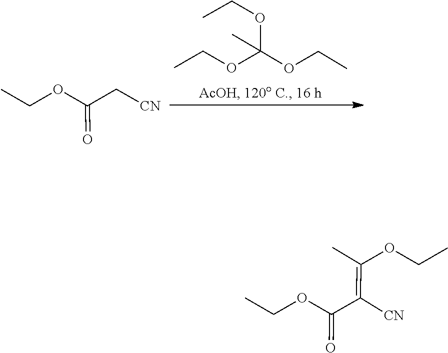

To a stirred solution of 1,1,1-triethoxyethane (14.7 mL, 80.1 mmol, 1 eq) and acetic acid (1.11 mL, 18.4 mmol, 0.23 eq) were sequentially added to ethyl cyanoacetate (4.53 mL, 40.1 mmol, 0.5 eq) and the reaction mixture was stirred at 120° C. for 16 h. After 16 h of stirring at 120° C., reaction mixture was cooled to rt and concentrated under vacuum to afford crude ethyl (E)-2-cyano-3-ethoxybut-2-enoate (14 g, crude). The crude residue was carried into the next reaction without further purification (as reported in US 2022/0288043 A1).

### Step 2: Synthesis of ethyl (2E,4E)-2-cyano-5-(dimethylamino)-3-ethoxypenta-2,4-dienoate

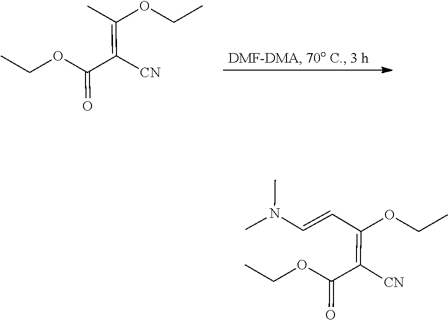 [SMILES: CCOC(=O)/C(C#N)=C(\C)OCC ; CCOC(=O)/C(C#N)=C(\C=C\N(C)C)OCC]

A mixture of ethyl (E)-2-cyano-3-ethoxybut-2-enoate (14 g, 76.4 mmol, 1 eq, crude-Theoretically) and N,N-dimethyl(dimethoxymethyl)amine (8 mL) was heated at 70° C. for 3 h. After 3 h of stirring at 70° C., the reaction mixture was concentrated under high vacuum to afford crude ethyl (2E,4E)-2-cyano-5-(dimethylamino)-3-ethoxypenta-2,4-dienoate (16 g, crude). The residue was carried to next step without further purification (as reported in US Publication No. 2022/0288043).

### Step 3: Synthesis of ethyl 4-ethoxy-2-oxo-1,2-dihydropyridine-3-carboxylate

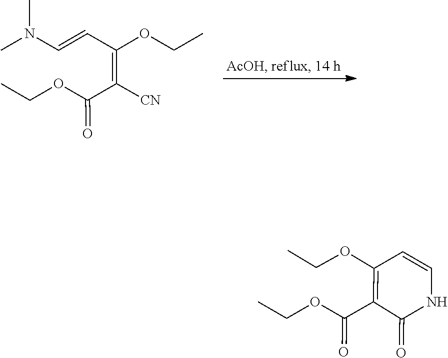 [SMILES: CCOC(=O)/C(C#N)=C(\C=C\N(C)C)OCC ; CCOC(=O)c1c(OCC)cc[nH]c1=O]

A mixture of ethyl (2E,4E)-2-cyano-5-(dimethylamino)-3-ethoxypenta-2,4-dienoate (16 g, 67.1 mmol, 0.89 eq) and acetic acid (35 mL) was refluxed for 16 h. Progress of reaction was monitored by TLC. After completion of reaction, the reaction mixture was concentrated to dryness under high vacuum, treated with water (50 mL) and washed with EtOAc (30 mL) to remove the impurities. The pH of the aqueous layer was adjusted to pH 9-10 with aq. NaHCO₃ solution. The mixture was extracted with DCM (3×50 mL). The combined organic layer was washed with brine, dried over Na₂SO₄ and concentrated under reduced pressure to get crude residue. The residue was purified by silica gel chromatography to afford desired compound ethyl 4-ethoxy-2-oxo-1,2-dihydropyridine-3-carboxylate (2.5 g, 15.67% yield) as yellow solid.

¹H NMR (400 MHz, DMSO-d₆): δ=11.65 (s, 1H), 7.48 (d, J=7.60 Hz, 1H), 6.23 (d, J=7.20 Hz, 1H), 4.19-4.11 (m, 4H), 1.27-1.21 (m, 6H), LCMS: 212.1 (M+H)⁺.

### Step 4: Synthesis of ethyl 4-ethoxy-1-(4-fluorophenyl)-2-oxo-1,2-dihydropyridine-3-carboxylate

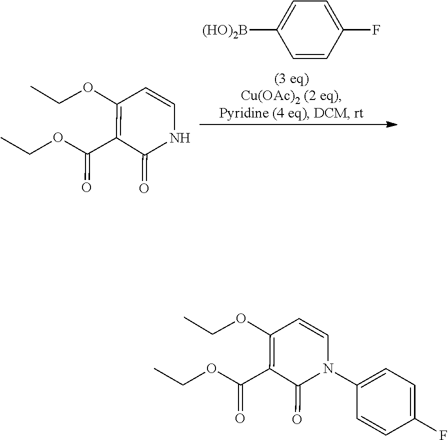 [SMILES: OB(O)c1ccc(F)cc1 ; CCOC(=O)c1c(OCC)cc[nH]c1=O ; CCOC(=O)c1c(OCC)ccn(-c2ccc(F)cc2)c1=O]

To a stirred solution of ethyl 4-ethoxy-2-oxo-1,2-dihydropyridine-3-carboxylate (2 g, 9.47 mmol, 1 eq), (p-fluorophenyl)boranediol (3.97 g, 28.4 mmol, 3 eq) and copper(II)acetate (3.44 g, 18.9 mmol, 2 eq) in DCM (20 mL) was added pyridine (3.06 mL, 37.9 mmol, 4 eq). The reaction mixture was stirred at room temperature for 16 h in presence of air. Reaction progress was monitored by TLC. After completion of reaction, reaction mixture was diluted with DCM (100 mL) and filtered through a pad of diatomaceous earth (i.e., Celite®) and washed with DCM (50 mL). The combined organic layer was washed with water (30 mL), brine (30 mL) then dried over Na₂SO₄ and concentrated under reduced pressure to get crude residue. The crude compound was purified by flash column chromatography using 50-100% EtOAc in hexane to afford title compound ethyl 4-ethoxy-1-(4-fluorophenyl)-2-oxo-1,2-dihydropyridine-3-carboxylate (1.5 g, 51.89% yield) as yellow solid.

¹H NMR (400 MHz, DMSO-d₆): δ=7.81 (d, J=7.60 Hz, 1H), 7.46-7.42 (m, 2H), 7.34 (t, J=8.80 Hz, 2H), 6.44 (d, J=8.00 Hz, 1H), 4.24-4.15 (m, 4H), 1.28 (t, J=7.2 Hz, 3H), 1.22 (t, J=7.2 Hz, 3H), LCMS: 306.1 (M+H)⁺.

### Step 5: Synthesis of 4-ethoxy-1-(4-fluorophenyl)-2-oxo-1,2-dihydropyridine-3-carboxylic acid

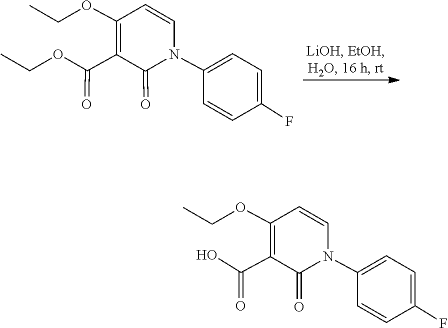 [SMILES: CCOC(=O)c1c(OCC)ccn(-c2ccc(F)cc2)c1=O ; CCOc1ccn(-c2ccc(F)cc2)c(=O)c1C(=O)O]

To a stirred suspension of ethyl 4-ethoxy-1-(4-fluorophenyl)-2-oxo-1,2-dihydropyridine-3-carboxylate (1 g, 3.28 mmol, 1 eq) in ethanol (15 mL) and water (7.5 mL) was added lithium hydroxide monohydrate (550 mg, 13.1 mmol, 4 eq). The reaction mixture was stirred at room temperature for 16 h. Reaction progress was monitored by TLC. After completion of reaction, total solvent evaporated under reduced pressure and residue was diluted with water (20 mL), cooled to 0° C. and acidified with 1N HCl and stirred for 10 min. The obtained solid was filtered, washed with water (20 mL), Diethyl ether (30 mL). Then dried under vacuum to afford title compound 4-ethoxy-1-(4-fluorophenyl)-2-oxo-1,2-dihydropyridine-3-carboxylic acid (650 mg, 71.58% yield) as white solid.

¹H NMR (400 MHz, DMSO-d₆): δ=13.81 (s, 1H), 7.98 (d, J=8.00 Hz, 1H), 7.53-7.49 (m, 2H), 7.38 (t, J=8.40 Hz, 2H), 6.61 (d, J=8.00 Hz, 1H), 4.31 (q, J=6.8 Hz, 2H), 1.35 (t, J=6.8 Hz, 3H), LCMS: 278.1 (M+H)⁺.

### Step 6: Synthesis of N-{4-[6-amino-5-(dimethylphosphoryl)-4-pyrimidinyloxy]-3-fluorophenyl}-4-ethoxy-1-(p-fluorophenyl)-2-oxo-1,2-dihydronicotinamide

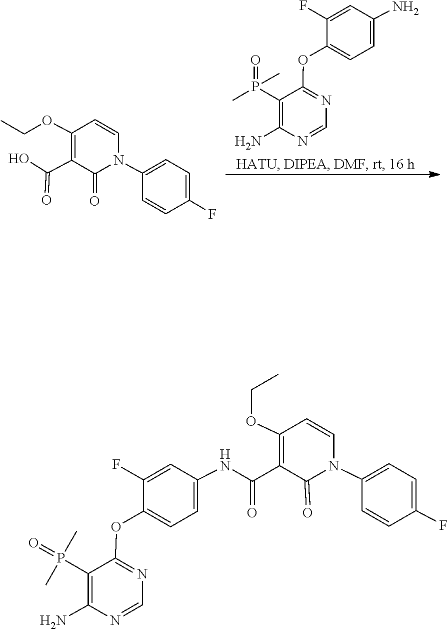 [SMILES: CCOc1ccn(-c2ccc(F)cc2)c(=O)c1C(=O)O ; CP(C)(=O)c1c(N)ncnc1Oc1ccc(N)cc1F ; CCOc1ccn(-c2ccc(F)cc2)c(=O)c1C(=O)Nc1ccc(Oc2ncnc(N)c2P(C)(C)=O)c(F)c1]

To a stirred solution of 4-ethoxy-1-(p-fluorophenyl)-2-oxo-1,2-dihydronicotinic acid (187 mg, 0.675 mmol) in dimethylformamide (6.67 mL) was added N,N-ethylbis(propan-2-yl)amine (0.362 mL, 3 eq., 2.03 mmol) and [bis(dimethylamino)methylidene]({3H-[1,2,3]triazolo[4,5-b]pyridin-3-yl})oxidanium (318 mg, 2 eq., 1.35 mmol). The reaction mixture was stirred for 5 min then [6-amino-4-(4-amino-2-fluorophenoxy)-5-pyrimidinyl]dimethylphosphine oxide (0.2 g, 0.675 mmol, 1 eq) was added. The reaction mixture was stirred at room temperature for 16 h. Reaction progress was monitored by TLC. After completion of reaction, reaction mixture was quenched with ice cold water (15 mL) and extracted with EtOAc (3×15 mL). The combined organic layer was washed with brine solution (15 mL) and dried over Na₂SO₄ and concentrated under reduced pressure to get crude residue. The crude compound was purified by Prep-HPLC using 0.1% TFA in water and acetonitrile to get the title compound N-{4-[6-amino-5-(dimethylphosphoryl)-4-pyrimidinyloxy]-3-fluorophenyl}-4-ethoxy-1-(p-fluorophenyl)-2-oxo-1,2-dihydronicotinamide (80 mg, 21.33% yield) as white solid.

¹H NMR (400 MHz, DMSO-d₆): δ 10.52 (s, 1H), 8.66 (d, J=3.2 Hz, 1H), 8.07 (d, J=2.0 Hz, 1H), 7.85 (d, J=8.00 Hz, 1H), 7.79 (dd, J=2.0 Hz, 12.80 Hz, 2H), 7.51-7.43 (m, 3H), 7.39-7.34 (m, 3H), 7.29 (t, J=8.80 Hz, 1H), 6.50 (d, J=8.00 Hz, 1H), 4.24 (q, J=6.8 Hz, 2H), 1.82 (d, J=13.60 Hz, 6H), 1.29 (t, J=6.8 Hz, 3H), LCMS: 556.36 (M+H)⁺.

### Synthetic Example 38

### Synthetic Scheme for N-(4-((2-((3-(2-(4-Acryloylpiperazin-1-Yl)-2-Oxoethyl)Phenyl)Amino)-3-(Dimethylphosphoryl)Pyridin-4-Yl)Oxy)-3-Fluorophenyl)-1-(4-Fluorophenyl)-5-(Trifluoromethyl)-1H-Pyrazole-4-Carboxamide (Compound 57)

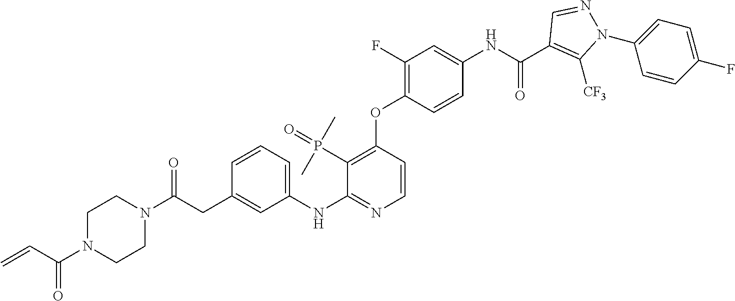 [SMILES: C=CC(=O)N1CCN(C(=O)Cc2cccc(Nc3nccc(Oc4ccc(NC(=O)c5cnn(-c6ccc(F)cc6)c5C(F)(F)F)cc4F)c3P(C)(C)=O)c2)CC1]

### Step 1: Synthesis of tert-butyl 4-(2-(3-bromophenyl)acetyl)piperazine-1-carboxylate

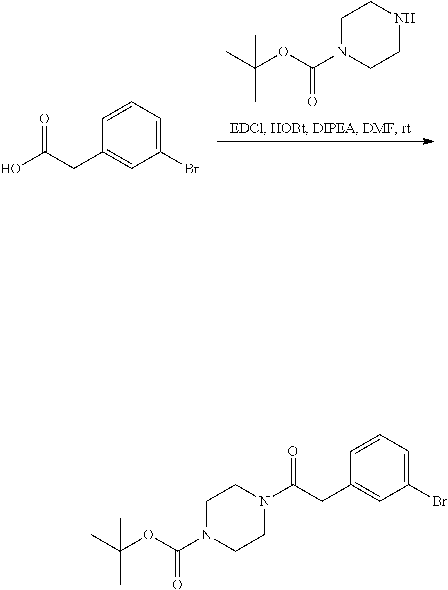 [SMILES: CC(C)(C)OC(=O)N1CCNCC1 ; O=C(O)Cc1cccc(Br)c1 ; CC(C)(C)OC(=O)N1CCN(C(=O)Cc2cccc(Br)c2)CC1]

To a stirred solution of (m-bromophenyl)acetic acid (1 g, 4.65 mmol) and tert-butyl 1-piperazinecarboxylate (1.04 g, 1.2 eq., 5.58 mmol) in DMF (5 mL) was added 1H-1,2,3-benzotriazol-1-ol-water (1/1) (855 mg, 1.2 eq., 5.58 mmol) and N-ethylbis(isopropyl)amine (1.62 mL, 2 eq., 9.3 mmol). After 5 min of stirring 2-methyl-2,6,8-triaza-6,7-decadiene to hydrogen chloride (1:1) (1.07 g, 1.2 eq., 5.58 mmol) was added at room temperature. The reaction mixture was stirred at room temperature for 16 h. Progress of the reaction was monitored by TLC & LCMS. After completion of reaction, water (30 mL) was added to reaction mixture and extracted with ethyl acetate (3×30 mL). Combined organic layer was dried over Na₂SO₄ and concentrated under reduced pressure to get crude compound tert-butyl 4-[2-(m-bromophenyl)acetyl]-1-piperazinecarboxylate (1.4 g, 3.65 mmol) as a colorless gummy solid. The crude compound was carried to next step as such.

¹H NMR (400 MHz, DMSO-d₆): δ=7.44 (s, 1H), 7.42 (m, 1H), 7.29-7.21 (m, 2H), 3.75 (s, 2H), 3.50-3.42 (m, 4H), 3.29 (m, 4H), 1.40 (s, 9H).

### Step 2: Synthesis of 2-(3-bromophenyl)-1-(piperazin-1-yl)ethan-1-one (4)

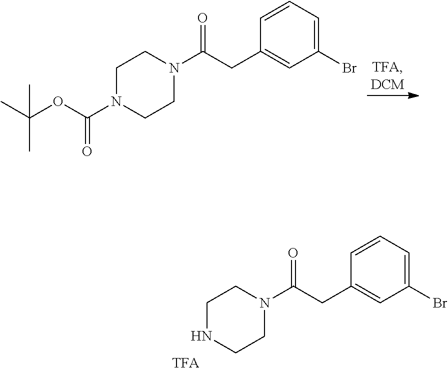 [SMILES: CC(C)(C)OC(=O)N1CCN(C(=O)Cc2cccc(Br)c2)CC1 ; O=C(Cc1cccc(Br)c1)N1CCNCC1]

To a stirred solution of tert-butyl 4-[2-(m-bromophenyl)acetyl]-1-piperazinecarboxylate (1.4 g, 3.65 mmol) in dichloromethane (15.7 mL, 245 mmol) was added trifluoroacetic acid (5 mL, 10 eq., 36.5 mmol) at 0° C., and temperature raised to rt. Then reaction mixture was stirred at rt for 1 hour. Progress of the reaction was monitored by TLC. After completion of the reaction, the reaction mixture was concentrated under reduced pressure to get crude compound, which was washed with diethyl ether (2×20 mL) to get title compound 2-(m-bromophenyl)-1-(1-piperazinyl)-1-ethanone (1 g, 3.53 mmol, 96.68% yield) as a colorless gummy. The crude compound was carried to next step as such.

¹H NMR (400 MHz, DMSO-d₆): δ=10.63 (brs, 1H), 9.06 (s, 2H), 7.44-7.42 (m, 2H), 7.27 (t, J=8.00 Hz, 1H), 7.21 (d, J=7.60 Hz, 1H), 3.78 (s, 2H), 3.72-3.65 (m, 4H), 3.16-3.05 (m, 4H).

### Step 3: Synthesis of 1-(4-(2-(3-bromophenyl)acetyl)piperazin-1-yl)prop-2-en-1-one

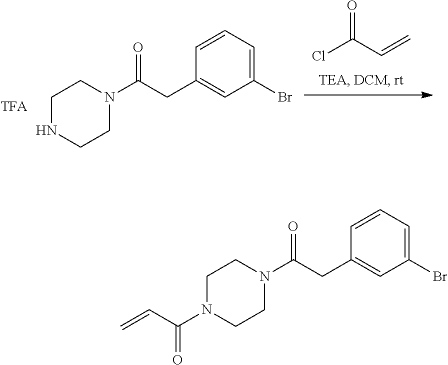

To a stirred solution of 2-(m-bromophenyl)-1-(1-piperazinyl)-1-ethanone (1 g, 3.53 mmol) in dichloromethane (10 mL, 156 mmol) at 10° C., triethylamine (1.07 g, 3 eq., 10.6 mmol) was added drop wise. After 10 min, acryloyl chloride (320 mg, 3.53 mmol) was added dropwise and the reaction was stirred at room temperature for 10 min. Progress of the reaction was monitored by TLC. After completion of reaction, the reaction mixture was poured into the ice cold NaHCO₃ solution (3×20 mL) and then extracted with DCM (3×30 mL). The combined organic layers were collected, dried over Na₂SO₄ and concentrated in vacuum to get crude residue. Crude compound was purified by combi-flash to get title compound 1-(4-acryloyl-1-piperazinyl)-2-(m-bromophenyl)-1-ethanone (450 mg, 1.33 mmol, 37.79% yield) as a white color solid.

¹H NMR (400 MHz, DMSO-d₆): δ=7.44-7.42 (m, 2H), 7.29-7.22 (m, 2H), 6.84-6.77 (m, 1H), 6.13 (dd, J=2.00 Hz, 16.60 Hz, 1H), 5.70 (dd, J=2.40 Hz, 10.40 Hz, 1H), 3.77 (s, 2H), 3.53-3.49 (m, 8H).

### Step 4: Synthesis of N-(4-((2-((3-(2-(4-acryloylpiperazin-1-yl)-2-oxoethyl)phenyl)amino)-3-(dimethyl phosphoryl)pyridin-4-yl)oxy)-3-fluorophenyl)-1-(4-fluorophenyl)-5-(trifluoromethyl)-1H-pyrazole-4-carboxamide

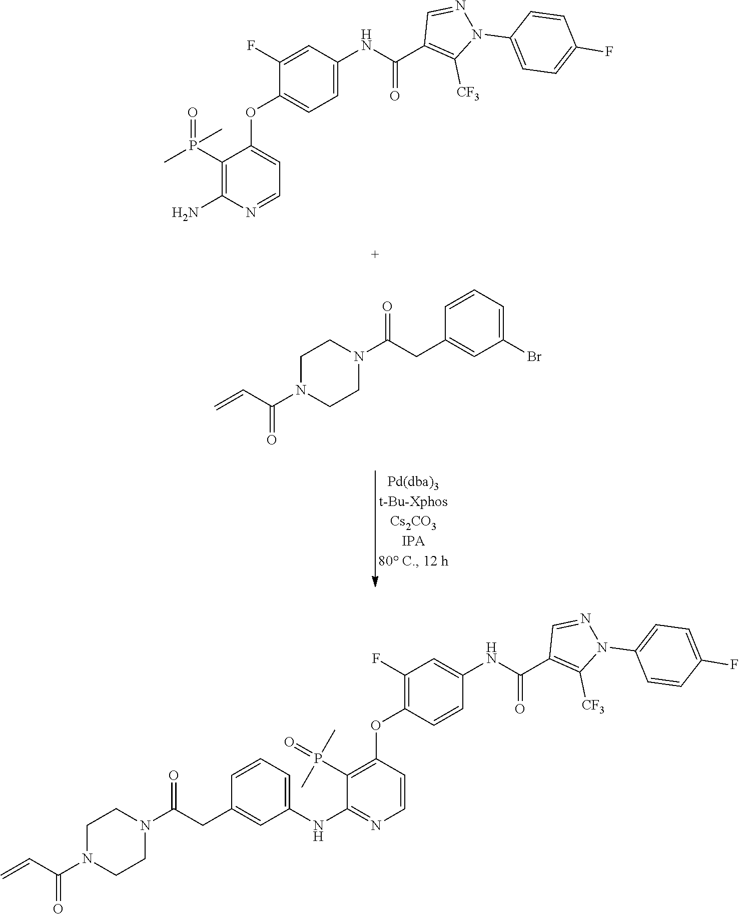 [SMILES: CP(C)(=O)c1c(Oc2ccc(NC(=O)c3cnn(-c4ccc(F)cc4)c3C(F)(F)F)cc2F)ccnc1N ; C=CC(=O)N1CCN(C(=O)Cc2cccc(Br)c2)CC1 ; C=CC(=O)N1CCN(C(=O)Cc2cccc(Nc3nccc(Oc4ccc(NC(=O)c5cnn(-c6ccc(F)cc6)c5C(F)(F)F)cc4F)c3P(C)(C)=O)c2)CC1]

To stirred a solution of N-{4-[2-amino-3-(dimethylphosphoryl)-4-pyridyloxy]-3-fluorophenyl}-1-(p-fluorophenyl)-5-(trifluoromethyl)-4-pyrazolecarboxamide (0.1 g, 181 μmol), 1-(1-acryloyl-4-piperidyl)-2-(m-bromophenyl)-1-ethanone (85.4 mg, 1.4 eq., 254 μmol), dicesium carbonate (118 mg, 2 eq., 363 μmol) in isopropanol (10 mL, 131 mmol) Argon gas was purged for 20 min. Then bis(tert-butyl)[2′,4′,6′-tris(isopropyl)-2-biphenylyl]phosphine (7.7 mg, 0.1 eq., 18.1 μmol) and (1E,4E)-1,5-diphenyl-1,4-pentadien-3-one 1,5-diphenyl-1,4-pentadien-3-one-palladium (1/2/2) (8.3 mg, 0.05 eq., 9.07 μmol) were added to the reaction mixture at rt and the reaction mixture was purged for 15 min with argon gas again. The reaction mixture was stirred at 80° C. for 12 h. The reaction progress was monitored by TLC and LCMS. After completion of the reaction, the reaction mixture was cooled to rt then diluted with Acetone (15 mL) and stirred the reaction mixture for 10 min then filtered through a pad of diatomaceous earth (i.e., Celite®). The filtrate was concentrated under vacuum to obtained crude compound. Crude compound was purified by Prep-HPLC to afford title compound N-(4-((2-((3-(2-(4-acryloylpiperazin-1-yl)-2-oxoethyl)phenyl)amino)-3-(dimethylphosphoryl)pyridin-4-yl)oxy)-3-fluorophenyl)-1-(4-fluorophenyl)-5-(trifluoromethyl)-1H-pyrazole-4-carboxamide (45 mg, 55.8 μmol, 30.76% yield) as a white color solid.

¹H NMR (400 MHz, DMSO-d₆): δ=11.48 (s, 1H), 10.86 (s, 1H), 8.35 (s, 1H), 8.14 (d, J=6.00 Hz, 1H), 7.92 (dd, J=2.00 Hz, 12.80 Hz, 1H), 7.65-7.55 (m, 4H), 7.46 (d, J=8.80 Hz, 3H), 7.40 (s, 1H), 7.22 (t, J=7.60 Hz, 1H), 6.83-6.75 (m, 2H), 6.13-6.08 (m, 2H), 5.68 (d, J=10.00 Hz, 1H), 3.73 (s, 2H), 3.53-3.47 (m, 8H), 1.92 (d, J=14.40 Hz, 6H), LCMS: 808.75 (M+H)⁺.

### Synthetic Example 40

### Synthesis of N-(4-((2-Amino-3-(Dimethylphosphoryl)Pyridin-4-Yl)Oxy)-3-Fluorophenyl)-4-Ethoxy-1-(4-Fluorophenyl)-2-Oxo-1,2-Dihydropyridine-3-Carboxamide (Compound 58)

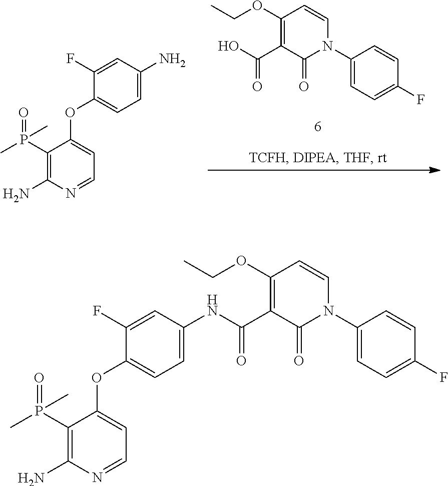 [SMILES: CP(C)(=O)c1c(Oc2ccc(N)cc2F)ccnc1N ; CCOc1ccn(-c2ccc(F)cc2)c(=O)c1C(=O)O ; CCOc1ccn(-c2ccc(F)cc2)c(=O)c1C(=O)Nc1ccc(Oc2ccnc(N)c2P(C)(C)=O)c(F)c1]

To a stirred solution of [2-amino-4-(4-amino-2-fluorophenoxy)-3-pyridyl]dimethylphosphine oxide (0.2 g, 677 μmol, 1 eq) and 2-ethoxy-5-(p-fluorophenyl)-6-oxo-1,3-cyclohexadiene-1-carboxylic acid (187 mg, 677 μmol, 1 eq) in tetrahydrofuran (85.5 mL, 1.05 mol) was added chloro(dimethylamino)methylenebis(methyl)azanium hexafluoridophosphate(1-) (570 mg, 3 eq, 2.03 mmol) and N-ethylbis(isopropyl)amine (605 μL, eq, 3.39 mmol) at 0° C. Then the reaction mixture was stirred at room temperature for 16 h. The reaction progress was monitored by TLC & LCMS. After completion of reaction, water (50 mL) was added to reaction mixture and extracted with ethyl acetate (3×60 mL). Combined organic layer was dried under Na₂SO₄ and concentrated under reduced pressure to get crude compound. The crude compound was purified by combi flash using 3% MeOH in DCM as eluent to afford title compound (with 59% purity by LCMS). Then compound was again purified by Prep-HPLC to afford pure title compound N-{4-[2-amino-3-(dimethylphosphoryl)-4-pyridyloxy]-3-fluorophenyl}-4-ethoxy-1-(4-fluorophenyl)-2-oxo-1,2-dihydronicotinamide (45 mg, 11.98% yield) as a white color solid.

¹H NMR (400 MHz, DMSO-d₆): δ=10.57 (s, 1H), 7.90-7.85 (m, 3H), 7.48-7.43 (m, 3H), 7.39-7.31 (m, 3H), 6.51 (d, J=8.00 Hz, 3H), 4.25 (q, J=6.80 Hz, 2H), 1.79 (d, J=14.00 Hz, 6H), LCMS: 555.51 (M+H)⁺.

### Synthetic Example 39A

### Synthesis of N-[4-(2-{M-[2-(4-Acryloyl-1-Piperazinyl)Ethyl]Phenylamino}-4-Pyrimidinyloxy)-3-Fluorophenyl]-1-(P-Fluorophenyl)-5-(Trifluoromethyl)-4-Pyrazolecarboxamide (Compound 43)

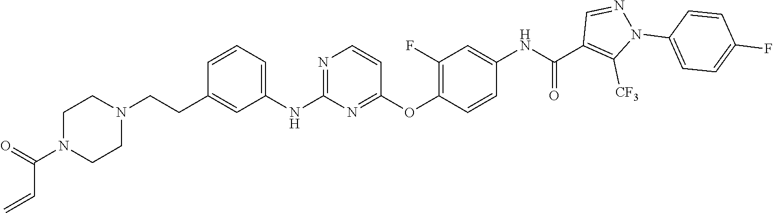 [SMILES: C=CC(=O)N1CCN(CCc2cccc(Nc3nccc(Oc4ccc(NC(=O)c5cnn(-c6ccc(F)cc6)c5C(F)(F)F)cc4F)n3)c2)CC1]

### Step 1: Synthesis of 1-(mesyloxy)-2-(m-nitrophenyl)ethane

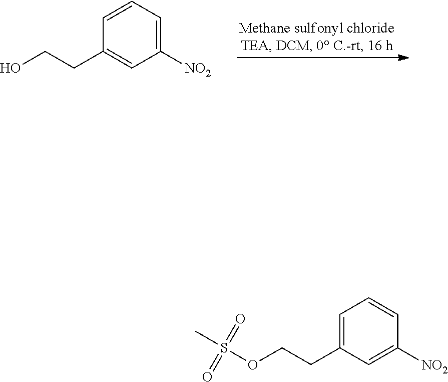 [SMILES: O=[N+]([O-])c1cccc(CCO)c1 ; CS(=O)(=O)OCCc1cccc([N+](=O)[O-])c1]

To a stirred solution of 2-(m-nitrophenyl)ethanol (1 g, 5.98 mmol) in DCM (15 mL), triethylamine (1.08 mL, 1.3 eq, 7.78 mmol) was added at 0° C. under nitrogen atmosphere. Then (chlorosulfonyl)methane (926 μL, 2 eq, 12 mmol) was added dropwise and the reaction temperature was increased to room temperature and stirred for another 16 h. Progress of reaction was monitored by TLC. After completion of reaction solvent was removed and the residue was dissolved in DCM (100 mL) and then washed with water (50 mL), dried over anhydrous Na₂SO₄ and concentrated under reduced pressure to afford title compound 1-(mesyloxy)-2-(m-nitrophenyl)ethane as color less liquid (1.2 g, 81.79% yield).

¹H NMR (400 MHz, DMSO-d₆): δ=8.14 (d, J=8.8 Hz, 2H), 7.61 (d, J=7.6 Hz, 1H), 7.53 (t, J=8.0 Hz 1H), 4.48 (t, J=6.8 Hz, 2H), 3.19 (t, J=6.8 Hz, 2H), 2.96 (s, 3H).

### Step 2: Synthesis of tert-butyl 4-[2-(m-nitrophenyl)ethyl]-1-piperazinecarboxylate

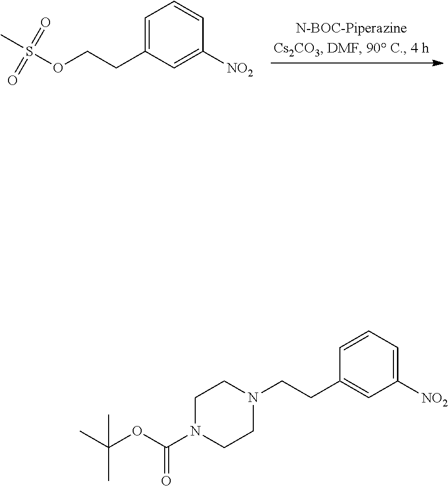 [SMILES: CS(=O)(=O)OCCc1cccc([N+](=O)[O-])c1 ; CC(C)(C)OC(=O)N1CCN(CCc2cccc([N+](=O)[O-])c2)CC1]

To a stirred solution of 1-(mesyloxy)-2-(m-nitrophenyl)ethane (1.5 g, 6.12 mmol) in dimethylformamide (15 mL, 194 mmol), was added dicesium carbonate (4.98 g, 2.5 eq, 15.3 mmol) at room temperature. After a while, tert-butyl 1-piperazinecarboxylate (1.37 g, 1.2 eq., 7.34 mmol) was added and the reaction mixture was heated to 100° C. further continued for 4 h. Progress of the reaction was monitored by TLC & LCMS. After completion of reaction, reaction mixture was quenched with ice water (20 mL) and extracted with ethyl acetate (3×30 mL). The combined organic layer was dried over anhydrous Na₂SO₄ and concentrated under reduced pressure to get crude compound. The crude compound was purified by flash chromatography using 30% EA in Hexane to afford title compound tert-butyl 4-[2-(m-nitrophenyl)ethyl]-1-piperazinecarboxylate (460 mg, 22.42% yield).

¹H NMR (400 MHz, DMSO-d₆): δ=8.13 (s, 1H), 8.06 (d, J=7.6 Hz, 1H), 7.73 (d, J=7.6 Hz, 1H), 7.58 (t, J=8.0 Hz, 1H), 3.42-3.40 (m, 2H), 3.34-3.30 (m, 2H), 3.08-3.05 (m, 2H), 2.88 (m, 2H), 2.60-2.56 (m, 2H), 2.39 (m, 2H), 1.40 (s, 9H). LCMS: 336.2 (M+H)⁺.

### Step-3: tert-butyl 4-[2-(m-aminophenyl)ethyl]-1-piperazinecarboxylate

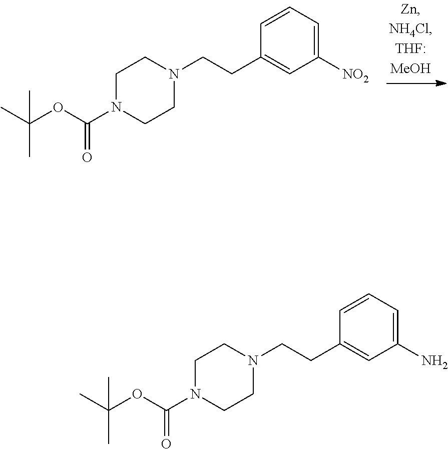 [SMILES: CC(C)(C)OC(=O)N1CCN(CCc2cccc([N+](=O)[O-])c2)CC1 ; CC(C)(C)OC(=O)N1CCN(CCc2cccc(N)c2)CC1]

To a stirred solution of tert-butyl 4-[2-(m-nitrophenyl)ethyl]-1-piperazinecarboxylate (360 mg, 1.07 mmol) in ethanol (7 mL, 120 mmol) and water (5.5 mL, 305 mmol), iron (599 mg, eq., 10.7 mmol) and ammonium chloride (1.15 g, 20 eq., 21.5 mmol) were added at 0° C. After that the temperature increased to rt and dimethylformamide (2.5 mL, 32.3 mmol) was added and the reaction was refluxed for 2 h. Progress of reaction was monitored by TLC. After completion of reaction, the reaction mixture cooled and dried under vacuum. After that the reaction mass was dissolved in ethyl acetate (30 mL) and washed with water (40 mL×3). The combined organic layer was dried over anhydrous Na₂SO₄ and concentrated in vacuum to afford the crude. The crude was purified by flash chromatography using 30% ethyl acetate in Hexane to afford title compound tert-butyl 4-[2-(m-aminophenyl)ethyl]-1-piperazinecarboxylate (150 mg, 45.8% yield).

¹H NMR (400 MHz, DMSO-d₆): δ=6.89 (t, J=7.6 Hz, 1H), 6.39-6.32 (m, 3H), 3.30 (m, 4H), 2.58-2.54 (m, 2H), 2.47-2.43 (m, 2H), 2.37-2.34 (m, 4H), 1.39 (s, 9H). LCMS: 306.15 (M+H)⁺.

### Step 4: Synthesis of N-[3-fluoro-4-(2-{m-[2-(1-piperazinyl)ethyl]phenylamino}-4-pyrimidinyloxy) phenyl]-1-(p-fluorophenyl)-5-(trifluoromethyl)-4-pyrazolecarboxamide

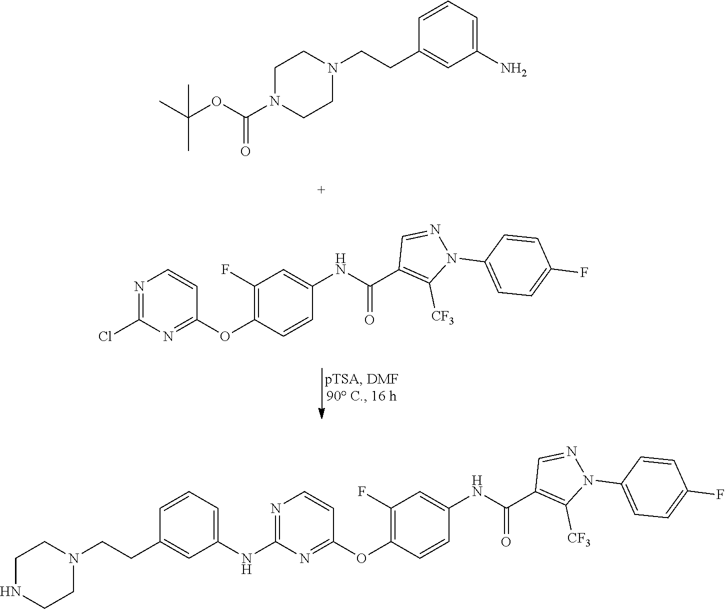 [SMILES: CC(C)(C)OC(=O)N1CCN(CCc2cccc(N)c2)CC1 ; O=C(Nc1ccc(Oc2ccnc(Cl)n2)c(F)c1)c1cnn(-c2ccc(F)cc2)c1C(F)(F)F ; O=C(Nc1ccc(Oc2ccnc(Nc3cccc(CCN4CCNCC4)c3)n2)c(F)c1)c1cnn(-c2ccc(F)cc2)c1C(F)(F)F]

To a stirred solution of N-[4-(2-chloro-4-pyrimidinyloxy)-3-fluorophenyl]-1-(p-fluorophenyl)-5-(trifluoromethyl)-4-pyrazolecarboxamide (0.1 g, 202 μmol) and tert-butyl 4-[2-(m-aminophenyl)ethyl]-1-piperazinecarboxylate (73.9 mg, 1.2 eq., 242 μmol) in dimethylformamide (4 mL, 51.7 mmol), 4-methylbenzene-1-sulfonic acid hydrate (153 mg, 4 eq., 807 μmol) was added. The reaction mixture was stirred for 10 h at 90° C. Progress of the reaction was monitored by LCMS. After completion of reaction, the reaction mixture was dissolved in ethyl acetate (30 mL) and washed with water (20 mL×3). Organic layer was dried over anhydrous Na₂SO₄ and concentrated under vacuum to afford the crude compound. The crude was purified by flash chromatography using 5% MeOH in DCM as eluent to afford title compound N-[3-fluoro-4-(2-{m-[2-(1-piperazinyl)ethyl]phenylamino}-4-pyrimidinyloxy)phenyl]-1-(p-fluorophenyl)-5-(trifluoromethyl)-4-pyrazolecarboxamide (80 mg, 59% yield) as brown color solid.

¹H NMR (400 MHz, DMSO-d₆): δ=11.79 (brs, 1H), 8.43 (s, 1H), 8.40 (d, J=6.0 Hz, 1H), 7.91 (dd, J=2.4, 12.6 Hz, 1H), 7.66-7.58 (m, 3H), 7.49-7.36 (m, 5H), 7.06 (t, J=8.0 Hz, 1H), 6.82 (d, J=7.6 Hz, 1H), 6.59 (d, J=5.6 Hz, 1H), 3.77-3.58 (m, 2H), 3.56-3.50 (m, 2H), 3.42-3.40 (m, 2H), 3.39-3.17 (m, 4H), 2.97-2.93 (m, 2H).

### Step 5: Synthesis of N-[4-(2-{m-[2-(4-acryloyl-1-piperazinyl)ethyl]phenylamino}-4-pyrimidinyloxy)-3-fluorophenyl]-1-(p-fluorophenyl)-5-(trifluoromethyl)-4-pyrazolecarboxamide

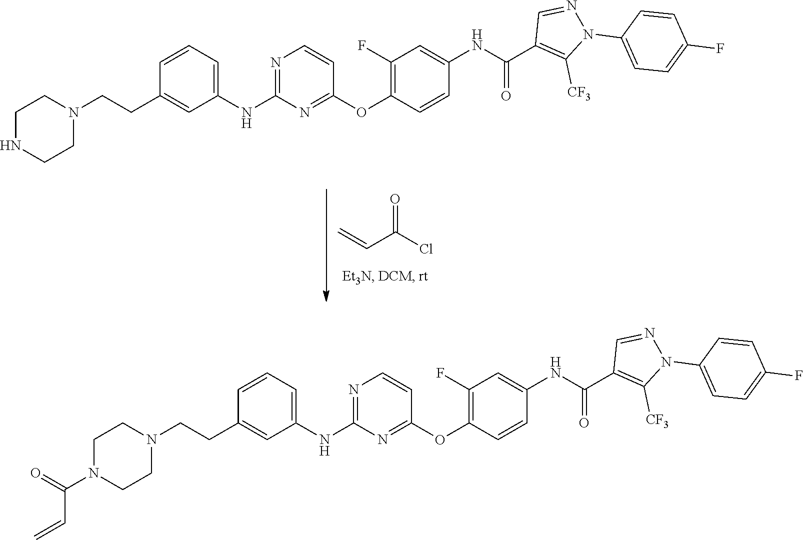

To a stirred solution of N-[3-fluoro-4-(2-{m-[2-(1-piperazinyl)ethyl]phenylamino}-4-pyrimidinyloxy)phenyl]-1-(p-fluorophenyl)-5-(trifluoromethyl)-4-pyrazolecarboxamide (40 mg, 60.2 μmol, 1 eq.) was added triethylamine (25.2 μL, 181 μmol, 3 eq.) at 0° C. and then stirred for 2 min, then acryloyl chloride (4.38 μL, 54.2 μmol, 0.9 eq.) was added to reaction mixture at 0° C. and the reaction mixture was stirred 5 min. Progress of reaction was monitored by TLC & LCMS. After completion of reaction, reaction mixture was diluted in dichloromethane, and washed with ice cold water (20 mL), Organic layer was separated, and aqueous layer again extracted with dichloromethane (2×10 mL) again. The combined organic layer was washed with NaHCO₃, dried over Na₂SO₄ and concentrated under reduced pressure to get crude residue. Crude compound was purified by prep-HPLC to afford the desired product N-[4-(2-{m-[2-(4-acryloyl-1-piperazinyl)ethyl]phenylamino}-4-pyrimidinyloxy)-3-fluorophenyl]-1-(p-fluorophenyl)-5-(trifluoromethyl)-4-pyrazolecarboxamide (12 mg, 27.9% yield) as off white solid.

¹H NMR (400 MHz, DMSO-d₆): δ=10.82 (s, 1H), 9.58 (s, 1H), 8.39 (d, J=5.60 Hz, 1H), 8.35 (s, 1H), 7.90-7.85 (m, 1H), 7.66-7.62 (m, 2H), 7.54-7.52 (m, 1H), 7.49-7.40 (m, 3H), 7.32 (s, 1H), 7.26 (d, J=5.6 Hz, 1H), 6.98 (t, J=8.0 Hz, 1H), 6.80-6.72 (m, 2H), 6.57 (d, J=5.60 Hz, 1H), 6.08 (dd, J=2.40, 16.8 Hz, 1H), 5.65 (dd, J=2.0, 10.6 Hz, 1H), 3.51-3.47 (m, 4H), 2.56-2.54 (m, 2H), 2.44-2.40 (m, 2H), 2.36-2.33 (m, 4H). LCMS: 719.92 (M+H)⁺.

### Synthetic Example 39B

### Alternate Synthetic Route for N-[4-(2-{M-[2-(4-Acryloyl-1-Piperazinyl)Ethyl]Phenylamino}-4-Pyrimidinyloxy)-3-Fluorophenyl]-1-(P-Fluorophenyl)-5-(Trifluoromethyl)-4-Pyrazolecarboxamide (Compound 43)

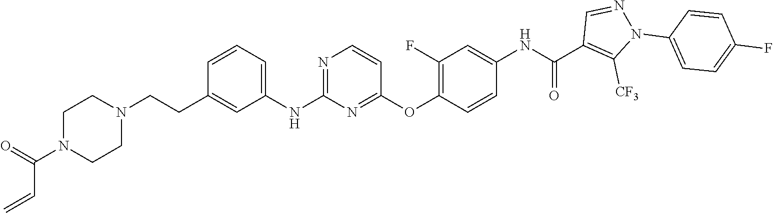 [SMILES: C=CC(=O)N1CCN(CCc2cccc(Nc3nccc(Oc4ccc(NC(=O)c5cnn(-c6ccc(F)cc6)c5C(F)(F)F)cc4F)n3)c2)CC1]

### Step 1: tert-butyl 4-[2-(m-nitrophenyl)acetyl]-1-piperazinecarboxylate

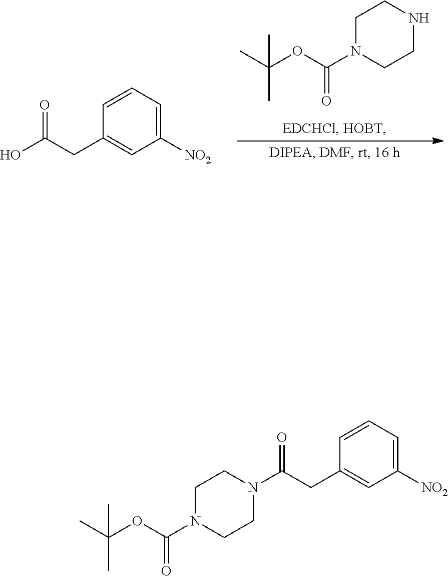 [SMILES: O=C(O)Cc1cccc([N+](=O)[O-])c1 ; CC(C)(C)OC(=O)N1CCNCC1 ; CC(C)(C)OC(=O)N1CCN(C(=O)Cc2cccc([N+](=O)[O-])c2)CC1]

To a stirred solution of (m-nitrophenyl)acetic acid (10.0 g, 55.2 mmol, 1.0 eq) and tert-butyl 1-piperazinecarboxylate (11.3 g, 60.7 mmol, 1.1 eq) in DMF (100 mL) was added 1H-1,2,3-benzotriazol-1-ol to water (1:1) (10.1 g, 66.2 mmol, 1.2 eq) and followed by added N-ethylbis(isopropyl)amine (19.2 mL, 110 mmol, 2 eq). After that EDC HCl (12.7 g, 66.2 mmol, 1.2 eq) was added to above reaction mixture at 0° C. Then the reaction mass was stirred at room temperature for 16 h. Progress of the reaction was monitored by TLC. After completion of reaction, water (250 mL) was added to reaction mixture thus solid formed was filtered. The solid compound was dissolved in DCM (150 mL) and washed with NaHCO₃ solution (60 mL) and brine solution (40 mL). Organic layers was dried over Na₂SO₄ and concentrated under reduced pressure to afford pure compound as tert-butyl 4-[2-(m-nitrophenyl)acetyl]-1-piperazinecarboxylate as an off white solid (10.0 g, 51.85% yield).

¹H NMR (400 MHz, DMSO-d₆): δ=8.10 (d, J=7.2 Hz, 2H), 7.68 (d, J=7.6 Hz, 1H), 7.60 (t, J=7.6 Hz 1H), 3.92 (s, 2H), 3.54-3.51 (m, 2H), 3.47-3.44 (m, 2H), 3.35-3.29 (m, 4H), 1.41 (s, 9H).

### Step 2: tert-butyl 4-[2-(m-nitrophenyl)ethyl]-1-piperazinecarboxylate

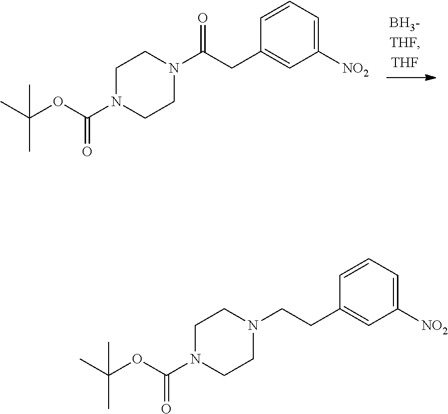 [SMILES: CC(C)(C)OC(=O)N1CCN(C(=O)Cc2cccc([N+](=O)[O-])c2)CC1 ; CC(C)(C)OC(=O)N1CCN(CCc2cccc([N+](=O)[O-])c2)CC1]

To a stirred solution of tert-butyl 4-[2-(m-nitrophenyl)acetyl]-1-piperazinecarboxylate (10.0 g, 28.6 mmol, 1 eq) in THF (100 mL) was added 1M borane-tetrahydrofuran (60 mL, 57.2 mmol, 2 eq) dropwise at 0° C., Then the reaction mass was refluxed for 2 h. Progress of the reaction was monitored by TLC. After completion of reaction, the reaction mixture was cooled to 0° C. and quenched with methanol. After that reaction mixture was concentrated under reduced pressure to get crude compound. Crude obtained was purified by combi-flash using 50% EtOAc in Hexane as eluent to get title compound of tert-butyl 4-[2-(m-nitrophenyl)ethyl]-1-piperazinecarboxylate as an off white solid (6.3 g, 65.63% yield).

¹H NMR (400 MHz, DMSO-d₆): δ=8.17 (d, J=1.60 Hz, 1H), 8.10 (dt, J=0.80, 8.13 Hz, 1H), 7.75 (d, J=7.60 Hz, 1H), 7.62 (t, J=7.60 Hz, 1H), 3.54-3.58 (m, 4H), 3.18-3.22 (m, 2H), 3.01-3.05 (m, 2H), 2.88 (s, 4H), 1.41 (s, 9H), LCMS: 336.2 (M+H)⁺.

### Step 3:1-[2-(m-nitrophenyl)ethyl]piperazine TFA salt

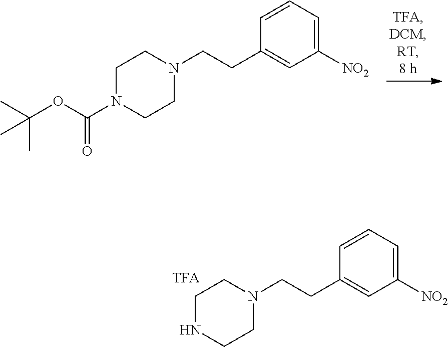 [SMILES: CC(C)(C)OC(=O)N1CCN(CCc2cccc([N+](=O)[O-])c2)CC1 ; O=[N+]([O-])c1cccc(CCN2CCNCC2)c1]

To a stirred solution of tert-butyl 4-[2-(m-nitrophenyl)ethyl]-1-piperazinecarboxylate (6.3 g, 18.8 mmol, 1 eq) in trifluoroacetic acid/dichloromethane(1:1) (20 mL) and reaction mixture was stirred for 8 hours at room temperature. After completion of reaction on TLC, the reaction mixture was concentrated in vacuo and the residue obtained was triturated with diethyl ether & pentane to afford the title compound 1-[2-(m-nitrophenyl)ethyl]piperazine-TFA Salt (6 g crude) as off white solid.

¹H NMR (400 MHz, DMSO-d₆): δ=8.18 (s, 1H), 8.11 (dd, J=1.60, Hz, 1H), 7.74 (d, J=7.60 Hz, 1H), 7.63 (t, J=8.00 Hz, 1H), 3.23-3.29 (m, 1OH), 3.04-3.07 (m, 2H), Mass: 236.2 (M+H)⁺.

### Step 4: 1-{4-[2-(m-nitrophenyl)ethyl]-1-piperazinyl}-2-propen-1-one

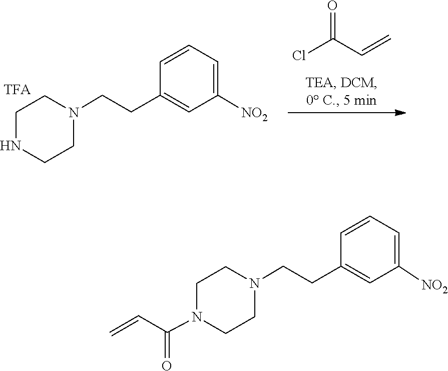

To a stirred solution of 1-[2-(m-nitrophenyl)ethyl]piperazine (1 g, 4.25 mmol, 1 eq) in DCM (10 mL) and was added triethylamine (2.96 mL, 5 eq., 21.3 mmol) at 0° C. and stirred for 20 min, then acryloyl chloride (343 μL, 4.25 mmol, 1 eq) was added at 0° C. The reaction mixture was stirred for 30 min at RT. Progress of the reaction was monitored by TLC. After completion of reaction, reaction mixture was quenched with water (50 mL) and extracted with EtOAc (3×50 mL) and washed with sat NaHCO₃ solution (50 mL). The combined organic layer was dried over Na₂SO₄ and concentrated under reduced pressure to get crude compound. The crude compound was purified by flash chromatography using 0-5% MeOH in DCM to afford 1-{4-[2-(m-nitrophenyl)ethyl]-1-piperazinyl}-2-propen-1-one as colorless gummy solid (400 mg, 32.53% yield).

¹H NMR (400 MHz, DMSO-d₆): δ=8.14 (s, 1H), 8.06 (dd, J=1.60, 8.20 Hz, 1H), 7.73 (d, J=7.60 Hz, 1H), 7.58 (t, J=7.60 Hz, 1H), 6.80 (dd, J=10.40, 16.80 Hz, 1H), 6.10 (dd, J=2.40, 16.80 Hz, 1H), 5.67 (dd, J=2.40, 10.60 Hz, 1H), 3.51-3.54 (m, 4H), 2.90 (t, J=7.60 Hz, 2H), 2.59 (t, J=7.20 Hz, 2H), 2.44 (s, 4H).

### Step 5: 1-{4-[2-(m-aminophenyl)ethyl]-1-piperazinyl}-2-propen-1-one

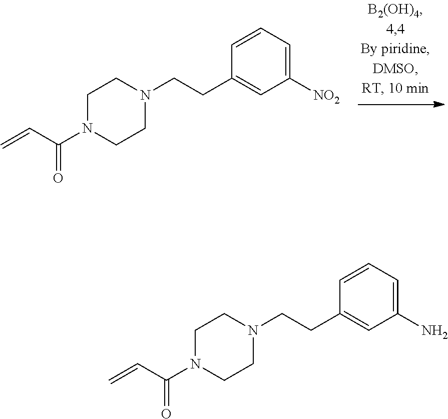 [SMILES: C=CC(=O)N1CCN(CCc2cccc([N+](=O)[O-])c2)CC1 ; C=CC(=O)N1CCN(CCc2cccc(N)c2)CC1]

To the stirred solution of 1-{4-[2-(m-nitrophenyl)ethyl]-1-piperazinyl}-2-propen-1-one (0.4 g, 1.38 mmol, 1 eq) in dimethyl sulfoxide (4 mL) at 0° C., 4,4′-bipyridyl (32.4 mg, 0.15 eq., 207 μmol) was added. After 5 min stirring, 1,1,2,2-diboranetetrol(4) (496 mg, 4 eq., 5.53 mmol) was added in portions and the reaction was proceed at room temperature. The reaction was completed within 10 min on TLC. The reaction mixture was poured into the ice-cold water (50 mL) and extracted with ethyl acetate (50 mL×3). The combined organic layers were collected and dried over Na₂SO₄ and concentrated in vacuo to get crude compound. Crude obtained was purified by using column chromatography (with gradient elution of 0-6% MeOH in DCM) to afford the title compound 1-{4-[2-(m-aminophenyl)ethyl]-1-piperazinyl}-2-propen-1-one (350 mg, 97.62% yield) as gummy solid.

¹H NMR (400 MHz, DMSO-d₆): δ=6.90 (t, J=7.60 Hz, 1H), 6.73 (dd, J=10.40, 16.60 Hz, 1H), 6.36-6.41 (m, 3H), 6.08 (dd, J=2.40, 16.60 Hz, 1H), 5.68 (dd, J=2.00, 10.40 Hz, 1H), 3.52 (t, J=5.60 Hz, 4H), 2.50-2.56 (m, 6H), 2.39-2.43 (m, 4H), Mass: 260.2 (M+H)⁺.

### Step 6: N-[4-(2-{m-[2-(4-acryloyl-1-piperazinyl)ethyl]phenylamino}-4-pyrimidinyloxy)-3-fluorophenyl]-1-(p-fluorophenyl)-5-(trifluoromethyl)-4-pyrazolecarboxamide

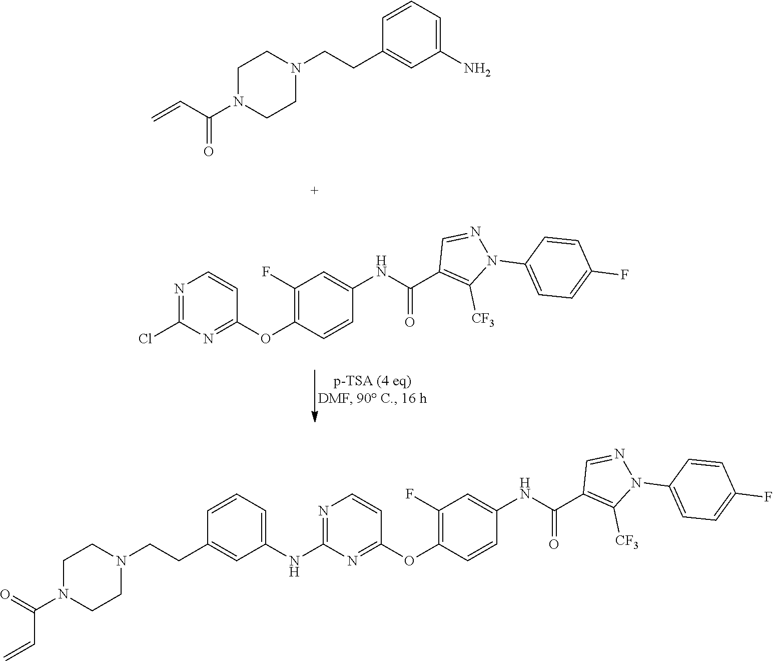 [SMILES: C=CC(=O)N1CCN(CCc2cccc(N)c2)CC1 ; O=C(Nc1ccc(Oc2ccnc(Cl)n2)c(F)c1)c1cnn(-c2ccc(F)cc2)c1C(F)(F)F ; C=CC(=O)N1CCN(CCc2cccc(Nc3nccc(Oc4ccc(NC(=O)c5cnn(-c6ccc(F)cc6)c5C(F)(F)F)cc4F)n3)c2)CC1]

To a stirred solution of N-[4-(2-chloro-4-pyrimidinyloxy)-3-fluorophenyl]-1-(p-fluorophenyl)-5-(trifluoromethyl)-4-pyrazolecarboxamide (0.5 g, 1.01 mmol, 1 eq) in DMF (10 mL) and 1-{4-[2-(m-aminophenyl)ethyl]-1-piperazinyl}-2-propen-1-one (314 mg, 1.2 eq., 1.21 mmol) was added 4-methylbenzene-1-sulfonic acid hydrate (767 mg, 4 eq., 4.03 mmol) at room temperature. The reaction mixture stirred for 16 h at 90° C. Progress of the reaction was monitored by TLC. After completion of reaction, water (30 mL) was added to reaction mixture and extracted with ethyl acetate (3×30 mL). Combined organic layer was dried over Na₂SO₄ and concentrated under reduced pressure to get crude compound. The crude was purified by prep-HPLC to afford title compound of N-[4-(2-{m-[2-(4-acryloyl-1-piperazinyl)ethyl]phenylamino}-4-pyrimidinyloxy)-3-fluorophenyl]-1-(p-fluorophenyl)-5-(trifluoromethyl)-4-pyrazolecarboxamide (52 mg, 72.4 μmol) as a white color solid (52 mg, 7.17% yield).

¹H NMR (400 MHz, DMSO-d₆): δ=10. 81 (s, 1H), 9.57 (s, 1H), 8.39 (d, J=5.60 Hz, 1H), 8.35 (s, 1H), 7.89-7.85 (m, 1H), 7.66-7.62 (m, 2H), 7.53 (d, J=8.0 Hz, 1H), 7.54-7.41 (m, 3H), 7.32 (s, 1H), 7.26 (d, J=6.8 Hz, 1H), 6.98 (t, J=7.6 Hz, 1H), 6.80-6.72 (m, 2H), 6.56 (d, J=5.60 Hz, 1H), 6.08 (dd, J=2.40, 16.8 Hz, 1H), 5.65 (dd, J=2.40, 10.4 Hz, 1H), 3.51 5-3.47 (m, 4H), 2.44-2.40 (m, 4H), 2.36-2.31 (m, 4H), LCMS: 719.3 (M+H)⁺.

### Synthetic Example 40

### Synthesis of N-[3-Fluoro-4-(2-{M-[2-(4-Propionyl-1-Piperazinyl)Ethyl]Phenylamino}-4-Pyrimidinyloxy) Phenyl]-1-(P-Fluorophenyl)-5-(Trifluoromethyl)-4-Pyrazolecarboxamide (Compound 59)

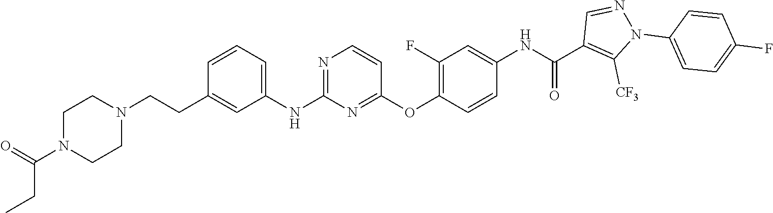 [SMILES: CCC(=O)N1CCN(CCc2cccc(Nc3nccc(Oc4ccc(NC(=O)c5cnn(-c6ccc(F)cc6)c5C(F)(F)F)cc4F)n3)c2)CC1]

### Step 1: 1-{4-[2-(m-nitrophenyl)ethyl]-1-piperazinyl}-1-propanone

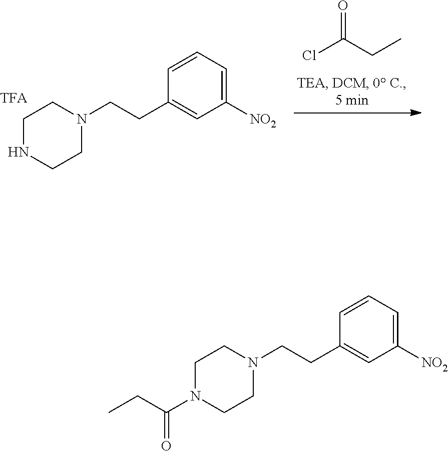

To a stirred solution of 1-[2-(m-nitrophenyl)ethyl]piperazine (1 g, 4.25 mmol, 1 eq) in DCM (10 mL) and was added triethylamine (2.96 mL, 5 eq., 21.3 mmol) at 0° C. and stirred for 20 min, then added propionyl chloride (443 μL, 1.2 eq., 5.1 mmol) at 0° C. The reaction mixture was stirred for 30 min at room temperature. Progress of the reaction was monitored by TLC. After completion of reaction, reaction mixture was quenched with water (50 mL) and extracted with EtOAc (3×50 mL), organic layer was washed with sat NaHCO₃. The combined organic layer was dried over Na₂SO₄ and concentrated under reduced pressure to get crude compound. The crude compound was purified by flash chromatography using 0-5% MeOH in DCM to afford desired compound 1-{4-[2-(m-nitrophenyl)ethyl]-1-piperazinyl}-1-propanone (630 mg, 50.88% yield) as colorless gummy solid.

¹H NMR (400 MHz, DMSO-d₆): δ=8.10 (s, 1H), 8.04 (dd, J=1.20, 7.60 Hz, 1H), 7.70 (d, J=7.60 Hz, 1H), 7.56 (t, J=7.60 Hz, 1H), 3.39 (s, 4H), 2.88 (t, J=7.20 Hz, 2H), 2.54 (t, J=7.60 Hz, 2H), 2.43-2.41 (m, 4H), 2.31-2.25 (m, 2H), 0.99-0.94 (m, 3H), LCMS: 292.1 (M+H)⁺.

### Step 2: 1-{4-[2-(m-aminophenyl)ethyl]-1-piperazinyl}-1-propanone

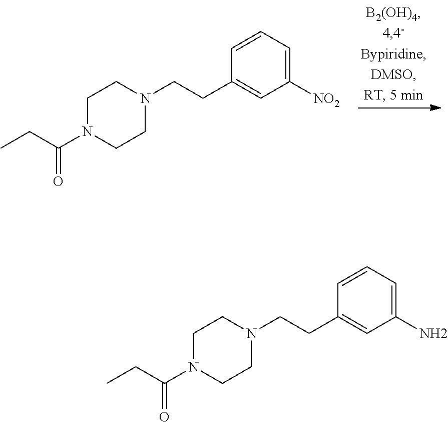 [SMILES: CCC(=O)N1CCN(CCc2cccc([N+](=O)[O-])c2)CC1 ; CCC(=O)N1CCN(CCc2cccc(N)c2)CC1]

To the stirred solution of 1-{4-[2-(m-nitrophenyl)ethyl]-1-piperazinyl}-1-propanone (530 mg, 1.82 mmol, 1 eq) in dimethyl sulfoxide (5 mL) at 0° C. Then 4,4′-bipyridyl (42.6 mg, 0.15 eq., 273 μmol) was added to the reaction mixture. After 5 min stirring 1,1,2,2-diboranetetrol(4) (652 mg, 4 eq., 7.28 mmol) was added in portions and the reaction was stirred at room temperature. The reaction was completed within 10 min on TLC. After completion of reaction, the reaction mixture was poured into the ice-cold water (50 mL) and extracted with ethyl acetate (50 mL×3). The combined organic layers were collected and dried over Na₂SO₄ and concentrated in vacuo. The crude obtained was purified by column chromatography (with gradient elution of 0-6% MeOH in DCM) to afford the title compound as 1-{4-[2-(m-aminophenyl)ethyl]-1-piperazinyl}-1-propanone (250 mg, 52.58% yield) as gummy solid.

¹H NMR (400 MHz, DMSO-d₆): δ=6.90 (t, J=7.60 Hz, 1H), 6.41-6.35 (m, 3H), 3.41 (d, J=4.0 Hz, 4H), 2.57-2.48 (m, 4H), 2.40-2.36 (m, 4H), 2.34-2.27 (m, 2H), 0.95 (t, J=7.2 Hz, 3H), Mass: 262.1 (M+H)⁺.

### Step 3: N-[3-fluoro-4-(2-{m-[2-(4-propionyl-1-piperazinyl)ethyl]phenylamino}-4-pyrimidinyloxy)phenyl]-1-(p-fluorophenyl)-5-(trifluoromethyl)-4-pyrazolecarboxamide

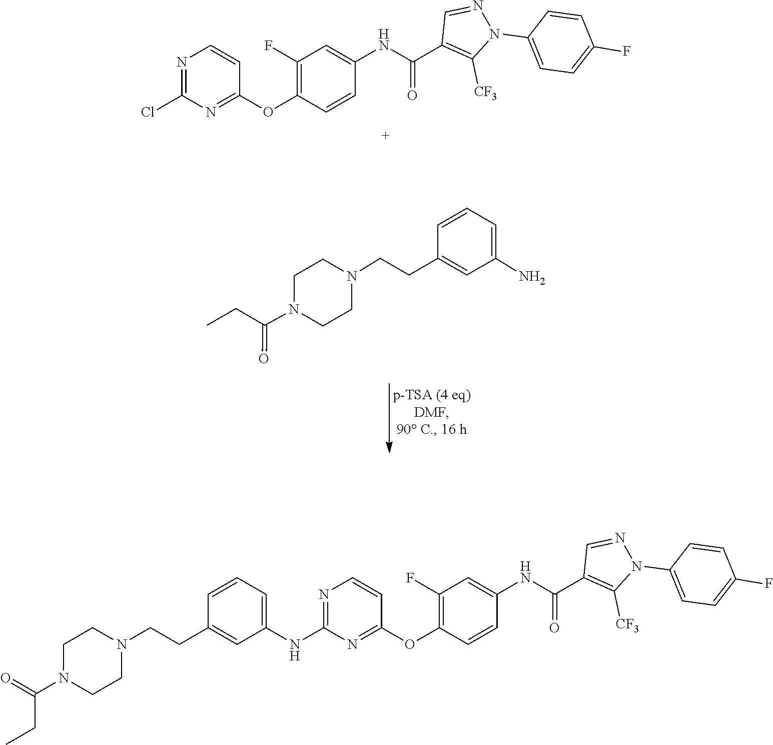 [SMILES: O=C(Nc1ccc(Oc2ccnc(Cl)n2)c(F)c1)c1cnn(-c2ccc(F)cc2)c1C(F)(F)F ; CCC(=O)N1CCN(CCc2cccc(N)c2)CC1 ; CCC(=O)N1CCN(CCc2cccc(Nc3nccc(Oc4ccc(NC(=O)c5cnn(-c6ccc(F)cc6)c5C(F)(F)F)cc4F)n3)c2)CC1]

To a stirred solution of N-[4-(2-chloro-4-pyrimidinyloxy)-3-fluorophenyl]-1-(p-fluorophenyl)-5-(trifluoromethyl)-4-pyrazolecarboxamide (250 mg, 504 μmol, 1 eq), and 1-{4-[2-(m-aminophenyl)ethyl]-1-piperazinyl}-1-propanone (198 mg, 1.5 eq., 756 μmol) in DMF (4 5 mL) and 4-methylbenzene-1-sulfonic acid hydrate (384 mg, 4 eq., 2.02 mmol), was added at room temperature and the reaction mixture stirred for 16 h at 90° C. Progress of the reaction was monitored by TLC & LCMS. After completion of reaction, water (20 mL) was added to reaction mixture and extracted with ethyl acetate (3×20 mL). Combined organic layer was dried over Na₂SO₄ and concentrated under reduced pressure to get crude compound. The crude was purify by prep-HPLC to afford title compound of N-[3-fluoro-4-(2-{m-[2-(4-propionyl-1-piperazinyl)ethyl]phenylamino}-4-pyrimidinyloxy)phenyl]-1-(p-fluorophenyl)-5-(trifluoromethyl)-4-pyrazolecarboxamide (60 mg, 16.51% yield) as an off white color solid.

¹H NMR (400 MHz, DMSO-d₆): δ=8.37 (d, J=5.60 Hz, 1H), 8.31 (s, 1H), 7.86 (dd, J=2.40, 12.60 Hz, 1H), 7.60-7.64 (m, 1H), 7.53 (d, J=8.40 Hz, 1H), 7.39-7.48 (m, 3H), 7.26 (s, 1H), 7.20 (d, J=8.40 Hz, 1H), 6.98 (t, J=7.60 Hz, 1H), 6.72 (d, J=7.60 Hz, 1H), 6.57 (d, J=5.60 Hz, 1H), 3.37 (s, 4H), 2.41 (d, J=8.00 Hz, 2H), 2.34-2.38 (m, 2H), 2.27-2.31 (m, 2H), 2.23-2.25 (m, 2H), LCMS: 721.3 (M+H)⁺.

### Synthetic Example 41

### Synthesis of N-[3-Fluoro-4-(2-{M-[2-(1-Piperazinyl)Ethyl]Phenylamino}-4-Pyrimidinyloxy)Phenyl]-1-(P-Fluorophenyl)-5-(Trifluoromethyl)-4-Pyrazolecarboxamide (Compound 60) & N-[3-Fluoro-4-(2-{M-[2-(4-Methyl-1-Piperazinyl) Ethyl]Phenylamino}-4-Pyrimidinyloxy) Phenyl]-1-(P-Fluorophenyl)-5-(Trifluoromethyl)-4-Pyrazolecarboxamide (Compound 45)

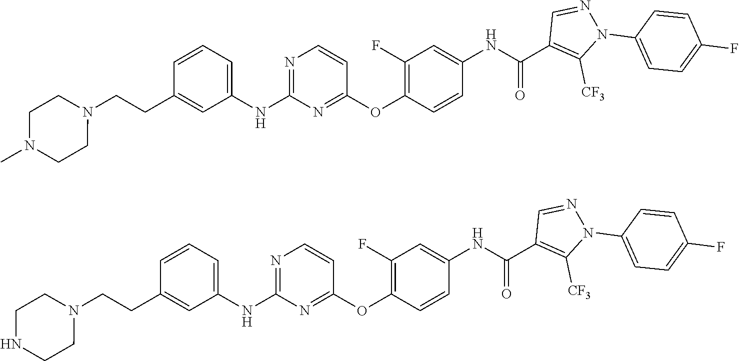 [SMILES: CN1CCN(CCc2cccc(Nc3nccc(Oc4ccc(NC(=O)c5cnn(-c6ccc(F)cc6)c5C(F)(F)F)cc4F)n3)c2)CC1 ; O=C(Nc1ccc(Oc2ccnc(Nc3cccc(CCN4CCNCC4)c3)n2)c(F)c1)c1cnn(-c2ccc(F)cc2)c1C(F)(F)F]

### Step 1: tert-butyl 4-[2-(m-amino phenyl) ethyl]-1-piperazinecarboxylate

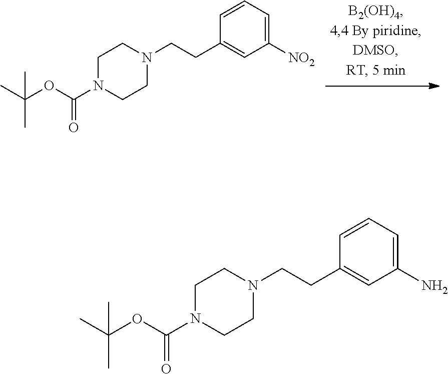 [SMILES: CC(C)(C)OC(=O)N1CCN(CCc2cccc([N+](=O)[O-])c2)CC1 ; CC(C)(C)OC(=O)N1CCN(CCc2cccc(N)c2)CC1]

To the stirred solution of tart-butyl 4-[2-(m-nitro phenyl)ethyl]-1-piperazinecarboxylate (0.8 g, 2.39 mmol, 1 eq) in dimethyl sulfoxide (8 mL, 112 mmol) at 0° C., 4,4′-bipyridyl (55.9 mg, 0.15 eq., 358 μmol) was added to the reaction mixture. After 5 min of stirring, 1, 1, 2, 2-diboranetetrol (4) (855 mg, 4 eq., 9.54 mmol) was added in portions and the reaction was stirred at room temperature. The reaction was completed within 10 min on TLC. Then the reaction mixture was poured into the ice-cold water (40 mL) and extracted with ethyl acetate (50 mL×3). The combined organic layer was collected and dried over Na₂SO₄ and concentrated in vacuum. Crude compound obtained was purified by column chromatography (with gradient elution of 0-6% MeOH in DCM) to afford the title compound as tert-butyl 4-[2-(m-amino phenyl)ethyl]-1-piperazinecarboxylate (0.6 g, 82.36% yield) as gummy solid.

¹H NMR (400 MHz, DMSO-d₆): δ=6.89 (t, J=7.60 Hz, 1H), 6.39-6.33 (m, 3H), 4.92 (s, 2H), 3.30 (s, 4H), 2.56-2.54 (m, 2H), 2.47-2.45 (m, 2H), 2.36 (s, 4H), 1.39 (s, 9H).

### Step 2: N-[3-fluoro-4-(2-{m-[2-(1-piperazinyl)ethyl]phenylamino}-4-pyrimidinyloxy)phenyl]-1-(p-fluorophenyl)-5-(trifluoromethyl)-4-pyrazolecarboxamide

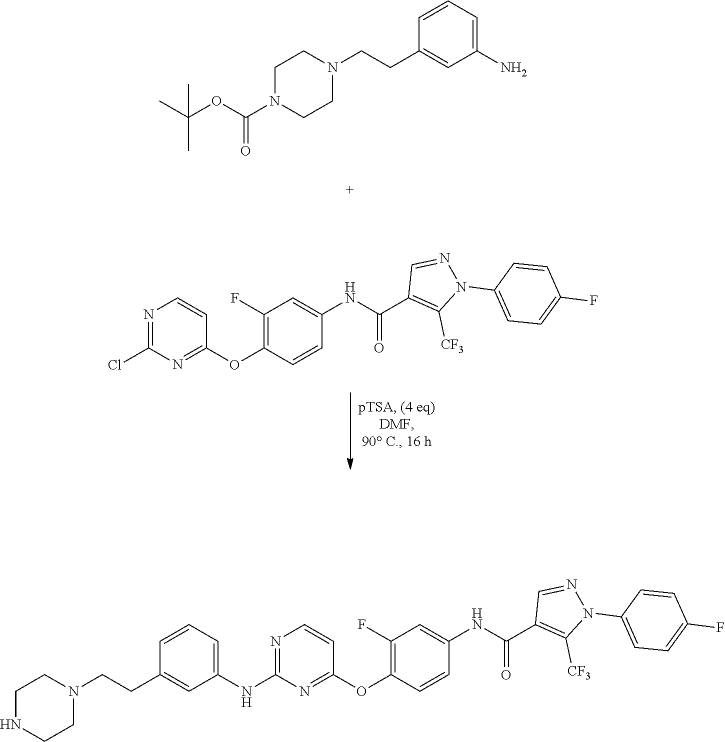 [SMILES: CC(C)(C)OC(=O)N1CCN(CCc2cccc(N)c2)CC1 ; O=C(Nc1ccc(Oc2ccnc(Cl)n2)c(F)c1)c1cnn(-c2ccc(F)cc2)c1C(F)(F)F ; O=C(Nc1ccc(Oc2ccnc(Nc3cccc(CCN4CCNCC4)c3)n2)c(F)c1)c1cnn(-c2ccc(F)cc2)c1C(F)(F)F]

To a stirred solution of N-[4-(2-chloro-4-pyrimidinyloxy)-3-fluorophenyl]-1-(p-fluorophenyl)-5-(trifluoromethyl)-4-pyrazolecarboxamide (350 mg, 706 μmol, 1 eq), 4-fluoroaniline(44.8 mg, 403 μmol, 1 eq) in DMF (5 mL) and 4-methylbenzene-1-sulfonic acid hydrate (537 mg, 4 eq, 2.82 mmol), was added at room temperature, then the reaction mixture stirred for 16 h at 90° C. Progress of the reaction was monitored by TLC & LCMS. After completion of reaction, water (30 mL) was added to reaction mixture and extracted with ethyl acetate (3×30 mL). Combined organic layer was dried over Na₂SO₄ and concentrated under reduced pressure to get crude compound. The crude was purified by prep-HPLC to get title compound of N-[3-fluoro-4-(2-{m-[2-(1-piperazinyl) ethyl]phenyl amino}-4-pyrimidinyloxy) phenyl]-1-(p-fluorophenyl)-5-(trifluoromethyl)-4-pyrazolecarboxamide (70 mg, 14.92% yield) as an off-white color solid.

¹H NMR (400 MHz, DMSO-d₆): δ=8.29 (d, J=5.60 Hz, 1H), 8.06 (s, 1H), 7.69-7.75 (m, 1H), 7.44-7.47 (m, 2H), 7.37 (m, 1H), 7.30 (s, 1H), 7.20 (q, J=8.00 Hz, 3H), 7.10 (t, J=8.00 Hz, 1H), 6.98-7.00 (m, 1H), 6.75 (d, J=8.00 Hz, 1H), 6.46 (d, J=5.60 Hz, 1H), 2.83 (s, 4H), 2.48-2.52 (m, 8H), LCMS: 665.0 (M+H)⁺.

### Step 3: N-[3-fluoro-4-(2-{m-[2-(4-methyl-1-piperazinyl) ethyl]phenylamino}-4-pyrimidinyloxy) phenyl]-1-(p-fluorophenyl)-5-(trifluoromethyl)-4-pyrazolecarboxamide

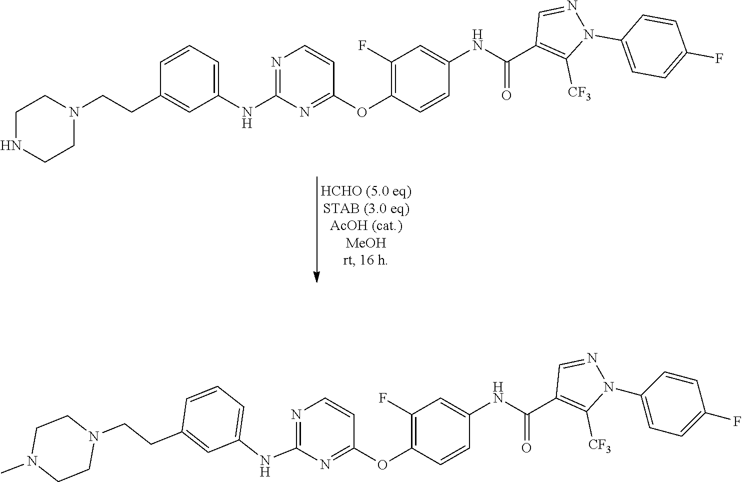 [SMILES: O=C(Nc1ccc(Oc2ccnc(Nc3cccc(CCN4CCNCC4)c3)n2)c(F)c1)c1cnn(-c2ccc(F)cc2)c1C(F)(F)F ; CN1CCN(CCc2cccc(Nc3nccc(Oc4ccc(NC(=O)c5cnn(-c6ccc(F)cc6)c5C(F)(F)F)cc4F)n3)c2)CC1]

To a stirred solution of N-[3-fluoro-4-(2-{m-[2-(1-piperazinyl)ethyl]phenylamino}-4-pyrimidinyloxy)phenyl]-1-(p-fluorophenyl)-5-(trifluoromethyl)-4-pyrazolecarboxamide (60 mg, 90.3 μmol, 1 eq) in methanol (2 mL) was added formaldehyde (44.9 μL, 5 eq., 451 μmol) and acetic acid (2.01 mL, 35.1 mmol) at 0° C., then the reaction mixture stirred for 30 min at 0° C. After 30 min of stirring sodium bis(acetyloxy)boranuidyl acetate (57.4 mg, 3 eq., 271 μmol) was added to reaction mixture at room temperature. Then the reaction mixture stirred for 16 h at room temperature. Progress of the reaction was monitored by TLC. Reaction mixture was direct concentrated under reduced pressure, obtained crude was diluted with ethyl acetate (10 mL) and washed with sat NaHCO₃ solution (10 mL). Combined organic layer was dried over Na₂SO₄ and concentrated under reduced pressure to get the crude. The crude was purified by prep-HPLC to get title compound N-[3-fluoro-4-(2-{m-[2-(4-methyl-1-piperazinyl)ethyl]phenylamino}-4-pyrimidinyloxy)phenyl]-1-(p-fluorophenyl)-5-(trifluoromethyl)-4-pyrazolecarboxamide (12 mg, 19.59% yield) as an off white color solid.

¹H NMR (400 MHz, DMSO-d₆): δ=8.29 (d, J=5.60 Hz, 1H), 8.17 (s, 1H), 7.85 (d, J=12.0 Hz, 1H), 7.59-7.56 (m, 2H), 7.47 (d, J=8.4 Hz, 1H), 7.37-7.22 (m, 5H), 7.04 (t, J=8.00 Hz, 1H), 6.78 (d, J=7.60 Hz, 1H), 6.49 (d, J=5.60 Hz, 1H), 3.47-3.42 (m, 1H), 3.24-3.14 (m, 1H), 2.79-2.69 (m, 10H), 2.51 (m, 3H), LCMS: 679.3 (M+H)⁺.

### Synthetic Example 42

### Synthesis of N-(4-((2-((3-(2-(4-Ethylpiperazin-1-Yl)Ethyl)Phenyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 46)

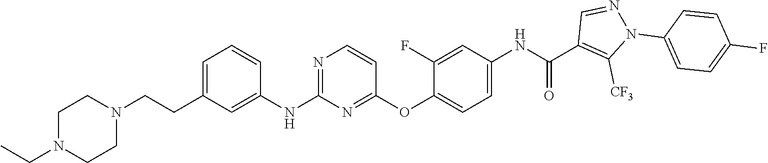 [SMILES: CCN1CCN(CCc2cccc(Nc3nccc(Oc4ccc(NC(=O)c5cnn(-c6ccc(F)cc6)c5C(F)(F)F)cc4F)n3)c2)CC1]

### Step 1: Synthesis of 1-ethyl-4-(3-nitrophenethyl)piperazine

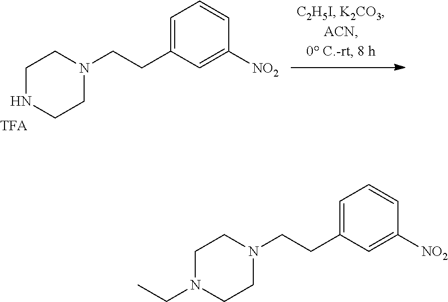 [SMILES: O=[N+]([O-])c1cccc(CCN2CCNCC2)c1 ; CCN1CCN(CCc2cccc([N+](=O)[O-])c2)CC1]

To the stirred solution of 1-[2-(m-nitrophenyl)ethyl]piperazine (1 g, 4.25 mmol) in acetonitrile, K₂CO₃ (1.76 g, 4.25 mmol) was added and stirred for 15 min at 10° C. Then, 1-iodoethane (683 μL, 4.25 mmol) was added drop wise to the solution and stirred at RT for 3h. The reaction mixture was diluted with ethyl acetate (20 mL) and washed with water (3×10 mL). The combined organic layer was collected, dried over Na₂SO₄ and concentrated under vacuum to get crude compound. The obtained residue was purified by flash chromatography (3% MeOH in DCM) to give 1-ethyl-4-(3-nitrophenethyl)piperazine (2) (0.56 g, yield 50%) as a liquid.

¹H NMR (400 MHz, CDCl₃): δ 8.08-8.05 (m, 2H), 7.54-7.52 (m, 1H), 7.46-7.42 (m, 1H), 2.92-2.88 (m, 2H), 2.66-2.60 (m, 6H), 2.48-2.42 (m, 2H), 1.28 (s, 2H), 1.12-1.09 (m, 3H).

### Step 2: Synthesis of 3-(2-(4-ethylpiperazin-1-yl)ethyl)aniline

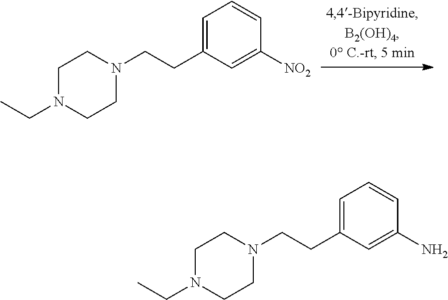 [SMILES: CCN1CCN(CCc2cccc([N+](=O)[O-])c2)CC1 ; CCN1CCN(CCc2cccc(N)c2)CC1]

To a stirred solution of the 4-ethyl-1-[2-(m-nitrophenyl)ethyl]piperazine (0.56 g, 2.13 mmol) in DMSO under nitrogen atmosphere, 4,4′-bipyridine (49.8 mg, 2.13 mmol) was added. After 5 min, B₂(OH)₄ (763 mg, 2.13 mmol) was added slowly and stirred for 5-10 min till the completion of the reaction (progress of the reaction was monitored by TLC). Then, after completion of reaction ice water (10 mL) was added to the reaction mixture and extracted with ethyl acetate (3×20 mL). The organic layers were collected, dried over Na₂SO₄ and concentrated under reduced pressure to get crude residue. The crude was washed with pentane to afford 3-(2-(4-ethylpiperazin-1-yl)ethyl)aniline (120 mg, yield 24.1%) as sticky solid.

¹H NMR (400 MHz, DMSO-d₆): δ 6.91-6.87 (m, 1H), 6.38-6.33 (m, 1H), 4.93 (s, 2H), 1.90 (s, 1H), 1.23 (s, 1H), 1.01 (s, 3H).

### Step 3: Synthesis of N-(4-((2-((3-(2-(4-ethylpiperazin-1-yl)ethyl)phenyl)amino)pyrimidin-4-yl)oxy)-3-fluorophenyl)-1-(4-fluorophenyl)-5-(trifluoromethyl)-1H-pyrazole-4-carboxamide

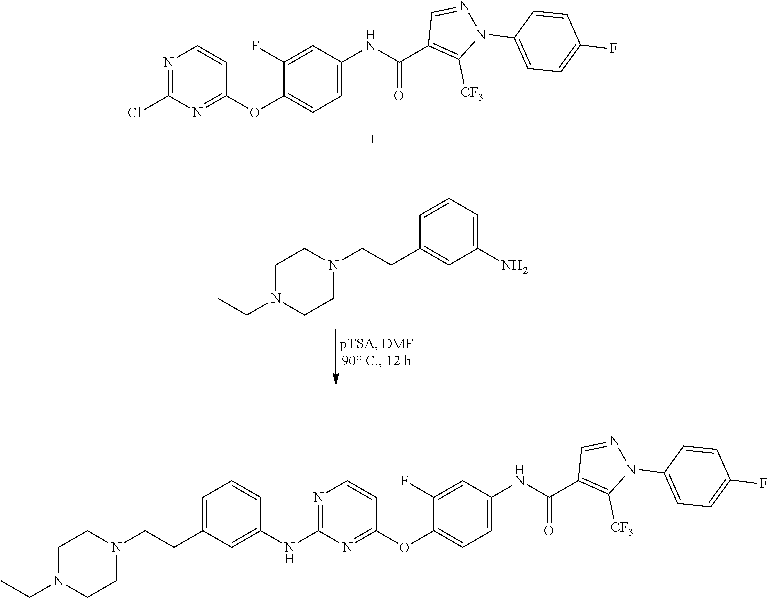 [SMILES: O=C(Nc1ccc(Oc2ccnc(Cl)n2)c(F)c1)c1cnn(-c2ccc(F)cc2)c1C(F)(F)F ; CCN1CCN(CCc2cccc(N)c2)CC1 ; CCN1CCN(CCc2cccc(Nc3nccc(Oc4ccc(NC(=O)c5cnn(-c6ccc(F)cc6)c5C(F)(F)F)cc4F)n3)c2)CC1]

In a seal tube (N-[4-(2-chloro-4-pyrimidinyloxy)-3-fluorophenyl]-1-(p-fluorophenyl)-5-(trifluoromethyl)-4-pyrazolecarboxamide) (0.2 g, 403 μmol) was dissolved in the DMF (5 mL). To the above solution, pTSA (278 mg, 1.61 mmol) and, 3-(2-(4-ethylpiperazin-1-yl)ethyl)aniline (3) (120 mg, 444 μmol) were added subsequently. After reagent addition completion, the reaction was heated to 90° C. and stirred overnight till the complete consumption of the starting material (progress of the reaction was monitored by TLC). Then after completion of reaction ice water (10 mL) was added to reaction mixture and extracted with ethyl acetate (3×25 mL). The combined organic layer was dried over Na₂SO₄ and concentrated in vacuum to get crude compound. The crude was purified by Prep HPLC to afford the desired product (14 mg, yield 14.11%) as white fluffy solid.

¹H NMR (400 MHz, CD₃OD); δ 8.28 (d, J=5.6 Hz, 1H), 8.16 (s, 1H), 7.84 (dd, J=2.4, 12.4 Hz, 1H), 7.58-7.55 (m, 2H), 7.47 (d, J=8.0 Hz, 2H), 7.36-7.21 (m, 5H), 7.02 (t, J=8.0 Hz, 1H), 6.77 (d, J=7.6 Hz, 1H), 6.48 (d, J=5.6 Hz, 1H), 2.68-2.63 (m, 12H), 1.15 (t, J=7.2 Hz, 3H). LCMS: 693.3 [M+H]⁺.

### Synthetic Example 43

### Synthesis of N-(3-Fluoro-4-((2-((3-(2-(4-Propylpiperazin-1-Yl)Ethyl)Phenyl)Amino)Pyrimidin-4-Yl)Oxy)Phenyl)-1-(4-Fluorophenyl)-5-(Trifluoromethyl)-1H-Pyrazole-4-Carboxamide (Compound 47)

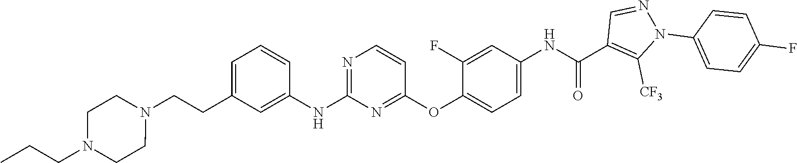 [SMILES: CCCN1CCN(CCc2cccc(Nc3nccc(Oc4ccc(NC(=O)c5cnn(-c6ccc(F)cc6)c5C(F)(F)F)cc4F)n3)c2)CC1]

### Step 1: Synthesis of 1-(3-nitrophenethyl)-4-propylpiperazine

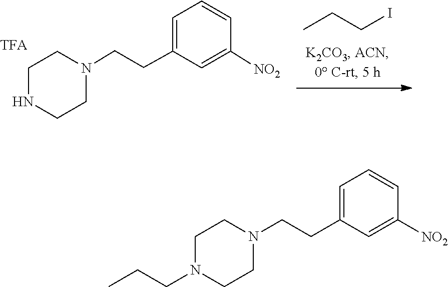

To the stirred solution of 1-[2-(m-nitrophenyl)ethyl]piperazine. TFA salt (0.5 g, 2.13 mmol) in acetonitrile, K₂CO₃ (0.88 g, 2.13 mmol) was added and stirred for 10 min at 10° C. Then, 1-iodopropane (206 μL, 2.13 mmol) was added drop wise to the solution and stirred at RT for 5 h. The reaction mixture was diluted with ethyl acetate (30 mL) and washed with water (3×15 mL). The organic layer was collected, dried over Na₂SO₄ and concentrated under vacuum to get crude compound. The obtained residue was purified by flash chromatography (5% MeOH in DCM) to afford title compound 1-(3-nitrophenethyl)-4-propylpiperazine (3) (0.53 g, yield 44.96%) as sticky mass.

¹H NMR (400 MHz, DMSO-d₆): δ 8.09-8.05 (m, 2H), 7.55-7.53 (m, 1H), 7.46-7.43 (m, 1H), 2.93-2.89 (m, 2H), 2.66-2.59 (m, 8H), 2.47-2.41 (m, 2H), 1.82 (s, 2H), 1.25 (s, 1H), 1.12-1.08 (m, 3H) ppm.

### Step 2: Synthesis of 3-(2-(4-propylpiperazin-1-yl)ethyl)aniline

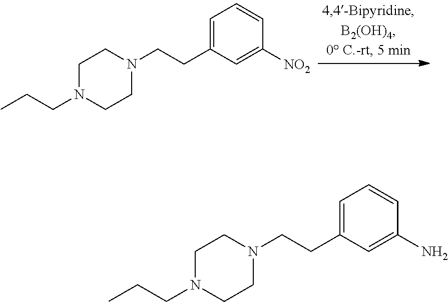 [SMILES: CCCN1CCN(CCc2cccc([N+](=O)[O-])c2)CC1 ; CCCN1CCN(CCc2cccc(N)c2)CC1]

To a stirred solution of 1-(3-nitrophenethyl)-4-propylpiperazine (3) (0.7 g, 2.52 mmol) in DMSO (5 mL) under nitrogen atmosphere, 4,4′-bipyridine (59.1 mg, 379 μmol) was added. After 5 min, B₂(OH)₄ (905 mg, 10.1 mmol) was added slowly and stirred for 5-10 min till the completion of the reaction (Progress of the reaction was monitored by TLC). Then, added ice water (10 mL) to the reaction mixture and extracted with ethyl acetate (3×20 mL). The organic layers were collected, dried over Na₂SO₄ and concentrated under reduced pressure to get crude compound. The crude residue was washed with pentane (10-15 mL) to afford m-[2-(4-propyl-1-piperazinyl)ethyl]aniline (340 mg, yield 54.46%) as sticky solid.

¹H NMR (400 MHz, CDCl₃): δ 7.08-7.04 (m, 1H), 6.59 (d, J=8.0 Hz, 1H), 6.53-6.51 (m, 2H), 3.58 (s, 1H), 2.74-2.70 (m, 2H), 2.62-2.58 (m, 9H), 2.37-2.33 (m, 2H), 2.01 (s, 1H), 1.56-1.51 (m, 2H), 1.28 (s, 1H), 0.92-0.87 (m, 3H), LCMS: 248.3 [M+H]+.

### Step 3: Synthesis of N-(3-fluoro-4-((2-((3-(2-(4-propylpiperazin-1-yl)ethyl)phenyl)amino) pyrimidin-4-yl)oxy) phenyl)-1-(4-fluorophenyl)-5-(trifluoromethyl)-1H-pyrazole-4-carboxamide

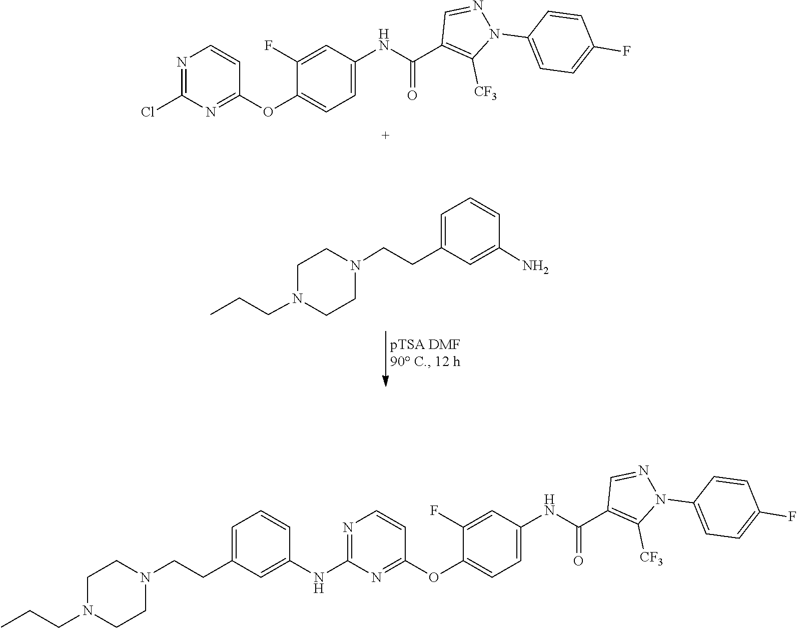 [SMILES: O=C(Nc1ccc(Oc2ccnc(Cl)n2)c(F)c1)c1cnn(-c2ccc(F)cc2)c1C(F)(F)F ; CCCN1CCN(CCc2cccc(N)c2)CC1 ; CCCN1CCN(CCc2cccc(Nc3nccc(Oc4ccc(NC(=O)c5cnn(-c6ccc(F)cc6)c5C(F)(F)F)cc4F)n3)c2)CC1]

In a seal tube, N-[4-(2-chloro-4-pyrimidinyloxy)-3-fluorophenyl]-1-(p-fluorophenyl)-5-(trifluoromethyl)-4-pyrazolecarboxamide (0.5 g, 1.01 mmol) was dissolved in DMF (5 mL). To the solution, pTSA (695 mg, 4.03 mmol) and, 3-(2-(4-propylpiperazin-1-yl)ethyl)aniline (299 mg, 1.21 mmol) were added subsequently. After completion of reagent addition, the reaction was heated to 90° C. and stirred overnight till the complete consumption of the starting material (Progress of the reaction was monitored by TLC). Then, ice water (10 mL) was added to reaction mixture and extracted with ethyl acetate (3×30 mL). The combined organic layer was dried over anhydrous Na₂SO₄ and concentrated under vacuum to get crude residue. The crude compound was purified by Prep HPLC to afford title compound N-(3-fluoro-4-((2-((3-(2-(4-propylpiperazin-1-yl)ethyl)phenyl)amino) pyrimidin-4-yl)oxy) phenyl)-1-(4-fluorophenyl)-5-(trifluoromethyl)-1H-pyrazole-4-carboxamide (80 mg, yield 11.22%) as white solid.

¹H NMR (400 MHz, DMSO-d₆); δ 10.86 (s, 1H), 9.62 (s, 1H), 8.40 (d, J=5.6 Hz, 1H), 8.36 (s, 1H), 7.88 (dd, J=2.4, 12.4 Hz, 1H), 7.66-7.62 (m, 2H), 7.53-7.40 (m, 4H), 7.36 (m, 2H), 7.03 (t, J=7.6 Hz, 1H), 6.78 (d, J=7.2 Hz, 1H), 6.58 (d, J=5.6 Hz, 1H), 3.55-3.48 (m, 14H), 3.25-2.7 (m, 10H), 1.61-1.58 (m, 2H), 0.89 (t, J=7.6 Hz, 3H). LCMS: 707.3 [M+H]+.

### Synthetic Example 44

### Synthesis of N-[3-Fluoro-4-(2-{M-[2-(4-Isopropyl-1-Piperazinyl)Ethyl]Phenylamino}-4-Pyrimidinyloxy)Phenyl]-1-(P-Fluorophenyl)-5-(Trifluoromethyl)-4-Pyrazolecarboxamide (Compound 48)

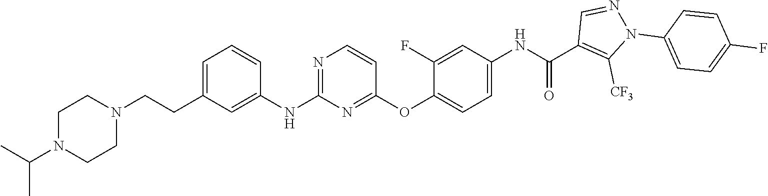 [SMILES: CC(C)N1CCN(CCc2cccc(Nc3nccc(Oc4ccc(NC(=O)c5cnn(-c6ccc(F)cc6)c5C(F)(F)F)cc4F)n3)c2)CC1]

### Step 1: Synthesis of 1-isopropyl-4-(3-nitrophenethyl)piperazine

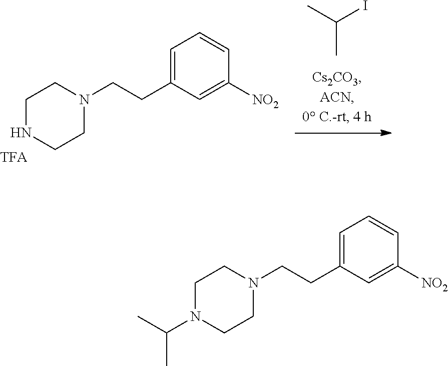

To the stirring solution of 1-[2-(m-nitrophenyl)ethyl]piperazine-TFA salt (0.5 g, 2.13 mmol) in acetonitrile, cesium carbonate (4.15 g, 3 eq., 12.8 mmol) and 2-iodopropane (509 μL, 5.1 mmol) were added at room temperature. Then, the reaction mass was refluxed for 4 h. Progress of the reaction was monitored by TLC. After completion of reaction on TLC, the reaction mixture was cooled to room temperature, and it was filtered through a pad of diatomaceous earth (i.e., Celite®) and washed with EtOAc (20 mL). The collected filtrate was evaporated under reduced pressure to get crude residue. The crude compound was purified by flash chromatography using 0-50% EtOAc in hexanes as eluent to afford the 1-isopropyl-4-(3-nitrophenethyl)piperazine (2) (520 mg, yield 44.11%) as gummy solid.

¹H NMR (400 MHz, DMSO-d₆): δ 8.12 (s, 1H), 8.05 (dd, J=1.6 Hz, 8.4 Hz, 1H), 7.71 (d, J=8.0 Hz, 1H), 7.57 (t, J=8.0 Hz, 1H), 2.86 (t, J=7.2 Hz, 2H), 2.55 (t, J=6.8 Hz, 3H), 2.50-2.44 (m, 5H), 0.97 (bs, 6H), LCMS: 278.38 [M+H]⁺.

### Step 2: Synthesis of 3-(2-(4-isopropylpiperazin-1-yl)ethyl)aniline

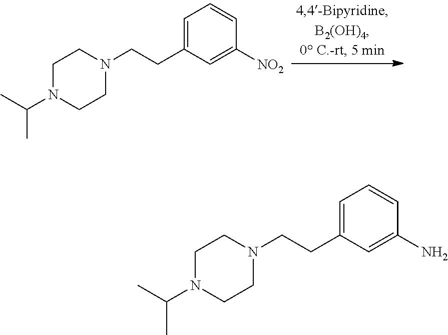 [SMILES: CC(C)N1CCN(CCc2cccc([N+](=O)[O-])c2)CC1 ; CC(C)N1CCN(CCc2cccc(N)c2)CC1]

To a stirred solution of 1-isopropyl-4-(3-nitrophenethyl)piperazine (520 mg, 1.87 mmol) in DMSO (5 mL) under nitrogen atmosphere, 4,4′-bipyridine (43.9 mg, 281 μmol) was added. After 5 min of stirring, B₂(OH)₄ (672 mg, 7.5 mmol) was added slowly and stirred for 5-10 min till the completion of the reaction (progress of reaction was monitored by TLC). After completion of reaction, ice water (10 mL) was added to the reaction mixture and extracted with ethyl acetate (3×20 mL). The combined organic layers were collected, dried over Na₂SO₄ and concentrated under reduced pressure to get crude residue. The crude compound was purified by flash chromatography using 10% MeOH in DCM to afford title compound 3-(2-(4-isopropylpiperazin-1-yl)ethyl)aniline (360 mg, yield 77.62%) as yellow solid.

¹H NMR (400 MHz, DMSO-d₆): δ 6.89 (t, J=7.6 Hz, 1H), 6.39-6.32 (m, 3H), 4.92 (s, 2H), 2.61-2.54 (m, 4H), 2.50-2.33 (m, 8H), 1.90 (s, 1H), 0.96 (d, J=6.8 Hz, 6H) ppm, Mass: 248.2 [M+H]⁺.

### Step 3: Synthesis of N-[3-fluoro-4-(2-{m-[2-(4-isopropyl-1-piperazinyl)ethyl]phenylamino}-4-pyrimidinyloxy)phenyl]-1-(p-fluorophenyl)-5-(trifluoromethyl)-4-pyrazolecarboxamide

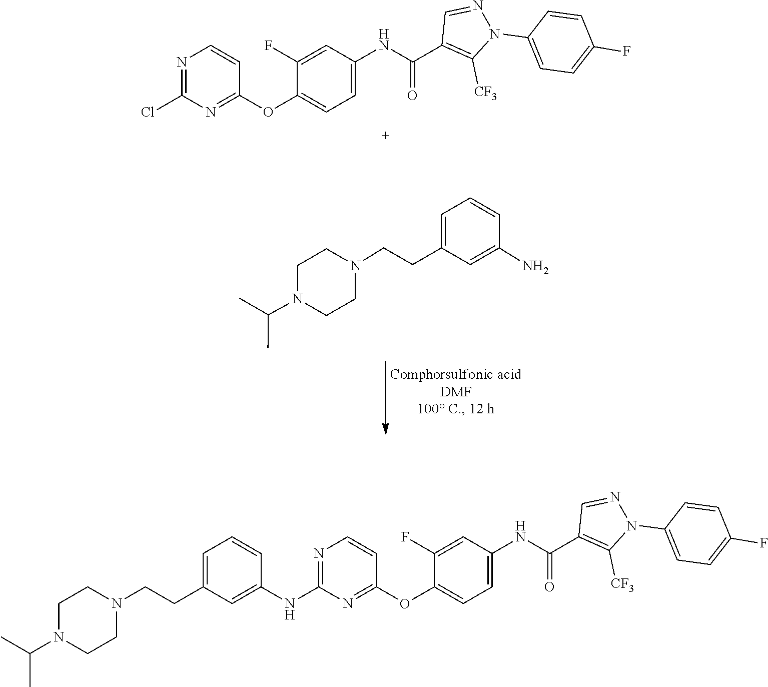 [SMILES: O=C(Nc1ccc(Oc2ccnc(Cl)n2)c(F)c1)c1cnn(-c2ccc(F)cc2)c1C(F)(F)F ; CC(C)N1CCN(CCc2cccc(N)c2)CC1 ; CC(C)N1CCN(CCc2cccc(Nc3nccc(Oc4ccc(NC(=O)c5cnn(-c6ccc(F)cc6)c5C(F)(F)F)cc4F)n3)c2)CC1]

In a seal tube, RT-001 (N-[4-(2-chloro-4-pyrimidinyloxy)-3-fluorophenyl]-1-(p-fluorophenyl)-5-(trifluoromethyl)-4-pyrazolecarboxamide) (460 mg, 928 μmol) was dissolved in the DMF (5 mL). To the above stirred solution, Camphor sulfonic acid (862 mg, 3.71 mmol) and 3-(2-(4-isopropylpiperazin-1-yl)ethyl)aniline (298 mg, 1.21 mmol) were added subsequently. After completion of reagent addition, the reaction mixture was heated to 100° C. and stirred overnight. Progress of reaction was monitored by TLC. After completion of reaction, ice water (10 mL) was added and extracted with ethyl acetate (3×30 mL). The combined organic layer was dried over anhydrous Na₂SO₄ and concentrated under vacuum to get crude residue. The crude was purified by Prep HPLC to afford title compound N-[3-fluoro-4-(2-{m-[2-(4-isopropyl-1-piperazinyl)ethyl]phenylamino}-4-pyrimidinyloxy)phenyl]-1-(p-fluorophenyl)-5-(trifluoromethyl)-4-pyrazolecarboxamide (50 mg, yield 7.63%) as white solid.

¹H NMR (400 MHz, DMSO-d₆); δ 10. 82 (s, 1H), 9.57 (s, 1H), 8.39 (d, J=5.6 Hz, 1H), 8.36 (s, 1H), 7.85 (dd, J=2.0 Hz, 9.6 Hz, 1H), 7.65-7.62 (m, 2H), 7.54-7.41 (m, 4H), 7.29-7.24 (m, 2H), 6.98 (t, J=7.6 Hz, 1H), 6.72 (d, J=7.2 Hz, 1H), 6.57 (d, J=5.6 Hz, 1H), 2.54-2.50 (m, 3H), 2.38-2.34 (m, 10H), 0.92 (d, J=6.4 Hz, 6H), LCMS: 707.3 [M+H]⁺.

### Synthetic Example 45

### Synthesis of N-(4-((2-((3-(2-(4-Cyclopropylpiperazin-1-Yl)Ethyl)Phenyl)Amino)Pyrimidin-4-Yl)Oxy)-3-Fluorophenyl)-1-(4-Fluorophenyl)-5-(Trifluoromethyl)-1H-Pyrazole-4-Carboxamide (Compound 49)

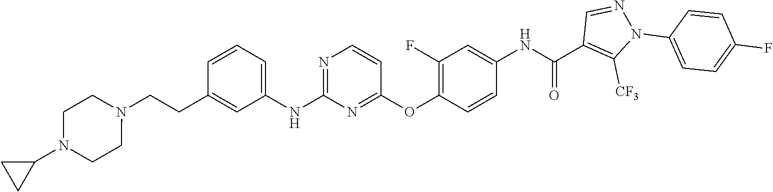 [SMILES: O=C(Nc1ccc(Oc2ccnc(Nc3cccc(CCN4CCN(C5CC5)CC4)c3)n2)c(F)c1)c1cnn(-c2ccc(F)cc2)c1C(F)(F)F]

### Step 1: Synthesis of 1-cyclopropyl-4-(3-nitrophenethyl)piperazine

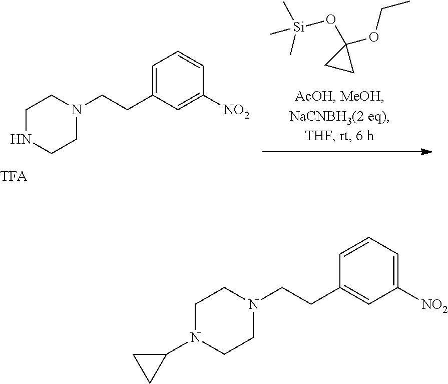 [SMILES: O=[N+]([O-])c1cccc(CCN2CCNCC2)c1 ; CCOC1(O[Si](C)(C)C)CC1 ; O=[N+]([O-])c1cccc(CCN2CCN(C3CC3)CC2)c1]

To a stirred solution of 1-[2-(m-nitrophenyl)ethyl]piperazine-trifluoroacetic acid (1/1) (1.0 g, 2.86 mmol) in mixture of solvents tetrahydrofuran (15 mL), MeOH (15 mL) were added (1-ethoxycyclopropoxy) tris(methyl)silane (1.16 mL, 2 eq., 5.73 mmol) and sodium cyano borohydride (270 mg, 1.5 eq., 4.29 mmol) followed by acetic acid (258 μL, 1.5 eq., 4.29 mmol). The reaction mixture was stirred at 60° C. for 6 h. Reaction progress was monitored by TLC. After completion of reaction, reaction mixture was cooled to room temperature, and water (10 mL) was added then neutralized with 1N NaOH and extracted with DCM (3×10 mL). The combined organic layer was dried over Na₂SO₄ and concentrated under reduced pressure. The crude compound was purified by flash silica gel column chromatography using 3-5% MeOH in DCM to afford colorless oil of 4-cyclopropyl-1-[2-(m-nitrophenyl)ethyl]piperazine (450 mg, 57.09% yield.).

¹H NMR (400 MHz, DMSO-d₆); δ 8.09-8.06 (m, 2H), 7.54 (d, J=7.6 Hz, 1H), 7.44 (t, J=7.6 Hz, 1H), 2.93-2.89 (m, 2H), 2.68 (bs, 3H), 2.63-2.60 (m, 2H), 2.59-2.54 (m, 4H), 1.63-1.60 (m, 1H), 0.48-0.39 (m, 4H), LCMS: 276.35 [M+H]⁺.

### Step 2: Synthesis of 3-(2-(4-cyclopropylpiperazin-1-yl)ethyl)aniline

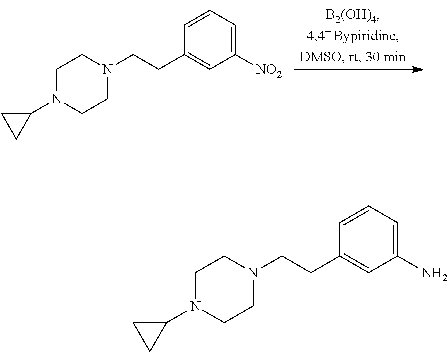 [SMILES: O=[N+]([O-])c1cccc(CCN2CCN(C3CC3)CC2)c1 ; Nc1cccc(CCN2CCN(C3CC3)CC2)c1]

To a stirred solution of 1-cyclopropyl-4-(3-nitrophenethyl)piperazine (0.45 g, 1.66 mmol, 1 eq) in dimethyl sulfoxide (10 mL) was added 4,4′-bipyridyl (38.6 mg, 0.15 eq., 0.245 mmol) and stirred for 5 min then added 1,1,2,2-diboranetetrol (591 mg, 4 eq., 6.54 mmol) at 0° C. The reaction mixture was stirred at room temperature for 10 min. Progress of the reaction was monitored by TLC. Reaction mixture was diluted with water (10 mL) and extracted with ethyl acetate (2×10 mL). The combined organic layer was dried over Na₂SO₄ and concentrated under reduced pressure to get crude residue. The crude residue was purified by flash column chromatography (with gradient elution of 5-10% MeOH in DCM) to afford the title compound 3-(2-(4-cyclopropylpiperazin-1-yl)ethyl)aniline (180 mg, 45.0% yield) as a pale yellow solid.

¹H NMR (400 MHz, DMSO-d₆); δ 6.89 (t, J=7.6 Hz, 1H), 6.39-6.33 (m, 3H), 4.92 (s, 1H), 2.55-2.50 (m, 6H), 2.42-2.33 (m, 6H), 1.58 (m, 1H), 0.40-0.37 (m, 2H), 0.28-0.26 (m, 2H).

### Step 3: Synthesis of N-(4-((2-((3-(2-(4-cyclopropylpiperazin-1-yl)ethyl)phenyl)amino)pyrimidin-4-yl)oxy)-3-fluorophenyl)-1-(4-fluorophenyl)-5-(trifluoromethyl)-1H-pyrazole-4-carboxamide

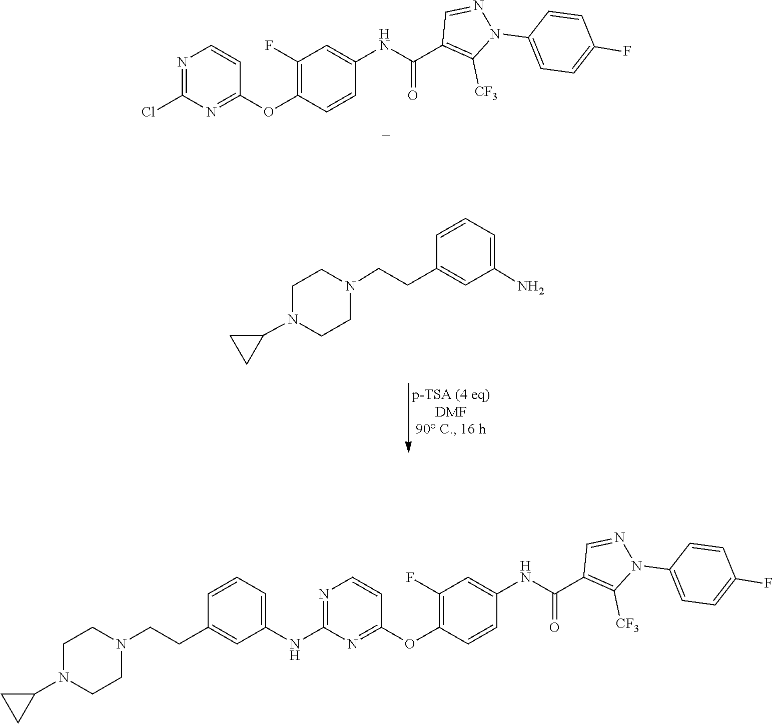 [SMILES: O=C(Nc1ccc(Oc2ccnc(Cl)n2)c(F)c1)c1cnn(-c2ccc(F)cc2)c1C(F)(F)F ; Nc1cccc(CCN2CCN(C3CC3)CC2)c1 ; O=C(Nc1ccc(Oc2ccnc(Nc3cccc(CCN4CCN(C5CC5)CC4)c3)n2)c(F)c1)c1cnn(-c2ccc(F)cc2)c1C(F)(F)F]

To a stirred solution of N-[4-(2-chloro-4-pyrimidinyloxy)-3-fluorophenyl]-1-(p-fluorophenyl)-5-(trifluoromethyl)-4-pyrazolecarboxamide (242 mg, 0.489 mmol) and 3-(2-(4-cyclopropylpiperazin-1-yl)ethyl)aniline (180 mg, 1.5 eq., 0.734 mmol) in dimethylformamide (6 mL), 4-methylbenzene-1-sulfonic acid hydrate (334 mg, 4 eq., 1.96 mmol) was added at room temperature. The reaction mixture stirred for 16 h at 90° C. Progress of the reaction was monitored by TLC. After completion of reaction, water (10 mL) was added to reaction mixture and extracted with ethyl acetate (3×15 mL). Combined organic layer was dried over Na₂SO₄ and concentrated under reduced pressure to get crude compound. The crude compound was purified by prep HPLC using in 0.1% FA in acetonitrile to get title compound of N-[4-(2-{m-[2-(4-acryloyl-1-piperazinyl)-2-oxoethyl]phenylamino}-4-pyrimidinyloxy)-3-fluorophenyl]-1-(p-fluorophenyl)-5-(trifluoromethyl)-4-pyrazolecarboxamide (100 mg, 19.34% yield) as a white color solid.

¹H NMR (400 MHz, DMSO-d₆); δ 10. 85 (s, 1H), 9.64 (s, 1H), 8.40 (d, J=5.6 Hz, 1H), 8.36 (s, 1H), 7.87 (dd, J=2.0 Hz, 12.8 Hz, 1H), 7.66-7.62 (m, 2H), 7.53-7.36 (m, 6H), 7.05 (t, J=7.6 Hz, 1H), 6.78 (d, J=7.6 Hz, 1H), 6.58 (d, J=5.6 Hz, 1H), 3.44 (m, 2H), 3.21-3.09 (m, 4H), 2.95-2.93 (m, 2H), 2.85-2.81 (m, 2H), 2.62-2.59 (m, 2H), LCMS: 705.76 [M+H]⁺.

### Synthetic Example 46

### Synthesis of N-[4-(2-{M-[2-(4-Acryloyl-1-Piperazinyl)-2-Oxoethyl]Phenylamino}-4-Pyrimidinyloxy)-3-Fluorophenyl]-1-(P-Fluorophenyl)-5-(Trifluoromethyl)-4-Pyrazolecarboxamide (Compound 50)

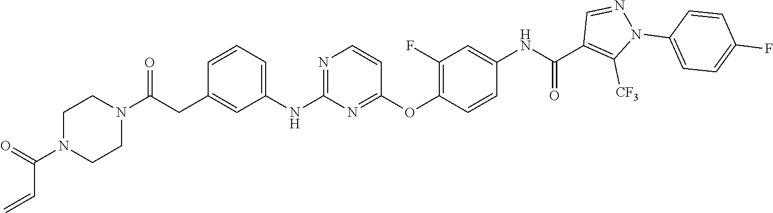 [SMILES: C=CC(=O)N1CCN(C(=O)Cc2cccc(Nc3nccc(Oc4ccc(NC(=O)c5cnn(-c6ccc(F)cc6)c5C(F)(F)F)cc4F)n3)c2)CC1]

### Step 1: 1-(4-acryloyl-1-piperazinyl)-2-(m-nitrophenyl)-1-ethanone

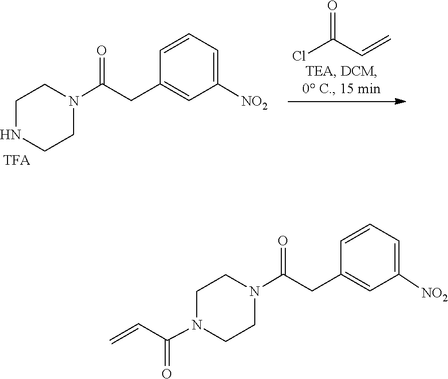

The stirred solution of 2-(m-nitrophenyl)-1-(1-piperazinyl)-1-ethanone to trifluoroacetic acid (1:1) (0.5 g, 1.38 mmol, 1 eq) in dichloromethane (25 mL) at 10° C., Triethylamine (0.959 mL, 5 eq., 6.88 mmol) was added dropwise to the reaction mixture maintaining the temperature of the reaction. After 10 min, acryloyl chloride (0.133 mL, 1.2 eq., 1.65 mmol) was added drop wise and the reaction proceeded at room temperature. The reaction was stirred for 1 h, progress of reaction was monitored by TLC. The reaction mixture was poured into the ice cold NaHCO₃ solution (3×30 mL) and the extracted with DCM (3×40 mL). The combined organic layers were collected, dried over Na₂SO₄ and concentrated in vacuum to get the crude compound of 1-(4-acryloyl-1-piperazinyl)-2-(m-nitrophenyl)-1-ethanone (500 mg). Crude compound used for next step as such.

¹H NMR (400 MHz, DMSO-d₆); δ 8.12 (s, 1H), 8.10 (s, 1H), 7.68 (d, J=7.2 Hz, 1H), 7.6t (t, J=7.6 Hz, 1H), 6.85-6.81 (m, 1H), 6.13 (dd, J=2.4 Hz, 16.4 Hz, 1H), 5.76-5.69 (m, 1H), 3.95 (s, 2H), 3.57-3.33 (m, 8H).

### Step 2: 1-(4-acryloyl-1-piperazinyl)-2-(m-aminophenyl)-1-ethanone

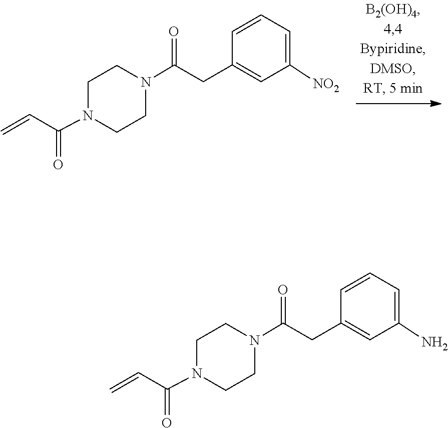 [SMILES: C=CC(=O)N1CCN(C(=O)Cc2cccc([N+](=O)[O-])c2)CC1 ; C=CC(=O)N1CCN(C(=O)Cc2cccc(N)c2)CC1]

To a stirred solution of 1-(4-acryloyl-1-piperazinyl)-2-(m-nitrophenyl)-1-ethanone (0.5 g, 1.65 mmol, 1 eq) in dimethyl sulfoxide (10 mL) was added 4,4′-bipyridyl (38.6 mg, 0.15 eq., 0.247 mmol) and stirred for 5 min then added 1,1,2,2-diboranetetrol (591 mg, 4 eq., 6.59 mmol) at 0° C. The reaction mixture was stirred at room temperature for 10 min. Progress of the reaction was monitored by TLC. Reaction mixture was diluted with water (10 mL) and extracted with ethyl acetate (2×10 mL). The combined organic layer was dried over Na₂SO₄ and concentrated under reduced pressure to get crude residue. The crude residue was purified by flash column chromatography (with gradient elution of 5-10% MeOH in DCM) to afford the title compound 1-(4-acryloyl-1-piperazinyl)-2-(m-aminophenyl)-1-ethanone (160 mg, 35.51% yield) as a pale-yellow solid.

¹H NMR (400 MHz, DMSO-d₆); δ 6.93 (t, J=7.6 Hz, 1H), 6.81-6.74 (m, 1H), 6.42-6.35 (m, 3H), 6.00 (dd, J=2.0 Hz, 16.4 Hz, 1H), 5.68 (d, J=10.0 Hz, 1H), 5.03 (m, 2H), 3.57 (s, 2H), 3.48-3.41 (m, 8H) Mass: 274.2 [M+H]⁺.

### Step 3: N-[4-(2-{m-[2-(4-acryloyl-1-piperazinyl)-2-oxoethyl]phenylamino}-4-pyrimidinyloxy)-3-fluorophenyl]-1-(p-fluorophenyl)-5-(trifluoromethyl)-4-pyrazolecarboxamide

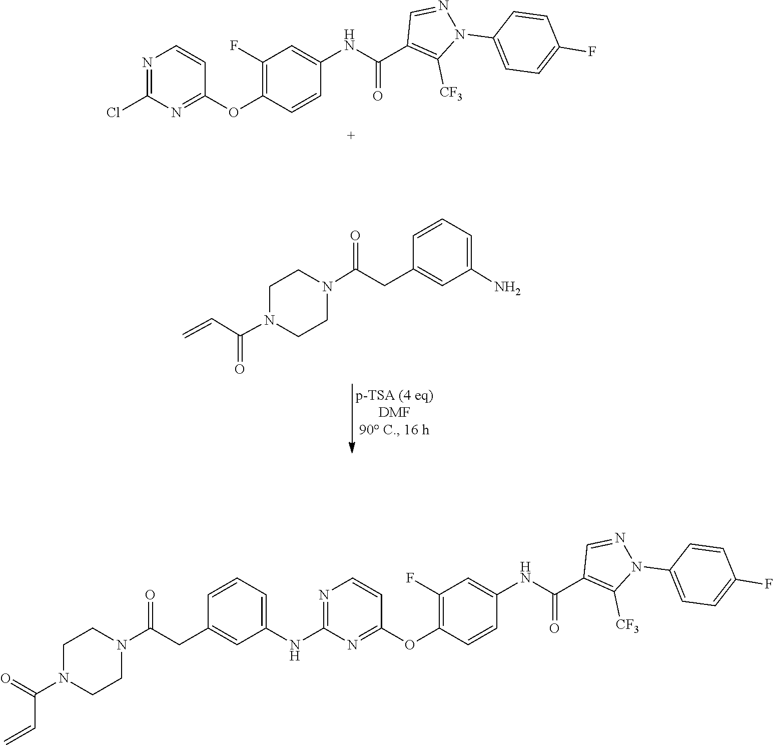 [SMILES: O=C(Nc1ccc(Oc2ccnc(Cl)n2)c(F)c1)c1cnn(-c2ccc(F)cc2)c1C(F)(F)F ; C=CC(=O)N1CCN(C(=O)Cc2cccc(N)c2)CC1 ; C=CC(=O)N1CCN(C(=O)Cc2cccc(Nc3nccc(Oc4ccc(NC(=O)c5cnn(-c6ccc(F)cc6)c5C(F)(F)F)cc4F)n3)c2)CC1]

To a stirred solution of N-[4-(2-chloro-4-pyrimidinyloxy)-3-fluorophenyl]-1-(p-fluorophenyl)-5-(trifluoromethyl)-4-pyrazolecarboxamide (150 mg, 0.303 mmol) and 1-(4-acryloyl-1-piperazinyl)-2-(m-aminophenyl)-1-ethanone (124 mg, 1.5 eq., 0.454 mmol) in dimethylformamide (6 mL), 4-methylbenzene-1-sulfonic acid hydrate (230 mg, 4 eq., 1.21 mmol) was added at room temperature. The reaction mixture stirred for 16 h at 90° C. Progress of the reaction was monitored by TLC. After completion of reaction, water (10 mL) was added to reaction mixture and extracted with ethyl acetate (3×15 mL). Combined organic layer was dried over Na₂SO₄ and concentrated under reduced pressure to get crude compound. The crude compound was purified by prep HPLC using in 0.1% FA in acetonitrile to get title compound of N-[4-(2-{m-[2-(4-acryloyl-1-piperazinyl)-2-oxoethyl]phenylamino}-4-pyrimidinyloxy)-3-fluorophenyl]-1-(p-fluorophenyl)-5-(trifluoromethyl)-4-pyrazolecarboxamide (45 mg, 20.3% yield) as a white color solid.

¹H NMR (400 MHz, DMSO-d₆); δ 10. 87 (s, 1H), 9.65 (s, 1H), 8.39 (d, J=5.6 Hz, 1H), 8.34 (s, 1H), 7.88 (d, J=12.8 Hz, 1H), 7.66-7.63 (m, 2H), 7.54 (d, J=8.8 Hz, 1H), 7.50-7.42 (m, 3H), 7.34-7.31 (m, 2H), 7.02 (t, J=7.6 Hz, 1H), 6.78-6.70 (m, 2H), 6.58 (d, J=5.6 Hz, 1H), 6.10 (d, J=16.8 Hz, 1H), 5.67 (s, 1H), 3.56 (s, 2H), 3.51-3.34 (m, 8H), LCMS: 733.2 [M+H]⁺.

### Synthetic Example 47

### Synthesis of N-[4-(2-{M-[2-(4-Acryloyl-2-Oxo-1-Piperazinyl)Ethyl]Phenylamino}-4-Pyrimidinyloxy)-3-Fluorophenyl]-1-(P-Fluorophenyl)-5-(Trifluoromethyl)-4-Pyrazolecarboxamide (Compound 51)

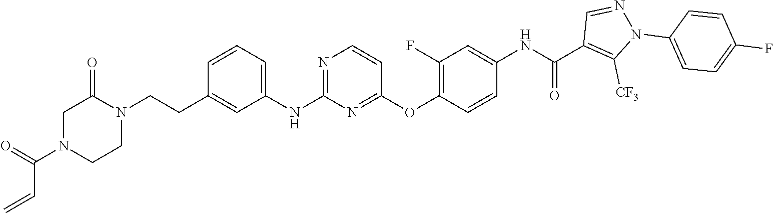 [SMILES: C=CC(=O)N1CCN(CCc2cccc(Nc3nccc(Oc4ccc(NC(=O)c5cnn(-c6ccc(F)cc6)c5C(F)(F)F)cc4F)n3)c2)C(=O)C1]

### Step 1: Synthesis of 2-(m-nitrophenyl)ethanol

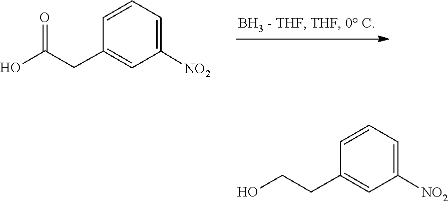 [SMILES: O=C(O)Cc1cccc([N+](=O)[O-])c1 ; O=[N+]([O-])c1cccc(CCO)c1]

To a stirred solution of (m-nitrophenyl)acetic acid (5 g, 27.6 mmol) in THE (50 mL) was added tetrahydrofuran—boron (1/1) (55 mL, 2 eq., 55.2 mmol) 1 M in THE at 0° C., The reaction mass was reflux for 2 h. Progress of the reaction was monitored by TLC & LCMS. After completion of reaction, reaction mixture was quenched with methanol at 0° C. Then concentrate under reduced pressure to get crude. The crude compound was purified by combi-flash using 50% EtOAc in Hexane as eluent to get title compound of 2-(m-nitrophenyl)ethanol (4.5 g, 97.53% yield) as an off-white solid.

¹H NMR (400 MHz, DMSO-d₆): δ=8.13-8.10 (m, 2H), 7.59 (d, J=7.6 Hz, 1H), 7.49 (t, J=7.6 Hz, 1H), 3.94 (t, J=6.0 Hz, 2H), 2.99 (t, J=6.4 Hz, 2H), 1.47 (s, 1H).

### Step 2: 1-(mesyloxy)-2-(m-nitrophenyl)ethane

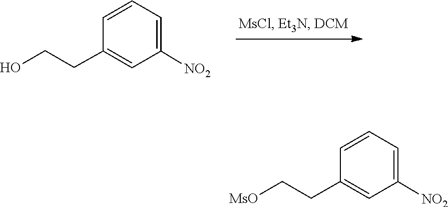 [SMILES: O=[N+]([O-])c1cccc(CCO)c1 ; CS(=O)(=O)OCCc1cccc([N+](=O)[O-])c1]

To a solution of 2-(m-nitrophenyl)ethanol (3.8 g, 22.7 mmol, 1 eq) in dichloromethane (38 mL), was added triethylamine (4.12 mL, 1.3 eq., 29.6 mmol) at 0° C. under a nitrogen atmosphere. After 30 min of stirring at 0° C. (chlorosulfonyl)methane (3.52 mL, 2 eq., 45.5 mmol) was added dropwise to above reaction mixture. After the addition is complete, the reaction is allowed to warm to room temperature, and it was stirred for another 2 h. After completion of reaction on TLC, the solvent was removed, and the residue was dissolved in DCM (100 mL). The organic layer was washed with water (40 mL) then dried over Na₂SO₄ and concentrated to afford 1-(mesyloxy)-2-(m-nitrophenyl)ethane (6 g crude, 24.5 mmol) without further purification.

¹H NMR (400 MHz, DMSO-d₆): δ=8.16-8.13 (m, 2H), 7.60 (d, J=7.6 Hz, 1H), 7.53 (t, J=7.6 Hz, 1H), 4.48 (t, J=6.4 Hz, 2H), 3.19 (t, J=6.4 Hz, 2H), 2.97 (s, 3H).

### Step 3: Synthesis of 2-(m-nitrophenyl)ethanamine

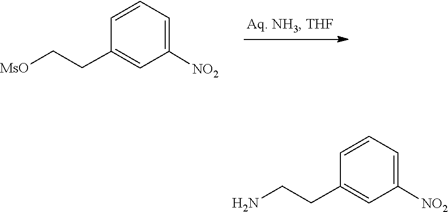 [SMILES: CS(=O)(=O)OCCc1cccc([N+](=O)[O-])c1 ; NCCc1cccc([N+](=O)[O-])c1]

The compound 1-(mesyloxy)-2-(m-nitrophenyl)ethane (5 g, 20.4 mmol) was taken with 25% amoniumhydroxide (50 mL) and stirred for 1 h at 80° C. Progress of reaction was monitored by TLC. After completion of reaction on TLC, the solvent was removed, and the crude residue was dissolved in DCM (100 mL). The organic layer is washed with water (50 mL), dried over Na₂SO₄ and concentrated to afford crude 2-(m-nitrophenyl)ethanamine (2.6 g crude, 76.74% yield) as gummy solid. This was used to next step without further purification.

¹H NMR (400 MHz, DMSO-d₆): δ=8.07-8.05 (m, 2H), 7.68 (d, J=7.2 Hz, 1H), 7.58 (t, J=7.6 Hz, 1H), 2.82-2.78 (m, 4H), LCMS: 167.1 (M+H)⁺.

### Step 4: Synthesis of tert-butyl-2-[2-(m-nitrophenyl)ethylamino] ethylaminoformylate

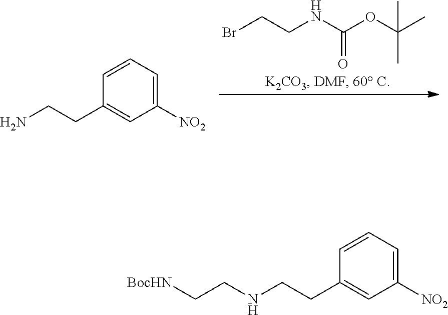 [SMILES: NCCc1cccc([N+](=O)[O-])c1 ; CC(C)(C)OC(=O)NCCBr ; CC(C)(C)OC(=O)NCCNCCc1cccc([N+](=O)[O-])c1]

To a stirred solution of 2-(m-nitrophenyl)ethanamine (2 g, 12 mmol, 1 eq) in DMF (10 mL) was added, dipotassium carbonate (4.99 g, 3 eq., 36.1 mmol) at room temperature. Then 2-bromoethylamino-tert-butylformylate (3.24 g, 1.2 eq., 14.4 mmol) was added to the reaction mixture and stirred for 4 h at 60° C. Progress of the reaction was monitored by TLC & LCMS. After completion of reaction on TLC, the reaction mixture was diluted with water (100 mL) and extracted with EtOAc (100 mL×2). Combined organic layer was dried over Na₂SO₄ and concentrated under reduced pressure to get crude compound. The crude compound was purified by column chromatography using gradient elution of 0-5% MeOH in DCM to afford the title compound as tert-butyl-2-[2-(m-nitrophenyl)ethylamino]ethylaminoformylate (900 mg, 24.17% yield) as brown color solid.

¹H NMR (400 MHz, DMSO-d₆): δ=8.37 (d, J=8.0 Hz, 2H), 7.66 (d, J=7.6 Hz, 1H), 7.55 (t, J=7.6 Hz, 1H), 6.70 (s, 1H), 2.97 (t, J=6.4 Hz, 2H), 2.82 (d, J=6.4 Hz, 2H), 2.75-2.72 (m, 2H), 2.53 (d, J=6.4 Hz, 2H), 1.37 (s, 9H), LCMS: 310.2 (M+H)⁺.

### Step 5: Synthesis of 2-{[2-(m-nitrophenyl)ethyl](bromomethyl)carbonylamino}ethyl 2-methyl-2-propanecarbamate

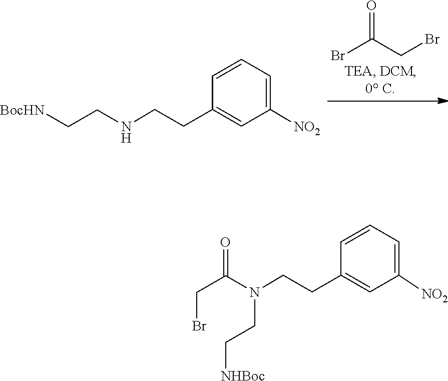 [SMILES: O=C(Br)CBr ; CC(C)(C)OC(=O)NCCNCCc1cccc([N+](=O)[O-])c1 ; CC(C)(C)OC(=O)NCCN(CCc1cccc([N+](=O)[O-])c1)C(=O)CBr]

To a stirred solution of 2-[2-(m-nitrophenyl)ethylamino]ethyl 2-methyl-2-propanecarbamate (0.9 g, 2.91 mmol, 1 eq) in DCM (10 mL) was added triethylamine (1.22 mL, 3 eq., 8.73 mmol) at 0° C., then stirred for 20 min and bromoacetyl bromide (304 μL, 1.2 eq., 3.49 mmol) was added at 0° C. The reaction mixture was stirred for 30 min at RT. Progress of the reaction was monitored by TLC & LCMS. After completion of reaction on TLC, rection mixture dilute with water (30 mL) and extract with DCM (3×30 mL). Combined organic layer was dried with Na₂SO₄ and concentrated under reduced pressure. The crude compound obtained was purified by column chromatography using gradient elution of 25-75% EtOAc in hexane to afford the title compound as 2-{[2-(m-nitrophenyl)ethyl](bromomethyl)carbonylamino}ethyl 2-methyl-2-propanecarbamate (600 mg, 47.93% yield) as yellow color solid.

¹H NMR (400 MHz, DMSO-d₆): δ=8.24-8.07 (m, 2H), 7.78-7.69 (m, 1H), 7.65-7.57 (m, 1H), 6.99-6.85 (m, 1H), 4.07 (s, 2H), 3.58-3.47 (m, 2H), 3.27-3.25 (m, 2H), 3.10-3.05 (m, 2H), 2.97-2.90 (m, 2H), 1.35 (s, 9H), LCMS: 332.1 (M+H-Boc)⁺.

### Step 6: Synthesis of N-2-aminoethyl-N-[2-(m-nitrophenyl)ethyl]bromoacetamide

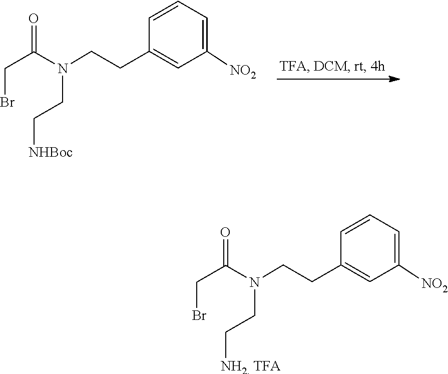

The compound 2-{[2-(m-nitrophenyl)ethyl](bromomethyl)carbonylamino}ethyl-2-methyl-2-propanecarbamate (0.6 g, 1.39 mmol, 1 eq) was taken in trifluoroacetic acid:dichloromethane (1:1) (12 mL) and stirred for 2 hours at room temperature. After completion of reaction on TLC, the reaction was concentrated in vacuo and residue obtained was triturated with diethyl ether (10 mL) and pentane (15 mL) to afford the title compound N-2-aminoethyl-N-[2-(m-nitrophenyl)ethyl]bromoacetamide-TFA Salt (0.5 g, 95% yield) as yellow solid.

¹H NMR (400 MHz, DMSO-d₆): δ=8.17 (t, J=7.6 Hz, 1H), 7.81-7.79 (m, 1H), 7.73-7.68 (m, 1H), 7.66-7.61 (m, 1H), 4.26 (s, 2H), 3.61-3.37 (m, 5H), 3.07-2.90 (m, 3H), LCMS: 332.1 (M+H)⁺.

### Step 7: Synthesis of 1-[2-(m-nitrophenyl)ethyl]-2-piperazinone

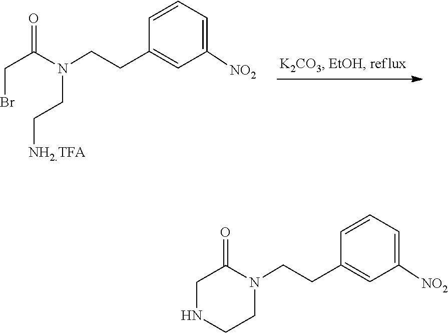

To a stirred solution of N-2-aminoethyl-N-[2-(m-nitrophenyl)ethyl]bromoacetamide (0.5 g, 1.51 mmol) in ethanol (5 mL) was added dipotassium carbonate (1.05 g, 5 eq., 7.57 mmol) at room temperature. The reaction mixture stirred for 20 h at 80° C. Progress of the reaction was monitored by TLC & LCMS. After completion of reaction, the reaction mixture was dilute with water (20 mL) and extract with EtOAc (3×25 mL) and dried with Na₂SO₄, then concentrated under reduced pressure. The crude compound obtained was purified by column chromatography using gradient elution of 0-5% MeOH in DCM to afford the title compound 1-[2-(m-nitrophenyl)ethyl]-2-piperazinone (350 mg, 92.72% yield) as yellow color solid.

¹H NMR (400 MHz, DMSO-d₆): δ=8.10-8.07 (m, 2H), 7.70 (d, J=7.6 Hz, 1H), 7.59 (t, J=8.0 Hz, 1H), 3.51 (t, J=7.2 Hz, 2H), 3.19 (t, J=5.2 Hz, 2H), 3.15 (s, 2H), 2.93 (t, J=7.2 Hz, 2H), 2.79 (t, J=5.2 Hz, 2H), LCMS: 250.15 (M+H)⁺.

### Step 8: Synthesis of 4-acryloyl-1-[2-(m-nitrophenyl)ethyl]-2-piperazinone

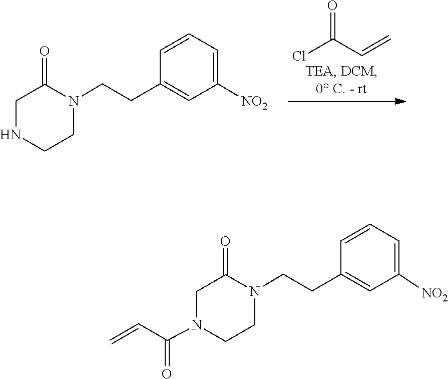

To a stirred solution of 1-[2-(m-nitrophenyl)ethyl]-2-piperazinone (350 mg, 1.4 mmol) in dichloromethane (4 mL) at 0° C., was added triethylamine (587 μL, 3 eq., 4.21 mmol). The reaction mixture was stirred for 20 min then added acryloyl chloride (136 μL, 1.2 eq., 1.68 mmol) at 0° C. and reaction mixture was stirred for 30 min at RT. The progress of reaction was monitored by TLC. After completion of reaction, the reaction mixture was diluted with water (20 mL) and extract with DCM (2×25 mL). Combined organic layer was washed with NaHCO₃ solution total organic layer dried on Na₂SO₄ and concentrated in vacuo to afford crude product. The crude compound was purified by Combi-flash using 1-6% MeOH in DCM as a eluent to afford desired product 4-acryloyl-1-[2-(m-nitrophenyl)ethyl]-2-piperazinone (0.2 g, 46.96% yield) as a yellow color solid.

¹H NMR (400 MHz, DMSO-d₆): δ=8.12-8.07 (m, 2H), 7.70 (d, J=7.6 Hz, 1H), 7.58 (t, J=8.0 Hz, 1H), 6.84-6.70 (m, 1H), 6.15 (d, J=16.8 Hz, 1H), 5.76-5.71 (m, 1H), 4.16-4.02 (m, 2H), 3.77 (s, 1H), 3.67 (s, 1H), 3.59 (t, J=7.2 Hz, 2H), 3.32 (s, 2H), 2.96 (t, J=7.2 Hz, 2H), 3.15 (s, 2H), 2.93 (t, J=7.2 Hz, 2H), 2.79 (t, J=5.2 Hz, 2H).

### Step 9: Synthesis of 4-acryloyl-1-[2-(m-aminophenyl)ethyl]-2-piperazinone

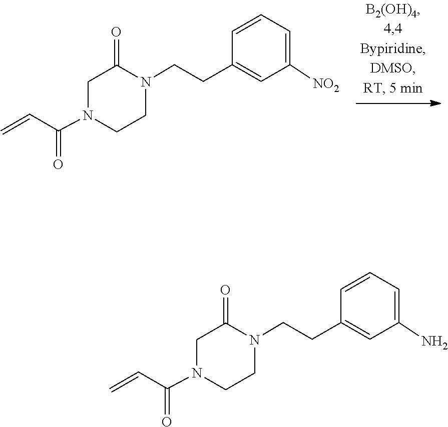 [SMILES: C=CC(=O)N1CCN(CCc2cccc([N+](=O)[O-])c2)C(=O)C1 ; C=CC(=O)N1CCN(CCc2cccc(N)c2)C(=O)C1]

To the stirred solution of 4-acryloyl-1-[2-(m-nitrophenyl)ethyl]-2-piperazinone (0.2 g, 659 μmol, 1 eq.) in dimethyl sulfoxide (2 mL) at 0° C., 4,4′-bipyridyl (15.4 mg, 0.15 eq., 98.9 μmol) was added. After 5 min of stirring, 1,1,2,2-diboranetetrol(4) (236 mg, 4 eq., 2.64 mmol) was added in portions and the reaction was stirred at room temperature. The reaction was completed within 10 min by TLC. After that the reaction mixture was poured into the ice-cold water (30 mL) and extracted with ethyl acetate (30 mL×2). The combined organic layers were dried over Na₂SO₄ and concentrated in vacuo. The crude compound obtained was purified by using gradient elution of 0-6% MeOH in DCM to afford the title compound as 4-acryloyl-1-[2-(m-aminophenyl)ethyl]-2-piperazinone (90 mg, 49.94% yield) as gummy solid.

¹H NMR (400 MHz, DMSO-d₆): δ=6.91 (t, J=7.6 Hz, 1H), 6.77-6.73 (m, 1H), 6.41-6.39 (m, 2H), 6.35 (d, J=7.6 Hz, 1H), 6.14 (d, J=16.0 Hz, 1H), 5.72 (dd, J=2.0, 10.4 Hz, 1H), 4.18-4.05 (m, 2H), 3.73-3.65 (m, 2H), 3.44 (t, J=7.6 Hz, 2H), 3.25 (s, 2H), 2.64-2.58 (m, 2H), LCMS: 274.26 (M+H)⁺.

### Step 10: N-[4-(2-{m-[2-(4-acryloyl-2-oxo-1-piperazinyl)ethyl]phenylamino}-4-pyrimidinyloxy)-3-fluorophenyl]-1-(p-fluorophenyl)-5-(trifluoromethyl)-4-pyrazolecarboxamide

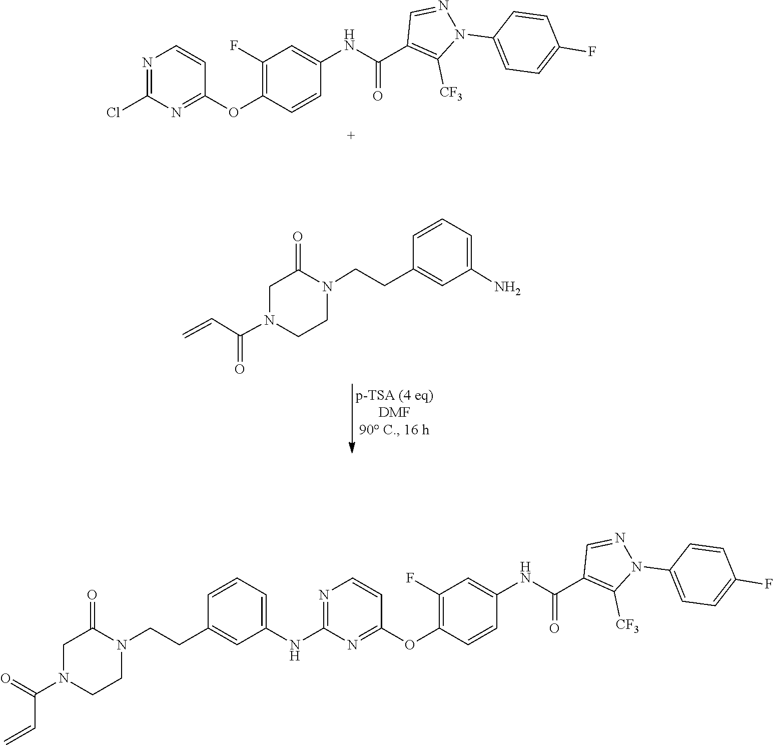 [SMILES: O=C(Nc1ccc(Oc2ccnc(Cl)n2)c(F)c1)c1cnn(-c2ccc(F)cc2)c1C(F)(F)F ; C=CC(=O)N1CCN(CCc2cccc(N)c2)C(=O)C1 ; C=CC(=O)N1CCN(CCc2cccc(Nc3nccc(Oc4ccc(NC(=O)c5cnn(-c6ccc(F)cc6)c5C(F)(F)F)cc4F)n3)c2)C(=O)C1]

To a stirred solution of N-[4-(2-chloro-4-pyrimidinyloxy)-3-fluorophenyl]-1-(p-fluorophenyl)-5-(trifluoromethyl)-4-pyrazolecarboxamide (90 mg, 182 μmol) in dimethylformamide (2 mL), 4-acryloyl-1-[2-(m-aminophenyl)ethyl]-2-piperazinone (74.4 mg, 1.5 eq., 272 μmol) was added at room temperature. The reaction mixture stirred for 16 h at 90° C. Progress of the reaction was monitored by TLC & LCMS. After completion of reaction, water (20 mL) was added to reaction mixture and extracted with ethyl acetate (3×20 mL). Combined organic layer was dried over Na₂SO₄ and concentrated under reduced pressure to get crude compound. The crude was purify by prep-HPLC by using 0.1% formic acid buffer in water and acetonitrile to get title compound N-[4-(2-{m-[2-(4-acryloyl-2-oxo-1-piperazinyl)ethyl]phenylamino}-4-pyrimidinyloxy)-3-fluorophenyl]-1-(p-fluorophenyl)-5-(trifluoromethyl)-4-pyrazolecarboxamide (5 mg, 3.76% o yield) as a white color solid.

¹H NMR (400 MHz, DMSO-d₆). δ=10.88 (s, 1H), 9.61 (s, 1H), 8.39 (d, J=5.6 Hz, 1H), 8.36 (s, 1H), 7.88 (dd, J=2.0, 12.6 Hz, 1H), 7.66-7.63 (m, 2H), 7.54 (d, J=7.6 Hz, 1H), 7.49 15-7.41 (m, 4H), 7.34-7.28 (m, 2H), 7.00 (t, J=8.0 Hz, 1H), 6.78-6.69 (m, 2H), 6.57 (d, J=5.6 Hz, 1H), 6.11 (d, J=16.8 Hz, 1H), 5.68 (dd, J=2.4, 10.4 Hz, 1H), 4.17-4.02 (m, 2H), 3.72-3.60 (m, 2H), 3.43-3.39 (m, 2H), 3.16 (t, J=5.6 Hz, 2H), 2.60 (t, J=7.6 Hz, 2H), LCMS: 733.2 (M+H)⁺.

### Biological Example 1

### Biochemical Assay of the Compounds

### RON Kinase ADP Glo Assay

### Materials:

Assay Buffer: 40 mM Tris-HCl, pH7.5, 20 mM MgCl₂, 0.1 mg/mL BSA
RON Kinase+ADP-Glo™ Assay (Promega #: V8071)
Substrate: 1 g/μL Axltide
Enzyme: 100 ng/μL RON Kinase
ATP: 10 mM
ADP-Glo™ Reagent
Kinase Detection Reagent
384-well white assay plates

### Method:

Kinase assay was done by following instructions from the kit. Briefly, a ten-point serial dilution of compound was prepared at 5× in assay buffer with the final assay concentrations starting at 300 nM, 100 nM, 30 nM, 10 nM . . . 0 nM. Enzyme, substrate, and ATP were used at 25 ng, 2000 ng, and 25 M, respectively. The assay plate was set up by mixing the components in a total reaction volume of 10 μL per well. The plate was centrifuged gently for 10 seconds and incubated at room temperature (RT) for 60 minutes in the dark. The ADP-Glo Reagent and kinase detection reagent were added and incubated as recommended. The reaction was quantified by measuring luminescence on the Perkin Elmer Envision plate reader.

### MET Kinase ADP Glo Assay

### Materials:

Assay Buffer: 40 mM Tris-HCl, pH 7.5, 20 mM MgCl₂, 0.1 mg/mL BSA
MET Kinase Assay (Promega #: V3361)
Substrate: 1 g/μL Poly E4Y1 substrate
Enzyme: 100 ng/μL MET Kinase
ATP: 10 mM
ADP-Glo™ Reagent
Kinase Detection Reagent
384-well white assay plates

### Method:

Kinase assay was done by following instructions from the kit. Briefly, a ten point serial dilution of compound was prepared at 5× in assay buffer with the final assay concentrations starting at 3000 nM, 1000 nM, 300 nM, 100 nM . . . 0 nM. Enzyme, substrate and ATP were used at 25 ng, 2000 ng, and 25 M, respectively. The assay plate was set up by mixing the components in a total reaction volume of 10 μL per well. The plate was centrifuged gently for 10 seconds and incubated at room temperature for 60 minutes in the dark. The ADP-Glo Reagent and kinase detection reagent were added and incubated as recommended. The reaction was quantified by measuring luminescence on the Perkin Elmer Envision plate reader.

TABLE 2
Activity of Representative Compounds

| | RON | cMET | Fold Selectivity |
|---|---|---|---|
| Compound | IC₅₀ (μM) | IC₅₀ (μM) | (cMET/RON) |
| 1 | *** | ** | +++ |
| 2 | *** | *** | +++ |
| 3 | − | * | − |
| 4 | ** | ** | ++ |
| 5 | *** | *** | +++ |
| 6 | ** | ** | ++ |
| 7 | *** | ** | +++ |
| 8 | *** | * | +++ |
| 9 | − | − | − |
| 10 | − | − | − |
| 11 | − | − | − |
| 12 | ** | * | +++ |
| 13 | − | − | − |
| 14 | − | − | − |
| 15 | − | − | − |
| 16 | − | − | − |
| 17 | *** | ** | +++ |
| 18 | − | − | − |
| 19 | − | − | − |
| 20 | − | − | − |
| 21 | ** | ** | + |
| 22 | − | − | − |
| 23 | * | − | − |
| 24 | ** | * | +++ |
| 25 | * | − | − |
| 26 | *** | ** | +++ |
| 27 | * | − | − |
| 28 | * | − | − |
| 29 | ** | − | − |
| 30 | ** | − | − |
| 31 | ** | − | − |
| 32 | ** | − | − |
| 33 | ** | − | − |
| 34 | * | − | − |
| 35 | *** | * | +++ |
| 36 | * | − | − |
| 37 | * | − | − |
| 38 | *** | * | +++ |
| 39 | * | − | − |
| 40 | *** | − | − |
| 41 | ** | − | − |
| 42 | *** | − | − |
| 43 | *** | *** | +++ |
| 44 (peak 1) | *** | ** | +++ |
| 44 (peak 2) | *** | ** | +++ |
| 45 | *** | − | − |
| 46 | *** | − | − |
| 47 | *** | ** | +++ |
| 48 | *** | *** | ++ |
| 49 | *** | *** | ++ |
| 50 | *** | ** | +++ |
| 51 | *** | − | − |
| 52 | *** | * | +++ |
| 53 | − | − | − |
| 54 | *** | ** | +++ |
| 55 | *** | − | − |
| 56 | ** | − | − |
| 57 | ** | − | − |
| 58 | *** | *** | + |
| 59 | − | − | − |
| 60 | − | − | − |

For RON IC₅₀ activity in Table 2:
* represents a value greater than 5 μM up to 60 μM
** represents a value greater than 1 μM up to 5 μM
*** represents a value below 1 μM
− denotes a value was not determined
For cMET IC₅₀ activity in Table 2:
* represents a value greater than 5 μM up to 65 μM
** represents a value greater than 1 μM up to 5 μM
*** represents a value below 1 μM
− denotes a value was not determined
For fold selectivity (cMET/RON) activity in Table 2:
+++ represents a value greater than 3 up to 50
++ represents a value greater than 0.5 up to 3
+ represents a value below 0.5
− denotes a value was not determined

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/413,543 filed Oct. 5, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following Structure (I) 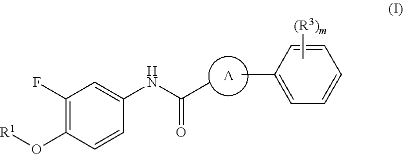
wherein:
R¹ has one of the following structures 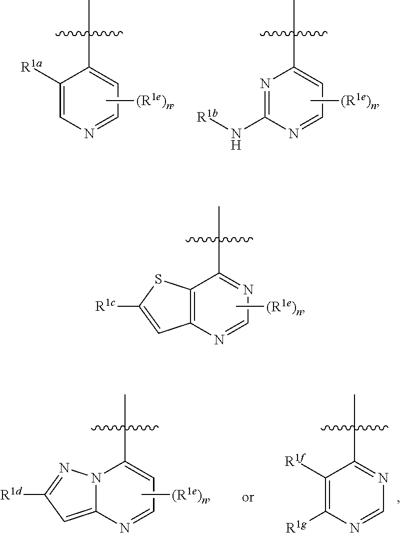
wherein:
R^{1a} is optionally substituted cycloalkyl, optionally substituted heterocyclyl, optionally substituted aryl, optionally substituted 5-membered heteroaryl, or —P(═O)R^{a}R^{b} wherein R^{a} and R^{b} are each independently alkyl or R^{a} and R^{b} join, together with the P atom to which they are attached to form optionally substituted heterocyclyl;
R^{1b} is optionally substituted cycloalkyl, optionally substituted heterocyclyl, optionally substituted aryl, optionally substituted heteroaryl, unsubstituted arylalkyl, or optionally substituted heterocyclylalkyl;
R^{1c} is optionally substituted cycloalkyl, optionally substituted heterocyclyl, optionally substituted aryl, optionally substituted heteroaryl, or —P(═O)R^{c}R^{d} wherein R^{c} and R^{d} are each independently alkyl;
R^{1d} is halo, optionally substituted cycloalkyl, optionally substituted heterocyclyl, optionally substituted aryl, optionally substituted heteroaryl, or —P(═O)R^{e}R^{f} wherein R^{e} and R^{f} are each independently alkyl;
each occurrence of R^{1e} is independently alkyl, halo, haloalkyl, cycloalkyl, hydroxyl, amino, cyano, or optionally substituted —NH-aryl-alkyl-heterocyclyl-alkenyl;
R^{1f} is —P(═O)R^{e}R^{f} wherein R^{e} and R^{f} are each independently alkyl;
R^{1g} is hydrogen or amino;
each occurrence of R³ is independently alkyl, halo, haloalkyl, alkoxy, haloalkoxy, cycloalkyl, hydroxyl, amino, or cyano; 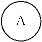
has one of e following structures 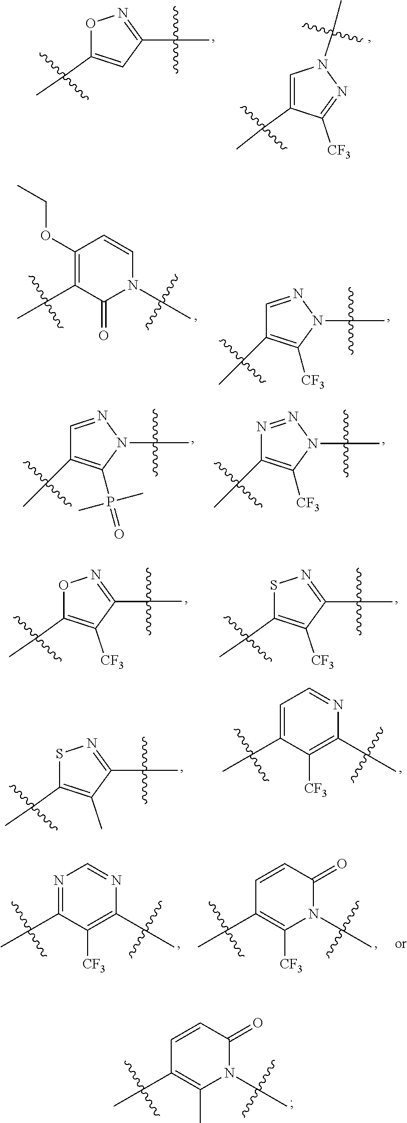
n is 0, 1, 2, or 3; and
m is 0, 1, 2, 3, 4, or 5,
as a stereoisomer, enantiomer, or tautomer thereof or a mixture thereof, or a pharmaceutically acceptable salt, solvate, or prodrug thereof.

2. The compound of claim 1, wherein R¹ has the following structure 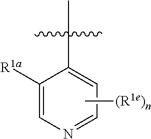
as a stereoisomer, enantiomer, or tautomer thereof or a mixture thereof, or a pharmaceutically acceptable salt, solvate, or prodrug thereof.

3. The compound of claim 1, wherein R^{1a} is optionally substituted 5-membered heteroaryl, as a stereoisomer, enantiomer, or tautomer thereof or a mixture thereof; or a pharmaceutically acceptable salt, solvate, or prodrug thereof.

4. The compound of claim 1, wherein R^{1a} is optionally substituted 5-membered N-heteroaryl, as a stereoisomer, enantiomer, or tautomer thereof or a mixture thereof; or a pharmaceutically acceptable salt, solvate, or prodrug thereof.

5. The compound of claim 1, wherein R^{1a} is optionally substituted imidazolyl or optionally substituted pyrazolyl, as a stereoisomer, enantiomer, or tautomer thereof or a mixture thereof, or a pharmaceutically acceptable salt, solvate, or prodrug thereof.

6. The compound of claim 1, wherein R^{1a} has one of the following structures 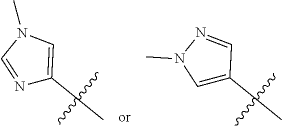
as a stereoisomer, enantiomer, or tautomer thereof or a mixture thereof, or a pharmaceutically acceptable salt, solvate, or prodrug thereof.

7. The compound of claim 1, wherein R^{1a} has the following structure 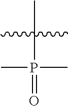
as a stereoisomer, enantiomer, or tautomer thereof or a mixture thereof, or a pharmaceutically acceptable salt, solvate, or prodrug thereof.

8. The compound of claim 1, wherein R^{1a} is optionally substituted cycloalkyl, optionally substituted heterocyclyl, or optionally substituted aryl, as a stereoisomer, enantiomer, or tautomer thereof or a mixture thereof; or a pharmaceutically acceptable salt, solvate, or prodrug thereof.

9. The compound of claim 8, wherein R^{1a} is optionally substituted with alkyl, halo, haloalkyl, cycloalkyl, hydroxyl, amino, or cyano, as a stereoisomer, enantiomer, or tautomer thereof or a mixture thereof; or a pharmaceutically acceptable salt, solvate, or prodrug thereof.

10. The compound of claim 1, wherein R¹ has the following structure 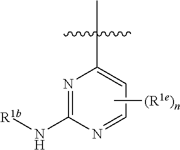
as a stereoisomer, enantiomer, or tautomer thereof or a mixture thereof, or a pharmaceutically acceptable salt, solvate, or prodrug thereof.

11. The compound of claim 1, wherein R^{1b} is optionally substituted cycloalkyl, optionally substituted heterocyclyl, optionally substituted aryl, unsubstituted arylalkyl, or optionally substituted heterocycylylalkyl, as a stereoisomer, enantiomer, or tautomer thereof or a mixture thereof; or a pharmaceutically acceptable salt, solvate, or prodrug thereof.

12. The compound of claim 1, wherein R^{1b} is optionally substituted cyclohexyl, optionally substituted piperidinyl, optionally substituted phenyl, unsubstituted benzyl, or optionally substituted —(CH₂)₂-morpholino, as a stereoisomer, enantiomer, or tautomer thereof or a mixture thereof; or a pharmaceutically acceptable salt, solvate, or prodrug thereof.

13. The compound of claim 1, wherein R^{1b} is cyclohexyl, piperidinyl, or phenyl substituted with one or more substituents selected from the group consisting of alkyl, alkoxy, haloalkoxy, halo, —P(═O)—(CH₃)₂, —NH—C(═O)-alkenyl, —NH—C(═O)-alkenyl-N(CH₃)₂, —NH—C(═O)-alkyl-N(CH₃)—C(═O) alkenyl-N(CH₃)₂, optionally substituted heterocyclyl, and optionally substituted heterocyclylalkyl, a heterocyclyl, as a stereoisomer, enantiomer, or tautomer thereof or a mixture thereof, or a pharmaceutically acceptable salt, solvate, or prodrug thereof.

14. The compound of claim 1, wherein R^{1b} has one of the following structures 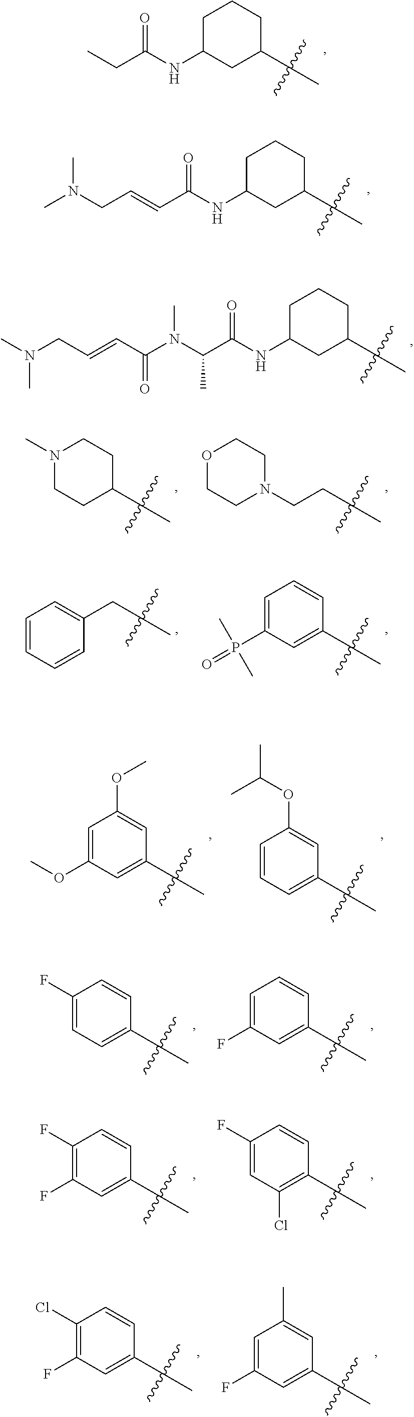 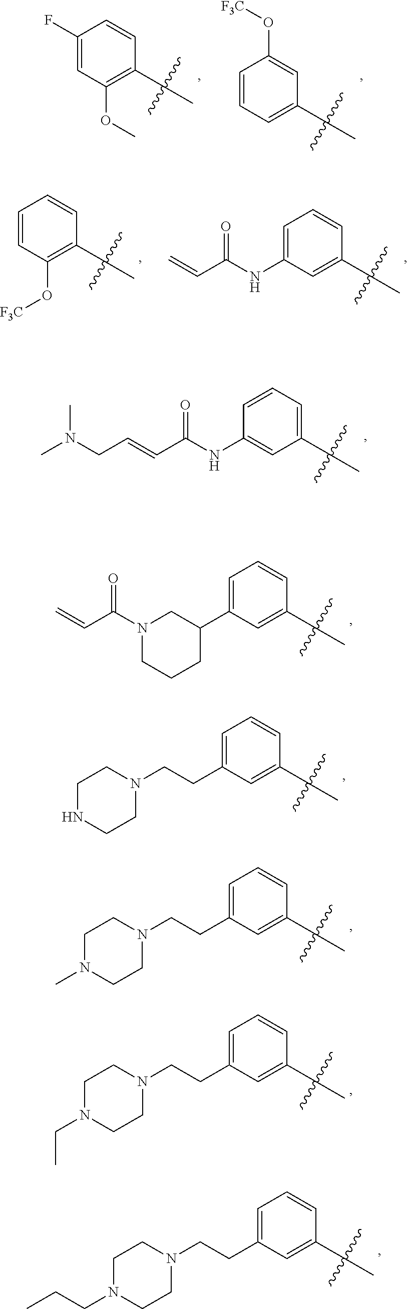 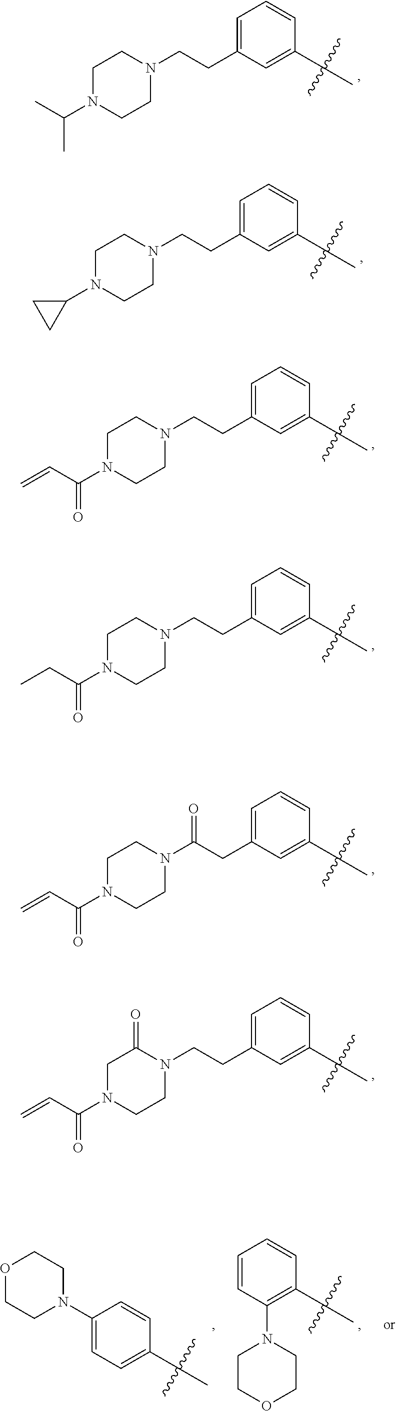 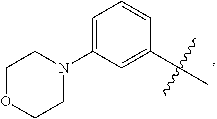 [SMILES: *c1cccc(N2CCOCC2)c1]
as a stereoisomer, enantiomer, or tautomer thereof or a mixture thereof, or a pharmaceutically acceptable salt, solvate, or prodrug thereof.

15. The compound of claim 1, wherein R^{1b} is optionally substituted cycloalkyl, optionally substituted heterocyclyl, or optionally substituted heteroaryl, as a stereoisomer, enantiomer, or tautomer thereof or a mixture thereof; or a pharmaceutically acceptable salt, solvate, or prodrug thereof.

16. The compound of claim 1, wherein R^{1b} is optionally substituted with alkyl, halo, haloalkyl, cycloalkyl, hydroxyl, amino, or cyano, as a stereoisomer, enantiomer, or tautomer thereof or a mixture thereof; or a pharmaceutically acceptable salt, solvate, or prodrug thereof.

17. The compound of claim 1, wherein R¹ has the following structure 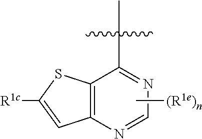
as a stereoisomer, enantiomer, or tautomer thereof or a mixture thereof; or a pharmaceutically acceptable salt, solvate, or prodrug thereof.

18. The compound of claim 1, wherein R^{1c} is optionally substituted 5-membered heteroaryl, as a stereoisomer, enantiomer, or tautomer thereof or a mixture thereof; or a pharmaceutically acceptable salt, solvate, or prodrug thereof.

19. The compound of claim 1, wherein R^{1c} is optionally substituted 5-membered N-heteroaryl, as a stereoisomer, enantiomer, or tautomer thereof or a mixture thereof; or a pharmaceutically acceptable salt, solvate, or prodrug thereof.

20. The compound of claim 1, wherein R^{1c} is optionally substituted imidazolyl or optionally substituted pyrazolyl, as a stereoisomer, enantiomer, or tautomer thereof or a mixture thereof, or a pharmaceutically acceptable salt, solvate, or prodrug thereof.

21. The compound of claim 1, wherein R^{1c} has one of the following structures 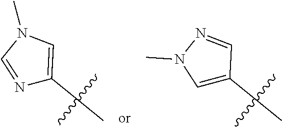
as a stereoisomer, enantiomer, or tautomer thereof or a mixture thereof, or a pharmaceutically acceptable salt, solvate, or prodrug thereof.

22. The compound of claim 1, wherein R^{1c} has the following structure 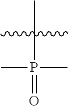
as a stereoisomer, enantiomer, or tautomer thereof or a mixture thereof, or a pharmaceutically acceptable salt, solvate, or prodrug thereof.

23. The compound of claim 1, wherein R¹ has the following structure 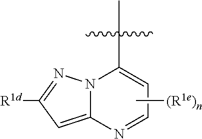
as a stereoisomer, enantiomer, or tautomer thereof or a mixture thereof, or a pharmaceutically acceptable salt, solvate, or prodrug thereof.

24. The compound of claim 1, wherein R^{1d} is optionally substituted 5-membered heteroaryl, as a stereoisomer, enantiomer, or tautomer thereof or a mixture thereof; or a pharmaceutically acceptable salt, solvate, or prodrug thereof.

25. The compound of claim 1, wherein R^{1d} is optionally substituted 5-membered N-heteroaryl, as a stereoisomer, enantiomer, or tautomer thereof or a mixture thereof; or a pharmaceutically acceptable salt, solvate, or prodrug thereof.

26. The compound of claim 1, wherein R^{1d} is optionally substituted imidazolyl or optionally substituted pyrazolyl, as a stereoisomer, enantiomer, or tautomer thereof or a mixture thereof, or a pharmaceutically acceptable salt, solvate, or prodrug thereof.

27. The compound of claim 1, wherein R^{1d} has the following structure 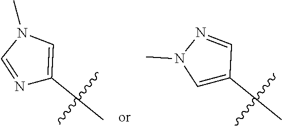
as a stereoisomer, enantiomer, or tautomer thereof or a mixture thereof; or a pharmaceutically acceptable salt, solvate, or prodrug thereof.

28. The compound of claim 1, wherein R^{1d} has the following structure 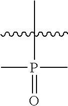
as a stereoisomer, enantiomer, or tautomer thereof or a mixture thereof, or a pharmaceutically acceptable salt, solvate, or prodrug thereof.

29. The compound of claim 1, wherein R^{1d} is bromo, as a stereoisomer, enantiomer, or tautomer thereof or a mixture thereof; or a pharmaceutically acceptable salt, solvate, or prodrug thereof.

30. The compound of any one of claims 1-29, wherein n is 1, 2, 3 and each occurrence of R^{1e} is independently halo, hydroxyl, amino or —NH-aryl-alkyl-heterocyclyl-alkenyl, as a stereoisomer, enantiomer, or tautomer thereof or a mixture thereof, or a pharmaceutically acceptable salt, solvate, or prodrug thereof.

31. The compound of any one of claims 1-29, wherein n is 1 and R^{1e} is amino, or has the following structure 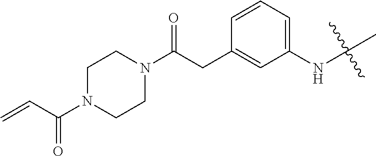 [SMILES: *Nc1cccc(CC(=O)N2CCN(C(=O)C=C)CC2)c1]
as a stereoisomer, enantiomer, or tautomer thereof or a mixture thereof, or a pharmaceutically acceptable salt, solvate, or prodrug thereof.

32. The compound of any one of claims 1-29, wherein n is 0, as a stereoisomer, enantiomer, or tautomer thereof or a mixture thereof; or a pharmaceutically acceptable salt, solvate, or prodrug thereof.

33. The compound of claim 1, wherein R¹ has the following structure 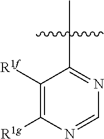
as a stereoisomer, enantiomer, or tautomer thereof or a mixture thereof, or a pharmaceutically acceptable salt, solvate, or prodrug thereof.

34. The compound of claim 1, wherein R^{1f} has the following structure 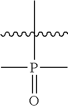
as a stereoisomer, enantiomer, or tautomer thereof or a mixture thereof, or a pharmaceutically acceptable salt, solvate, or prodrug thereof.

35. The compound of claim 1, wherein Rig is amino, as a stereoisomer, enantiomer, or tautomer thereof or a mixture thereof; or a pharmaceutically acceptable salt, solvate, or prodrug thereof.

36. The compound of claim 1, wherein R¹ has one the following structures 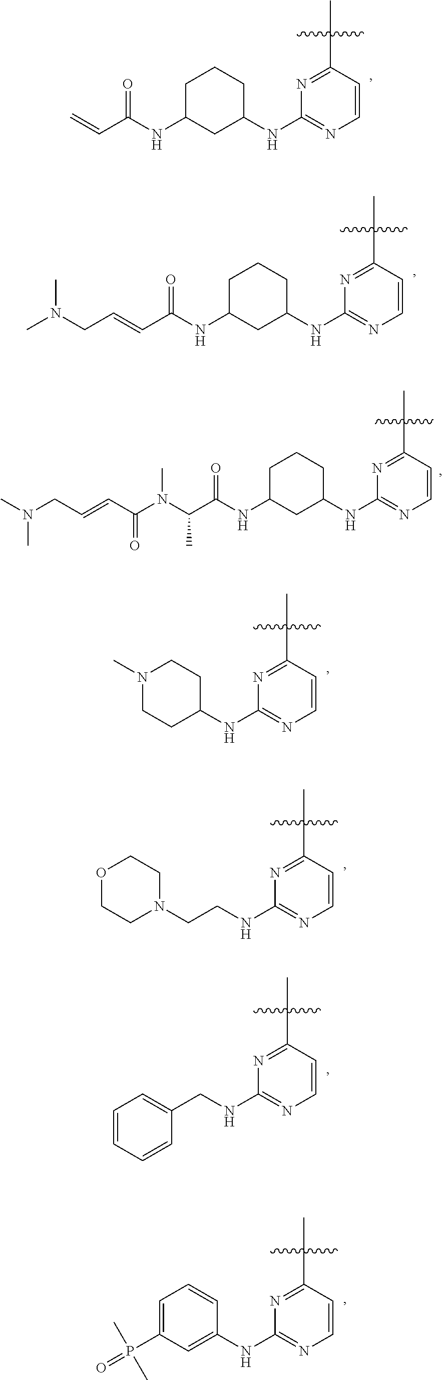 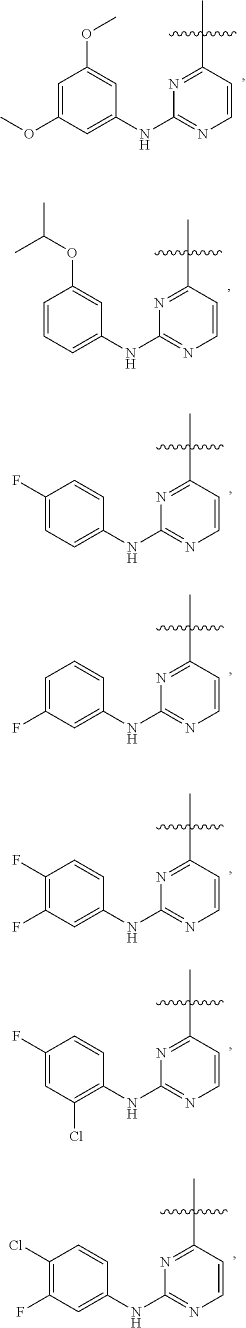 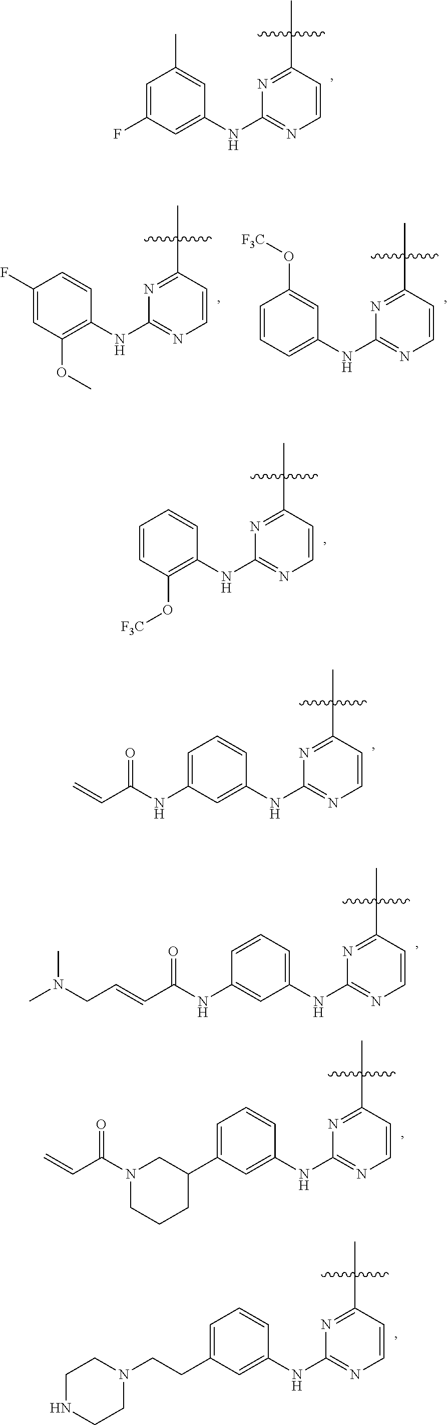 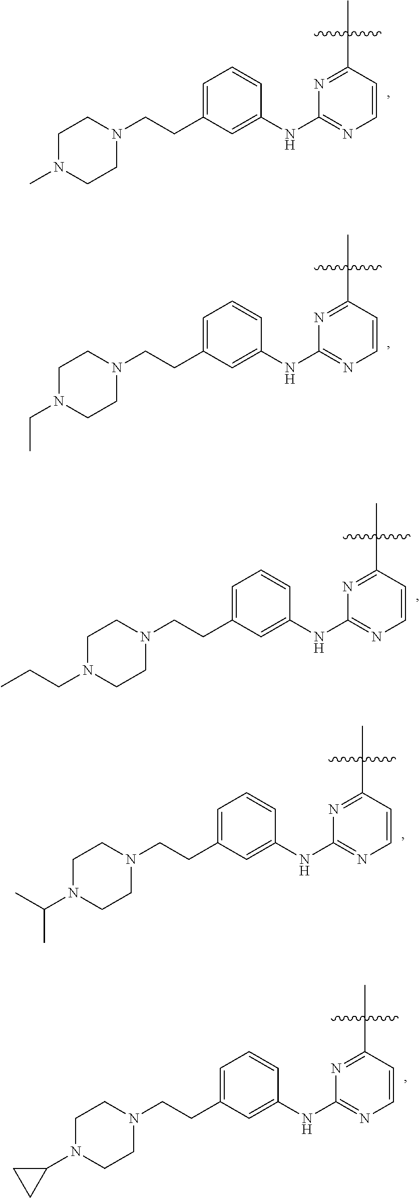 [SMILES: *c1ccnc(Nc2cccc(CCN3CCN(C)CC3)c2)n1 ; *c1ccnc(Nc2cccc(CCN3CCN(CC)CC3)c2)n1 ; *c1ccnc(Nc2cccc(CCN3CCN(CCC)CC3)c2)n1 ; *c1ccnc(Nc2cccc(CCN3CCN(C(C)C)CC3)c2)n1 ; *c1ccnc(Nc2cccc(CCN3CCN(C4CC4)CC3)c2)n1] 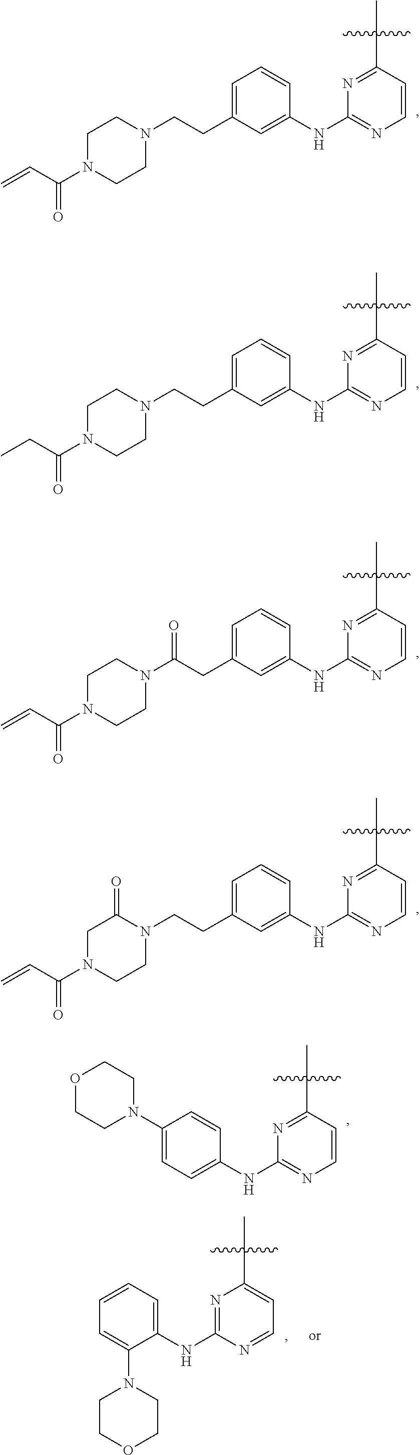 [SMILES: *c1ccnc(Nc2cccc(CCN3CCN(C(=O)C=C)CC3)c2)n1 ; *c1ccnc(Nc2cccc(CCN3CCN(C(=O)CC)CC3)c2)n1 ; *c1ccnc(Nc2cccc(CC(=O)N3CCN(C(=O)C=C)CC3)c2)n1 ; *c1ccnc(Nc2cccc(CCN3CCN(C(=O)C=C)CC3=O)c2)n1 ; *c1ccnc(Nc2ccc(N3CCOCC3)cc2)n1 ; *c1ccnc(Nc2ccccc2N2CCOCC2)n1] 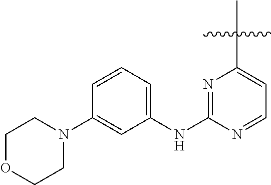 [SMILES: *c1ccnc(Nc2cccc(N3CCOCC3)c2)n1]
as a stereoisomer, enantiomer, or tautomer thereof or a mixture thereof; or a pharmaceutically acceptable salt, solvate, or prodrug thereof

37. The compound of claim 1, wherein R¹ has one of the following structures 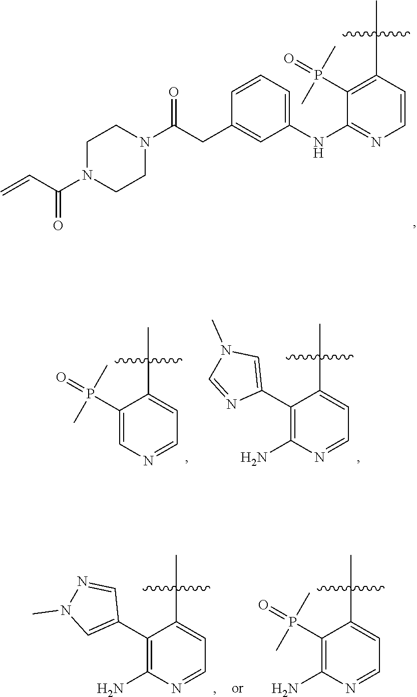
as a stereoisomer, enantiomer, or tautomer thereof or a mixture thereof, or a pharmaceutically acceptable salt, solvate, or prodrug thereof.

38. The compound of claim 1, wherein R¹ has one of the following structures 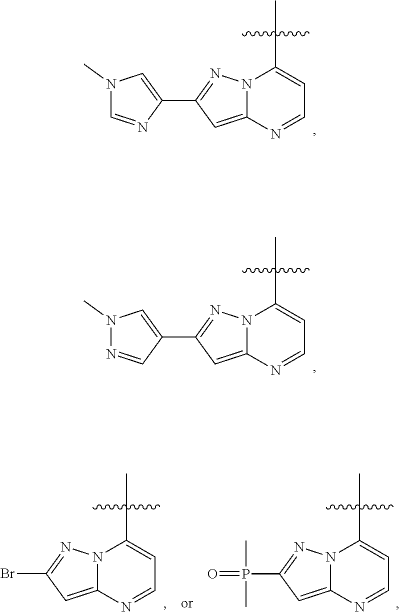 [SMILES: *c1ccnc2cc(-c3cn(C)cn3)nn12 ; *c1ccnc2cc(-c3cnn(C)c3)nn12 ; *c1ccnc2cc(Br)nn12 ; *c1ccnc2cc(P(C)(C)=O)nn12]
as a stereoisomer, enantiomer, or tautomer thereof or a mixture thereof, or a pharmaceutically acceptable salt, solvate, or prodrug thereof.

39. The compound of claim 1, wherein R¹ has one of the following structures: 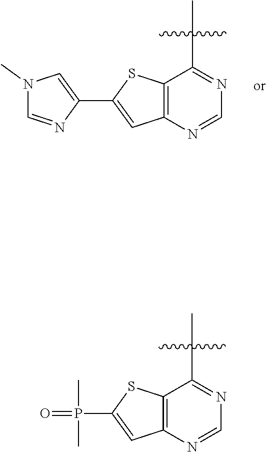 [SMILES: *c1ncnc2cc(-c3cn(C)cn3)sc12 ; *c1ncnc2cc(P(C)(C)=O)sc12]
as a stereoisomer, enantiomer, or tautomer thereof or a mixture thereof, or a pharmaceutically acceptable salt, solvate, or prodrug thereof.

40. The compound of claim 1, wherein R¹ has one of the following structures 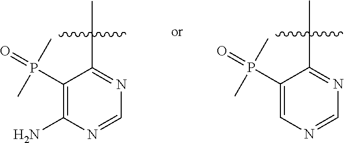 [SMILES: *c1ncnc(N)c1P(C)(C)=O ; *c1ncncc1P(C)(C)=O]
as a stereoisomer, enantiomer, or tautomer thereof or a mixture thereof, or a pharmaceutically acceptable salt, solvate, or prodrug thereof

41. The compound of any one of claims 1-40, wherein m is 1, 2, 3, 4, or 5, as a stereoisomer, enantiomer, or tautomer thereof or a mixture thereof, or a pharmaceutically acceptable salt, solvate, or prodrug thereof.

42. The compound of any one of claims 1-41, wherein each occurrence of R³ is independently halo or haloalkyl, as a stereoisomer, enantiomer, or tautomer thereof or a mixture thereof, or a pharmaceutically acceptable salt, solvate, or prodrug thereof.

43. The compound of any one of claims 1-42, wherein each occurrence of R³ is halo, as a stereoisomer, enantiomer, or tautomer thereof or a mixture thereof, or a pharmaceutically acceptable salt, solvate, or prodrug thereof.

44. The compound of any one of claims 1-43, wherein m is 1 and R³ is fluoro, as a stereoisomer, enantiomer, or tautomer thereof or a mixture thereof, or a pharmaceutically acceptable salt, solvate, or prodrug thereof.

45. The compound of any one of claims 1-44, wherein 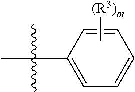
has the following structure 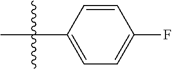 [SMILES: *c1ccc(F)cc1]
as a stereoisomer, enantiomer, or tautomer thereof or a mixture thereof, or a pharmaceutically acceptable salt, solvate, or prodrug thereof.

46. The compound of any one of claims 1-40, wherein m is 0, as a stereoisomer, enantiomer, or tautomer thereof or a mixture thereof; or a pharmaceutically acceptable salt, solvate, or prodrug thereof.

47. A compound having a structure in Table 1 as a stereoisomer, enantiomer, or tautomer thereof or a mixture thereof, or a pharmaceutically acceptable salt, solvate, or prodrug thereof.

48. A pharmaceutical composition comprising a compound of any one of claims 1-47 as a stereoisomer, enantiomer, or tautomer thereof or a mixture thereof or a pharmaceutically acceptable salt, solvate, or prodrug thereof and a pharmaceutically acceptable excipient, diluent, or carrier.

49. A method of treating a disease or disorder, the method comprising administering the compound of any one of claims 1-47 as a stereoisomer, enantiomer, or tautomer thereof or a mixture thereof or a pharmaceutically acceptable salt, solvate, or prodrug thereof or the pharmaceutical composition of claim 48 to a subject in need thereof.

50. The method of claim 49, wherein the disease is cancer.

51. The method of claim 50, wherein the cancer is skin cancer, breast cancer, prostate cancer, lung cancer, pancreatic cancer, colon cancer, bone cancer, bladder cancer, rectal cancer, stomach cancer, esophageal cancer, tracheal cancer, throat cancer, neck cancer, liver cancer, kidney cancer, brain cancer, thyroid cancer, testicle cancer, ovarian cancer, and cervical cancer.

52. The method of claim 50, wherein the cancer is carcinoma, sarcoma, lymphoma, leukemia, blastoma, or germ cell tumor.

53. The method of claim 50, wherein the cancer is bone cancer.

54. The method of claim 50, wherein the cancer includes a bone tumor.

55. The method of claim 49, wherein the disease is osteolysis or osteoporosis.
